(19) United States
(12) Patent Application Publication
(10) Pub. No.: US 2022/0081422 A1
(43) Pub. Date: Mar. 17, 2022

(54) DIHYDROOROTATE DEHYDROGENASE INHIBITORS

(71) Applicant: Janssen Biotech, Inc., Horsham, PA (US)

(72) Inventors: Justin Cisar, North Wales, PA (US); Scott Kuduk, Harleysville, PA (US); Zhuming Zhang, Hillsborough, NJ (US); Aihua Wang, Jamison, PA (US); Yvan Simonnet, Princeton, NJ (US)

(21) Appl. No.: 17/428,997

(22) PCT Filed: Feb. 6, 2020
(86) PCT No.: PCT/IB2020/050955

§ 371 Date: Aug. 6, 2021

Related U.S. Application Data
(60) Provisional application No. 62/802,319, filed on Feb. 7, 2019.

(51) Int. Cl. C07D 403/10; C07D 471/04; C07D 401/10; C07D 401/14

(52) U.S. Cl. CPC C07D 403/10; C07D 401/14; C07D 401/10; C07D 471/04

(57) ABSTRACT

Disclosed are compounds, compositions and methods for treating diseases, disorders, or medical conditions that are affected by the modulation of DHODH. Embodiments of such compounds are represented by Formula (I) as follows:

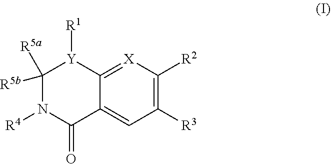

wherein R¹, R², R³, R⁴, R^{5a}, R^{5b}, X and Y, are defined herein.

### CROSS-REFERENCE TO RELATED APPLICATIONS

This application claims the benefit of priority of U.S. Provisional Application No. 62/802,319, filed Feb. 7, 2019, which is incorporated by reference herein, in its entirety and for all purposes.

### FIELD OF THE INVENTION

The present invention relates to novel compounds that are dihydroorotate dehydrogenase (DHODH) inhibitors. These compounds may be useful for the treatment of a disease, disorder, or medical condition where there is an advantage in inhibiting DHODH. The invention also relates to pharmaceutical compositions comprising one or more of such compounds, to processes to prepare such compounds and compositions, and to the use of such compounds or pharmaceutical compositions for the method of treatment of cancer, and autoimmune and inflammatory diseases, syndromes, and disorders.

### BACKGROUND OF THE INVENTION

Acute myelogenous leukemia (AML) is a clonal disease of the blood and bone marrow resulting from mutations that occur in normal hematopoietic stem cells. AML is a heterogenous disease in that it presents with a range of cytogenetic, morphological and immunophenotypic features, and is characterized by an accumulation of clonal, abnormal myeloid progenitor cells, known as myeloblasts. These cells demonstrate disruption of normal myeloid differentiation and excessive proliferation, resulting in the decreased formation of hematopoietic cells. Disease remission can be achieved with standard induction chemotherapy, but refractory and relapsed disease remains a challenge due to persistence of leukemic stem cells. Therefore, AML represents an unmet medical need with >20,000 new cases per year in the US with 5-year overall survival below 30% (Stein E T et al., Health Qual Life Outcomes 16: 193, 2018).

Differentiation therapy is considered an attractive approach to AML treatment based on the knowledge that differentiation and loss of stem cell self-renewal are coupled in normal cells. Treatment of acute promyelocytic leukemia, which represents 10-15% of all AML, with all-trans retinoic acid is the paradigm for differentiation therapy. Retinoic acid targets the promyelocytic leukemia protein (PML)-retinoic acid receptor-a (RAR-a) fusion protein encoded by a t(15,17) chromosomal translocation. Targeting PML-RAR specifically lifts the transcriptionally mediated differentiation block induced by the fusion protein and early clinical trials with single agent ATRA demonstrated complete hematologic remission in all treated patients (McCulloch D et al. Onco Targets Ther 2017; 10: 1585-1601; Nowak D et al. Blood 113: 3655, 2009).

Although differentiation therapy is successful, it is only applicable to a small population of AML patients. Research efforts have aimed at identifying additional differentiation inducing agents, but with limited success. Recently dihydroorotate dehydrogenase (DHODH) emerged as a potentially more broadly applicable differentiation target in a phenotypic screen aimed at identifying small molecules that overcome blockade of the maturation of primary murine bone marrow cells expressing the homeobox protein HoxA9. This protein is a key transcription factor involved in balancing stem cell maintenance/differentiation and is normally expressed in hematopoietic progenitor cells and downregulated upon induction of differentiation and has been found to be widely overexpressed in AML (Sykes et al., Cell 167: 171, 2016).

DHODH is a flavin mononucleotide (FMN) flavoprotein located in the inner mitochondrial membrane that catalyzes the oxidation of dihydroorotate to orotate, the fourth step in the de novo pyrimidine biosynthesis pathway. Inhibition of DHODH leads to decreased pyrimidine synthesis important precursors for nucleotide synthesis, but also glycoprotein and phospholipid biosynthesis (Reis RAG et al., Archives Biochem Biophysics 632: 175, 2017; Vyas VK et al., Mini Rev Med Chem 11: 1039, 2011). DHODH is a validated target for the treatment of autoimmune diseases with the FDA approved small molecule DHODH inhibitors leflunomide and teriflunomide for rheumatoid arthritis and multiple sclerosis, respectively (Lolli ML et al., Recent patents on Anti-Cancer Drug Discovery 13: 86, 2018).

Since the first observation by Sykes et al. demonstrating that DHODH inhibition drives AML differentiation in vitro, as evidenced by upregulation of the differentiation markers CD11b and CD14, and results in dose dependent anti-leukemic effects, decreased leukemic stem cells and prolonged survival in vivo, additional evidence emerged demonstrating that small molecule DHODH inhibitors mediate antiproliferative activity against AML cells with concomitant cell cycle arrest, upregulation of CD11b and CD14, and induction of apoptosis (Wu D et al.. Haematologica 103: 1472, 2018; Sainas S et al., J Med Chem 61: 6034, 2018; Cao Let al., Mol Cancer Ther, October 23rd Epub ahead of print). Moreover, preclinical solid tumor in vitro and in vivo models demonstrated effectiveness of DHODH inhibition and DHODH was identified as a synthetic lethality in PTEN and KRAS mutant solid tumors (Pharmacology and Therapeutics, Epub October 19th, 2018; Mathur D et al., Cancer Discovery 7: 1, 2017; Cell Chemical Biology 25: 1, 2018).

Thus, there remains a need for DHODH inhibitors that provide a therapeutic benefit to patients suffering from cancer and/or inflammatory and immunological diseases.

### SUMMARY OF THE INVENTION

Embodiments of the present invention relate to compounds, pharmaceutical compositions containing them, methods of making and purifying them, methods of using them as inhibitors of DHODH enzymatic activity and methods for using them in the treatment of a subject suffering from or diagnosed with a disease, disorder, or medical condition such as autoimmune or inflammatory disorders, or diseases such as cancer.

Embodiments of this invention are compounds of Formula (I),

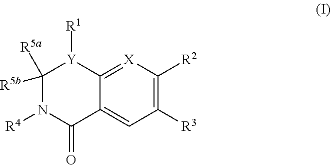

wherein
X is CH or N;
Y is CH or N;
R¹ is selected from the group consisting of: C₁₋₆alkyl; C₁₋₆alkyl substituted with OH, OCH₃, C₃₋₆cycloalkyl or C₃₋₆heterocycloalkyl; C₂₋₆alkenyl; C₂₋₆alkenyl substituted with one, two or three halo members; C₁₋₆haloalkyl; C₁₋₆haloalkyl substituted with OH, or OCH₃; C₃₋₆cycloalkyl; C₃₋₆heterocycloalkyl; and phenyl;
R² is

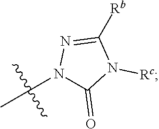

wherein
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, and C₃₋₆cycloalkyl;
R³ is H or F;
R⁴ is selected from the group consisting of:

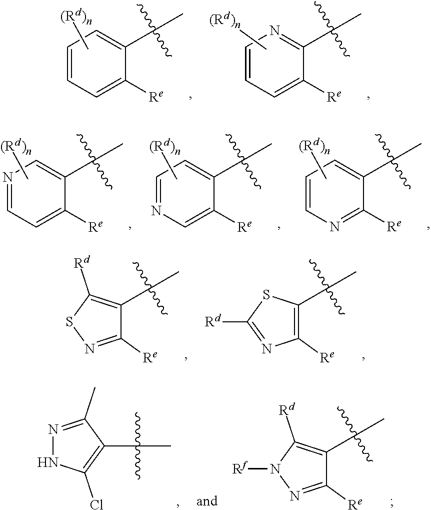

wherein each R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
R^{e} is selected from the group consisting of: H; halo; CN; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; R^{5a} and R^{5b} are each independently H or CH₃; or R^{5a} and R^{5b} come together to form ═O; and

n is 1 or 2; or
a pharmaceutically acceptable salt, isotope, N-oxide, solvate, or stereoisomer thereof.

The present invention further provides methods for treating or ameliorating a disease, syndrome, condition, or disorder in a subject, including a mammal and/or human in which the disease, syndrome, condition, or disorder is affected by the inhibition of DHODH enzymatic activity, including but not limited to, cancer and/or inflammatory or immunological diseases, using a compound of Formula (I) or a pharmaceutically acceptable salt, isotope, N-oxide, solvate, or stereoisomer thereof.

Additional embodiments, features, and advantages of the invention will be apparent from the following detailed description and through practice of the invention.

### DETAILED DESCRIPTION OF THE INVENTION

Unless defined otherwise, all technical and scientific terms used herein have the same meaning as is commonly understood by one of ordinary skill in art. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

With reference to substituents, the term “independently” refers to the situation where when more than one substituent is possible, the substituents may be the same or different from each other.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

Unless qualified specifically in particular instances of use, the term “alkyl” refers to a straight- or branched-chain alkyl group having from 1 to 8 carbon atoms in the chain. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. “C₁₋₆alkyl” refers to straight- or branched-chain alkyl group having from 1 to 6 carbon atoms in the chain. “C₁₋₄alkyl” refers to straight- or branched-chain alkyl group having from 1 to 4 carbon atoms in the chain.

The term “alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl, etc.). The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂₋₆ for straight chain, C₃₋₆ for branched chain).

The term “cycloalkyl” refers to a saturated or partially saturated, monocyclic, fused polycyclic, or spiro polycyclic carbocycle having from 3 to 12 ring atoms per carbocycle. “C₃₋₆cycloalkyl” refers to a carbocycle having from 3 to 6 ring atoms per carbocycle. Illustrative examples of cycloalkyl groups include the following entities, in the form of properly bonded moieties:

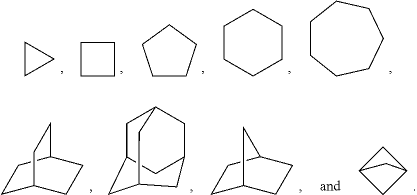

The term “halogen” or “halo” represents chlorine, fluorine, bromine, or iodine. The term “haloalkyl” refers to a straight- or branched-chain alkyl group having from 1 to 6 carbon atoms in the chain optionally substituting hydrogens with halogens. The term “C₁₋₆haloalkyl” as used here refers to a straight- or branched-chain alkyl group having from 1 to 6 carbon atoms in the chain, optionally substituting hydrogens with halogens. The term “C₁₋₄haloalkyl” as used here refers to a straight- or branched-chain alkyl group having from 1 to 4 carbon atoms in the chain, optionally substituting hydrogens with halogens. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples.

The term “aryl” refers to a monocyclic, aromatic carbocycle (ring structure having ring atoms that are all carbon) having 6 atoms per ring. (Carbon atoms in the aryl groups are sp2 hybridized.) The term “phenyl” represents the following moiety:

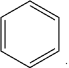

The terms “4- to 8-membered heterocycloalkyl” and “4- to 6-membered heterocycloalkyl” mean a monocyclic, bicyclic, or bridged, saturated heterocycle with 4, 5, 6, 7 or 8 or, respectively, 4, 5, or 6, ring atoms in total, which contains one or two identical or different ring heteroatoms from the series N, O and S being possible for said heterocycloalkyl group to be attached to the rest of the molecule via any one of the carbon atoms or, if present and not excluded otherwise, a nitrogen atom. Illustrative examples of heterocycloalkyl groups include the following entities, in the form of properly bonded moieties:

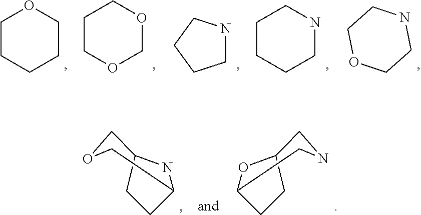

Those skilled in the art will recognize that the species of heterocycloalkyl, cycloalkyl, and aryl groups listed or illustrated above are not exhaustive, and that additional species within the scope of these defined terms may also be selected.

The term “pyridinyl” or “pyridyl” represents the following moiety:

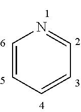

The pyridinyl or pyridyl moiety can be attached through any one of the 2-, 3-, 4-, 5-, or 6-position carbon atoms.

The term “imidazolyl” represents the following moiety:

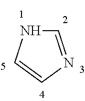

The imidazolyl moiety can be attached through any one of the 1-, 2-, 3-, 4-, or 5-position carbon atoms.

The term “heteroaryl” refers to a monocyclic or fused bicyclic heterocycle (ring structure having ring atoms selected from carbon atoms and up to four heteroatoms selected from nitrogen, oxygen, and sulfur) having from 3 to 9 ring atoms per heterocycle. Illustrative examples of heteroaryl groups include the following entities, in the form of properly bonded moieties:

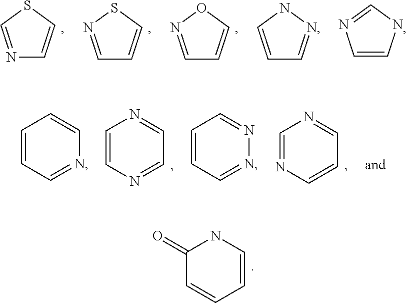

The term “ tautomeric ” or “ tautomeric form ” refers to structural isomers of different energies that are interconvertible through low energy barriers. For example, proton tautomers (also known as proton tautomers) include interconversions through the transfer of protons, such as keto-enol and imine-enamine isomerization. The valence tautomers include interconversions by restructuring some bond electrons.

For example, hydroxypyridine or the tautomeric pyridone is represented below.

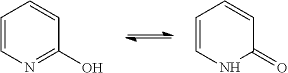 [SMILES: Oc1ccccn1 ; O=c1cccc[nH]1]

For example, pyrazole tautomers are represented below.

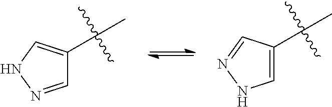 [SMILES: *c1cn[nH]c1 ; *c1cn[nH]c1]

Those skilled in the art will recognize that the species of heterocycloalkyl, cycloalkyl, heteroaryl and aryl groups listed or illustrated above are not exhaustive, and that additional species within the scope of these defined terms may also be selected.

The term “chiral” refers to a molecule that has the property of not being superimposable with a mirror image partner, while the term “achiral” refers to a molecule that is superimposable with its mirror image partner.

The term “stereoisomer” refers to compounds that have the same chemical configuration but differ with respect to the arrangement of atoms or groups in space.

The term “enantiomer” as used herein refers to two stereoisomers of a compound.

The term “atropisomer” means that rotation around a single bond within a molecule is prevented or greatly delayed as a result of steric interaction with the rest of the molecule, and substitutions at both ends of a single bond Conformational stereoisomers that occur when a group is asymmetric, ie optical activity occurs without the need for asymmetric carbon centers or stereocenters. If the rotation barrier around a single bond is high enough and the interconversion between conformations is slow enough, separation and isolation of isomeric species may be possible. Atropisomers are enantiomers that do not have a single asymmetric atom. Atropisomers are when the barrier to interconversion is high enough to allow the atropisomer to undergo little or no interconversion at room temperature for at least one week, preferably at least one year. It is considered stable. In some embodiments, an atropisomer of the present invention is the opposite atropisomer at room temperature for a week when the atropisomer is in a substantially pure form (generally in the solid state). No interconversion exceeding about 5%. In some embodiments, the atropisomeric compounds of the invention do not undergo greater than about 5% interconversion to the opposite atropisomer at room temperature (approximately 25° C.) for 1 year. Preferably, the atropisomeric compounds of the present invention are sufficiently stable to undergo no more than about 5% interconversion in aqueous pharmaceutical formulations held at 0° C. for at least 1 week.

The term “variable point of attachment” means that a group is allowed to be attached at more than one alternative position in a structure. The attachment will always replace a hydrogen atom on one of the ring atoms. In other words, all permutations of bonding are represented by the single diagram, as shown in the illustrations below.

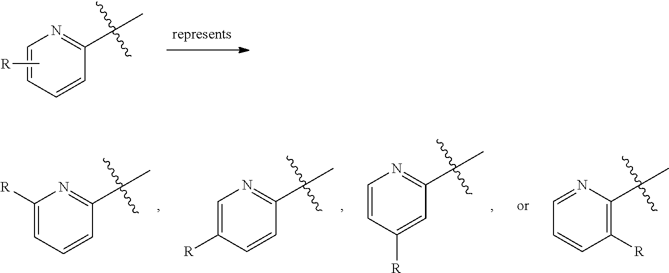

Those skilled in the art will recognize that that if more than one such substituent is present for a given ring, the bonding of each substituent is independent of all of the others. The groups listed or illustrated above are not exhaustive.

As used herein, the term “or” means “and/or” unless stated otherwise.

As used herein, the terms “including”, “containing” and “comprising” are used in their open, non-limiting sense.

As used herein, the term “composition” is intended to encompass a product comprising the specified ingredients in the specified amounts, as well as any product which results, directly or indirectly, from combination of the specified ingredients in the specified amounts.

As used herein, the term “treat”, “treating”, or “treatment” of any disease, condition, syndrome or disorder refers, in one embodiment, to ameliorating the disease, condition, syndrome or disorder (i.e. slowing or arresting or reducing the development of the disease or at least one of the clinical symptoms thereof). In another embodiment, “treat”, “treating”, or “treatment” refers to alleviating or ameliorating at least one physiological or biochemical parameter associated with or causative of the disease, condition, syndrome or disorder, including those which may not be discernible by the patient. In a further embodiment, “treat”, “treating”, or “treatment” refers to modulating the disease, condition, syndrome or disorder either physically (e.g. stabilization of a discernible symptom), physiologically, (e.g. stabilization of a physical parameter), or both. In yet another embodiment, “treat”, “treating”, or “treatment” refers to preventing or delaying the onset or development or progression of the disease, condition, syndrome or disorder.

The terms “subject” and “patient” are used interchangeably herein and may refer to an animal, preferably a mammal, most preferably a human.

As used herein, the terms active compound, pharmaceutical agent and active ingredient are used interchangeably to refer to a pharmaceutically active compound.

Other ingredients in a drug composition, such as carriers, diluents or excipients, may be substantially or completely pharmaceutically inert. A pharmaceutical composition (also referred to herein as a composition or formulation) may comprise the active ingredient in combination with one or more carriers and/or one or more excipients and/or one or more diluents.

The term “therapeutically effective amount” (used interchangeably herein with “effective amount”) refers to an amount (e.g., of an active compound or pharmaceutical agent, such as a compound of the present invention), which elicits the biological or medicinal response in a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease. Stated another way, the term therapeutically effective amount may refer to an amount that, when administered to a particular subject, achieves a therapeutic effect by inhibiting, alleviating or curing a disease, condition, syndrome or disorder in the subject or by prophylactically inhibiting, preventing or delaying the onset of a disease, condition, syndrome or disorder, or symptom(s) thereof. A therapeutically effective amount may be an amount which relieves to some extent one or more symptoms of a disease, condition, syndrome or disorder in a subject; and/or returns to normal either partially or completely one or more physiological or biochemical parameters associated with or causative of the disease, condition, syndrome or disorder; and/or reduces the likelihood of the onset of the disease, condition, syndrome or disorder, or symptom(s) thereof

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

A “pharmaceutically acceptable salt” is intended to mean a salt of an acid or base of a compound represented by Formula (I) (as well as compounds of Formula (IA), and (IB)) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response.

Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methane-sulfonates, propanesulfonates, naphthalene-1-sulfonates, naphthalene-2-sulfonates, and mandelates.

A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

Compounds of Formula (I) may contain at least one nitrogen of basic character, so desired pharmaceutically acceptable salts may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents.

Compounds of Formula (I) may contain a carboxylic acid moiety, a desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, piperazine, N-methyl-glucamine and tromethamine and inorganic salts derived from sodium, calcium, potassium, magnesium, manganese, iron, copper, zinc, aluminum, and lithium.

Each compound used herein may be discussed interchangeably with respect to its chemical formula, chemical name, abbreviation, etc.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of such formula. The compounds of this invention may possess one or more asymmetric centers; such compounds can therefore be produced as individual (R)- or (S)-stereoisomers or as mixtures thereof. Thus, any formula given herein is intended to represent a racemate, one or more of its enantiomeric forms, one or more of its diastereomeric forms, and mixtures thereof. Additionally, any formula given herein is intended to refer also to any one of: hydrates, solvates, polymorphs and of such compounds, and mixtures thereof, even if such forms are not listed explicitly. The term “R” at a stereocenter designates that the stereocenter is purely of the

R-configuration as defined in the art; likewise, the term “S” means that the stereocenter is purely of the S-configuration. As used herein, the term “RS” refers to a stereocenter that exists as a mixture of the R- and S-configurations.

Compounds containing one stereocenter drawn without a stereo bond designation are a mixture of 2 enantiomers. Compounds containing 2 stereocenters both drawn without stereo bond designations are a mixture of 4 diastereomers. Compounds with 2 stereocenters both labeled “RS” and drawn with stereo bond designations are a 2-component mixture with relative stereochemistry as drawn. Unlabeled stereocenters drawn without stereo bond designations are a mixture of the R- and S-configurations. For unlabeled stereocenters drawn with stereo bond designations, the absolute stereochemistry is as depicted.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Reference to a compound herein stands for a reference to any one of: (a) the recited form of such compound, and (b) any of the forms of such compound in the medium in which the compound is being considered when named. For example, reference herein to a compound such as R—COOH, encompasses reference to any one of: for example, R—COOH(s), R—COOH(sol), and R—COO-(sol). In this example, R—COOH(s) refers to the solid compound, as it could be for example in a tablet or some other solid pharmaceutical composition or preparation; R—COOH(sol) refers to the undissociated form of the compound in a solvent; and R—COO-(sol) refers to the dissociated form of the compound in a solvent, such as the dissociated form of the compound in an aqueous environment, whether such dissociated form derives from R—COOH, from a salt thereof, or from any other entity that yields R—COO— upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH(aq) and/or R—COO-(aq), where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number in an enriched form. Examples of isotopes that can be incorporated into compounds of the invention in a form that exceeds natural abundances include isotopes of hydrogen, carbon, nitrogen, oxygen, phosphorous, fluorine, chlorine, and iodine, such as ²H (or chemical symbol D), ³H (or chemical symbol T), ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H, or D) may afford certain therapeutic advantages resulting from greater metabolic stability, for example increased in vivo half-life or reduced dosage requirements. Isotopically labeled compounds of this invention can generally be prepared by carrying out the procedures disclosed in the schemes or in the examples and preparations described below by substituting a readily available isotopically labeled reagent for a non-isotopically labeled reagent.

The term Cₙ₋ₘ alkyl refers to an aliphatic chain, whether straight or branched, with a total number N of carbon members in the chain that satisfies n≤N≤m, with m>n.

When the same plurality of substituents is assigned to various groups, the specific individual substituent assignment to each of such groups is meant to be independently made with respect to the specific individual substituent assignments to the remaining groups. By way of illustration, but not as a limitation, if each of groups Q and R can be H or F, the choice of H or F for Q is made independently of the choice of H or F for R, so the choice of assignment for Q does not determine or condition the choice of assignment for R, or vice-versa, unless it is expressly indicated otherwise. Illustrative claim recitation in this regard would read as “each of Q and R is independently H or F”, or “each of Q and R is independently selected from the group consisting of H and F”.

In another example, a zwitterionic compound would be encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts and dipolar ions in the known and well-established meanings of these terms are within the scope of this invention, as would in any case be so appreciated by those of ordinary skill in the art. Because there is no need to name each and every embodiment that would be recognized by those of ordinary skill in the art, no structures of the zwitterionic compounds that are associated with the compounds of this invention are given explicitly herein. They are, however, part of the embodiments of this invention. No further examples in this regard are provided herein because the interactions and transformations in a given medium that lead to the various forms of a given compound are known by any one of ordinary skill in the art.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S₁ and S²ₑₓₐₘₚₗₑ is S3; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein.

The nomenclature “C₁-Cⱼ” or “C₁₋ⱼ” with j>i, when applied herein to a class of substituents, is meant to refer to embodiments of this invention for which each and every one of the number of carbon members, from i to j including i and j, is independently realized. By way of example, the term C₁-C₃ refers independently to embodiments that have one carbon member (C₁), embodiments that have two carbon members (C₂), and embodiments that have three carbon members (C₃). Embodiments of this invention include compounds of Formula (I),

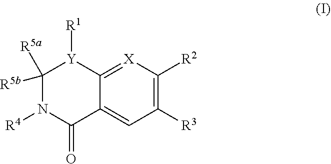

wherein
X is CH or N;
Y is CH or N;
R¹ is selected from the group consisting of: C₁₋₆alkyl; C₁₋₆alkyl substituted with OH, OCH₃, C₃₋₆cycloalkyl or C₃₋₆heterocycloalkyl; C₂₋₆alkenyl; C₂₋₆alkenyl substituted with one, two or three halo members; C₁₋₆haloalkyl; C₁₋₆haloalkyl substituted with OH, or OCH₃; C₃₋₆cycloalkyl; C₃₋₆heterocycloalkyl; and phenyl;
R² is

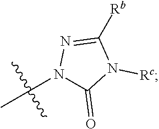

wherein
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, and C₃₋₆cycloalkyl;
R³ is H or F;
R⁴ is selected from the group consisting of:

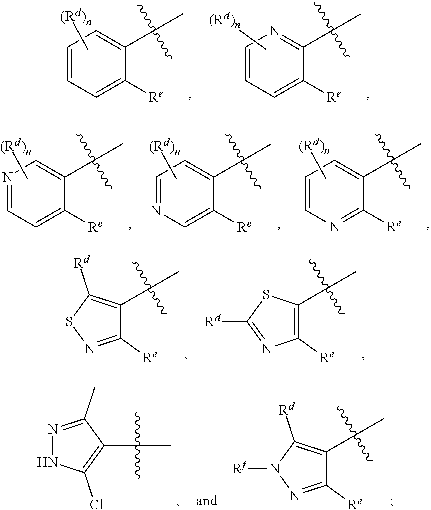

wherein
each R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
R^{e} is selected from the group consisting of: H; halo; CN; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃;
   R^{5a} and R^{5b} are each independently H or CH₃; or R^{5a} and R^{5b} come together to form ═O; and
   n is 1 or 2;
      or pharmaceutically acceptable salts, isotopes, N-oxides, solvates, or stereoisomers thereof; or pharmaceutically acceptable salts of the isotopes, N-oxides, solvates, or stereoisomers thereof.

An additional embodiment of the invention is a compound of Formula (I) wherein X is CH.

An additional embodiment of the invention is a compound of Formula (I) wherein X is N.

An additional embodiment of the invention is a compound of Formula (I) wherein Y is N.

An additional embodiment of the invention is a compound of Formula (I) wherein Y is CH.

An additional embodiment of the invention is a compound of Formula (I) wherein R¹ is selected from the group consisting of: R¹ is selected from the group consisting of: C₁₋₆alkyl; C₁₋₆alkyl substituted with OH, OCH₃, C₃₋₆cycloalkyl or C₃₋₆heterocycloalkyl; C₂₋₆alkenyl; C₂₋₆alkenyl substituted with one, two or three F members; C₁₋₆haloalkyl; C₁₋₆haloalkyl substituted with OH, or OCH₃; C₃₋₆cycloalkyl; tetrahydropyran-4-yl; and phenyl.

An additional embodiment of the invention is a compound of Formula (I) wherein R¹ is C₁₋₄alkyl; C₃₋₆cycloalkyl or C₃₋₆heterocycloalkyl;

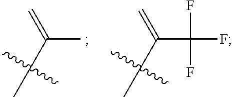

C₁₋₄haloalkyl; C₁₋₄haloalkyl substituted with OH, or OCH₃; tetrahydropyran-4-yl; or C₃₋₆cycloalkyl.

An additional embodiment of the invention is a compound of Formula (I) wherein Y is N and R¹ is CH(CH₃)₂, CH₂CH(CH₃)₂, CH₂CH₂CH₃, CH₂CH₂CH₂CH₃, CH(CH₃)CH₂CH₃, CH(CH₃)CH₂CH₂CH₃, CH(CH₃)(CF₃), CH(CH₃)CH₂OCH₃,

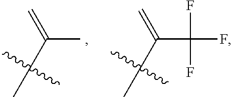

cyclopropyl, cyclobutyl,

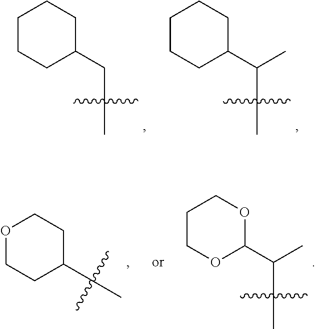

An additional embodiment of the invention is a compound of Formula (I) wherein Y is CH and R¹ is

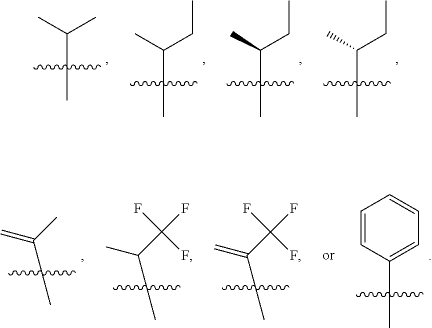

An additional embodiment of the invention is a compound of Formula (I) wherein
R² is

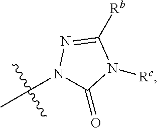

wherein
R^{b} is C₁₋₄alkyl substituted with OH, halo, CN, OC₁₋₄alkyl, OC₁₋₄haloalkyl or OC₃₋₆cycloalkyl; and W is C₁₋₄alkyl, C₁₋₄haloalkyl, or C₃₋₆cycloalkyl.

An additional embodiment of the invention is a compound of Formula (I) wherein R² is

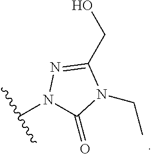 [SMILES: *n1nc(CO)n(CC)c1=O]

An additional embodiment of the invention is a compound of Formula (I) wherein R³ is H.

An additional embodiment of the invention is a compound of Formula (I) wherein R³ is F.

An additional embodiment of the invention is a compound of Formula (I) wherein R⁴ is

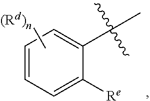

wherein
each R^{d} is independently selected from the group consisting of: H; halo; C₁₋₄alkyl; C₁₋₄alkyl substituted with OH, OCH₃, SCH₃, or OCF₃; C₁₋₄haloalkyl; C₁₋₄haloalkyl substituted with OH, OCH₃; and OC₁₋₄alkyl;
R^{e} is H; halo; CN; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃; and

n is 1 or 2.

An additional embodiment of the invention is a compound of Formula (I) wherein R⁴ is

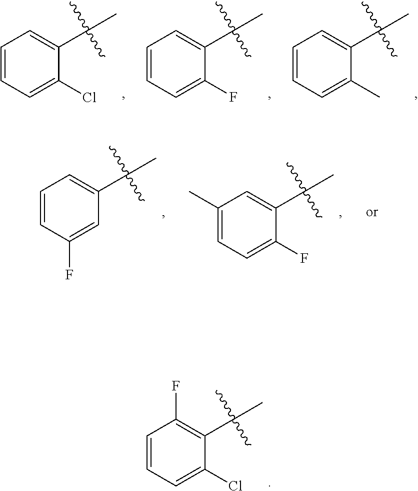

An additional embodiment of the invention is a compound of Formula (I) wherein

R⁴ is

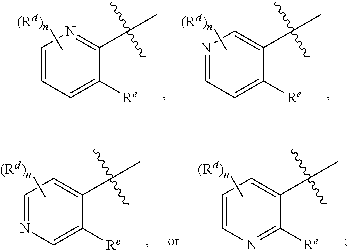

wherein

each R^{d} is independently selected from the group consisting of: H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; and OC₁₋₄alkyl;

R^{e} is H; halo; CN; C₁₋₄alkyl; C₁₋₄alkyl substituted with OH, OCH₃, SCH₃, or OCF₃;

C₁₋₄haloalkyl; or C₁₋₄haloalkyl substituted with OH, or OCH₃; and

n is 1 or 2.

An additional embodiment of the invention is a compound of Formula (I) wherein R⁴ is

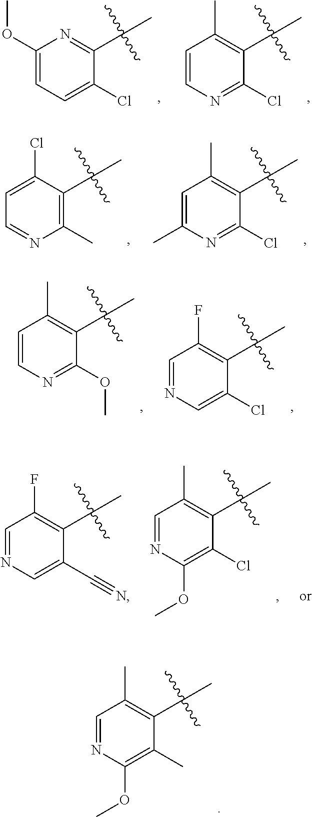

An additional embodiment of the invention is a compound of Formula (I) wherein

R⁴ is

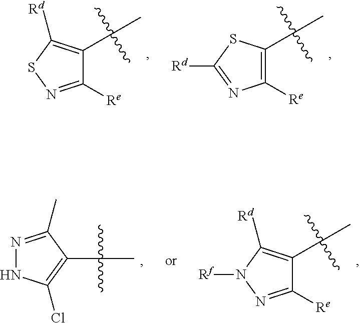

wherein
R^{d} is H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; or OC₁₋₄alkyl;
R^{e} is H; halo; CN; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃; and
R^{f} is H; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃.
An additional embodiment of the invention is a compound of Formula (I) wherein

R⁴ is

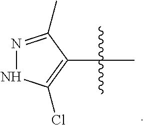 [SMILES: *c1c(C)n[nH]c1Cl]

An additional embodiment of the invention is a compound of Formula (I) wherein R^{5a} and R^{5b} are each H.
An additional embodiment of the invention is a compound of Formula (I) wherein R^{5a} and R^{5b} are each CH₃.
An additional embodiment of the invention is a compound of Formula (I) wherein R^{5a} is H, and R^{5b} is CH₃.
An additional embodiment of the invention is a compound of Formula (I) wherein R^{5a} and R^{5b} come together to form ═O.

Further embodiments of the current invention are selected from compounds as shown below in Table 1, and pharmaceutically acceptable salts, isotopes, N-oxides, solvates, and stereoisomers thereof:

TABLE 1

| Example | |
|---|---|
| # | Compound Name |
| 1 | 3-(2-Chloro-6-fluorophenyl)-7-(4-ethyl-3-(hydroxymethyl)-5-oxo-4,5-dihydro- |
| | 1H-1,2,4-triazol-1-yl)-6-fluoro-1-isopropyl-2,3-dihydroquinazolin-4(1H)-one; |
| 2 | 3-(2-Chloro-6-fluorophenyl)-7-(4-ethyl-3-(hydroxymethyl)-5-oxo-4,5-dihydro-1H- |
| | 1,2,4-triazol-1-yl)-6-fluoro-1-isopropyl-2-methyl-2,3-dihydroquinazolin-4(1H)-one; |
| 3 | 3-(2-Chloro-6-fluorophenyl)-7-(4-ethyl-3-(hydroxymethyl)-5-oxo-4,5-dihydro-1H-1,2, |
| | 4-triazol-1-yl)-6-fluoro-1-isopropyl-2,2-dimethyl-2,3-dihydroquinazolin-4(1H)-one; |
| 4 | 3-(2-Chloro-6-fluorophenyl)-7-(4-ethyl-3-(hydroxymethyl)-5-oxo-4,5-dihydro- |
| | 1H-1,2,4-triazol-1-yl)-6-fluoro-1-isopropylquinazoline-2,4(1H,3H)-dione. |
| 5 | 3-(2-Chloro-6-fluorophenyl)-7-(4-ethyl-3-(hydroxymethyl)-5-oxo-4,5-dihydro-1H-1,2, |
| | 4-triazol-1-yl)-6-fluoro-1-isopropyl-2,3-dihydropyrido[2,3-d]pyrimidin-4(1H)-one; |
| 6 | 3-(2-Chloro-6-fluorophenyl)-7-(4-ethyl-3-(hydroxymethyl)-5-oxo-4,5-dihydro-1H-1,2, |
| | 4-triazol-1-yl)-6-fluoro-1-(tetrahydro-2H-pyran-4-yl)-2,3-dihydropyrido[2,3-d] |
| | pyrimidin-4(1H)-one; |
| 7 | 3-(2-chloro-6-fluorophenyl)-1-cyclobutyl-7-(4-ethyl-3-(hydroxymethyl)-5-oxo-4,5- |
| | dihydro-1H-1,2,4-triazol-1-yl)-6-fluoro-2,3-dihydropyrido[2,3-d]pyrimidin-4(1H)-one; |
| 8 | (S)-3-(2-Chloro-6-fluorophenyl)-7-(4-ethyl-3-(hydroxymethyl)-5-oxo-4,5-dihydro- |
| | 1H-1,2,4-triazol-1-yl)-6-fluoro-1-(1,1,1-trifluoropropan-2-yl)-2,3-dihydropyrido |
| | [2,3-d]pyrimidin-4(1H)-one; |
| 9 | 3-(2-Chloro-6-fluorophenyl)-1-cyclopropyl-7-(4-ethyl-3-(hydroxymethyl)-5-oxo- |
| | 4,5-dihydro-1H-1,2,4-triazol-1-yl)-6-fluoro-2,3-dihydropyrido[2,3-d] |
| | pyrimidin-4(1H)-one; |
| 10 | Racemic-6-(4-Ethyl-3-(hydroxymethyl)-5-oxo-4,5-dihydro-1H-1,2,4-triazol- |
| | 1-yl)-2-(3-fluorophenyl)-4-isopropyl-3,4-dihydroisoquinolin-1(2H)-one; |
| 11 | Racemic-2-(2-Chloro-6-fluorophenyl)-6-(4-ethyl-3-(hydroxymethyl)-5-oxo-4,5- |
| | dihydro-1H-1,2,4-triazol-1-yl)-4-(prop-1-en-2-yl)-3,4-dihydroisoquinolin-1(2H)-one; |
| 12 | Racemic-6-(4-Ethyl-3-(hydroxymethyl)-5-oxo-4,5-dihydro-1H-1,2,4-triazol-1-yl)-2- |
| | (2-fluorophenyl)-4-isopropyl-3,4-dihydroisoquinolin-1(2H)-one; |
| 13 | Racemic-2-(2-Chloro-6-fluorophenyl)-6-(4-ethyl-3-(hydroxymethyl)-5-oxo-4,5- |
| | dihydro-1H-1,2,4-triazol-1-yl)-4-isopropyl-3,4-dihydroisoquinolin-1(2H)-one; |
| 14 | Racemic-6-(4-Ethyl-3-(hydroxymethyl)-5-oxo-4,5-dihydro-1H-1,2,4-triazol-1-yl)-2- |
| | (3-fluorophenyl)-4-phenyl-3,4-dihydroisoquinolin-1(2H)-one; |
| 15 | (S)-3-(2-Chloro-6-fluorophenyl)-7-(4-ethyl-3-(hydroxymethyl)-5-oxo-4,5-dihydro- |
| | 1H-1,2,4-triazol-1-yl)-6-fluoro-1-(pentan-2-yl)-2,3-dihydroquinazolin-4(1H)-one; |
| 16 | (S)-1-(sec-Butyl)-3-(2-chloro-6-fluorophenyl)-7-(4-ethyl-3-(hydroxymethyl)-5-oxo-4,5- |
| | dihydro-1H-1,2,4-triazol-1-yl)-6-fluoro-2,3-dihydroquinazolin-4(1H)-one; |
| 17 | (S)-3-(2-Chloro-6-fluorophenyl)-1-(1-cyclohexylethyl)-7-(4-ethyl-3-(hydroxymethyl)- |
| | 5-oxo-4,5-dihydro-1H-1,2,4-triazol-1-yl)-6-fluoro-2,3-dihydroquinazolin-4(1H)-one; |
| 18 | 3-(2-Chloro-6-fluorophenyl)-7-(4-ethyl-3-(hydroxymethyl)-5-oxo-4,5-dihydro- |
| | 1H-1,2,4-triazol-1-yl)-6-fluoro-1-isobutyl-2,3-dihydroquinazolin-4(1H)-one; |
| 19 | 3-(2-Chloro-6-fluorophenyl)-1-cyclobutyl-7-(4-ethyl-3-(hydroxymethyl)- |
| | 5-oxo-4,5-dihydro-1H-1,2,4-triazol-1-yl)-6-fluoro-2,3-dihydroquinazolin- |
| | 4(1H)-one; |
| 20 | 1-Butyl-3-(2-chloro-6-fluorophenyl)-7-(4-ethyl-3-(hydroxymethyl)-5- |
| | oxo-4,5-dihydro-1H-1,2,4-triazol-1-yl)-6-fluoro-2,3-dihydroquinazolin- |
| | 4(1H)-one; |
| 21 | 3-(2-Chloro-6-fluorophenyl)-1-(cyclohexylmethyl)-7-(4-ethyl-3-(hydroxymethyl)- |
| | 5-oxo-4,5-dihydro-1H-1,2,4-triazol-1-yl)-6-fluoro-2,3-dihydroquinazolin- |
| | 4(1H)-one; |
| 22 | 3-(2-Chloro-6-fluorophenyl)-7-(4-ethyl-3-(hydroxymethyl)-5-oxo-4,5-dihydro-1H- |
| | 1,2,4-triazol-1-yl)-6-fluoro-1-propyl-2,3-dihydroquinazolin-4(1H)-one; |
| 23 | (S)-1-(1-(1,3-Dioxan-2-yl)ethyl)-3-(2-chloro-6-fluorophenyl)-7-(4-ethyl-3- |
| | (hydroxymethyl)-5-oxo-4,5-dihydro-1H-1,2,4-triazol-1-yl)-6-fluoro-2,3- |
| | dihydropyrido[2,3-d]pyrimidin-4(1H)-one; |
| 24 | (S)-3-(2-Chloro-6-fluorophenyl)-7-(4-ethyl-3-(hydroxymethyl)-5-oxo-4,5-dihydro- |
| | 1H-1,2,4-triazol-1-yl)-6-fluoro-1-(pentan-2-yl)-2,3-dihydropyrido[2,3-d] |
| | pyrimidin-4(1H)-one; |
| 25 | 2-(4-Chloro-2-methylpyridin-3-yl)-6-(4-ethyl-3-(hydroxymethyl)-5-oxo-4,5- |
| | dihydro-1H-1,2,4-triazol-1-yl)-7-fluoro-4-(prop-1-en-2-yl)-3,4- |
| | dihydroisoquinolin-1(2H)-one; |
| 26 | 7-(4-Ethyl-3-(hydroxymethyl)-5-oxo-4,5-dihydro-1H-1,2,4-triazol-1-yl)-6- |
| | fluoro-3-(2-fluoro-5-methylphenyl)-1-isopropyl-2,3-dihydroquinazolin-4(1H)-one; |
| 27 | 4-(7-(4-Ethyl-3-(hydroxymethyl)-5-oxo-4,5-dihydro-1H-1,2,4-triazol-1-yl)-6-fluoro- |
| | 1-isopropyl-4-oxo-1,4-dihydroquinazolin-3(2H)-yl)-5-fluoronicotinonitrile; |
| 28 | 3-(2-Chloro-4-methylpyridin-3-yl)-7-(4-ethyl-3-(hydroxymethyl)-5-oxo-4,5- |
| | dihydro-1H-1,2,4-triazol-1-yl)-6-fluoro-1-isopropyl-2,3-dihydroquinazolin- |
| | 4(1H)-one; |
| 29 | 3-(3-Chloro-5-fluoropyridin-4-yl)-7-(4-ethyl-3-(hydroxymethyl)-5-oxo-4,5- |
| | dihydro-1H-1,2,4-triazol-1-yl)-6-fluoro-1-isopropyl-2,3-dihydroquinazolin- |
| | 4(1H)-one; |
| 30 | 3-(3-Chloro-6-methoxypyridin-2-yl)-7-(4-ethyl-3-(hydroxymethyl)-5-oxo-4,5- |
| | dihydro-1H-1,2,4-triazol-1-yl)-6-fluoro-1-isopropyl-2,3-dihydroquinazolin- |
| | 4(1H)-one; |
| 31 | (S)-1-(sec-Butyl)-3-(2-chloro-6-fluorophenyl)-7-(4-ethyl-3-(hydroxymethyl)-5- |
| | oxo-4,5-dihydro-1H-1,2,4-triazol-1-yl)-6-fluoro-2,3-dihydropyrido[2,3-d] |
| | pyrimidin-4(1H)-one; |
| 32 | 3-(3-Chloro-2-methoxy-5-methylpyridin-4-yl)-7-(4-ethyl-3-(hydroxymethyl)-5- |
| | oxo-4,5-dihydro-1H-1,2,4-triazol-1-yl)-6-fluoro-1-isopropyl-2,3- |
| | dihydroquinazolin-4(1H)-one; |
| 33 | (S*)-6-[4-Ethyl-3-(hydroxymethyl)-5-oxo-1,2,4-triazol-1-yl]-7-fluoro-4-isopropenyl- |
| | 2-(o-tolyl)-3,4-dihydroisoquinolin-1-one; |
| 34 | (S*)-6-[4-Ethyl-3-(hydroxymethyl)-5-oxo-1,2,4-triazol-1-yl]-7-fluoro-4-isopropenyl- |
| | 2-(o-tolyl)-3,4-dihydroisoquinolin-1-one; |
| 35 | (R*)-6-[4-Ethyl-3-(hydroxymethyl)-5-oxo-1,2,4-triazol-1-yl]-7-fluoro-4-isopropyl- |
| | 2-(o-tolyl)-3,4-dihydroisoquinolin-1-one; |
| 36 | (S*)-2-(2-Chlorophenyl)-6-(4-ethyl-3-(hydroxymethyl)-5-oxo-4,5-dihydro-1H- |
| | 1,2,4-triazol-1-yl)-7-fluoro-4-isopropyl-3,4-dihydroisoquinolin-1(2H)-one; |
| 37 | (R*)-2-(2-Chlorophenyl)-6-(4-ethyl-3-(hydroxymethyl)-5-oxo-4,5-dihydro-1H- |
| | 1,2,4-triazol-1-yl)-7-fluoro-4-isopropyl-3,4-dihydroisoquinolin-1(2H)-one; |
| 38 | Racemic 6-(4-Ethyl-3-(hydroxymethyl)-5-oxo-4,5-dihydro-1H-1,2,4-triazol-1-yl)- |
| | 7-fluoro-2-(2-fluoro-5-methylphenyl)-4-(prop-1-en-2-yl)-3,4-dihydroisoquinolin- |
| | 1(2H)-one; |
| 39 | (S*)-6-(4-Ethyl-3-(hydroxymethyl)-5-oxo-4,5-dihydro-1H-1,2,4-triazol-1-yl)- |
| | 7-fluoro-2-(2-fluoro-5-methylphenyl)-4-(prop-1-en-2-yl)-3,4-dihydroisoquinolin- |
| | 1(2H)-one; |
| 40 | (R*)-6-(4-Ethyl-3-(hydroxymethyl)-5-oxo-4,5-dihydro-1H-1,2,4-triazol-1-yl)- |
| | 7-fluoro-2-(2-fluoro-5-methylphenyl)-4-(prop-1-en-2-yl)-3,4-dihydroisoquinolin- |
| | 1(2H)-one; |
| 41 | (S*)-6-(4-Ethyl-3-(hydroxymethyl)-5-oxo-4,5-dihydro-1H-1,2,4-triazol-1-yl)- |
| | 7-fluoro-4-(prop-1-en-2-yl)-2-(o-tolyl)-3,4-dihydroisoquinolin-1(2H)-one; |
| 42 | (R*)-6-(4-Ethyl-3-(hydroxymethyl)-5-oxo-4,5-dihydro-1H-1,2,4-triazol-1-yl)- |
| | 7-fluoro-4-(prop-1-en-2-yl)-2-(o-tolyl)-3,4-dihydroisoquinolin-1(2H)-one; |
| 43 | (S*)-6-(4-Ethyl-3-(hydroxymethyl)-5-oxo-4,5-dihydro-1H-1,2,4-triazol-1-yl)- |
| | 7-fluoro-4-isopropyl-2-(o-tolyl)-3,4-dihydroisoquinolin-1(2H)-one; |
| 44 | (R*)-6-(4-Ethyl-3-(hydroxymethyl)-5-oxo-4,5-dihydro-1H-1,2,4-triazol-1-yl)- |
| | 7-fluoro-4-isopropyl-2-(o-tolyl)-3,4-dihydroisoquinolin-1(2H)-one; |
| 45 | Racemic 2-(2-Chloro-4-methylpyridin-3-yl)-6-(4-ethyl-3-(hydroxy methyl)- |
| | 5-oxo-4,5-dihydro-1H-1,2,4-triazol-1-yl)-7-fluoro-4-(prop-1-en-2-yl)- |
| | 3,4-dihydroisoquinolin-1(2H)-one; |
| 46 | (S*)-2-(2-Chloro-4-methylpyridin-3-yl)-6-(4-ethyl-3-(hydroxymethyl)-5-oxo-4,5- |
| | dihydro-1H-1,2,4-triazol-1-yl)-7-fluoro-4-(prop-1-en-2-yl)-3,4- |
| | dihydroisoquinolin-1(2H)-one; |
| 47 | (R*)-2-(2-Chloro-4-methylpyridin-3-yl)-6-(4-ethyl-3-(hydroxymethyl)-5-oxo-4,5- |
| | dihydro-1H-1,2,4-triazol-1-yl)-7-fluoro-4-(prop-1-en-2-yl)-3,4- |
| | dihydroisoquinolin-1(2H)-one; |
| 48 | (S*)-2-(5-Chloro-3-methyl-1H-pyrazol-4-yl)-6-(4-ethyl-3-(hydroxymethyl)- |
| | 5-oxo-4,5-dihydro-1H-1,2,4-triazol-1-yl)-7-fluoro-4-(prop-1-en-2-yl)-3,4- |
| | dihydroisoquinolin-1(2H)-one; |
| 49 | (R*)-2-(5-Chloro-3-methyl-1H-pyrazol-4-yl)-6-(4-ethyl-3-(hydroxymethyl)-5-oxo- |
| | 4,5-dihydro-1H-1,2,4-triazol-1-yl)-7-fluoro-4-(prop-1-en-2-yl)-3,4- |
| | dihydroisoquinolin-1(2H)-one; |
| 50 | Atropisomer 1, (S*)-6-(4-Ethyl-3-(hydroxymethyl)-5-oxo-4,5-dihydro-1H-1,2,4- |
| | triazol-1-yl)-7-fluoro-2-(2-methoxy-4-methylpyridin-3-yl)-4-(prop-1-en-2-yl)- |
| | 3,4-dihydroisoquinolin-1(2H)-one; |
| 51 | Atropisomer 2, (S*)-6-(4-Ethyl-3-(hydroxymethyl)-5-oxo-4,5-dihydro-1H-1,2,4- |
| | triazol-1-yl)-7-fluoro-2-(2-methoxy-4-methylpyridin-3-yl)-4-(prop-1-en-2-yl)- |
| | 3,4-dihydroisoquinolin-1(2H)-one; |
| 52 | Atropisomer 1, (R*)-6-(4-Ethyl-3-(hydroxymethyl)-5-oxo-4,5-dihydro-1H-1,2,4- |
| | triazol-1-yl)-7-fluoro-2-(2-methoxy-4-methylpyridin-3-yl)-4-(prop-1-en-2-yl)- |
| | 3,4-dihydroisoquinolin-1(2H)-one; |
| 53 | Atropisomer 2, (R*)-6-(4-Ethyl-3-(hydroxymethyl)-5-oxo-4,5-dihydro-1H-1,2,4- |
| | triazol-1-yl)-7-fluoro-2-(2-methoxy-4-methylpyridin-3-yl)-4-(prop-1-en-2-yl)- |
| | 3,4-dihydroisoquinolin-1(2H)-one; |
| 54 | Atropisomer 1, (S*)-6-(4-Ethyl-3-(hydroxymethyl)-5-oxo-4,5-dihydro-1H-1,2,4- |
| | triazol-1-yl)-7-fluoro-2-(2-methoxy-3,5-dimethylpyridin-4-yl)-4-(prop-1-en-2-yl)- |
| | 3,4-dihydroisoquinolin-1(2H)-one; |
| 55 | Atropisomer 1, (R*)-6-(4-Ethyl-3-(hydroxymethyl)-5-oxo-4,5-dihydro-1H-1,2,4- |
| | triazol-1-yl)-7-fluoro-2-(2-methoxy-3,5-dimethylpyridin-4-yl)-4-(prop-1-en-2-yl)- |
| | 3,4-dihydroisoquinolin-1(2H)-one; |
| 56 | Atropisomer 2, (S*)-6-(4-Ethyl-3-(hydroxymethyl)-5-oxo-4,5-dihydro-1H-1,2,4- |
| | triazol-1-yl)-7-fluoro-2-(2-methoxy-3,5-dimethylpyridin-4-yl)-4-(prop-1-en-2-yl)- |
| | 3 ,4-dihydroisoquinolin-1(2H)-one; |
| 57 | Atropisomer 2, (R*)-6-(4-Ethyl-3-(hydroxymethyl)-5-oxo-4,5-dihydro-1H-1,2,4- |
| | triazol-1-yl)-7-fluoro-2-(2-methoxy-3,5-dimethylpyridin-4-yl)-4-(prop-1-en-2-yl)- |
| | 3,4-dihydroisoquinolin-1(2H)-one; |
| 58 | Atropisomer 1, (S*)-2-(2-Chloro-4,6-dimethylpyridin-3-yl)-6-(4-ethyl-3- |
| | (hydroxymethyl)-5-oxo-4,5-dihydro-1H-1,2,4-triazol-1-yl)-7-fluoro-4-(prop- |
| | 1-en-2-yl)-3,4-dihydroisoquinolin-1(2H)-one; |
| 59 | Atropisomer 1, (R*)-2-(2-Chloro-4,6-dimethylpyridin-3-yl)-6-(4-ethyl-3- |
| | (hydroxymethyl)-5-oxo-4,5-dihydro-1H-1,2,4-triazol-1-yl)-7-fluoro-4-(prop- |
| | 1-en-2-yl)-3,4-dihydroisoquinolin-1(2H)-one; |
| 60 | Atropisomer 1, (S*)-2-(2-Chloro-4,6-dimethylpyridin-3-yl)-6-(4-ethyl-3- |
| | (hydroxymethyl)-5-oxo-4,5-dihydro-1H-1,2,4-triazol-1-yl)-7-fluoro-4- |
| | (prop-1-en-2-yl)-3,4-dihydroisoquinolin-1(2H)-one; |
| 61 | Atropisomer 2, (R*)-2-(2-Chloro-4,6-dimethylpyridin-3-yl)-6-(4-ethyl-3- |
| | (hydroxymethyl)-5-oxo-4,5-dihydro-1H-1,2,4-triazol-1-yl)-7-fluoro-4-(prop- |
| | 1-en-2-yl)-3,4-dihydroisoquinolin-1(2H)-one; |
| 62 | Atropisomer 1, (S*)-2-(3-Chloro-2-methoxy-5-methylpyridin-4-yl)-6-(4-ethyl-3- |
| | (hydroxymethyl)-5-oxo-4,5-dihydro-1H-1,2,4-triazol-1-yl)-7-fluoro-4-(prop-1-en- |
| | 2-yl)-3,4-dihydroisoquinolin-1(2H)-one; |
| 63 | Atropisomer 1, (R*)-2-(3-Chloro-2-methoxy-5-methylpyridin-4-yl)-6-(4-ethyl-3- |
| | (hydroxymethyl)-5-oxo-4,5-dihydro-1H-1,2,4-triazol-1-yl)-7-fluoro-4-(prop- |
| | 1-en-2-yl)-3,4-dihydroisoquinolin-1(2H)-one; |
| 64 | Atropisomer 2, (S*)-2-(3-Chloro-2-methoxy-5-methylpyridin-4-yl)-6-(4-ethyl-3- |
| | (hydroxymethyl)-5-oxo-4,5-dihydro-1H-1,2,4-triazol-1-yl)-7-fluoro-4-(prop- |
| | 1-en-2-yl)-3,4-dihydroisoquinolin-1(2H)-one; |
| 65 | Atropisomer 2, (R*)-2-(3-Chloro-2-methoxy-5-methylpyridin-4-yl)-6-(4-ethyl-3- |
| | (hydroxymethyl)-5-oxo-4,5-dihydro-1H-1,2,4-triazol-1-yl)-7-fluoro-4-(prop- |
| | 1-en-2-yl)-3,4-dihydroisoquinolin-1(2H)-one; |
| 66 | (S*)-2-(3-Chloro-2-methoxy-5-methylpyridin-4-yl)-6-(4-ethyl-3-(hydroxymethyl)- |
| | 5-oxo-4,5-dihydro-1H-1,2,4-triazol-1-yl)-7-fluoro-4-isopropyl-3,4- |
| | dihydroisoquinolin-1(2H)-one; |
| 67 | (R*)-2-(3-Chloro-2-methoxy-5-methylpyridin-4-yl)-6-(4-ethyl-3-(hydroxymethyl)- |
| | 5-oxo-4,5-dihydro-1H-1,2,4-triazol-1-yl)-7-fluoro-4-isopropyl-3,4- |
| | dihydroisoquinolin-1(2H)-one; |
| 68 | (R*)-2-(2-Chloro-6-fluorophenyl)-6-(4-ethyl-3-(hydroxymethyl)-5-oxo-4,5-dihydro- |
| | 1H-1,2,4-triazol-1-yl)-7-fluoro-4-(prop-1-en-2-yl)-3,4-dihydroisoquinolin- |
| | 1(2H)-one; |
| 69 | (S*)-2-(2-Chloro-6-fluorophenyl)-6-(4-ethyl-3-(hydroxymethyl)-5-oxo-4,5-dihydro- |
| | 1H-1,2,4-triazol-1-yl)-7-fluoro-4-(prop-1-en-2-yl)-3,4-dihydroisoquinolin- |
| | 1(2H)-one; |
| 70 | (S*)-4-((RS)-sec-Butyl)-2-(2-chloro-6-fluorophenyl)-6-(4-ethyl-3-(hydroxymethyl)- |
| | 5-oxo-4,5-dihydro-1H-1,2,4-triazol-1-yl)-7-fluoro-3,4-dihydroisoquinolin- |
| | 1(2H)-one; |
| 71 | (R*)-4-((S*)-sec-Butyl)-2-(2-chloro-6-fluorophenyl)-6-(4-ethyl-3-(hydroxymethyl)- |
| | 5-oxo-4,5-dihydro-1H-1,2,4-triazol-1-yl)-7-fluoro-3,4-dihydroisoquinolin- |
| | 1(2H)-one; |
| 72 | (R*)-4-((R*)-sec-Butyl)-2-(2-chloro-6-fluorophenyl)-6-(4-ethyl-3-(hydroxymethyl)- |
| | 5-oxo-4,5-dihydro-1H-1,2,4-triazol-1-yl)-7-fluoro-3,4-dihydroisoquinolin- |
| | 1(2H)-one; |
| 73 | Racemic 2-(2-Chloro-6-fluorophenyl)-6-(4-ethyl-3-(hydroxymethyl)-5-oxo-4,5-dihydro- |
| | 1H-1,2,4-triazol-1-yl)-4-(3,3,3-trifluoroprop-1-en-2-yl)-3,4-dihydroisoquinolin- |
| | 1(2H)-one; |
| 74 | 6-(4-Ethyl-3-(hydroxymethyl)-5-oxo-4,5-dihydro-1H-1,2,4-triazol-1-yl)-2-(2- |
| | fluorophenyl)-4-(1,1,1-trifluoropropan-2-yl)-3,4-dihydroisoquinolin-1 |
| | (2H)-one; and |
| 75 | 2-(2-Chloro-6-fluorophenyl)-6-(4-ethyl-3-(hydroxymethyl)-5-oxo-4,5-dihydro-1H- |
| | 1,2,4-triazol-1-yl)-4-(1,1,1-trifluoropropan-2-yl)-3,4-dihydroisoquinolin-1(2H)-one; |

or pharmaceutically acceptable salts, isotopes, N-oxides, solvates, or stereoisomers thereof; or pharmaceutically acceptable salts of the isotopes, N-oxides, solvates, or stereoisomers thereof.

An additional embodiment of the invention is a compound of Formula (I) having the Formula (IA):

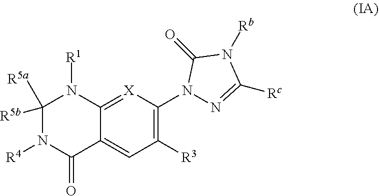

wherein
X is CH or N;
W is selected from the group consisting of: C₁₋₆alkyl; C₁₋₆alkyl substituted with OH, OCH₃, C₃₋₆cycloalkyl or C₃₋₆heterocycloalkyl; C₂₋₆alkenyl; C₂₋₆alkenyl substituted with one, two or three F members; C₁₋₆haloalkyl; C₁₋₆haloalkyl substituted with OH, or OCH₃; C₃₋₆cycloalkyl; C₃₋₆heterocycloalkyl; and phenyl;
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, and C₃₋₆cycloalkyl;
R³ is H or F;
R⁴ is selected from the group consisting of:

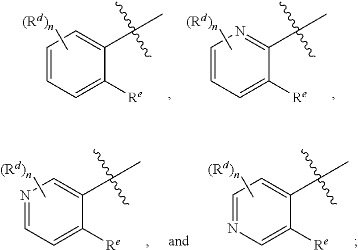

wherein
each R^{d} is independently a member selected from the group consisting of: H; halo; C₁₋₄alkyl; C₁₋₄alkyl substituted with a member selected from the group consisting of: OH, OCH₃, SCH₃, and OCF₃; C₁₋₄haloalkyl; C₁₋₄haloalkyl substituted with OH, or OCH₃; and OC₁₋₄alkyl;
R^{e} is a member selected from the group consisting of: H; halo; CN; C₁₋₄alkyl; C₁₋₄alkyl substituted with OH, OCH₃, SCH₃, and OCF₃; C₁₋₄haloalkyl; and C₁₋₄haloalkyl substituted with OH, or OCH₃;

n is 1 or 2; and
R^{5a} and R^{5b} are each independently H or CH₃; or R^{5a} and R^{5b} come together to form ═O.
An additional embodiment of the invention is a compound of Formula (I) having the Formula (IA), wherein
R¹ is

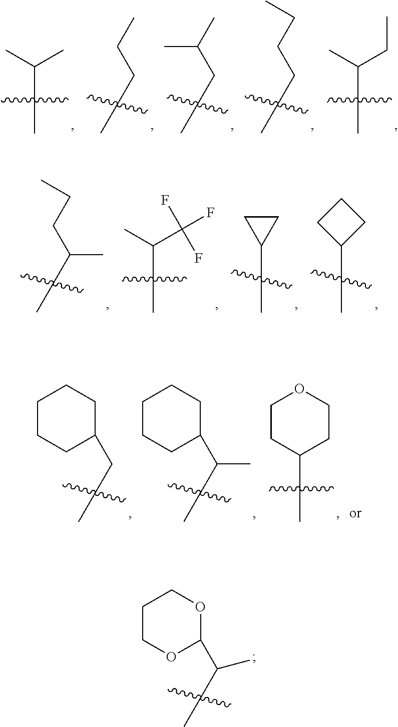

R^{b} is CH₂CH₃;
R^{c} is CH₂OH;
X is CH or N;
R³ is F;
R⁴ is selected from the group consisting of:

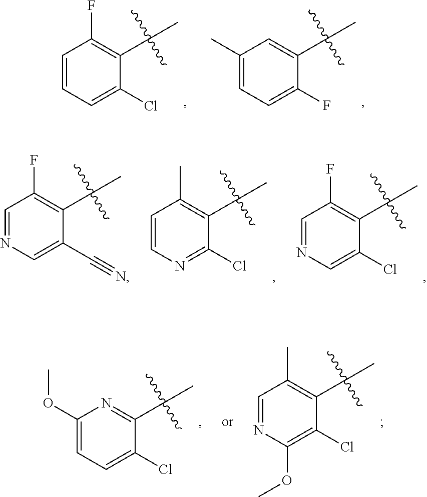

and

R^{5a} and R^{5b} are each independently H or CH₃; or R^{5a} and R^{5b} come together to form ═O

An additional embodiment of the invention is a compound of Formula (I) having the Formula (IB):

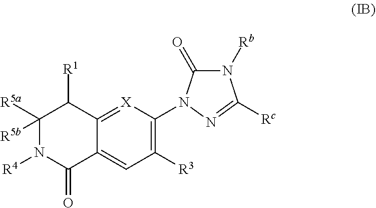

wherein
X is CH or N;
R¹ is selected from the group consisting of: C₁₋₄alkyl; C₁₋₄alkyl substituted with OH, OCH₃, C₃₋₆cycloalkyl or C₃₋₆heterocycloalkyl; C₂₋₆alkenyl; C₂₋₆alkenyl substituted with one, two or three F members; C₁₋₄haloalkyl; C₁₋₄haloalkyl substituted with OH, or OCH₃; C₃₋₆cycloalkyl; C₃₋₆heterocycloalkyl; and phenyl;
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, and C₃₋₆cycloalkyl;
R³ is H or F;
R⁴ is selected from the group consisting of:

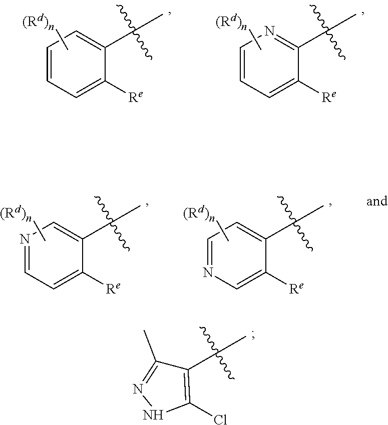

wherein
each R^{d} is independently selected from the group consisting of: H; halo; C₁₋₄alkyl; C₁₋₄alkyl substituted with a member selected from the group consisting of: OH, OCH₃, SCH₃, and OCF₃; C₁₋₄haloalkyl; C₁₋₄haloalkyl substituted with OH, or OCH₃; and OC₁₋₄alkyl; and
R^{e} is selected from the group consisting of: H; halo; CN; C₁₋₄alkyl; C₁₋₄alkyl substituted with OH, OCH₃, SCH₃, and OCF₃; C₁₋₄haloalkyl; and C₁₋₄haloalkyl substituted with OH, or OCH₃;

n is 1 or 2; and
R^{5a} and R^{5b} are each independently H or CH₃; or R^{5a} and R^{5b} come together to form ═O.
An additional embodiment of the invention is a compound of Formula (I) having the Formula (IB), wherein
R¹ is

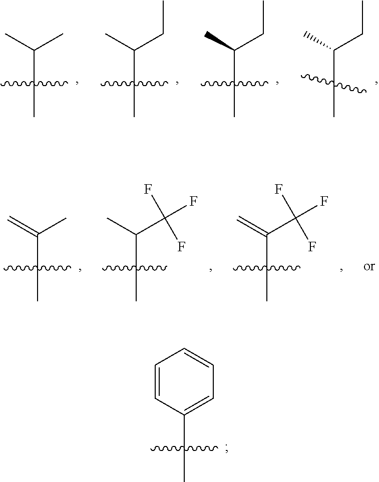

R^{b} is CH₂CH₃;
R^{c} is CH₂OH;
X is CH;
R³ is H or F;
R⁴ is selected from the uoup consisting of:

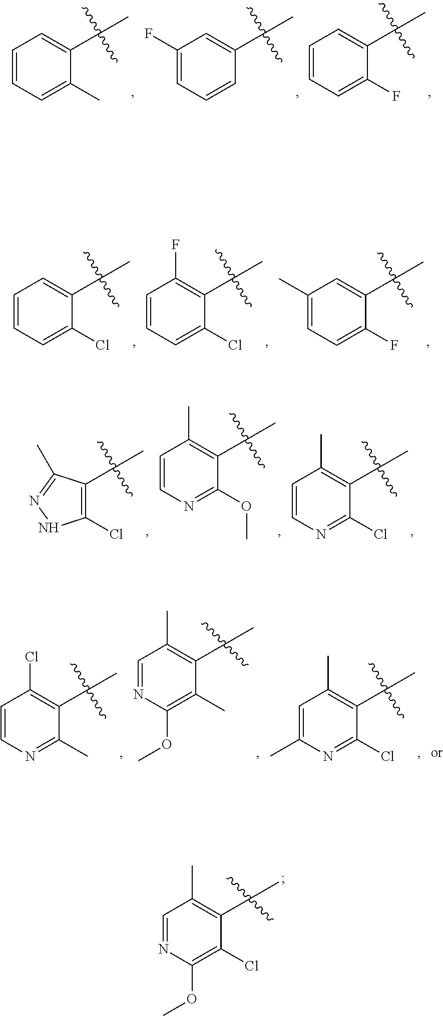

and

R^{5a} and R^{5b} are H.

An additional embodiment of the invention is a compound of Formula (I) having the Formula (IA), wherein X is N.

An additional embodiment of the invention is a compound of Formula (I) having the Formula (IA), wherein X is CH.

An additional embodiment of the invention is a compound of Formula (I) having the Formula (IB), wherein X is CH.

Also within the scope of the invention are enantiomers and diastereomers of the compounds of Formula (I) (as well as Formulas (IA), and (IB)). Also within the scope of the invention are the pharmaceutically acceptable salts, N-oxides or solvates of the compounds of Formula (I) (as well as Formulas (IA), and (IB)). Also within the scope of the invention are the pharmaceutically acceptable prodrugs of compounds of Formula (I) (as well as Formulas (IA), and (IB)), and pharmaceutically active metabolites of the compounds of Formula (I) (as well as Formulas (IA), and (IB)).

Also within the scope of the invention are isotopic variations of compounds of Formula (I) ((as well as Formulas (IA), and (IB)), such as, e.g., deuterated compounds of Formula (I). Also within the scope of the invention are the pharmaceutically acceptable salts, N-oxides or solvates of the isotopic variations of the compounds of Formula (I) (as well as Formulas (IA), and (IB)). Also within the scope of the invention are the pharmaceutically acceptable prodrugs of the isotopic variations of the compounds of Formula (I) (as well as Formulas (IA), and (IB)), and pharmaceutically active metabolites of the isotopic variations of the compounds of Formula (I) (as well as Formulas (IA), and (IB)).

Even though the compounds of embodiments of the present invention (including their pharmaceutically acceptable salts and pharmaceutically acceptable solvates) can be administered alone, they will generally be administered in admixture with a pharmaceutically acceptable carrier, a pharmaceutically acceptable excipient and/or a pharmaceutically acceptable diluent selected with regard to the intended route of administration and standard pharmaceutical or veterinary practice.

Thus, particular embodiments of the present invention are directed to pharmaceutical and veterinary compositions comprising compounds of Formula (I) and at least one pharmaceutically acceptable carrier, pharmaceutically acceptable excipient, and/or pharmaceutically acceptable diluent. By way of example, in the pharmaceutical compositions of embodiments of the present invention, the compounds of Formula (I) may be admixed with any suitable binder(s), lubricant(s), suspending agent(s), coating agent(s), solubilizing agent(s), and combinations thereof.

An embodiment of the invention relates to a pharmaceutical composition comprising an effective amount of at least one compound selected from compounds of Formula (I), and pharmaceutically acceptable salts, isotopes, N-oxides, solvates, and stereoisomers thereof, in accordance with any embodiment described herein; and at least one pharmaceutically acceptable excipient.

An additional embodiment of the invention is a pharmaceutical composition comprising:
(A) an effective amount of at least one compound selected from compounds of Formula (I)

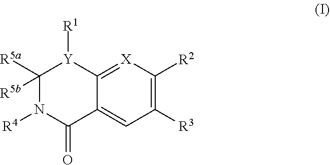

wherein
X is CH or N;
Y is CH or N;
R¹ is selected from the group consisting of: C₁₋₆alkyl; C₁₋₆alkyl substituted with OH, OCH₃, C₃₋₆cycloalkyl or C₃₋₆heterocycloalkyl; C₂₋₆alkenyl; C₂₋₆alkenyl substituted with one, two or three halo members; C₁₋₆haloalkyl; C₁₋₆haloalkyl substituted with OH, or OCH₃; C₃₋₆cycloalkyl; C₃₋₆heterocycloalkyl; and phenyl;

R² is

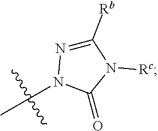

wherein
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, and C₃₋₆cycloalkyl;
R³ is H or F;
R⁴ is selected from the group consisting of:

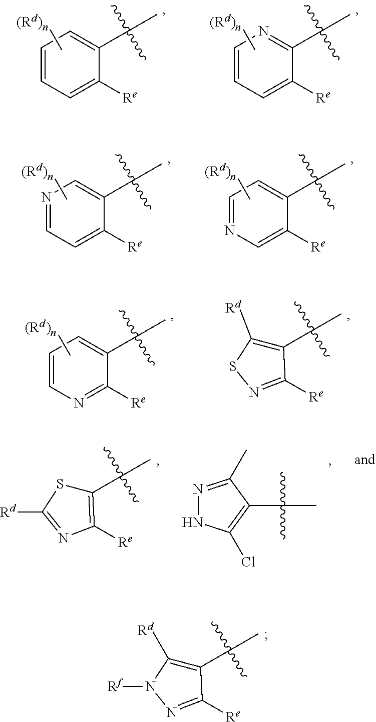

wherein
each R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
R^{e} is selected from the group consisting of: H; halo; CN; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃;
R^{5a} and R^{5b} are each independently H or CH₃; or R^{5a} and R^{5b} come together to form ═O; and

n is 1 or 2;

or pharmaceutically acceptable salts, isotopes, N-oxides, solvates, or stereoisomers of a compound of Formula (I);

and (B) at least one pharmaceutically acceptable excipient.

An additional embodiment of the invention is a pharmaceutical composition comprising an effective amount of a compound shown in Table 1 (e.g., a compound selected from Examples 1-75), or a pharmaceutically acceptable salt, isotope, N-oxide, solvate, or stereoisomer of the compound of Table 1, a pharmaceutically acceptable prodrug of the compound of Table 1, or a pharmaceutically active metabolite of the compound of Table 1; and at least one pharmaceutically acceptable excipient.

Solid oral dosage forms such as, tablets or capsules, containing one or more compounds of the present invention may be administered in at least one dosage form at a time, as appropriate. It is also possible to administer the compounds in sustained release formulations.

Additional oral forms in which the present inventive compounds may be administered include elixirs, solutions, syrups, and suspensions; each optionally containing flavoring agents and coloring agents.

Alternatively, one or more compounds of Formula (I) can be administered by inhalation (intratracheal or intranasal) or in the form of a suppository or pessary, or they may be applied topically in the form of a lotion, solution, cream, ointment or dusting powder. For example, they can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a wax or soft paraffin base together with any stabilizers and preservatives as may be required. An alternative means of administration includes transdermal administration by using a skin or transdermal patch.

The pharmaceutical compositions of the present invention (as well as the compounds of the present invention alone) can also be injected parenterally, for example, intracavernosally, intravenously, intramuscularly, subcutaneously, intradermally, or intrathecally. In this case, the compositions will also include at least one of a suitable carrier, a suitable excipient, and a suitable diluent.

For parenteral administration, the pharmaceutical compositions of the present invention are best used in the form of a sterile aqueous solution that may contain other substances, for example, enough salts and monosaccharides to make the solution isotonic with blood.

For buccal or sublingual administration, the pharmaceutical compositions of the present invention may be administered in the form of tablets or lozenges, which can be formulated in a conventional manner.

By way of further example, pharmaceutical compositions containing at least one of the compounds of Formula (I) as the active ingredient can be prepared by mixing the compound(s) with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, etc.). Thus, for liquid oral preparations such as, suspensions, syrups, elixirs and solutions, suitable carriers, excipients and diluents include water, glycols, oils, alcohols, flavoring agents, preservatives, stabilizers, coloring agents and the like; for solid oral preparations such as, powders, capsules, and tablets, suitable carriers, excipients and diluents include starches, sugars, diluents, granulating agents, lubricants, binders, disintegrating agents and the like. Solid oral preparations also may be optionally coated with substances such as, sugars, or be enterically coated so as to modulate the major site of absorption and disintegration. For parenteral administration, the carrier, excipient and diluent will usually include sterile water, and other ingredients may be added to increase solubility and preservation of the composition. Injectable suspensions or solutions may also be prepared utilizing aqueous carriers along with appropriate additives such as, solubilizers and preservatives.

According to particular embodiments, a therapeutically effective amount of a compound of Formula (I) or a pharmaceutical composition thereof may comprise a dose range from about 0.1 mg to about 3000 mg, or any particular amount or range therein, in particular from about 1 mg to about 1000 mg, or any particular amount or range therein, or, more particularly, from about 10 mg to about 500 mg, or any particular amount or range therein, of active ingredient in a regimen of about 1 to about (4×) per day for an average (70 kg) human; although, it is apparent to one skilled in the art that the therapeutically effective amount for a compound of Formula (I) will vary as will the diseases, syndromes, conditions, and disorders being treated.

For oral administration, a pharmaceutical composition may be provided in the form of one or more tablets containing about 1.0, about 10, about 50, about 100, about 150, about 200, about 250, or about 500 milligrams of a compound of Formula (I).

An embodiment of the present invention is directed to a pharmaceutical composition for oral administration, comprising a compound of Formula (I) in an amount of from about 1 mg to about 500 mg.

Advantageously, a compound of Formula (I) may be administered in a single daily dose, or the total daily dosage may be administered in divided doses of two, three and (4×) daily.

Optimal dosages of a compound of Formula (I) to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

Compounds of Formula (I) may be administered in any of the foregoing compositions and dosage regimens or by means of those compositions and dosage regimens established in the art whenever use of a compound of Formula (I) is administered to a subject in need thereof.

According to particular embodiments, one or more compounds of Formula (I) are useful in methods for treating, ameliorating and/or preventing a disease, a syndrome, a condition or a disorder that is affected by the inhibition of DHODH enzymatic activity.

An additional embodiment of the invention relates to the use of compounds of Formula (I), e.g., by inhibiting dihydroorotate oxygenase enzyme activity, in treating disorders like inflammatory disorders, autoimmune disorders, or cancer;

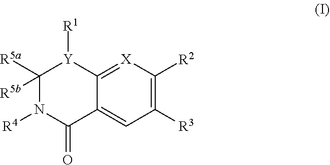

wherein

X is CH or N;

Y is CH or N;

R¹ is selected from the group consisting of: C₁₋₆alkyl; C₁₋₆alkyl substituted with OH, OCH₃, C₃₋₆cycloalkyl or C₃₋₆heterocycloalkyl; C₂₋₆alkenyl; C₂₋₆alkenyl substituted with one, two or three halo members; C₁₋₆haloalkyl; C₁₋₆haloalkyl substituted with OH, or OCH₃; C₃₋₆cycloalkyl; C₃₋₆heterocycloalkyl; and phenyl;

R² is

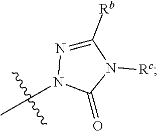

wherein
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, and C₃₋₆cycloalkyl;
R³ is H or F;
R⁴ is selected from the group consisting of:

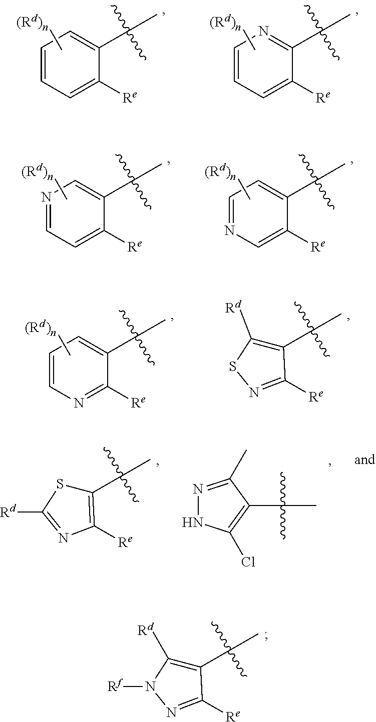

wherein
each R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
R^{e} is selected from the group consisting of: H; halo; CN; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃;
R^{5a} and R^{5b} are each independently H or CH₃; or R^{5a} and R^{5b} come together to form ═O; and

n is 1 or 2;

or pharmaceutically acceptable salts, isotopes, N-oxides, solvates, or stereoisomers thereof.

In a further aspect the present invention provides a method for inhibiting or altering Dihydroorotate Dehydrogenase (DHODH) enzymatic activity, the method comprising contacting DHODH with any compound of Formula (I), aspect or embodiment disclosed herein, thereby inhibiting or otherwise altering DHODH enzymatic activity.

An additional embodiment of the present invention provides methods for treating diseases, disorders, or medical conditions mediated or otherwise affected by dihydroorotate dehydrogenase (DHODH) enzyme activity comprising administering a compound of Formula (I) to a subject in need thereof.

As used herein, the term “DHODH inhibitor” may refer to an agent that inhibits or reduces DHODH activity.

In one embodiment, the term “therapeutically effective amount” (or “effective amount”) refers to the amount of a compound of the present invention that, when administered to a subject, is effective to (1) at least partially alleviate, inhibit, prevent, and/ or ameliorate a condition, or a disorder or a disease (i) mediated by DHODH enzymatic activity; or (ii) associated with DHODH enzymatic activity; or (iii) characterized by activity (normal or abnormal) of DHODH enzyme; or (2) reduce or inhibit the activity of DHODH enzyme; or (3) reduce or inhibit the expression of DHODH; or (4) modify the protein levels of DHODH. Without being bound by a particular theory, DHODH inhibitors are believed to act by inhibiting nucleic acid synthesis, cell cycle arrest or altering post-translational glycosylation of proteins involved in regulating myeloid differentiation within progenitor tumor cells.

An additional embodiment of the invention is a method of treating a subject suffering from or diagnosed with a disease, disorder, or medical condition mediated or otherwise affected by DHODH enzymatic activity, comprising administering to a subject in need of such treatment an effective amount of at least one compound selected from: compounds of Formula (I) (as well as Formulas (IA), and (IB), such as a compound of Table 1), enantiomers and diastereomers of the compounds of Formula (I) (as well as Formulas (IA), and (IB), such as a compound of Table 1), isotopic variations of the compounds of Formula (I) (as well as Formulas (IA), and (IB), such as a compound of Table 1), and pharmaceutically acceptable salts of all of the foregoing. Stated another way, according to an embodiment, a method of treating a subject suffering from or diagnosed with a disease, disorder, or medical condition, such as cancer, comprises administering to the subject an effective amount of at least one compound selected from: compounds of Formula (I) (as well as Formulas (IA), and (IB), such as a compound of Table 1), and pharmaceutically acceptable salts of all the foregoing (e.g., by inhibiting or otherwise altering dihydroorotate oxygenase enzyme activity in the subject).

In another embodiment, inhibitors of DHODH of the present invention may be used for the treatment of immunological diseases including, but not limited to, autoimmune and inflammatory disorders, e.g. arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjogren's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including edema, embolism, fibrosis, sarcoidosis, hypertension and emphysema, silicosis, respiratory failure, acute respiratory distress syndrome, BENTA disease, berylliosis, and polymyositis.

As used herein, unless otherwise noted, the term “affect” or “affected” (when referring to a disease, disorder, or medical condition that is affected by the inhibition or alteration of DHODH enzymatic activity) includes a reduction in the frequency and/or severity of one or more symptoms or manifestations of said disease, syndrome, condition or disorder; and/or includes the prevention of the development of one or more symptoms or manifestations of said disease, syndrome, condition or disorder or the development of the disease, condition, syndrome or disorder.

An additional embodiment of the invention provides a method of treatment of cancer comprising administering to a subject in need thereof, a therapeutically effective amount of a compound of Formula (I), or a pharmaceutically acceptable salt, isotope, N-oxide, solvate, or stereoisomer thereof.

According to an embodiment, the cancer is selected from but not limited to, lymphomas, leukemias, carcinomas, and sarcomas.

An additional embodiment of the invention provides the use of a compound of Formula (I), or a pharmaceutically acceptable salt, isotope, N-oxide, solvate, or stereoisomer thereof, for the treatment of one or more cancer types.

According to particular embodiments, the uses and methods of treatment described herein are directed to the treatment of cancer, wherein the cancer is selected from but not limited to:
leukemias including but not limited to acute lymphoblastic leukemia (ALL), acute myeloid leukemia (AML), (acute) T-cell leukemia, acute monocytic leukemia, acute promyelocytic leukemia (APL), bisphenotypic B myelomonocytic leukemia, chronic myeloid leukemia (CML), chronic myelomonocytic leukemia (CMML), large granular lymphocytic leukemia, plasma cell leukemia, and also myelodysplastic syndrome (MDS), which can develop into an acute myeloid leukemia,
lymphomas including but not limited to AIDS-related lymphoma, Hodgkin lymphoma, non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell DLBCL, double-hit lymphoma and double-expressor lymphoma; anaplastic large cell lymphoma, marginal B cell lymphoma and primary mediastinal B-cell lymphoma, immunoblastic large cell lymphoma, Burkitt lymphoma, follicular lymphoma, hairy cell leukemia, Hodgkin's disease, mantle cell lymphoma (MCL), lymphoplasmatic lymphoma, precursor B-lymphoblastic lymphoma, lymphoma of the central nervous system, small lymphocytic lymphoma (SLL) and chronic lymphocytic leukemia (CLL); T-cell NHL such as precursor T-lymphoblastic lymphoma/leukemia, peripheral T-cell lymphoma (PTCL), cutaneous T-cell lymphoma (CTCL), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma
sarcomas including but not limited to sarcoma of the soft tissue, gliosarcoma, osteosarcoma, malignant fibrous histiocytoma, lymphosarcoma, and rhabdomyosarcoma;
   and
other cancers, such as solid tumors, including but not limited to breast cancer, colorectal carcinoma, gastric cancer, gliosarcoma, head & neck cancer, hepatocellular carcinoma, lung cancer, multiple myeloma, neuroblastoma, ovarian cancer, pancreatic cancer, prostate cancer, renal cell carcinoma and sarcoma.

In an embodiment, cancers that may benefit from a treatment with inhibitors of DHODH of the present invention include, but are not limited to, lymphomas, leukemias, carcinomas, and sarcomas, e.g. non-Hodgkin's lymphoma, diffuse large B-cell lymphoma (DLBCL), mantle cell lymphoma (MCL), follicular lymphoma (FL), marginal zone lymphoma, T-cell lymphoma, Hodgkin's lymphoma, Burkitt's lymphoma, multiple myeloma, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

In another embodiment of the present invention, the compounds of the present invention may be employed in combination with one or more other medicinal agents, more particularly with one or more anti-cancer agents, e.g. chemotherapeutic, anti-proliferative or immunomodulating agents, or with adjuvants in cancer therapy, e.g. immunosuppressive or anti-inflammatory agents. Additional non-limiting examples of anti-cancer agents that may be administered in combination with a compound of the present invention include biologic compounds, such as monoclonal antibodies (e.g., that mediate effector function upon binding to cancer cell-associated antigens, or block interaction of a receptor expressed on cancer cells with a soluble or cell bound ligand), bispecific antibodies that mediate immune cell redirection, etc. According to an embodiment, a method of treating cancer comprises administering an effective amount of a compound of the present invention (e.g., selected from compounds of Formula (I), such as a compound shown in Table 1, pharmaceutically acceptable salts, isotopes, N-oxides, solvates, and stereoisomers thereof) and an effective amount of one or more additional anti-cancer agents, wherein the method comprises administering the compound of the present invention and the additional anti-cancer agent(s) either simultaneously (e.g., as part of the same pharmaceutical composition) or sequentially. According to an embodiment, a pharmaceutical composition comprises an effective amount of a compound of the present invention (e.g., selected from compounds of Formula (I), such as a compound shown in Table 1, pharmaceutically acceptable salts, isotopes, N-oxides, solvates, and stereoisomers thereof), an effective amount of one or more additional anti-cancer agents, and optionally one or more excipients.

An additional embodiment of the invention provides the use of a compound of Formula (I), or pharmaceutically acceptable salts, isotopes, N-oxides, solvates, or stereoisomers thereof, as part of chemotherapeutic regimens for the treatment of cancers, lymphomas and leukemias alone or in combination with classic antitumoral compounds well known by the one skilled in the art.

### General Synthetic Methods

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Abbreviations used in the instant specification, particularly the schemes and examples, are as follows in Table 2:

TABLE 2

| Abbreviation | Name |
|---|---|
| CAN or MeCN | acetonitrile |
| ABPR | automated backpressure regulator |
| AcOH | glacial acetic acid |
| AcOK | Potassium acetate |
| AlMe₃ | Trimethylaluminium |
| aq. | aqueous |
| Bn or Bzl | benzyl |
| Boc | tert-butyloxycarbonyl |
| Boc₂O | Di-tert-butyl dicarbonate |
| Celite ® | Diatomaceous earth |
| Cu(OAc)2 | copper(II) acetate |
| conc. | Concentrated |
| Cy2NMe | N,N-Dicyclohexylmethylamine |
| DCM | dichloromethane |
| DIPEA or DIEA | diisopropyl-ethyl amine |
| DCIP | dichloroindophenol |
| DMA | dimethylaniline |
| DHO | dihydroorotate |
| DMP | Dess-martin periodinane |
| DMAP | 4-dimethylaminopyridine |
| DME | dimethoxyethane |
| DMF | N,N-dimethylformamide |
| DMSO | dimethylsulfoxide |
| EA | ethyl acetate |
| ESI | electrospray ionization |
| EtOAc or EA | ethyl acetate |
| EtOH | ethanol |
| FCC | Flash Column Chromatography |
| FMN | flavin mononucleotide |
| GCMS | gas chromatography-mass spectrometry |
| h or hr(s) | hour or hours |
| HPLC | high performance liquid chromatography |
| LiHMDS | Lithium bis(trimethylsilyl)amide |
| MeOH | methanol |
| MHz | megahertz |
| min | minute or minutes |
| MS | mass spectrometry |
| MsCl | Methanesulfonyl chloride or mesyl chloride |
| NIS | N-iodosuccinimide |
| NMP | N-Methyl-2-pyrrolidone |
| NMR | nuclear magnetic resonance |
| Pd(OAc)₂ | Palladium(II) acetate |
| PdCl₂(PPh₃)₂ | bis(triphenylphosphine)palladium(II) |
| | dichloride |
| PE | petrolum ether |
| PTSA or pTsO, or tosylic | p-toluenesulfonic acid |
| acid or TsOH or Tosic acid | |
| Py | pyridine |
| RP | reverse-phase |
| rt or RT | room temperature |
| Rₜ | retention time |
| Sec | second or seconds |
| TBAF | Tetra-n-butylammonium fluoride |
| TBDPS | tert-Butyldiphenylchlorosilane |
| TBS | tert-Butyldimethylsilyl |
| tBu3P-Pd-G2 | Chloro[(tri-tert-butylphosphine)-2- |
| | (2-aminobiphenyl)] palladium(II) |
| TES | triethylsilane |
| TIPS | triisopropylsilane |
| TEA or Et₃N or NEt₃ | triethylamine |
| TFA | trifluoroacetic acid |
| THF | tetrahydrofuran |
| TLC | thin layer chromatography |

### PREPARATIVE EXAMPLES

Exemplary compounds useful in methods of the invention will now be described by reference to the illustrative synthetic schemes for their general preparation below and the specific examples to follow.

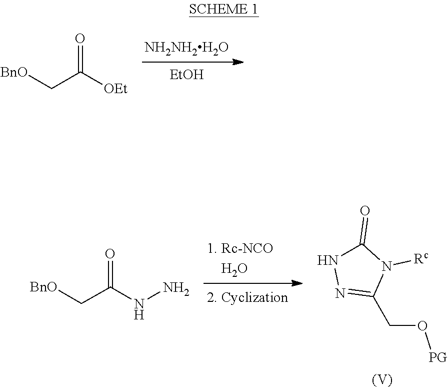

According to SCHEME 1, a 1,2,4-triazol-5(4H)-one compound of formula (V), where PG is Bn, is prepared from ethyl 2-(benzyloxy)acetate in three steps. In a first step 2-(benzyloxy)acetohydrazide is prepared by the reaction of ethyl 2-(benzyloxy)acetate with hydrazine hydrate, in a suitable solvent such as EtOH, and the like; at temperatures ranging from 70-85° C. Reaction of the hydrazide with an isocyanate of formula R^{c}—NCO, where R^{c} is C₁₋₆alkyl, in a suitable solvent such as water, and the like; provides the corresponding semicarbazide. Subsequent cyclization of the semicarbazide with a suitable base such as NaOH, in a suitable solvent such as water, provides a compound of formula (V), where PG is Bn.

Protecting group exchange of a compound of formula (V), where PG is Bn to a compound of formula (V) where PG is TBDPS, is achieved in two steps employing established methodologies, such as those described in T. W. Greene and P. G. M. Wuts, “Protective Groups in Organic Synthesis,” 3 ed., John Wiley & Sons, 1999. In a first step, deprotection of benzyl group is achieved under hydrogenolytic conditions known to one skilled in the art provides the alcohol. For example, deprotection is achieved employing a palladium catalyst such Pd/C, and the like; under H2; in a suitable solvent such as EtOH, MeOH, EtOAc, or a mixture thereof, preferably EtOH; with or without the presence HCl; for a period of 4 to 72 hrs. In a second step, protection of the corresponding alcohol as the silyl ether, is achieved with tent-butyldimethylsilyl chloride, a suitable base such as imidazole, dimethylaminopyridine, pyridine, and the like; in a solvent such as DMF, DCM, and the like; at temperatures ranging from 0° C. to room temperature; affords a compound of formula (V) where PG is TBDPS.

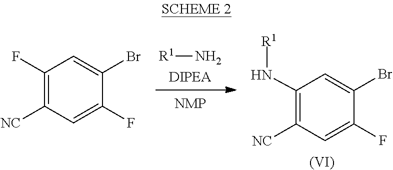

According to SCHEME 2, 4-bromo-2,5-difluorobenzonitrile is reacted with an amine of formula R¹−NH₂, where R¹ is C₁₋₆alkyl; in the presence of a suitable base such as DIPEA, and the like; in a suitable solvent such as NMP, and the like; to provide a compound of formula (VI), where R¹ is C₁₋₆alkyl.

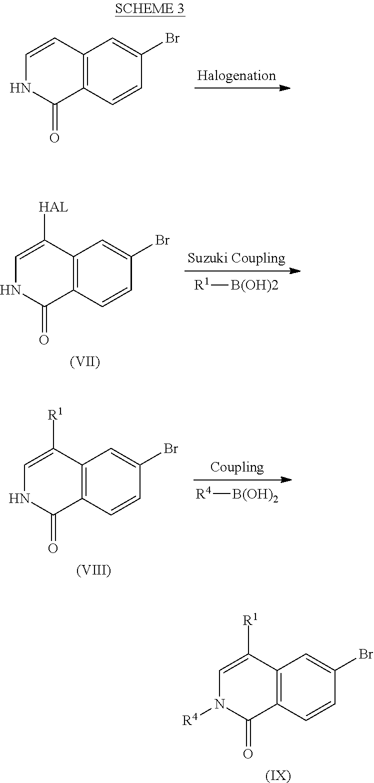

According to SCHEME 3, 6-bromoisoquinolin-1(2H)-one is treated with a halogenating reagent such as N-iodosuccinimide (NIS), and the like; in an aprotic solvent such as acetonitrile, and the like; under heating conditions; affords the halogenated compound of formula (VII), where HAL is I. A compound of formula R¹—B(OH)₂; is reacted under Suzuki coupling conditions known to one skilled in the art with a compound of formula (VII), to provide a compound of formula (VIII). For example, a compound of formula (VII), where HAL is I, is reacted a commercially available or synthetically accessible boronic acid (or boronic ester) such as R¹—B(OH)₂, where R¹ is an optionally substituted alkenyl or aryl as defined in claim 1; a palladium catalyst such as bis(triphenylphosphine)palladium(II) dichloride, and the like; a suitable base such a potassium phosphate, Cs₂CO₃, and the like; in a suitable solvent such as dioxane, water, ethanol, or a mixture thereof; to provide a compound of formula compound (VIII). A compound of formula (VIII) is reacted with a compound of formula R⁴—B(OH)₂; under copper (II) mediated Chan-Lam coupling conditions known to one skilled in the art, to provide a compound of formula (IX). For example, reaction of a compound of formula (VIII) with a compound of formula R⁴—B(OH)₂, where R⁴ is as defined in claim 1; with a catalyst such as copper(II) acetate, and the like; a base such as pyridine, NEt₃, and the like; in a suitable solvent such as DCM, ACN, dioxane, THF, and the like; affords a compound of formula (IX).

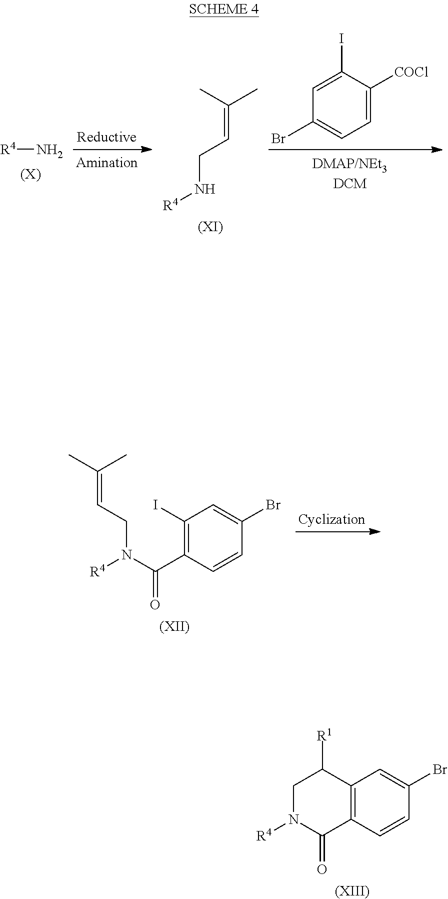

According to SCHEME 4, reductive amination of a compound of formula (X), with a β-unsaturated aldehyde such as 3-methylbut-2-enal; employing TiCl₄; and a base such as triethylamine; in an aprotic solvent such as dichloromethane (DCM), and the like; provides an enamine intermediate which is subsequently reduced employing a reducing agent such as NaBH₄, and the like; to afford a compound of formula (XI) where R⁴ is as defined in claim 1. A compound of formula (XI) is coupled with 4-bromo-2-iodobenzoyl chloride employing a base such as triethylamine and 4-dimethylaminopyridine (DMAP); in an anhydrous aprotic solvent such as dichloromethane (DCM), and the like; affords a compound of formula (XII). Treatment of a compound of formula (XII) with Palladium (II) acetate, tetrabutylammonium bromide, and potassium acetate under heating Heck reaction conditions, affords the intramolecular cyclized compound of formula (XIII), wherein R¹ is an optionally substituted alkenyl as described in claim 1.

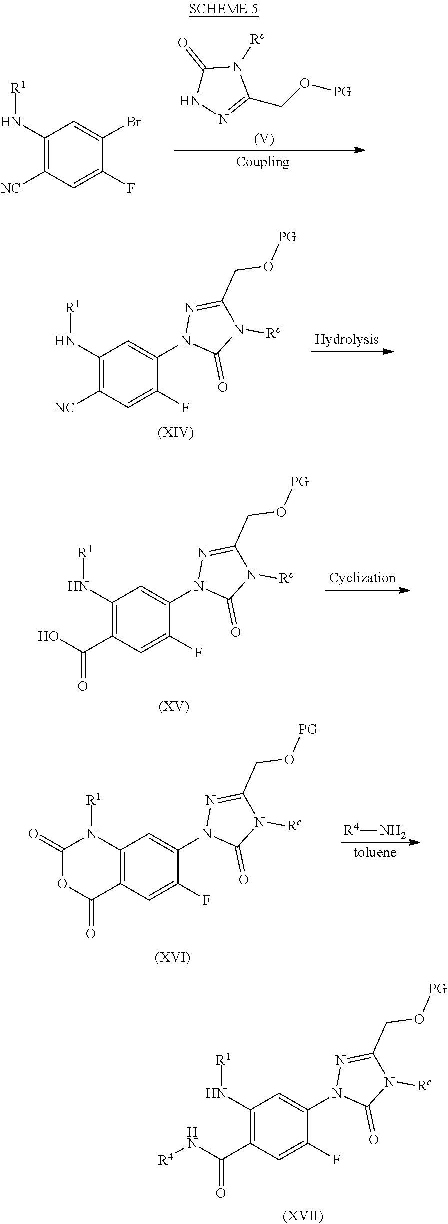

According to SCHEME 5, reaction of a compound of formula (VI), where R¹ is isopropyl; with a commercially available or synthetically accessible nucleophile compound of formula (V), such as suitably protected triazolones, where PG is selected from: benzyl, 4-methoxy benzyl, or an alkyl or aryl silane such as TPDPS, TBS, TES, or TIPS, and R^{c} is ethyl; in the presence of a base such as K₃PO₄, Cs₂CO₃, and the like; in the presence of KI; a suitable catalyst such as CuI, and the like; a suitable ligand such as N1,N2-dimethylcyclohexane-1,2-diamine, and the like; in a suitable solvent such as 1,4-dioxane, and the like; at elevated temperatures, preferably 100° C.; affords a compound of formula (XIV).

Hydrolysis of nitrile compound of formula (XIV), in the presence of a suitable base such as NaOH, LiOH, and the like; in a suitable solvent such as ethanol, methanol, THF, and the like; at elevated temperature, preferably 100° C.; affords a compound of formula (XV).

Reaction of compound of the formula (XV); with triphosgene, carbonyl diimidazole, and the like; in a suitable solvent such as DCM, DFM, THF, and the like; at ambient or reduced temperatures, preferably 0° C.; affords a compound of formula (XVI).

A compound of the formula (XVI) is reacted with the resulting solution of a compound of formula R⁴—NH₂, where R⁴ is 2-chloro-6-fluorophenyl, which has been reacted with trimethyl aluminum, in a suitable solvent such as dichloromethane, toluene, or a mixture thereof; to provide a compound of the formula (XVII), where R¹ is isopropyl, R^{c} is ethyl, and PG is benzyl.

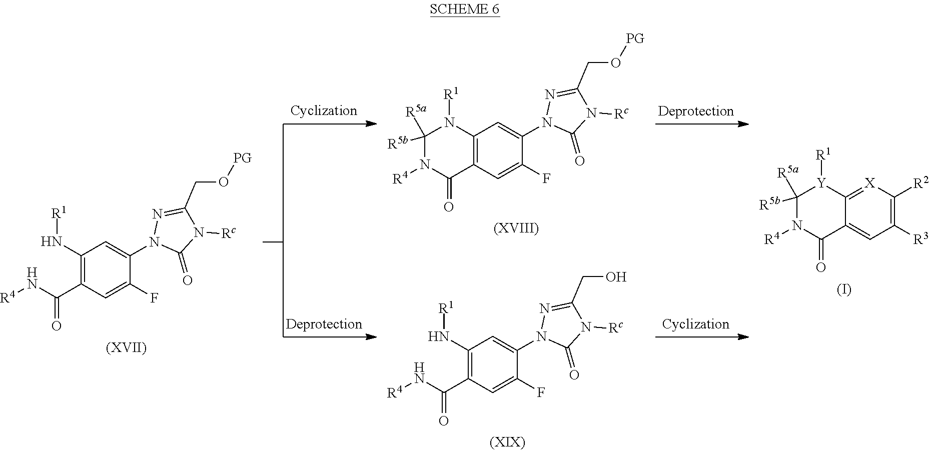

According to SCHEME 6, a compound of the formula (XVII) is reacted with an aldehyde such as formaldehyde or acetaldehyde, or an acetal such as 2,2-dimethoxypropane, 1,2,3-trioxane, or formaldehyde dimethyl acetal; in the presence or absence of a suitable acid such as p-toluenesulfonic acid (PTSA or pTsO, or tosylic acid or TsOH), and the like; in a suitable solvent such as ethanol, water, toluene, and the like; to provide a compound of formula (XVIII).

Cleavage of the benzyl protecting group of a compound of formula (XVIII), is achieved according to procedures known to one skilled in the art and employing established methodologies, such as those described in T. W. Greene and P. G. M. Wuts, “Protective Groups in Organic Synthesis,” 3 ed., John Wiley & Sons, 1999. For example, when PG is benzyl, deprotection is achieved employing Pd/C; under an H2; in a suitable solvent such as EtOH, MeOH, EtOAc, or a mixture thereof, preferably EtOH; with or without the presence HC1, preferably 0.75 equiv for 4 to 72 hrs, to provide a compound of Formula (I). Additionally, when PG is benzyl, deprotection can be employed using trifluoroacetic acid as solvent.

Alternatively, a compound of Formula (I) is prepared by first conversion of a compound of formula (XVII) to a compound of formula (XIX) using deprotection conditions detailed above; followed by conversion of a compound of (XIX) to a compound of Formula (I) using the cyclization conditions described above.

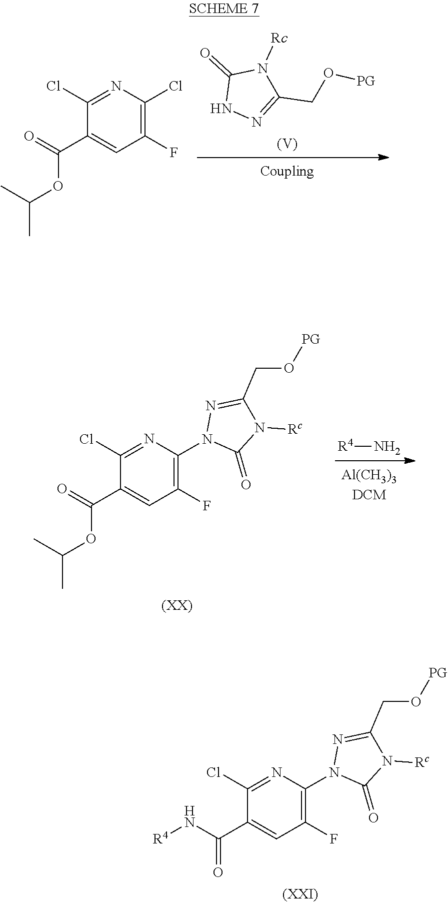

According to SCHEME 7, isopropyl 2,6-dichloro-5-fluoronicotinate is commercially available or synthetically accessible according to methods as described in WO2016097862, Pub. Jun. 23, 2016. Reaction of isopropyl 2,6-dichloronicotinate with a commercially available or synthetically accessible nucleophilic compound of formula (V), where PG is benzyl, and R^{c} is C₁₋₆alkyl; in the presence of a base such as K₂CO₃, Cs₂CO₃, NaHCO₃, triethylamine, and the like; in a suitable solvent such as dimethylsulfoxide (DMSO), DMF, THF, ACN, and the like; affords a compound of formula (XX).

A compound of formula R⁴—NH₂, where R⁴ is as defined in claim 1; is reacted with trimethyl aluminum; in a suitable solvent such as dichloromethane, toluene, or a mixture thereof; the resulting solution is combined with a compound of formula (XX); to provide a compound of formula (XXI).

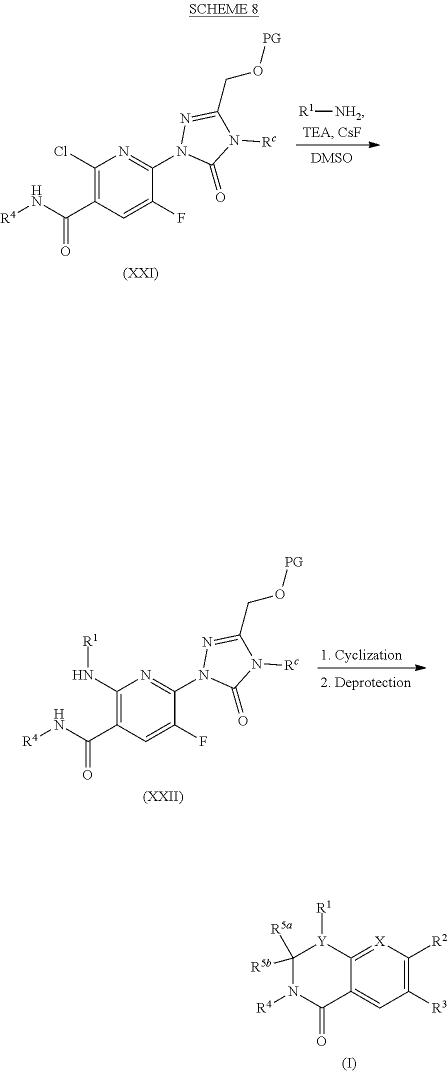

According to SCHEME 8, reaction of compound of formula (XXI) with an amine of formula R¹—NH₂, where R¹ is isopropyl, trifluoroisopropyl, tetrahydrofuranyl, cyclobutyl, and cyclopropyl; CsF; and a base such as TEA, and the like; in a suitable solvent such as dimethylsulfoxide (DMSO), dimethylformamide (DMF), or MeCN; at elevated temperatures such as 120° C., affords a compound of formula (XXII). Cyclization and deprotection of a compound of formula (XXII), according to procedures previously described such as those in SCHEME 6; provide a compound of Formula (I).

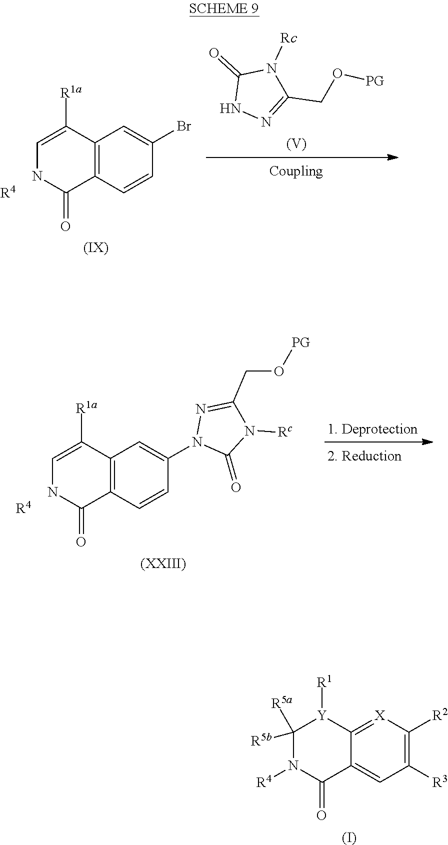

According to SCHEME 9, reaction of compound of formula (IX), where R^{1a} is isopropenyl and R⁴ is fluorophenyl; with a commercially available or synthetically accessible nucleophilic compound of formula (V); such as suitably protected triazolones, where PG is selected from: benzyl, 4-methoxy benzyl, or an alkyl or aryl silane such as TBDPS, TBS, TES, or TIPS; in the presence of a base such as K₂CO₃, Cs₂CO₃, NaHCO₃, triethylamine, and the like; in a suitable solvent such as dimethylsulfoxide (DMSO), DMF, THF, ACN, and the like; affords a compound of formula (XXIII). In a preferred method, PG is TBDPS, and R^{c} is C₁₋₆alkyl.

A compound of formula (XXIII), where PG is TBDPS, is deprotected employing conditions known to one skilled in the art, preferably with TBAF in a suitable solvent such as THF, and the like; then subsequently reduced in the presence of hydrogen gas, in the presence of a catalyst such as Palladium on carbon (Pd/C), to afford a compound of Formula (I).

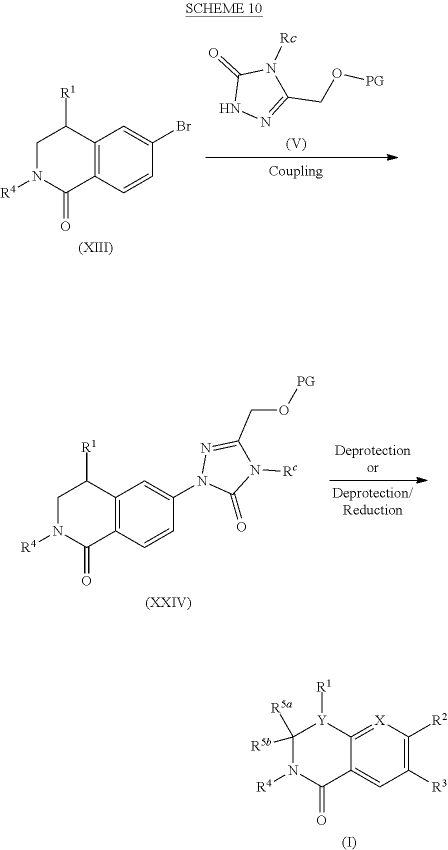

According to SCHEME 10, a compound of formula (XIII), where R¹ is isopropenyl and R⁴ is fluorophenyl or 2-chloro-6-fluoro-phenyl; is reacted with a triazolone of formula (V), where R^{c} is ethyl and PG is TBDPS, employing conditions previously described in SCHEME 9, to afford a compound of formula (XXIV). A compound of formula (XXIV) is deprotected and reduced employing conditions previously described, for example, the conditions as described in SCHEME 9, to afford a compound of Formula (I).

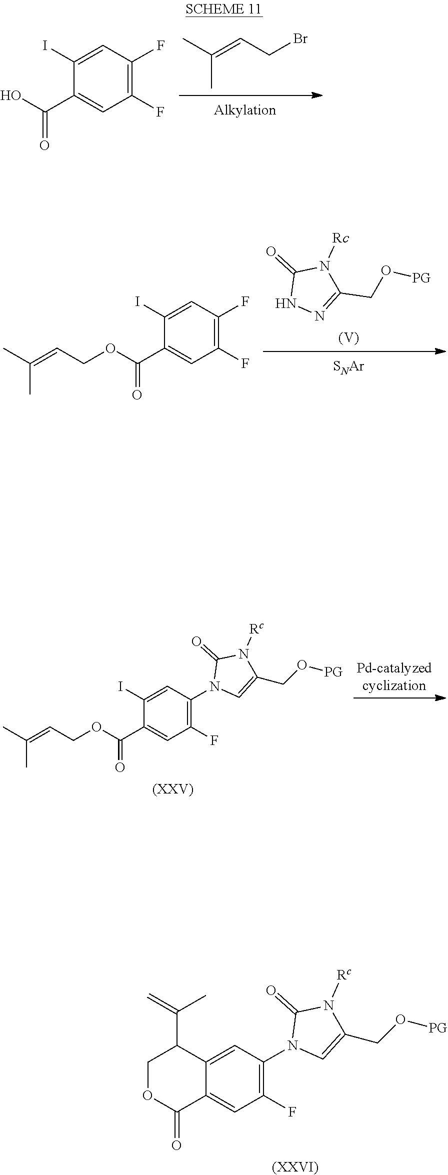

According to SCHEME 11, 4,5-difluoro-2-iodobenzoic acid is alkylated in the presence of 1-bromo-3-methylbut-2-ene, employing a base such as K₂CO₃, Cs₂CO₃, Na2CO3, triethylamine, and the like; in a suitable solvent such as dimethylsulfoxide (DMSO), DMF, THF, ACN, and the like; to afford 3-methylbut-2-en-1-yl 4,5-difluoro-2-iodobenzoate. 3-Methylbut-2-en-1-yl 4,5-difluoro-2-iodobenzoate is reacted with a compound of formula (V), where PG is Bn, in a SNAr reaction in the presence a suitable solvent such as dimethylsulfoxide (DMSO), DMF, THF, ACN, and the like; to afford a compound of formula (XXV). A compound of formula (XXV) is cyclized in the presence of a palladium catalyst such as tBu3P-Pd-G₂, Cy₂NMe, Pd(OAc)₂, and the like; in a suitable solvent such as toluene, benzene, and the like; at a temperature of about 80° C., for a period of 18 to 36 hrs; to afford a compound of formula (XXVI) either pure or as a mixture of olefinic isomers.

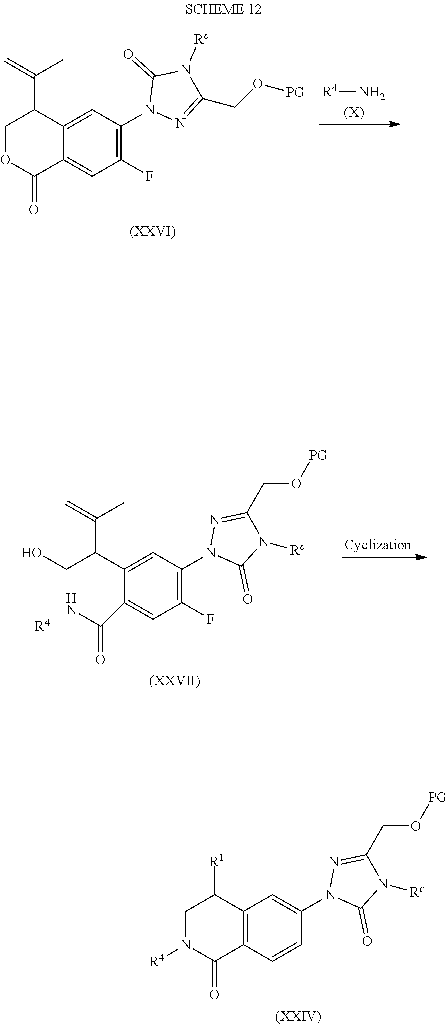

According to SCHEME 12, a compound of formula (XXVI) is reacted with the solution of a compound of formula R⁴—NH₂ (where R⁴ is defined in claim 1, which has been pre-reacted with trimethyl aluminum); in a suitable solvent such as dichloromethane, toluene, or a mixture thereof; to provide a compound of formula (XXVII). A compound of formula (XXVII) is cyclized by treatment of a suitable alcohol activating agent such as MsCl, p-toluenesulfonyl chloride, and the like, in the presence of a suitable base such as triethylamine, diisopropylethylamine, K₂CO₃, Cs₂CO₃, Na₂CO₃, and the like, in the presence of DMAP; in a suitable solvent such as CH₂Cl₂, THF, DMF, and the like; at a temperature ranging from 0° C. to rt; for a period of 18 hrs; to afford a compound of formula (XXIV).

Compounds of Formula (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

The following specific examples are provided to further illustrate the invention and various preferred embodiments.

### EXAMPLES

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on either:

METHOD A. A Gilson GX-281 semi-prep-HPLC with Phenomenex Synergi C18(10 μm, 150×25 mm), or Boston Green ODS C18 (5 μm, 150×30 mm), and mobile phase of 5-99% ACN in water (with 0.225%FA) over 10 min and then hold at 100% ACN for 2 min, at a flow rate of 25 mL/min.

or

METHOD B. A Gilson GX-281 semi-prep-HPLC with Phenomenex Synergi C18(10 μm, 150×25 mm), or Boston Green ODS C18 (5 μm, 150×30 mm), and mobile phase of 5-99% ACN in water(0.1%TFA) over 10 min and then hold at 100% ACN for 2 min, at a flow rate of 25 mL/min.

or

METHOD C. A Gilson GX-281 semi-prep-HPLC with Phenomenex Synergi C18(10 μm, 150×25 mm), or Boston Green ODS C18 (5 μm, 150×30 mm), and mobile phase of 5-99% ACN in water(0.05%HC1) over 10 min and then hold at 100% ACN for 2 min, at a flow rate of 25 mL/min.

or

METHOD D. a Gilson GX-281 semi-prep-HPLC with Phenomenex Gemini C18 (10 μm, 150×25 mm), AD(10 μm, 250 mm×30 mm), or Waters XBridge C18 column (5 μm, 150×30 mm), mobile phase of 0-99% ACN in water (with 0.05% ammonia hydroxide v/v) over 10 min and then hold at 100% ACN for 2 min, at a flow rate of 25 mL/min.

or

METHOD E. a Gilson GX-281 semi-prep-HPLC with Phenomenex Gemini C18 (10 μm, 150×25 mm), or Waters XBridge C18 column (5 μm, 150×30 mm), mobile phase of 5-99% ACN in water(10 mM NH₄HCO₃) over 10 min and then hold at 100% ACN for 2 min, at a flow rate of 25 mL/min.

Preparative supercritical fluid high performance liquid chromatography (SFC) was performed either on a Thar 80 Prep-SFC system, or Waters 80Q Prep-SFC system from Waters. The ABPR was set to 100bar to keep the CO₂ in SF conditions, and the flow rate may verify according to the compound characteristics, with a flow rate ranging from 50 g/min to 70 g/min. The column temperature was ambient temperature

Mass spectra (MS) were obtained on a SHIMADZU LCMS-2020 MSD or Agilent 1200\G6110A MSD using electrospray ionization (ESI) in positive mode unless otherwise indicated. Calculated (calcd.) mass corresponds to the exact mass.

Nuclear magnetic resonance (NMR) spectra were obtained on Bruker model AVIII 400 spectrometers. Definitions for multiplicity are as follows: s=singlet, d=doublet, t=triplet, q=quartet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 17.1 (CambridgeSoft Corp., Cambridge, MA) or OEMetaChem V1.4.0.4 (Open Eye).

Compounds designated as R* or S* are enantiopure compounds where the absolute configuration was not determined.

### Example 1

### 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one

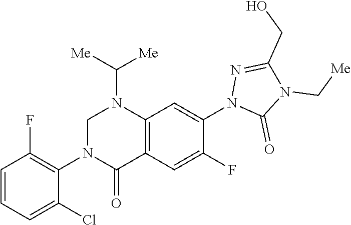 [SMILES: CCn1c(CO)nn(-c2cc3c(cc2F)C(=O)N(c2c(F)cccc2Cl)CN3C(C)C)c1=O]

Step A. 2-(Benzyloxy)acetohydrazide. To a solution of ethyl 2-(benzyloxy)acetate (55 g, 283.17 mmol) in EtOH (500 mL) was added NH₂NH₂.H₂O (28.3 g, 566 mmol, 27.5 mL). The mixture was heated reflux at 78° C. stirred for 6 hr. The reaction mixture was concentrated under reduced pressure to get the title product (52 g, crude) was obtained as a colorless oil, which was used directly to next step without further purification.

Step B. 3-((Benzyloxy)methyl)-4-ethyl-1H-1,2,4-triazol-5(4H)-one. To a solution of 2-(benzyloxy)acetohydrazide (52 g, 288 mmol) in H₂O (500 mL) was added dropwise isocyanatoethane (25.1 g, 346 mmol, 27.9 mL) at 0° C. After addition, the mixture was stirred at 25° C. for 12 hr. To the mixture was added H₂O (20 mL), and an aqueous solution of NaOH (57.7 g, 1.44 mol, in 120 mL of H₂O). The mixture was stirred at 95° C. for 12 hr. The reaction mixture was quenched with HCl (12M) at 0° C. and adjusted to pH to 6. The solid was filtered and dried under reduced pressure to get the title product (61 g, 261 mmol, 91% yield) as a white solid. ¹H NMR (400 MHz, CDCl₃) δ=9.23-9.09 (m, 1H), 7.41-7.31 (m, 5H), 4.58-4.53 (m, 2H), 4.45-4.42 (m, 2H), 3.82-3.75 (m, 2H), 1.33-1.29 (m, 3H).

Step C. 4-Bromo-5-fluoro-2-(isopropylamino)benzonitrile. To a solution of 4-bromo-2,5-difluorobenzonitrile (10 g, 45.9 mmol) in NMP (50 mL) was added DIPEA (8.89 g, 68.8 mmol, 12.0 mL) and propan-2-amine (4.07 g, 68.8 mmol, 5.91 mL). The mixture was stirred at 100° C. for 24 hr. The reaction mixture was diluted with water (50 mL) and extracted with ethyl acetate (50 mL×3). The combined organic layers were washed with brine (20 mL), dried over Na₂SO₄, filtered and concentrated under reduced pressure. The residue was purified via column chromatography (SiO₂, Petroleum ether/Ethyl acetate=1/0 to 100/1) to get the title product (9.8 g, 37.7 mmol, 83% yield) as a yellow solid. MS (ESI): mass calcd. for C₁₀H₁₀BrFN₂, 256.0; m/z found, 257.1 [M+H]⁺. ¹H NMR (400 MHz, CDCl₃) δ=7.14 (d, J=7.6 Hz, 1H), 6.84 (d, J=5.6 Hz, 1H), 4.28 (s, 1H), 3.66-3.61 (m, 1H), 1.27 (d, J=6.4 Hz, 6H).

Step D. 4-(3-((Benzyloxy)methyl)-4-ethyl-5-oxo-4,5-dihydro-1H-1,2,4-triazol-1-yl)-5-fluoro-2-(isopropylamino)benzonitrile. A mixture of 4-bromo-5-fluoro-2-(isopropylamino)benzonitrile (3 g, 11.7 mmol), 3-((benzyloxy)methyl)-4-ethyl-1H-1,2,4-triazol-5(4H)-one (3.27 g, 14.0 mmol), Cs₂CO₃ (6.84 g, 21.0 mmol), KI (1.36 g, 8.17 mmol), (1S,2S)-N1,N2-dimethylcyclohexane-1,2-diamine (995 mg, 7.00 mmol) and CuI (1.11 g, 5.83 mmol) and in dioxane (120 mL) was degassed and purged with N₂ for 3 times. The mixture was stirred at 100° C. for 16 hr under N₂ atmosphere. The reaction mixture was filtered and concentrated under reduced pressure. The residue was purified via column chromatography (SiO₂, Petroleum ether/Ethyl acetate=5:1 plate 1) to get the title product (4.2 g, 10.26 mmol, 87.91% yield, 100% purity) as yellow solid. MS (ESI): mass calcd. for C22H24FN502, 409.2; m/z found, 410.1 [M+H]⁺. ¹H NMR (400 MHz, CDCl₃) δ=7.43-7.32 (m, 5H), 7.24 (d, J=9.8 Hz, 1H), 6.93 (d, J=6.0 Hz, 1H), 4.61 (s, 2H), 4.51 (s, 2H), 4.34 (br d, J=7.6 Hz, 1H), 3.91-3.78 (m, 2H), 3.75-3.63 (m, 1H), 1.35 (t, J=7.2 Hz, 3H), 1.28 (d, J=6.4 Hz, 6H).

Step E. 4-(3-((Benzyloxy)methyl)-4-ethyl-5-oxo-4,5-dihydro-1H-1,2,4-triazol-1-yl)-5-fluoro-2-(isopropylamino)benzoic acid. To a solution of 4-(3-((benzyloxy)methyl)-4-ethyl-5-oxo-4,5-dihydro-1H-1,2,4-triazol-1-yl)-5-fluoro-2-(isopropylamino)benzonitrile (4.2 g, 10.26 mmol) in EtOH (20 mL) was added an aqueous solution of NaOH (1.86 g, 10.3 mmol). The mixture was stirred at 100° C. for 15 h. The reaction mixture was concentrated under reduced pressure to remove EtOH. The resulting residue was adjusted to pH-6 with 1 N HCl and extracted with EtOAc(80 mL×2). The combined organic layer was washed with brine(100 mL), dried over Na₂SO₄, filtered and concentrated under reduced pressure to afford the title product (4.17 g, 9.73 mmol, 95% yield) as yellow solid. ¹H NMR (400 MHz, CDCl₃) δ=7.78 (d, J=11.6 Hz, 1H), 7.46-7.30 (m, 5H), 6.94 (d, J=6.1 Hz, 1H), 4.61 (s, 2H), 4.53 (s, 2H), 3.87 (q, J=7.2 Hz, 2H), 3.70 (m, 1H), 1.37 (t, J=7.2 Hz, 3H), 1.28 (d, J=6.4 Hz, 6H).

Step F. 7-(3-((Benzyloxy)methyl)-4-ethyl-5-oxo-4,5-dihydro-1H-1,2,4-triazol-1-yl)-6-fluoro-1-isopropyl-1H-benzo[d][1,3]oxazine-2,4-dione. To a solution of 4-(3-((benzyloxy)methyl)-4-ethyl-5-oxo-4,5-dihydro-1H-1,2,4-triazol-1-yl)-5-fluoro-2-(isopropylamino)benzoic acid (2 g, 4.67 mmol) in DCM(20 mL) was added a solution of triphosgene (2.77 g, 9.34 mmol) in DCM(15 mL) at 0° C. under N₂. The mixture was stirred at 25° C. for 12 h. The reaction mixture was quenched by addition of H₂O (80 mL) at 0° C., and then extracted with DCM (50 mL×2). The combined organic layer was washed with brine (100 mL), dried over Na₂SO₄, filtered and concentrated under reduced pressure to afford the title product (2.18 g, crude) as yellow oil, which was used directly for the next step.

Step G. 4-(3-((Benzyloxy)methyl)-4-ethyl-5-oxo-4,5-dihydro-1H-1,2,4-triazol-1-yl)-N-(2-chloro-6-fluorophenyl)-5-fluoro-2-(isopropylamino)benzamide. To a solution of 2-chloro-6-fluoro-aniline (1.02 g, 7.00 mmol) in toluene(50 mL) was added AlMe₃ (2 M, 7.00 mL) at 0° C. under N₂, the mixture was stirred at 25° C. for 0.5 h. Then a solution of 7-(3-((benzyloxy)methyl)-4-ethyl-5-oxo-4,5-dihydro-1H-1,2,4-triazol-1-yl)-6-fluoro-1-isopropyl-1H-benzo[d][1,3]oxazine-2,4-dione (2.18 g, crude) in toluene(20 mL) was added. The mixture was stirred at 50° C. for 12 h. The reaction mixture was quenched by addition of 1 N HCl (30 mL) at 0° C., and then extracted with EtOAc (30 mL×3). The combined organic layer was washed with brine (40 mL×1), dried over Na₂SO₄, filtered and concentrated under reduced pressure. The residue was purified by column chromatography (SiO₂, Petroleum ether : Ethyl acetate=1:0 to 3:1) to afford the title product (1.8 g, 3.24 mmol, 69.32% yield) as yellow solid. ¹H NMR (400 MHz, CDCl₃) δ=7.78 (d, J=11.6 Hz, 1H), 7.46-7.30 (m, 5H), 6.94 (d, J=6.1 Hz, 1H), 4.61 (s, 2H), 4.53 (s, 2H), 3.87 (q, J=7.2 Hz, 2H), 3.70 (m, 1H), 1.37 (t, J=7.2 Hz, 3H), 1.28 (d, J=6.4 Hz, 6H).

Step H. 7-(3-((Benzyloxy)methyl)-4-ethyl-5-oxo-4,5-dihydro-1H-1,2,4-triazol-1-yl)-3-(2-chloro-6-fluorophenyl)-6-fluoro-1-isopropyl-2,3-dihydroquinazolin-4(1H)-one.

A mixture of 4-(3-((benzyloxy)methyl)-4-ethyl-5-oxo-4,5-dihydro-1H-1,2,4-triazol-1-yl)-N-(2-chloro-6-fluorophenyl)-5-fluoro-2-(isopropylamino)benzamide (60 mg, 108 μmol) in EtOH (5 mL) was added HCHO (88 mg, 1.08 mmol, 80 μL, 37% purity), and then the mixture was stirred at 80° C. for 16 hr under N₂ atmosphere. The mixture was poured into water (25 mL). The aqueous phase was extracted with ethyl acetate (30 mL×2). The combined organic phase was dried with anhydrous Na₂SO₄, filtered and concentrated in vacuum. The residue was purified by flash silica gel chromatography(SiO₂, Petroleum ether:Ethyl acetate=1:1.) to get the title product (53 mg, 93.3 μmol, 86% yield) as a colorless solid. ¹H NMR (400 MHz, CDCl₃) δ=7.95-7.89 (m, 1H), 7.43-7.28 (m, 7H), 7.22-7.19 (m, 1H), 7.17-7.11 (m, 1H), 4.83-4.75 (m, 2H), 4.62 (s, 2H), 4.53 (s, 2H), 4.07-3.99 (m, 1H), 3.87 (d, J=7.3 Hz, 2H), 1.37 (t, J=7.2 Hz, 3H), 1.31 (d, J=6.6 Hz, 3H), 1.25 (d, J=6.6 Hz, 3H).

Step I. 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one. A mixture of 7-(3-((benzyloxy)methyl)-4-ethyl-5-oxo-4,5-dihydro-1H-1,2,4-triazol-1-yl)-3-(2-chloro-6-fluorophenyl)-6-fluoro-1-isopropyl-2,3-dihydroquinazolin-4(1H)-one (53 mg, 93.3 μmol) in EtOH (5 mL) was added Pd/C (5 mg, 10% purity) and HCl (12 M, 5.7 μL). The mixture was stirred at 25° C. for 16 hr under H₂ (15 psi) atmosphere Pd/C (1 mg, 10% purity) was added to the mixture. The mixture was stirred at 25° C. for 5 hr under H₂ (15 psi). The mixture was filtered and concentrated under reduced pressure. The residue was purified by prep-HPLC (METHOD A) to get the title product (11.4 mg, 23.6 μmol, 25% yield) was obtained as a white solid. MS (ESI): mass calcd. for C₂₂H₂₂ClF₂N₅O₃, 477.2; m/z found, 478.2 [M+H]⁺. ¹H NMR (400 MHz, CDCl₃) δ=7.94-7.89 (m, 1H), 7.32 (br d, J=2.9 Hz, 2H), 7.24-7.20 (m, 1H), 7.18-7.11 (m, 1H), 4.79 (d, J=3.4 Hz, 2H), 4.68 (br s, 2H), 4.07-3.98 (m, 1H), 3.95-3.88 (m, 2H), 2.17-2.08 (m, 1H), 1.43 (t, J=7.2 Hz, 3H), 1.33-1.24 (m, 6H).

### Example 2

### 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2-methyl-2,3-dihydroquinazolin-4(1H)-one

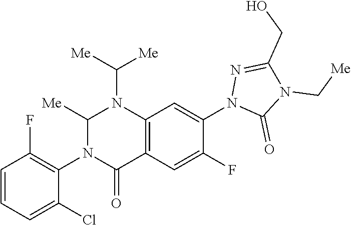 [SMILES: CCn1c(CO)nn(-c2cc3c(cc2F)C(=O)N(c2c(F)cccc2Cl)C(C)N3C(C)C)c1=O]

Step A: 7-(3-((Benzyloxy)methyl)-4-ethyl-5-oxo-4,5-dihydro-1H-1,2,4-triazol-1-yl)-3-(2-chloro-6-fluorophenyl)-6-fluoro-1-isopropyl-2-methyl-2,3-dihydroquinazolin-4(1H)-one. A mixture of 4-(3-((benzyloxy)methyl)-4-ethyl-5-oxo-4,5-dihydro-1H-1,2,4-triazol-1-yl)-N-(2-chloro-6-fluorophenyl)-5-fluoro-2-(isopropylamino)benzamide (100 mg, 179.86 μmol) in EtOH (10 mL) was added acetaldehyde (158 mg, 3.60 mmol, 202 μL), and then the mixture was stirred at 80° C. for 24 hr under N₂ atmosphere. Acetaldehyde (79 mg, 1.80 mmol, 101 μL) was added to the mixture and the mixture was stirred at 80° C. for 12 hr. Acetaldehyde (79 mg, 1.80 mmol, 101 μL) was added to the mixture and the mixture was stirred at 80° C. for 48 hr. The mixture was poured into water (30 mL). The aqueous phase was extracted with ethyl acetate (30 mL×2). The combined organic phase was dried with anhydrous Na₂SO₄, filtered and concentrated in vacuum. The residue was purified by flash silica gel chromatography(SiO₂, Petroleum ether : Ethyl acetate=1:1) to get the title product (59 mg, 101 μmol, 56% yield) as a white solid. MS (ESI): mass calcd. for C₃₀H₃₀ClF₂N₅O₃, 581.2; m/z found, 582.4 [M+H]⁺.

Step B: 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2-methyl-2,3-dihydroquinazolin-4(1H)-one. The title compound was prepared in a manner analogous to Example 1, Step I, except using 7-(3-((benzyloxy)methyl)-4-ethyl-5-oxo-4,5-dihydro-1H-1,2,4-triazol-1-yl)-3-(2-chloro-6-fluorophenyl)-6-fluoro-1-isopropyl-2-methyl-2,3-dihydroquinazolin-4(1H)-one instead of 7-(3-((benzyloxy)methyl)-4-ethyl-5-oxo-4,5-dihydro-1H-1,2,4-triazol-1-yl)-3-(2-chloro-6-fluorophenyl)-6-fluoro-1-isopropyl-2,3-dihydroquinazolin-4(1H)-one in Step A. MS (ESI): mass calcd. for C₂₃H₂₄ClF₂N₅O₃, 491.2; m/z found, 492.1 [M+H]⁺. ¹H NMR (400 MHz, CDCl₃) δ=7.89 (d, J=10.8 Hz, 1H), 7.37-7.30 (m, 2H), 7.18 (s, 1H), 7.15-7.12 (m, 1H), 5.11-5.02 (m, 1H), 4.68 (d, J=5.6 Hz, 2H), 4.12-3.98 (m, 1H), 3.91 (q, J=7.3 Hz, 2H), 2.33-2.21 (m, 1H), 1.45-1.24 (m, 12H).

### Example 3

### 3-(2-Chloro-6-fluorophenyl)-7-(4-ethyl-3-(hydroxymethyl)-5 -oxo-4,5-dihydro-1H-1,2,4-triazol-1-yl)-6-fluoro-1-isopropyl-2,2-dimethyl-2,3-dihydroquinazolin-4 (1H)-one

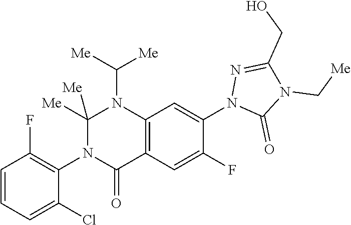 [SMILES: CCn1c(CO)nn(-c2cc3c(cc2F)C(=O)N(c2c(F)cccc2Cl)C(C)(C)N3C(C)C)c1=O]

Step A: 7-(3-((benzyloxy)methyl)-4-ethyl-5-oxo-4,5-dihydro-1H-1,2,4-triazol-1-yl)-3-(2-chloro-6-fluorophenyl)-6-fluoro-1-isopropyl-2,2-dimethyl-2,3-dihydroquinazolin-4(1H)-one. To a solution of 4-(3-((benzyloxy)methyl)-4-ethyl-5-oxo-4,5-dihydro-1H-1,2,4-triazol-1-yl)-N-(2-chloro-6-fluorophenyl)-5-fluoro-2-(isopropylamino)benzamide (100 mg, 179 μmol) in EtOH (10 mL) was added Pd/C (10 mg, 10% purity) and HCl (12 M, 11 μL). The mixture was stirred at 25° C. under H₂ (15 psi) for 12 h. The reaction mixture was filtered and concentrated under reduced pressure to get the title product (80 mg, 160 μmol, 89% yield, 93% purity) as yellow solid.

Step B: 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,2-dimethyl-2,3-dihydroquinazolin-4(1H)-one. To a solution of N-(2-chloro-6-fluorophenyl)-4-(4-ethyl-3-(hydroxymethyl)-5-oxo-4,5-dihydro-1H-1,2,4-triazol-1-yl)-5-fluoro-2-(isopropylamino)benzamide (28 mg, 60 μmol) in 2,2-dimethoxypropane (1.42 g, 13.63 mmol, 1.67 mL) was added a solution of 4-methylbenzenesulfonic acid (1 mg, 6 μmol) in toluene (1.5 mL), the mixture was stirred at 80° C. for 16 h in a sealed tube. The reaction mixture was quenched by addition H₂O (10 mL) at 0° C., and then extracted with EtOAc (10 mL×2). The combined organic layers were washed with brine (10 mL), dried over Na₂SO₄, filtered and concentrated under reduced pressure. The residue was purified by prep-HPLC (METHOD D) to give the title compound as white solid. MS (ESI): mass calcd. for C₂₄H₂₆ClF₂N₅O₃, 505.2; m/z found, 506.1 [M+H]⁺. ¹H NMR (400 MHz, CDCl₃) δ=7.88 (d, J=10.8 Hz, 1H), 7.43 (d, J=6.2 Hz, 1H), 7.36-7.29 (m, 2H), 7.19-7.07 (m, 1H), 4.69 (br s, 2H), 4.15 (td, J=6.7, 13.7 Hz, 1H), 3.92 (q, J=7.2 Hz, 2H), 2.19-2.10 (m, 1H), 1.56 (s, 6H), 1.43 (t, J=7.2 Hz, 3H), 1.30 (dd, J=6.8, 12.2 Hz, 6H).

### Example 4

### 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropylquinazoline-2,4(1H,3H)-dione

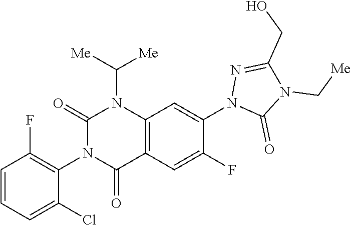 [SMILES: CCn1c(CO)nn(-c2cc3c(cc2F)c(=O)n(-c2c(F)cccc2Cl)c(=O)n3C(C)C)c1=O]

To a solution of N-(2-chloro-6-fluorophenyl)-4-(4-ethyl-3-(hydroxymethyl)-5-oxo-4,5-dihydro-1H-1,2,4-triazol-1-yl)-5-fluoro-2-(isopropylamino)benzamide (40 mg, 86 μmol) in THF (2 mL) was added triphosgene (51.0 mg, 172 μmol). The mixture was stirred at 25° C. for 1 h. The reaction mixture was concentrated under reduced pressure and kept at 0-5° C. The residue was purified by prep-HPLC (METHOD D) get the title product (10 mg, 20 μmol, 23% yield, 97% purity) as white solid. MS (ESI): mass calcd. for C₂₂H₂₀ClF₂N₅O₄, 491.1; m/z found, 492.1 [M+H]⁺. ¹H NMR (400 MHz, CDCl₃) δ=8.20 (br d, J=10.3 Hz, 1H), 7.74 (d, J=6.0 Hz, 1H), 7.25-7.20 (m, 1H), 7.09-7.02 (m, 2H), 4.79-4.67 (m, 3H), 3.98-3.89 (m, 2H), 2.15 (s, 1H), 1.61 (d, J=7.0 Hz, 6H), 1.44 (t, J=7.2 Hz, 3H).

### Example 5

### 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,3-dihydropyrido[2,3-d]pyrimidin-4(1H)-one

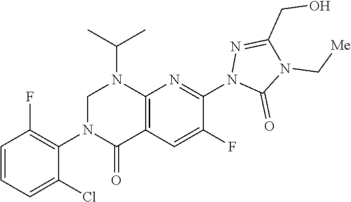 [SMILES: CCn1c(CO)nn(-c2nc3c(cc2F)C(=O)N(c2c(F)cccc2Cl)CN3C(C)C)c1=O]

Step A. 2,6-Dichloro-5-fluoronicotinoyl chloride. To a solution of 2,6-dichloro-5-fluoronicotinic acid (20 g, 95 mmol) in THF (200 mL) was added (COCl)₂ (12.7 g, 10.0 mmol, 8.75 mL) and DMF (69.6 mg, 952 μmol, 73 μL) at 0° C. dropwise. The mixture was stirred at 0° C. for 30 min, then warmed to 25° C., and stirred for 1 h. The reaction mixture was concentrated under reduced pressure to afford desired product (21.7 g, crude) as a colorless oil, which was used without further purification.

Step B. Isopropyl 2,6-dichloro-5-fluoronicotinate. To a mixture of propan-2-ol (8.56 g, 142 mmol, 10.9 mL) and pyridine solution (9.02 g, 114 mmol, 9.20 mL in 200 mL THF) was added a solution of 2,6-dichloro-5-fluoronicotinoyl chloride (21.7 g, 96.0 mmol)) in THF (50 mL) at 0° C. The mixture was stirred at 25° C. for 1 h. The mixture was poured into water (300 mL). The aqueous phase was extracted with ethyl acetate (300 mL). The combined organic phase was dried with anhydrous Na₂SO₄, filtered and concentrated in vacuum. The residue was purified by column chromatography (SiO₂, Petroleum ether/Ethyl acetate=1/1 to 10:1) to afford the title compound (21 g, 82.48 mmol, 86.82% yield, 99% purity). MS (ESI): mass calcd. for C₉H₈Cl₂FNO₂, 250.1; m/z found, 252.0 [M+H]⁺. ¹H NMR (400 MHz, CDCl₃) δ=7.97-7.95 (d, J=7.2 Hz, 1H), 5.32-5.25 (m, 1H), 1.58-1.39 (m, 6H).

Step C. Isopropyl 6-(3-((benzyloxy)methyl)-4-ethyl-5-oxo-4,5-dihydro-1H-1,2,4-triazol-1-yl)-2-chloro-5-fluoronicotinate. To a mixture of isopropyl 2,6-dichloro-5-fluoronicotinate (4 g, 15.87 mmol) in DMSO (40 mL) was added 3-((benzyloxy)methyl)-4-ethyl-1H-1,2,4-triazol-5(4H)-one (3.89 g, 16.66 mmol) and K₂CO₃ (3.29 g, 23.80 mmol). The mixture was stirred at 80° C. for 3 hr. The mixture was diluted with H₂O (30 mL) and extracted with EtOAc (50 mL*3). The combined organic layers were washed with brine (100 mL), dried over Na₂SO₄, filtered and concentrated under reduced pressure. The residue was purified by column chromatography (SiO₂, Petroleum ether/Ethyl acetate=1/0 to 1:1) to afford the title compound (5.7 g, 12.67 mmol, 79.86% yield). MS (ESI): mass calcd. for C₂₁H₂₂ClFN₄O₄, 448.1; m/z found, 449.2 [M+H]⁺. ¹H NMR (400 MHz, CDCl₃) δ=8.10 (d, J=8.8 Hz, 1H), 7.43-7.31 (m, 5H), 5.30 (td, J=6.3, 12.5 Hz, 1H), 4.61 (s, 2H), 4.54 (s, 2H), 3.85 (q, J=7.2 Hz, 2H), 1.41 (d, J=6.2 Hz, 6H), 1.37-1.31 (m, 3H).

Step D. 6-(3-((Benzyloxy)methyl)-4-ethyl-5-oxo-4,5-dihydro-1H-pyrazol-1-yl)-2-chloro-N-(2-chloro-6-fluorophenyl)-5-fluoronicotinamide. To a solution of isopropyl 6-(3-((benzyloxy)methyl)-4-ethyl-5-oxo-4,5-dihydro-1H-1,2,4-triazol-1-yl)-2-chloro-5-fluoronicotinate (200 mg, 0.466 mmol) and 2-chloro-6-fluoro-aniline (129 mg, 0.891 mmol) in DCM (2 mL) cooled to 0° C. was added trimethylaluminum (2M in THF, 0.091 mL, 1.8 mmol). The reaction was stirred at 0° C. for 30 min and 60° C. for 18 hr. The reaction mixture was quenched by addition of MeOH (1 mL) at 0° C., concentrated, and purified by column chromatography (SiO₂, heptane:EtOAc 1:0 to 1:1) to afford the title product (189 mg, 0.354 mmol, 79%) as a white solid. MS (ESI): mass calcd. for C₂₄H₁₉Cl₂F₂N₅O₃, 534.1; m/z found, 534.1 [M+H]⁺.

Step E. 6-(3-((Benzyloxy)methyl)-4-ethyl-5-oxo-4,5-dihydro-1H-1,2,4-triazol-1-yl)-N-(2-chloro-6-fluorophenyl)-5-fluoro-2-(isopropylamino)nicotinamide. To a solution of 6-(3-((benzyloxy)methyl)-4-ethyl-5-oxo-4,5-dihydro-1H-pyrazol-1-yl)-2-chloro-N-(2-chloro-6-fluorophenyl)-5-fluoronicotinamide (189 mg, 0.354 mmol) in DMSO (18.9 mL) was added isopropylamine (209 mg, 3.54 mmol), TEA (0.147 mL, 1.06 mmol), and CsF (161 mg, 1.06 mmol). The reaction was heated in a microwave to 130° C. for 3 hrs. The reaction was diluted in EtOAc (100 mL) and washed with brine (3×75 mL). The organics were dried, filtered and concentrated. The residue was purified via column chromatography (SiO₂, Heptane/Ethyl acetate=1/0 to 100/1) to afford the title product (108 mg, 0.194 mmol, 55%) as a white solid. MS (ESI): mass calcd. for C₂₇H₂₇ClF₂N₆O₃, 557.0; m/z found, 557.1 [M+H]⁺. ¹H NMR (400 MHz, CDCl₃) δ 7.93 (br d, J=6.85 Hz, 1H), 7.87 (d, J=9.78 Hz, 1H), 7.63 (s, 1H), 7.27-7.41 (m, 6H), 7.20-7.25 (m, 1H), 7.09-7.19 (m, 1H), 4.61 (s, 2H), 4.53 (s, 2H), 4.22-4.35 (m, 1H), 3.79 (q, J=7.34 Hz, 2H), 1.21-1.31 (m, 9H).

Step F. 7-(3-((Benzyloxy)methyl)-4-ethyl-5-oxo-4,5-dihydro-1H-1,2,4-triazol-1-yl)-3-(2-chloro-6-fluorophenyl)-6-fluoro-1-isopropyl-2,3-dihydropyrido[2,3-d]pyrimidin-4(1H)-one. To a solution of 6-(3-((benzyloxy)methyl)-4-ethyl-5-oxo-4,5-dihydro-1H-1,2,4-triazol-1-yl)-N-(2-chloro-6-fluorophenyl)-5-fluoro-2-(isopropylamino)nicotinamide (43 mg, 0.077 mmol) in toluene (10 mL) was added p-toluene sulfonic acid monohydrate (7.3 mg, 0.039 mmol) and 1,3,5-trioxane (300 mg, 3.33 mmol). The reaction was heated to reflux for 2 hrs. The reaction was diluted in EtOAc (75 mL) and washed with brine (2×30 mL). The organics were dried, filtered, and concentrated. The residue was purified via column chromatography (SiO₂, Heptane/Ethyl acetate=1/0 to 2/3) to afford the title product (108 mg, 0.194 mmol, 55%) as a white solid. MS (ESI): mass calcd. for C₂₈H₂₇ClF₂N₆O₃, 569.0; m/z found, 569.3 [M+H]⁺. ¹H NMR (400 MHz, CDCl₃) δ 8.14 (d, J=9.29 Hz, 1H), 7.30-7.43 (m, 7H), 7.07-7.21 (m, 1H), 4.92-5.00 (m, J=6.80, 6.80, 13.70 Hz, 1H), 4.79-4.92 (m, 2H), 4.61 (s, 2H), 4.54 (s, 2H), 3.86 (q, J=7.17 Hz, 2H), 1.36 (t, J=7.34 Hz, 3H), 1.28-1.31 (m, J=6.80 Hz, 3H), 1.23 (d, J=6.85 Hz, 3H).

Step G. 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,3-dihydropyrido[2,3-d]pyrimidin-4(1H)-one. A solution of 7-(3-((benzyloxy)methyl)-4-ethyl-5-oxo-4,5-dihydro-1H-1,2,4-triazol-1-yl)-3-(2-chloro-6-fluorophenyl)-6-fluoro-1-isopropyl-2,3-dihydropyrido[2,3-d]pyrimidin-4(1H)-one (44 mg, 0.077 mmol) in TFA (2 mL) was heated to 70° C. for 18 hrs. The reaction was concentrated and diluted with DCM (4 mL) and 1 N NaOH (4 mL). The aqueous phase was extracted with DCM (3×4 mL). The organics were dried, filtered, and concentrated. The residue was purified via column chromatography (SiO₂, eptane/Ethyl acetate=1/0 to 1/5) to afford the title product (17 mg, 0.036 mmol, 37%) as a white solid. MS (ESI): mass calcd. for C₂₁H₂₁ClF₂N₆O₃, 478.9; m/z found, 479.3 [M+H]⁺. ¹H NMR (400 MHz, CDCl₃) δ 8.15 (d, J=9.29 Hz, 1H), 7.31-7.37 (m, 2H), 7.12-7.20 (range, 1H), 4.96 (td, J=6.66, 13.57 Hz, 1H), 4.77-4.90 (m, 2H), 4.68 (s, 2H), 3.91 (q, J=7.17 Hz, 2H), 1.42 (t, J=7.09 Hz, 3H), 1.27-1.31 (m, 3H), 1.25 (s, 1H), 1.23 (d, J=6.36 Hz, 3H).

### Example 6

### 3-(2-Chloro-6-fluorophenyl)-7-(4-ethyl-3-(hydroxymethyl)-5-oxo-4,5-dihydro-1H-1,2,4-triazol-1-yl)-6-fluoro-1-(tetrahydro-2H-pyran-4-yl)-2,3-dihydropyrido[2,3-d]pyrimidin-4(1H)-one

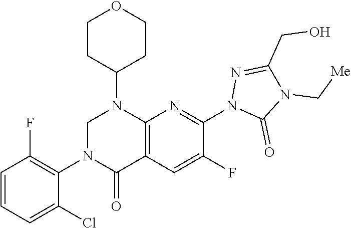 [SMILES: CCn1c(CO)nn(-c2nc3c(cc2F)C(=O)N(c2c(F)cccc2Cl)CN3C2CCOCC2)c1=O]

Step A. 6-(3-((Benzyloxy)methyl)-4-ethyl-5-oxo-4,5-dihydro-1H-1,2,4-triazol-1-yl)-N-(2-chloro-6-fluorophenyl)-5-fluoro-2-((tetrahydro-2H-pyran-4-yl)amino)nicotinamide. The title compound was prepared according to the representative procedures of

Example 5, Step E, except using tetrahydro-2H-pyran-4-amine instead of isopropylamine. MS (ESI): mass calcd. for C₂₉H₂₉ClF₂N₆O₄, 599.0; m/z found, 599.3 [M+H]⁺. ¹ H NMR(400 MHz, CDCl₃) δ 9.83 (s, 1H), 8.24 (d, J=9.29 Hz, 1H), 7.31-7.44 (m, 6H), 7.22-7.30 (m, 2H), 7.06-7.19 (m, 1H), 4.61 (s, 3H), 4.52 (s, 2H), 4.06-4.23 (m, 2H), 3.74-3.91 (m, 5H), 3.63-3.73 (m, 2H), 1.92-2.14 (m, 4H), 1.35 (t, J=7.09 Hz, 3H).

Step B. 7-(3-((Benzyloxy)methyl)-4-ethyl-5-oxo-4,5-dihydro-1H-1,2,4-triazol-1-yl)-3-(2-chloro-6-fluorophenyl)-6-fluoro-1-(tetrahydro-2H-pyran-4-yl)-2,3-dihydropyrido[2,3-d]pyrimidin-4(1H)-one. The title compound was prepared according to the representative procedure of Example 5, Step F, except using 6-(3-((benzyloxy)methyl)-4-ethyl-5-oxo-4,5-dihydro-1H-1,2,4-triazol-1-yl)-N-(2-chloro-6-fluorophenyl)-5-fluoro-2-((tetrahydro-2H-pyran-4-yl)amino)nicotinamide instead of tetrahydro-2H-pyran-4-amine for 6-(3-((benzyloxy)methyl)-4-ethyl-5-oxo-4,5-dihydro-1H-1,2,4-triazol-1-yl)-N-(2-chloro-6-fluorophenyl)-5-fluoro-2-(isopropylamino)nicotinamide. MS (ESI): mass calcd. for C₃₀H₂₉ClF₂N₆O₄, 611.0; m/z found, 611.3 [M+H]⁺. ¹ H NMR(400 MHz, CDCl₃) δ 8.16 (d, J=8.80 Hz, 1H), 7.30-7.41 (m, 7H), 7.12-7.25 (m, 1H), 4.83-4.93 (m, 2H), 4.70-4.83 (m, 1H), 4.63 (s, 2H), 4.54 (s, 2H), 3.99-4.06 (m, 2H), 3.85 (q, J=7.17 Hz, 2H), 3.48-3.58 (m, 2H), 1.78-1.90 (m, 3H), 1.65-1.78 (m, 1H), 1.36 (t, J=7.09 Hz, 3H).

Step C. 3-(2-Chloro-6-fluorophenyl)-7-(4-ethyl-3-(hydroxymethyl)-5-oxo-4,5-dihydro-1H-1,2,4-triazol-1-yl)-6-fluoro-1-(tetrahydro-2H-pyran-4-yl)-2,3-dihydropyrido[2,3-d]pyrimidin-4(1H)-one. The title compound was prepared according to the representative procedure of Example 5, Step G, except using 7-(3-((benzyloxy)methyl)-4-ethyl-5-oxo-4,5-dihydro-1H-1,2,4-triazol-1-yl)-3-(2-chloro-6-fluorophenyl)-6-fluoro-1-(tetrahydro-2H-pyran-4-yl)-2,3-dihydropyrido[2,3-d]pyrimidin-4(1H)-one instead of 7-(3-((benzyloxy)methyl)-4-ethyl-5-oxo-4,5-dihydro-1H-1,2,4-triazol-1-yl)-3-(2-chloro-6-fluorophenyl)-6-fluoro-1-isopropyl-2,3-dihydropyrido[2,3-d]pyrimidin-4(1H)-one. MS (ESI): mass calcd. for C₂₃H₂₃ClF₂N₆O₄, 520.9; m/z found, 521.3 [M+H]⁺. ¹H NMR (400 MHz, CDCl₃) δ 8.17 (d, J=9.29 Hz, 1H), 7.28-7.41 (m, 2H), 7.13-7.24 (m, 1H), 4.82-4.93 (m, 2H), 4.71-4.82 (m, 1H), 4.69 (d, J=6.36 Hz, 2H), 3.98-4.09 (m, 2H), 3.91 (q, J=7.34 Hz, 2H), 3.49-3.60 (m, 2H), 2.25 (t, J=6.36 Hz, 1H), 1.81-1.89 (m, 3H), 1.61-1.80 (m, 1H), 1.43 (t, J=7.09 Hz, 3H).

### Example 7

### 3-(2-chloro-6-fluorophenyl)-1-cyclobutyl-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one

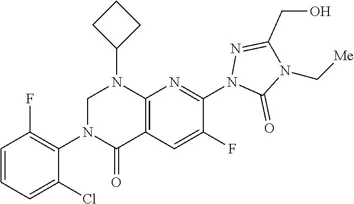 [SMILES: CCn1c(CO)nn(-c2nc3c(cc2F)C(=O)N(c2c(F)cccc2Cl)CN3C2CCC2)c1=O]

Step A. 6-(3-((Benzyloxy)methyl)-4-ethyl-5-oxo-4,5-dihydro-1H-1,2,4-triazol-1-yl)-N-(2-chloro-6-fluorophenyl)-2-(cyclobutylamino)-5-fluoronicotinamide

The title compound was prepared according to the representative procedures of

Example 5, Step E, except using cyclobutylamine instead of isopropylamine. MS (ESI): mass calcd. for C₂₈H₂₇ClF₂N₆O₃, 569.0; m/z found, 569.3 [M+H]⁺. ¹H NMR (400 MHz, CDCl₃) δ 8.42 (s, 1H), 8.30-8.37 (m, 1H), 8.06 (d, J=9.29 Hz, 1H), 7.29-7.44 (m, 5H), 7.17-7.25 (m, 2H), 7.05-7.15 (m, 1H), 4.60 (s, 2H), 4.52 (s, 3H), 3.52-3.74 (m, 2H), 2.29-2.47 (m, 2H), 1.80-2.00 (m, 2H), 1.64-1.77 (m, 2H), 1.06-1.20 (m, 3H).

Step B. 7-(3-((Benzyloxy)methyl)-4-ethyl-5-oxo-4,5-dihydro-1H-1,2,4-triazol-1-yl)-3-(2-chloro-6-fluorophenyl)-1-cyclobutyl-6-fluoro-2,3-dihydropyrido[2,3-d]pyrimidin-4(1H)-one. The title compound was prepared according to the representative procedure of Example 5, Step F, except using 6-(3-((benzyloxy)methyl)-4-ethyl-5-oxo-4,5-dihydro-1H-1,2,4-triazol-1-yl)-N-(2-chloro-6-fluorophenyl)-2-(cyclobutylamino)-5-fluoronicotinamide instead of 6-(3-((benzyloxy)methyl)-4-ethyl-5-oxo-4,5-dihydro-1H-1,2,4-triazol-1-yl)-N-(2-chloro-6-fluorophenyl)-5-fluoro-2-(isopropylamino)nicotinamide. MS (ESI): mass calcd. for C₂₉H₂₇ClF₂N₆O₃, 581.0; m/z found, 581.3 [M+H]⁺. ¹H NMR (400 MHz, CDCl₃) δ 8.13 (d, J=9.29 Hz, 1H), 7.29-7.46 (m, 7H), 7.08-7.21 (m, 1H), 5.25-5.33 (m, 1H), 4.82-4.83 (m, 1H), 4.87 (s, 1H), 4.68-4.82 (m, 1H), 4.62 (s, 2H), 4.54 (s, 2H), 4.07-4.18 (m, 1H), 3.78-3.92 (m, 2H), 2.26-2.45 (m, 2H), 2.06-2.25 (m, 2H), 1.63-1.82 (m, 2H), 1.36 (t, J=7.09 Hz, 3H), 1.18-1.30 (m, 2H).

Step C. 3-(2-chloro-6-fluorophenyl)-1-cyclobutyl-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one. The title compound was prepared according to the representative procedure of Example 5, Step G, except using 7-(3-((benzyloxy)methyl)-4-ethyl-5-oxo-4,5-dihydro-1H-1,2,4-triazol-1-yl)-3-(2-chloro-6-fluorophenyl)-1-cyclobutyl-6-fluoro-2,3-dihydropyrido[2,3-d]pyrimidin-4(1H)-one instead of 7-(3-((benzyloxy)methyl)-4-ethyl-5-oxo-4,5 -dihydro-1H-1,2,4-triazol-1-yl)-3 -(2-chloro-6-fluorophenyl)-6-fluoro-1-isopropyl-2,3-dihydropyrido[2,3-d]pyrimidin-4(1H)-one. MS (ESI): mass calcd. for C₂₂H₂₁ClF₂N₆O₃, 490.9; m/z found, 591.3 [M+H]⁺. ¹H NMR (400 MHz, CDCl₃) δ 8.14 (d, J=9.29 Hz, 1H), 7.31-7.37 (m, 2H), 7.10-7.24 (m, 1H), 4.85-4.91 (m, 2H), 4.72-4.84 (m, 1H), 4.68 (d, J=6.36 Hz, 2H), 3.91 (q, J=7.01 Hz, 2H), 2.26-2.41 (m, 2H), 2.06-2.24 (m, 3H), 1.66-1.77 (m, 2H), 1.42 (t, J=7.34 Hz, 3H).

### Example 8

### (S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(1,1,1-trifluoropropan-2-yl)-2,3-dihydropyrido[2,3-d]pyrimidin-4(1H)-one

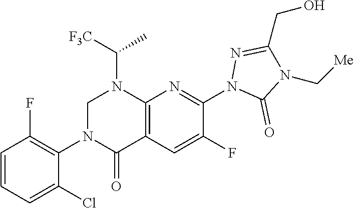 [SMILES: CCn1c(CO)nn(-c2nc3c(cc2F)C(=O)N(c2c(F)cccc2Cl)CN3[C@@H](C)C(F)(F)F)c1=O]

Step A. (S)-6-(3-((Benzyloxy)methyl)-4-ethyl-5-oxo-4,5-dihydro-1H-1,2,4-triazol-1-yl)-N-(2-chloro-6-fluorophenyl)-5-fluoro-2-((1,1,1-trifluoropropan-2-yl)amino)nicotinamide. To a solution of 6-(3-((benzyloxy)methyl)-4-ethyl-5-oxo-4,5-dihydro-1H-pyrazol-1-yl)-2-chloro-N-(2-chloro-6-fluorophenyl)-5 -fluoronicotinamide (20 mg, 0.037 mmol) in DMSO (0.5 mL) was added cesium fluoride (8 mg, 0.055 mmol), and (S)-2-amino-1,1,1-trifluoropropane (200 mg, 1.77 mmol). The reaction was heated to 120 C for 18 hrs. The reaction was diluted in EtOAc (100 mL) and washed with brine (3×75 mL). The organics were dried, filtered and concentrated. The residue was purified via column chromatography (SiO₂, Heptane/Ethyl acetate=1/0 to 3/2) to afford the title product (15.5 mg, 0.254 mmol, 68%) as a light yellow solid. MS (ESI): mass calcd. for C₂₇H₂₄ClF₅N₆O₃, 611.0; m/z found, 611.3 [M+H]⁺. ¹H NMR (400 MHz, CDCl₃) δ 8.53 (s, 1H), 8.44 (d, J=9.29 Hz, 1H), 8.15 (d, J=9.29 Hz, 1H), 7.29-7.43 (m, 5H), 7.21-7.26 (m, 2H), 7.09-7.16 (m, 1H), 5.11 (qd, J=7.32, 15.22 Hz, 1H), 4.60 (s, 2H), 4.51 (s, 2H), 3.62 (sxt, J=7.53 Hz, 2H), 1.37 (d, J=6.85 Hz, 3H), 1.13 (t, J=7.34 Hz, 3H).

Step B. (S)-7-(3-((Benzyloxy)methyl)-4-ethyl-5-oxo-4,5-dihydro-1H-1,2,4-triazol-1-yl)-3-(2-chloro-6-fluorophenyl)-6-fluoro-1-(1,1,1-trifluoropropan-2-yl)-2,3-dihydropyrido[2,3-d]pyrimidin-4(1H)-one. The title compound was prepared according to the representative procedure of Example 5, Step F, except using (S)-6-(3-((benzyloxy)methyl)-4-ethyl-5-oxo-4,5-dihydro-1H-1,2,4-triazol-1-yl)-N-(2-chloro-6-fluorophenyl)-5-fluoro-2-((1,1,1-trifluoropropan-2-y0amino)nicotinamide instead of 6-(3-((benzyloxy)methyl)-4-ethyl-5-oxo-4,5-dihydro-1H-1,2,4-triazol-1-yl)-N-(2-chloro-6-fluorophenyl)-5-fluoro-2-(isopropylamino)nicotinamide. MS (ESI): mass calcd. for C₂₈H₂₄ClF₅N₆O₃, 623.0; m/z found, 623.3 [M+H]⁺. ¹H NMR (400 MHz, CDCl₃) δ 8.20 (d, J=8.80 Hz, 1H), 7.34-7.41 (m, 6H), 7.08-7.28 (m, 2H), 5.49-5.63 (m, 1H), 4.86-5.05 (m, 2H), 4.62 (s, 2H), 4.50-4.57 (m, 2H), 3.86 (q, J=7.17 Hz, 2H), 1.49 (dd, J=7.34, 17.61 Hz, 3H), 1.36 (t, J=7.34 Hz, 3H).

Step C. (S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(1,1,1-trifluoropropan-2-yl)-2,3-dihydropyrido[2,3-d]pyrimidin-4(1H)-one. The title compound was prepared according to the representative procedure of Example 5, Step D, except using (S)-7-(3-((benzyloxy)methyl)-4-ethyl-5-oxo-4,5 -dihydro-1H-1,2,4-triazol-1-yl)-3 -(2-chloro-6-fluorophenyl)-6-fluoro-1-(1,1,1-trifluoropropan-2-yl)-2,3-dihydropyrido[2,3-d]pyrimidin-4(1H)-one instead of 7-(3-((benzyloxy)methyl)-4-ethyl-5-oxo-4,5-dihydro-1H-1,2,4-triazol-1-yl)-3-(2-chloro-6-fluorophenyl)-6-fluoro-1-isopropyl-2,3-dihydropyrido[2,3-d]pyrimidin-4(1H)-one. MS (ESI): mass calcd. for C₂₁H₁₈ClF₅N₆O₃, 532.9; m/z found, 533.1 [M+H]⁺. ¹H NMR (400 MHz, CDCl₃) δ 8.22 (br d, J=8.80 Hz, 1H), 7.38-7.58 (m, 2H), 7.25-7.38 (m, 1H), 5.55-5.71 (m, 1H), 5.13-5.30 (m, 1H), 4.97-5.12 (m, 1H), 4.62 (s, 2H), 3.82-4.02 (m, 2H), 1.51 (br dd, J=7.09, 16.63 Hz, 3H), 1.34-1.45 (m, 3H).

### Example 9

### 3-(2-Chloro-6-fluorophenyl)-1-cyclopropyl-7-(4-ethyl-3 -(hydroxymethyl)-5-oxo-4,5 -dihydro-1H-1,2,4-triazol-1-yl)-6-fluoro-2,3 -dihydropyrido[2,3-d]pyrimidin-4(1H)-one

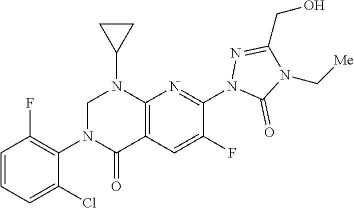 [SMILES: CCn1c(CO)nn(-c2nc3c(cc2F)C(=O)N(c2c(F)cccc2Cl)CN3C2CC2)c1=O]

Step A. 6-(3-((benzyloxy)methyl)-4-ethyl-5-oxo-4,5-dihydro-1H-1,2,4-triazol-1-yl)-N-(2-chloro-6-fluorophenyl)-2-(cyclopropylamino)-5-fluoronicotinamide.

The title compound was prepared according to the representative procedures of Example 5, Step E. MS (ESI): mass calcd. for C₂₇H₂₅ClF₂N₆O₃, 554.2; m/z found, 555.0 [M+H]⁺. ¹H NMR (400 MHz, CDCl₃) δ 8.14 (br s, 1H) 7.93 (d, J=9.29 Hz, 1H) 7.82 (br d, J=3.91 Hz, 1H) 7.31-7.42 (m, 5H) 7.13 (t, J=8.56 Hz, 1H) 4.61 (s, 2H) 4.53 (s, 2H) 3.75 (br d, J=6.85 Hz, 2H) 2.95 (br d, J=3.42 Hz, 1H) 1.25 (t, J=7.34 Hz, 3H) 0.76 (br d, J=6.85 Hz, 2H) 0.47-0.58 (m, 2H).

Step B. 7-(3-((Benzyloxy)methyl)-4-ethyl-5-oxo-4,5-dihydro-1H-1,2,4-triazol-1-yl)-3-(2-chloro-6-fluorophenyl)-1-cyclopropyl-6-fluoro-2,3-dihydropyrido[2,3-d]pyrimidin-4(1H)-one. The title compound was prepared according to the representative procedures of Example 5, Step F. MS (ESI): mass calcd. for C₂₈H₂₅ClF₂N₆O₃, 566.2; m/z found, 567.0 [M+H]⁺. ¹H NMR (400 MHz, CDCl₃) δ 8.14 (d, J=8.80 Hz, 1H) 7.36-7.40 (m, 4H) 7.33 (q, J=2.93 Hz, 3H) 7.11-7.19 (m, 1H) 4.92-4.99 (m, 1H) 4.83-4.88 (m, 1H) 4.62 (s, 2H) 4.54 (s, 2H) 3.86 (q, J=7.34 Hz, 2H) 2.62-2.73 (m, 1H) 1.64 (br s, 1H) 1.36 (t, J=7.09 Hz, 3H) 1.20-1.31 (m, 3H) 0.93-1.01 (m, 1H) 0.85-0.92 (m, 2H) 0.82 (br d, J=2.93 Hz, 2H).

Step C. 3-(2-Chloro-6-fluorophenyl)-1-cyclopropyl-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one. The title compound was prepared according to the representative procedure of Example 5, Step G. MS (ESI): mass calcd. for C₂₁H₁₉ClF₂N₆O₃, 476.1; m/z found, 477.0 [M+H]⁺.¹H NMR (400 MHz, CDCl₃) δ 7.31-7.37 (m, 2H) 7.15 (s, 1H) 4.93-4.98 (m, 1H) 4.84-4.88 (m, 1H) 4.69 (d, J=6.36 Hz, 2H) 3.91 (q, J=7.01 Hz, 2H) 2.62-2.71 (m, 1H) 2.00-2.06 (m, 1H) 1.42 (t, J=7.09 Hz, 3H) 0.81 (br s, 4H).

### Example 10

### Racemic-6-(4-Ethyl-3-(hydroxymethyl)-5-oxo-4,5-dihydro-1H-1,2,4-triazol-1-yl)-2-(3-fluorophenyl)-4-isopropyl-3,4-dihydroisoquinolin-1(2H)-one

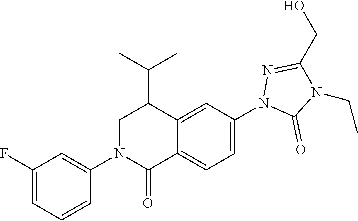 [SMILES: CCn1c(CO)nn(-c2ccc3c(c2)C(C(C)C)CN(c2cccc(F)c2)C3=O)c1=O]

Step A. 4-Ethyl-5-(hydroxymethyl)-2,4-dihydro-3H-1,2,4-triazol-3-one.

To a solution of 5-Rbenzyloxy)methyl1-4-methyl-2,4-dihydro-3H-1,2,4-triazol-3-one (8 g, 34.3 mmol, 1.0 eq.) in methanol (200 mL) was added Pd/C (2 g). The resulting mixture was maintained under hydrogen and stirred at rt for 6 h. Then the resulting mixture was filtered and the filtrate was concentrated to afford the crude product 4-ethyl-5-(hydroxymethyl)-2,4-dihydro-3H-1,2,4-triazol-3-one as a white solid (4.3 g, 88% yield). ¹H NMR (400 MHz, DMSO-d₆) δ 11.52 (s, 1H), 5.55 (t, J=5.50 Hz, 1H), 4.32 (d, J=5.50 Hz, 2H), 3.64 (q, J=6.97 Hz, 2H), 1.18 (t, J=6.97 Hz, 3H)

Step B. 5-(((tert-Butyldiphenylsilyl)oxy)methyl)-4-ethyl-2,4-dihydro-3H-1,2,4-triazol-3-one.

To a solution of 4-ethyl-5-(hydroxymethyl)-2,4-dihydro-3H-1,2,4-triazol-3-one (2.40 g, 16.8 mmol, 1.0 eq.) in DCM (50 mL) was added tert-butylchlorodiphenylsilane (6.9 mL, 25 mmol, 1.5 eq.) and imidazole (2.28 g, 33.5 mmol, 1.1 eq.). The resulting mixture was stirred at rt for overnight. The reaction mixture was quenched with water (100 mL). The resulting mixture was extracted with DCM (3×100 mL). The organic layers were combined, dried over anhydrous sodium sulfate, and concentrated. The residue was purified by silica gel chromatography (50-80% ethyl acetate/petroleum ether) to afford the 5-(((tert-butyldiphenylsily0oxy)methyl)-4-ethyl-2,4-dihydro-3H-1,2,4-triazol-3-one as a white solid (4.9 g, 76% yield). LCMS (ES-API): mass calcd. for C₂₁H₂₇N₃O₂Si, 381.2; m/z found, 382.2 [M+H]⁺. ¹H NMR (400 MHz, CDCl₃) δ 9.98 (s, 1H), 7.61-7.72 (m, 4H), 7.32-7.54 (m, 6H), 4.54 (s, 2H), 3.84 (q, J=7.34 Hz, 2H), 1.33 (t, J=7.34 Hz, 3H), 1.07 (s, 9H)

Step C. 6-Bromo-4-iodoisoquinolin-1(2H)-one. To a suspension of 6-bromoisoquinolin-1(2H)-one (2.90 g, 12.9 mmol) in anhydrous acetonitrile (50 mL) was added N-iodosuccinimide (NIS) (4.40 g, 19.4 mmol). The reaction mixture was heated and stirred under nitrogen at 80° C. for 2 h, then cooled to 25° C. The mixture was filtered through a sintered funnel, and the precipitate was collected, washed with water, and dried in vacuo to afford the desired product (3.5 g, crude, 77%) as a brown solid, which was used crude in the next step without further purification. LCMS (ES-API): mass calcd. for C₉H₅BrINO, 348.9; m/z found, 349.8 [M+H]⁺.

Step D. 6-Bromo-4-(prop-1-en-2-yl)isoquinolin-1(2H)-one. To a mixture of 6-bromo-4-iodoisoquinolin-1(2H)-one (0.7 g, 2 mmol) and Cs₂CO₃ (3.3 g, 10 mmol) in 1,4-dioxane (20 mL) and ethanol (20 mL) and water (10 mL) was added bis(triphenylphosphine)palladium(II) dichloride (0.7 g, 1 mmol) and isopropenylboronic acid pinacol ester (1.3 g, 4 mmol) respectively. The reaction mixture was degassed with nitrogen and then stirred at 25° C. for 24 h. The mixture was poured into water (50 mL). The aqueous phase was extracted with ethyl ether (100 mL) and ethyl acetate (EtOAc) (100 mL). The combined organic extract was washed with brine, dried over anhydrous Na₂SO₄, and concentrated. The residue was purified by chromatography (SiO₂, 10-70% Ethyl acetate in heptane) to afford the title compound as a white solid (0.12 g, 23% yield). LCMS (ES-API): mass calcd. for C₁₂H₁₀BrNO, 263.0; m/z found, 264.0 [M+H]⁺. ¹H NMR (400 MHz, CDCl₃) δ 11.15 (br s, 1H), 8.30 (d, J=8.42 Hz, 1H), 7.85 (d, J=1.59 Hz, 1H), 7.62 (br dd, J=1.59, 8.42 Hz, 1H), 7.06 (s, 1H), 5.37 (s, 1H), 5.09 (s, 1H), 2.11 (s, 3H) ppm.

Step E. 6-Bromo-2-(3-fluorophenyl)-4-(prop-1-en-2-yl)isoquinolin-1(2H)-one. To a mixture of 6-bromo-4-(prop-1-en-2-yl)isoquinolin-1(2H)-one (120 mg, 0.45 mmol) and 3-fluorophenylboronic acid (191 mg, 1.4 mmol) in dichloromethane (DCM) (20 mL) and triethylamine (3 mL) was added copper(II) acetate (82.5 mg, 0.45 mmol) and pyridine (0.11 mL, 1.36 mmol). The reaction mixture was stirred at 25° C. for 18 h. The mixture was filtered through a pad of silica gel and washed with ethyl acetate (100 mL). The filtrate was concentrated. The residue was purified by chromatography (SiO₂, 20-50% EtOAc in heptane) to afford the crude desired product as an oil (118 mg, 72%). LCMS (ES-API): mass calcd. for C₁₈H13BrFNO, 358.0; m/z found, 358.0 [M]⁺.

Step F: 6-(3-(((tert-Butyldiphenylsilyl)oxy)methyl)-4-ethyl-5-oxo-4,5-dihydro-1H-1,2,4-triazol-1-yl)-2-(3-fluorophenyl)-4-(prop-1-en-2-yl)isoquinolin-1(2H)-one. To a mixture of 6-bromo-2-(3-fluorophenyl)-4-(prop-1-en-2-yl)isoquinolin-1(2H)-one (114 mg, 0.32 mmol) and K₃PO₄ (338 mg, 1.6 mmol) in anhydrous 1,4-dioxane (6 mL) was added 5-(((tert-butyldiphenylsily0oxy)methyl)-4-ethyl-2,4-dihydro-3H-1,2,4-triazol-3-one (304 mg, 0.8 mmol), Cul (60 mg, 0.32 mmol), and trans-N,N′-dimethylcyclohexane-1,2-diamine (45 mg, 0.32 mmol), respectively. The mixture was slowly heated and stirred under nitrogen at 95° C. for 2 h, then cooled to 25° C. and quenched with addition of water. The mixture was extracted with ethyl acetate (50 mL×2). The combined organic extract was washed with brine, dried with anhydrous Na₂SO₄, and concentrated. The residue was purified by chromatography (20-50% EtOAc in heptane) to afford the desired product as a white solid (125 mg, 60%). LCMS (ES-API): mass calcd. for C₃₉H₃₉FN₄O₃Si, 658.3; m/z found, 659.4 [M+H]⁺.

Step G: 6-(4-Ethyl-3-(hydroxymethyl)-5-oxo-4,5-dihydro-1H-1,2,4-triazol-1-yl)-2-(3-fluorophenyl)-4-(prop-1-en-2-yflisoquinolin-1(2H)-one. To a solution of 6-(3-(((tert-butyldiphenylsilyl)oxy)methyl)-4-ethyl-5-oxo-4,5-dihydro-1H-1,2,4-triazol-1-yl)-2-(3-fluorophenyl)-4-(prop-1-en-2-yl)isoquinolin-1(2H)-one (92 mg, 0.14 mmol) in tetrahydrofuran (THF) (10 mL) was added a tetrahydrofuran (THF) solution (1 M) of tetrabutylammonium fluoride (0.14 mL, 0.14 mmol). The reaction mixture was stirred at 25° C. for 0.5 h. The mixture was diluted with H₂O (15 mL) and extracted with ethyl acetate (50 mL×2). The combined organic extract washed with brine (20 mL), dried over Na₂SO₄, and concentrated. The residue was purified by chromatography (30-100% EtOAc in heptane), then further purified by prep-HPLC (C18 column, 10-90% gradient MeCN in water) to afford the title compound as a white solid (35 mg, yield 60%). LCMS (ES-API): mass calcd. for C₂₃H₂₁FN₄O₃, 420.2; m/z found, 421.2 [M+H]⁺. ¹H NMR (400 MHz, DMSO-d₆) 8 8.40 (br s, 2H), 8.15 (br d, J=9.09 Hz, 1H), 7.55-7.59 (m, 1H), 7.49 (br d, J=9.60 Hz, 1H), 7.25-7.42 (m, 3H), 5.40 (br s, 1H), 5.17 (s, 1H), 4.53 (s, 2H), 3.81 (q, J=6.87 Hz, 2H), 2.14 (s, 3H), 1.29 (br t, J=6.87 Hz, 3H) ppm.

Step H: racemic-6-(4-Ethyl-3-(hydroxymethyl)-5-oxo-4,5-dihydro-1H-1,2,4-triazol-1-yl)-2-(3-fluorophenyl)-4-isopropyl-3,4-dihydroisoquinolin-1(2H)-one. To a solution of 6-(4-ethyl-3-(hydroxymethyl)-5-oxo-4,5-dihydro-1H-1,2,4-triazol-1-yl)-2-(3-fluorophenyl)-4-(prop-1-en-2-yl)isoquinolin-1(2H)-one (35 mg, 0.08 mmol) in methanol (5 mL) was added Pd/C (18 mg, 10%). The mixture was degassed and purged with hydrogen gas for 3 times. The reaction mixture was then stirred under hydrogen atmosphere (15 psi) at 25° C. for 12 h. The mixture was filtered through a short pad of Celite®. The filtrate was concentrated. The residue was purified by prep-HPLC (C18 column, 10-90% gradient MeCN in water) to afford the title compound as a white solid (15 mg, yield 42%). LCMS (ES-API): mass calcd. for C₂₃H₂₅FN₄O₃, 424.2; m/z found, 425.3 [M+H]⁺. ¹ H NMR(400 MHz, DMSO-d₆) δ 7.94-8.10 (m, 2H), 7.92 (s, 1H), 7.45-7.51 (m, 1H), 7.31 (br d, J=11.12 Hz, 1H), 7.26 (br d, J=8.08 Hz, 1H), 7.11 (br t, J=8.34 Hz, 1H), 5.82 (br s, 1H), 4.51 (s, 2H), 4.27 (br d, J=10.61 Hz, 1H), 3.59-3.98 (m, 3H), 1.92-2.16 (m, 1H), 1.28 (br t, J=6.82 Hz, 3H), 0.92 (br d, J=6.57 Hz, 3H), 0.88 (br d, J=6.57 Hz, 3H) ppm.

### Example 11

### Racemic-2-(2-Chloro-6-fluorophenyl)-6-(4-ethyl-3-(hydroxymethyl)-5-oxo-4,5 -dihydro-1H-1,2,4-triazol-1-yl)-4-(prop-1-en-2-yl)-3,4-dihydroisoquinolin-1(2H)-one

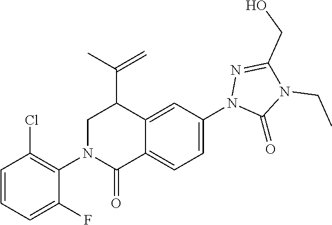 [SMILES: C=C(C)C1CN(c2c(F)cccc2Cl)C(=O)c2ccc(-n3nc(CO)n(CC)c3=O)cc21]

Step A. N-(2-Chloro-6-fluorophenyl)-3-methylbut-2-en-1-imine. To a solution of 2-chloro-6-fluoroaniline (2.9 g, 20 mmol) and 3-methyl-2-butenal (2 g, 23.9 mmol) in anhydrous dichloromethane (50 mL) at 0° C. was added triethylamine (8.3 mL, 60 mmol), followed by the addition of TiCl₄ (3 g, 16 mmol) dropwise. The reaction mixture was stirred at 0° C. for 1 h, then warmed to 25° C. and stirred for 4 h. The mixture was filtered through a short pad of Celite®, and the filtrate was partitioned between dichloromethane and water. The organic layer was separated, and the aqueous layer was extracted with dichloromethane. The combined organic extract was dried over MgSO₄ and concentrated to give the crude desired product as a yellow oil (3.3 g, 78%), which was used crude in the next step without further purification. ¹ H NMR(400 MHz, CDCl₃) δ 8.36 (dd, J=2.20, 9.66 Hz, 1H), 7.20 (d, J=7.83 Hz, 1H), 6.94-7.09 (m, 2H), 6.32 (br d, J=9.66 Hz, 1H), 5.30 (s, 1H), 2.00 (s, 6H).

Step B. 2-Chloro-6-fluoro-N-(3-methylbut-2-en-1-yl)aniline. To a solution of crude N-(2-chloro-6-fluorophenyl)-3-methylbut-2-en-1-imine (3.3 g, 15.6 mmol) in methanol (25 mL) was added NaBH4 (0.59 g, 15.6 mmol) at 25° C., and after 1 h interval, another batch of NaBH₄ (0.59 g, 15.6 mmol) was added. A total of three equivalents of NaBH₄ (1.8 g, 47 mmol) was added. The reaction mixture was stirred at 25° C. for 16 h. The mixture was concentrated to dryness. The mixture was partitioned between ethyl acetate and water. The organic layer was separated, and the aqueous layer was extracted with ethyl acetate. The combined organic extract was dried over MgSO₄ and concentrated. The residue was purified by chromatography (5-25% EtOAc in heptane) to give the desired product as a yellow oil (yield, 1.9 g, 57%). ¹H NMR (400 MHz, CDCl₃) δ 7.05 (br d, J=7.58 Hz, 1H), 6.83-6.97 (m, 1H), 6.55-6.73 (m, 1H), 5.32 (br s, 1H), 3.91 (br d, J=6.57 Hz, 2H), 3.82 (br s, 1H), 1.73 (s, 3H), 1.68 (s, 3H) ppm.

Step C. 4-Bromo-N-(2-chloro-6-fluorophenyl)-2-iodo-N-(3-methylbut-2-en-1-yl)benzamide. To a flask charged with 4-bromo-2-iodobenzoic acid (2.5 g, 7.7 mmol) was added SOCl₂ (7 mL). The mixture was heated at 80° C. for 15 min, then cooled to 25° C. and concentrated to afford the crude 4-bromo-2-iodobenzoyl chloride as an off white solid. The crude 4-bromo-2-iodobenzoyl chloride was dissolved into dichloromethane (10 mL), and then added slowly dropwise to a pre-cooled dichloromethane solution (20 mL) of 2-chloro-6-fluoro-N-(3-methylbut-2-en-1-yl)aniline (1.1 g, 5.1 mmol) and triethylamine (2.1 mmol, 15 mmol) at 0° C., followed by the addition of a catalytic amount of 4-dimethylaminopyridine (DMAP) (6 mg, 0.05 mmol). The reaction mixture was warmed and stirred at 25° C. for 3 h. The reaction was quenched by the addition of aqueous saturated NaHCO₃ solution. The organic layer was separated, and the aqueous layer was extracted with ethyl acetate. The combined organic extract was washed with water, brine, dried over MgSO₄, and concentrated to afford the desired crude product as a brown oil (2.4 g, 89%). LCMS (ES-API): mass calcd. for C₁₈H₁₈BrClFINO, 520.9; m/z found, 522.0 [M+H]⁺.

Step D: racemic-6-Bromo-2-(2-chloro-6-fluorophenyl)-4-(prop-1-en-2-yl)-3,4-dihydroisoquinolin-1(2H)-one. To a mixture of 4-bromo-N-(2-chloro-6-fluorophenyl)-2-iodo-N-(3-methylbut-2-en-1-yl)benzamide)-one (1.2 g, 2.3 mmol), tetrabutylammonium bromide (2.2 g, 6.9 mmol) and potassium acetate (0.45 g, 4.6 mmol) in anhydrous N,N-dimethylformamide (20 mL) was added palladium(II) acetate (0.51 g, 2.3 mmol) at 25° C. The reaction mixture was heated and stirred under nitrogen at 80° C. for 2 h, then cooled to 25° C. and water (100 mL) was added. The mixture was extracted with ethyl ether (100 mL) and ethyl acetate (100 mL). The combined organic extract was washed with brine, dried with anhydrous Na₂SO₄, and concentrated. The residue was purified by chromatography (30-60% EtOAc in heptane) to afford the desired product as a white solid (395 mg, 43%). LCMS (ES-API): mass calcd. for C₁₈H₁₄BrClFNO, 393.0; m/z found, 394.0 [M+H]⁺. ¹H NMR (400 MHz, CDCl₃) δ 8.05 (d, J=7.83 Hz, 1H), 7.55 (br d, J=8.31 Hz, 1H), 7.39 (s, 1H), 7.27-7.33 (m, 2H), 7.08-7.16 (m, 1H), 5.16 (br d, J=1.47 Hz, 1H), 4.87, 4.77 (2 s, 1H), 3.76-4.04 (m, 3H), 1.84, 1.81 (2 s, 3H) ppm.

Step E: Racemic-6-(3-(((tert-Butyldiphenylsilyl)oxy)methyl)-4-ethyl-5-oxo-4,5-dihydro-1H-1,2,4-triazol-1-yl)-2-(2-chloro-6-fluorophenyl)-4-(prop-1-en-2-yl)-3,4-dihydroisoquinolin-1(2H)-one. To a mixture of rac-6-bromo-2-(2-chloro-6-fluorophenyl)-4-(prop-1-en-2-yl)-3,4-dihydroisoquinolin-1(2H)-one (330 mg, 0.84 mmol) and K₃PO₄ (532 mg, 2.5 mmol) in anhydrous 1,4-dioxane (8 mL) was added 5-(((tert-butyldiphenylsilypoxy)methyl)-4-ethyl-2,4-dihydro-3H-1,2,4-triazol-3-one (638 mg, 1.7 mmol), CuI (159 mg, 0.84 mmol), and trans-N,N′-dimethylcyclohexane-1,2-diamine (119 mg, 0.84 mmol), respectively. The reaction mixture was slowly heated and stirred under nitrogen at 95° C. for 3 h, then cooled to 25° C. and quenched with addition of water (40 mL). The mixture was extracted with ethyl acetate (100 mL×2). The combined organic extract was washed with brine, dried with anhydrous Na₂SO₄, and concentrated. The residue was purified by chromatography (0-40% EtOAc in heptane) to afford the desired product as a white foam (290 mg, 50%). LCMS (ES-API): mass calcd. for C₃₉H₄₀ClFN₄O₃Si, 694.3; m/z found, 695.4 [M+H]⁺. ¹H NMR (400 MHz, CDCl₃) δ 8.23 (br d, J=9.09 Hz, 1H), 7.94 (br d, J=4.55 Hz, 2H), 7.69 (br d, J=7.07 Hz, 4H), 7.37-7.51 (m, 7H), 7.29 (br d, J=7.07 Hz, 2H), 7.11 (br t, J=7.58 Hz, 1H), 5.14 (br d, J=4.04 Hz, 1H), 4.86, 4.75 (2 s, 1H), 4.66 (s, 2H), 3.84-4.05 (m, 5H), 1.87, 1.84 (2 s, 3H), 1.37 (br t, J=6.82 Hz, 4H), 1.09 (s, 9H) ppm.

Step F: Racemic-2-(2-Chloro-6-fluorophenyl)-6-(4-ethyl-3-(hydroxymethyl)-5-oxo-4,5-dihydro-1H-1,2,4-triazol-1-yl)-4-(prop-1-en-2-yl)-3,4-dihydroisoquinolin-1(2H)-one. To a solution of rac-6-(3-(((tert-butyldiphenylsily0oxy)methyl)-4-ethyl-5-oxo-4,5-dihydro-1H-1,2,4-triazol-1-yl)-2-(2-chloro-6-fluorophenyl)-4-(prop-1-en-2-yl)-3,4-dihydroisoquinolin-1(2H)-one (280 mg, 0.4 mmol) in tetrahydrofuran (THF) (10 mL) was added a tetrahydrofuran (THF) solution (1 M) of tetrabutylammonium fluoride (0.4 mL, 0.4 mmol). The reaction mixture was stirred at 25° C. for 0.5 h. The mixture was diluted with H₂O and extracted with ethyl acetate. The combined organic extract washed with brine, dried over Na₂SO₄, and concentrated. The residue was purified by chromatography (50-100% EtOAc in heptane), then further purified by prep-HPLC (C18 column, 20-80% gradient MeCN in water) to afford the title compound as a white solid (130 mg, yield 70%). LCMS (ES-API): mass calcd. for C₂₃H₂₂ClFN₄O₃, 456.1; m/z found, 457.2[M+H]⁺. ¹ H NMR(400 MHz, CDCl₃) δ 8.24 (br d, J=9.09 Hz, 1H), 7.98 (br s, 2H), 7.27-7.35 (m, 2H), 7.06-7.16 (m, 1H), 5.15 (br d, J=4.04 Hz, 1H), 4.85, 4.75 (2 s, 1H), 4.68 (s, 2H), 3.97-4.07 (m, 1H), 3.84-3.95 (m, 4H), 1.87, 1.84 (2 s, 3H), 1.40 (br t, J=7.07 Hz, 3H) ppm.

### Example 12

### Racemic-6-(4-Ethyl-3-(hydroxymethyl)-5-oxo-4,5-dihydro-1H-1,2,4-triazol-1-yl)-2-(2-fluorophenyl)-4-isopropyl-3,4-dihydroisoquinolin-1(2H)-one

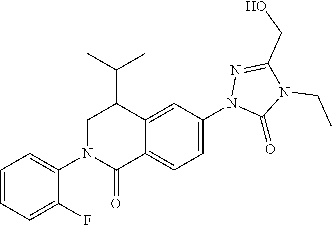 [SMILES: CCn1c(CO)nn(-c2ccc3c(c2)C(C(C)C)CN(c2ccccc2F)C3=O)c1=O]

To a solution of rac-2-(2-chloro-6-fluorophenyl)-6-(4-ethyl-3-(hydroxymethyl)-5-oxo-4,5-dihydro-1H-1,2,4-triazol-1-yl)-4-(prop-1-en-2-yl)-3,4-dihydroisoquinolin-1(2H)-one (Example 2, 60 mg, 0.13 mmol) in methanol (10 mL) was added Pd/C (28 mg, 10%). The mixture was degassed and purged with hydrogen gas for 3 times. The reaction mixture was then stirred under hydrogen atmosphere (15 psi) at 25° C. for 6 h. The mixture was filtered through a short pad of Celite®. The filtrate was concentrated. The residue was purified by first chromatography (30-100% EtOAc in heptane), then further purified by prep-HPLC (C18 column, 20-80% gradient MeCN in water) to afford the title compound as a white solid (23 mg, yield 41%). LCMS (ES-API): mass calcd. for C₂₃H₂₅FN₄O₃, 424.2; m/z found, 425.3 [M+H]⁺. ¹ H NMR(400 MHz, CDCl₃) δ 8.18 (d, J=8.59 Hz, 1H), 7.99 (s, 1H), 7.94 (br d, J=8.59 Hz, 1H), 7.27-7.42 (m, 2H), 7.11-7.23 (m, 2H), 4.68 (s, 2H), 4.23 (br d, J=12.63 Hz, 1H), 3.90 (br q, J=6.91 Hz, 2H), 3.78 (br d, J=12.63 Hz, 1H), 2.63 (br s, 1H), 2.14-2.29 (m, 1H), 1.41 (br t, J=6.91 Hz, 3H), 1.04 (br d, J=6.57 Hz, 3H), 0.89 (br d, J=6.57 Hz, 3H) ppm.

### Example 13

### Racemic-2-(2-Chloro-6-fluorophenyl)-6-(4-ethyl-3-(hydroxymethyl)-5-oxo-4,5-dihydro-1H-1,2,4-triazol-1-yl)-4-isopropyl-3,4-dihydroisoquinolin-1(2H)-one

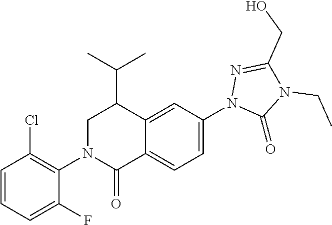 [SMILES: CCn1c(CO)nn(-c2ccc3c(c2)C(C(C)C)CN(c2c(F)cccc2Cl)C3=O)c1=O]

In the preparation of rac-6-(4-ethyl-3-(hydroxymethyl)-5-oxo-4,5-dihydro-1H-1,2,4-triazol-1-yl)-2-(2-fluorophenyl)-4-isopropyl-3,4-dihydroisoquinolin-1(2H)-one (Example 3), the title compound was obtained as the second product: a white solid (21 mg, yield 35%). LCMS (ES-API): mass calcd. for C₂₃H₂₄ClFN₄O₃, 458.2; m/z found, 459.1 [M+H]⁺. ¹ H NMR(400 MHz, CDCl₃) δ 8.16-8.23 (m, 1H), 8.00 (s, 1H), 7.95 (br d, J=8.08 Hz, 1H), 7.28-7.36 (m, 2H), 7.04-7.19 (m, 1H), 4.69 (s, 2H), 4.08-4.36 (m, 1H), 3.80-3.99 (m, 2H), 3.65-3.74 (m, 1H), 2.55-2.77 (m, 1H), 2.21-2.50 (m, 1H), 1.41 (br t, J=7.07 Hz, 3H), 1.06 (br d, J=6.57 Hz, 3H), 0.94 (br d, J=6.57 Hz, 3H) ppm.

### Example 14

### Racemic-6-(4-Ethyl-3-(hydroxymethyl)-5-oxo-4,5-dihydro-1H-1,2,4-triazol-1-yl)-2-(3-fluorophenyl)-4-phenyl-3,4-dihydroisoquinolin-1(2H)-one

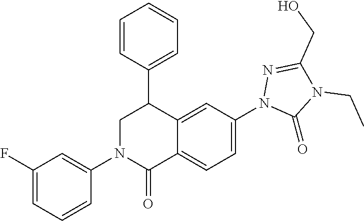 [SMILES: CCn1c(CO)nn(-c2ccc3c(c2)C(c2ccccc2)CN(c2cccc(F)c2)C3=O)c1=O]

Step A. 6-Bromo-4-phenylisoquinolin-1(2H)-one. To a mixture of 6-bromo-4-iodoisoquinolin-1(2H)-one (Example 1 Step A, 0.55 g, 1.7 mmol) and Cs₂CO₃ (1.3 g, 3.9 mmol) in 1,4-dioxane (20 mL) was added bis(triphenylphosphine)palladium(II) dichloride (0.22 g, 0.31 mmol) and phenylboronic acid (0.29 g, 2.4 mmol) respectively. The reaction mixture was degassed with nitrogen and then heated at 85° C. The mixture was cooled to 25° C. and filtered through a short pad of silica gel. The silica gel was washed with ethyl acetate. The combined filtrate was concentrated. The residue was purified by chromatography (SiO₂, 10-70% Ethyl acetate in heptane) to afford the desired product as a white solid (0.16 g, 33% yield). LCMS (ES-API): mass calcd. for C₁₅H₁₀BrNO, 299.0; m/z found, 298.1 [M−H]⁺. ¹ H NMR(400 MHz, DMSO-d₆) δ 11.65 (br d, J=4.04 Hz, 1H), 8.21 (d, J=8.59 Hz, 1H), 7.71 (br d, J=8.59 Hz, 1H), 7.56 (s, 1H), 7.41-7.53 (m, 5H), 7.18 (br d, J=5.56 Hz, 1H) ppm.

Step B. 6-Bromo-2-(3-fluorophenyl)-4-phenylisoquinolin-1(2H)-one. To a mixture of 6-bromo-4-phenylisoquinolin-1(2H)-one (160 mg, 0.53 mmol) and 3-fluorophenylboronic acid (224 mg, 1.6 mmol) in dichloromethane (DCM) (20 mL) and triethylamine (2 mL) was added copper(II) acetate (48 mg, 0.26 mmol) and pyridine (0.13 mL, 1.6 mmol). The reaction mixture was stirred at 25° C. for 48 h. The mixture was filtered through a short pad of silica gel and the silica gel was washed with ethyl acetate (100 mL). The filtrate was concentrated. The residue was purified by chromatography (SiO₂, 20-50%

EtOAc in heptane) to afford the crude desired product as a yellow amorphous gum (180 mg, 86%). LCMS (ES-API): mass calcd. for C₂₁Hi₃BrFNO, 393.0; m/z found, 394.0 [M+H]⁺.

Step C: 6-(3-(((tert-Butyldiphenylsilyl)oxy)methyl)-4-ethyl-5-oxo-4,5-dihydro-1H-1,2,4-triazol-1-yl)-2-(3-fluorophenyl)-4-phenylisoquinolin-1(2H)-one. To a mixture of 6-bromo-2-(3-fluorophenyl)-4-phenylisoquinolin-1(2H)-one (100 mg, 0.25 mmol) and K₃PO₄ (161 mg, 0.7 mmol) in anhydrous 1,4-dioxane (6 mL) was added 5-(((tert-butyldiphenylsilyl)oxy)methyl)-4-ethyl-2,4-dihydro-3H-1,2,4-triazol-3-one (194 mg, 0.5 mmol), CuI (48 mg, 0.25 mmol), and trans-N,N′-dimethylcyclohexane-1,2-diamine (36 mg, 0.25 mmol), respectively. The mixture was slowly heated and stirred under nitrogen at 95° C. for 2 h, then cooled to 25° C. and quenched with addition of water. The mixture was extracted with ethyl acetate (50 mL×2). The combined organic extract was washed with brine, dried with anhydrous Na₂SO₄, and concentrated. The residue was purified by chromatography (20-50% EtOAc in heptane) to afford the desired product as a white solid (120 mg, 68%). LCMS (ES-API): mass calcd. for C₄₂H₃₉FN₄O₃Si, 694.3; m/z found, 695.4 [M+H]⁺. ¹H NMR (400 MHz, CDCl₃) δ 8.60 (d, J=8.59 Hz, 1H), 8.28 (s, 1H), 8.15 (br d, J=8.59 Hz, 1H), 7.66 (br d, J=7.07 Hz, 4H), 7.34-7.53 (m, 12H), 7.27-7.33 (m, 2H), 7.08-7.19 (m, 2H), 4.62 (s, 2H), 3.88 (br q, J=6.82 Hz, 2H), 1.34 (br t, J=6.82 Hz, 3H), 1.07 (s, 9H) ppm.

Step D: 6-(4-Ethyl-3-(hydroxymethyl)-5-oxo-4,5-dihydro-1H-1,2,4-triazol-1-yl)-2-(3-fluorophenyl)-4-phenylisoquinolin-1(2H)-one. To a solution of 6-(3-(((tert-butyldiphenylsilyl)oxy)methyl)-4-ethyl-5-oxo-4,5-dihydro-1H-1,2,4-triazol-1-yl)-2-(3-fluorophenyl)-4-phenylisoquinolin-1(2H)-one (120 mg, 0.17 mmol) in tetrahydrofuran (THF) (10 mL) was added a tetrahydrofuran (THF) solution (1 M) of tetrabutylammonium fluoride (0.17 mL, 0.17 mmol). The reaction mixture was stirred at 25° C. for 0.5 h. The mixture was diluted with H₂O (15 mL) and extracted with ethyl acetate (50 mL×2). The combined organic extract washed with brine (20 mL), dried over Na₂SO₄, and concentrated. The residue was purified by chromatography (30-100% EtOAc in heptane) to afford the title compound as a white solid (50 mg, yield 63%). LCMS (ES-API): mass calcd. for C₂₆H₂₁FN₄O₃, 456.2; m/z found, 457.2 [M+H]⁺. ¹ H NMR(400 MHz, CDCl₃) δ 8.55-8.65 (m, 1H), 8.30 (s, 1H), 8.20 (br d, J=8.59 Hz, 1H), 7.39-7.56 (m, 6H), 7.28-7.32 (m, 2H), 7.17 (s, 1H), 7.10-7.16 (m, 1H), 4.64 (br d, J=6.19 Hz, 2H), 3.86 (q, J=7.07 Hz, 2H), 2.27 (t, J=6.19 Hz, 1H), 1.37 (br t, J=7.07 Hz, 3H) ppm.

Step E: racemic-6-(4-Ethyl-3-(hydroxymethyl)-5-oxo-4,5-dihydro-1H-1,2,4-triazol-1-yl)-2-(3-fluorophenyl)-4-phenyl-3,4-dihydroisoquinolin-1(2H)-one. To a solution of 6-(4-ethyl-3-(hydroxymethyl)-5-oxo-4,5-dihydro-1H-1,2,4-triazol-1-yl)-2-(3-fluorophenyl)-4-phenylisoquinolin-1(2H)-one (20 mg, 0.04 mmol) in methanol (5 mL) was added Pd/C (23 mg, 10%). The mixture was degassed and purged with hydrogen gas for 3 times. The reaction mixture was then stirred under hydrogen atmosphere (15 psi) at 25° C. for 18 h. The mixture was filtered through a short pad of Celite®. The filtrate was concentrated. The residue was purified by prep-HPLC (C18 column, 20-80% gradient MeCN in water) to afford the title compound as a white solid (7 mg, yield 35%). LCMS (ES-API): mass calcd. for C₂₆H₂₃FN₄O₃, 458.2; m/z found, 459.3 [M+H]⁺. ¹H NMR (400 MHz, CDCl₃) δ 8.24-8.35 (m, 1H), 8.07 (br d, J=9.09 Hz, 1H), 7.83 (s, 1H), 7.29-7.37 (m, 4H), 7.18 (br d, J=7.07 Hz, 2H), 6.84-6.98 (m, 3H), 4.64 (s, 2H), 4.45 (br s, 1H), 4.29-4.39 (m, 1H), 4.02 (dd, J=5.56, 12.13 Hz, 1H), 3.85 (q, J=7.24 Hz, 2H), 1.36 (t, J=7.24 Hz, 4H) ppm.

### Example 15

### (S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(pentan-2-yl)-2,3-dihydroquinazolin-4(1H)-one

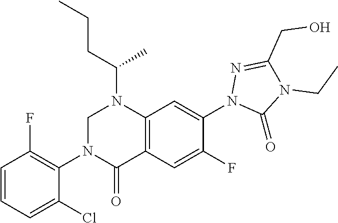 [SMILES: CCC[C@H](C)N1CN(c2c(F)cccc2Cl)C(=O)c2cc(F)c(-n3nc(CO)n(CC)c3=O)cc21]

Step A. (S)-4-Bromo-5-fluoro-2-(pentan-2-ylamino)benzonitrile. To a solution of 4-bromo-2,5-difluorobenzonitrile (1 g, 4.59 mmol) in NMP (5 mL) was added (5)-pentan-2-amine (850.63 mg, 6.88 mmol, HCl salt) and DIPEA (1.78 g, 13.76 mmol, 2.40 mL). The mixture was stirred at 100° C. for 24 h. The mixture was poured into water (50 mL). The aqueous phase was extracted with ethyl acetate (30 mL×2). The combined organic layers were washed with brine (20 mL×3), dried over Na₂SO₄, filtered and concentrated in vacuo. The residue was purified by column chromatography (SiO₂, petroleum ether/ethyl acetate=1/0 to 1/0) to give the title compound (1.6 g, 3.93 mmol, 43% yield, 70% purity) as white solid. MS (ESI): mass calcd. for C₁₂H₁₄BrFN₂, 284.0; m/z found, 285.0 [M+H]⁻¹. ¹H NMR (400 MHz, CDCl₃) δ=7.71 (d, J=8.8 Hz, 1H), 7.20 (d, J=6.0 Hz, 1H), 5.78 (d, J=8.8 Hz, 1H), 3.78-3.67 (m, 1H), 1.72-1.61 (m, 1H), 1.56-1.37 (m, 3H), 1.21 (d, J=6.2 Hz, 3H), 1.00-0.95 (m, 3H).

Step B. (S)-4-(3-((benzyloxy)methyl)-4-ethyl-5-oxo-4,5-dihydro-1H-1,2,4-triazol-1-yl)-5-fluoro-2-(pentan-2-ylamino)benzonitrile. To a solution of (S)-4-bromo-5-fluoro-2-(pentan-2-ylamino)benzonitrile (1.4 g, 3.44 mmol) in dioxane (40 mL) was added 3-((benzyloxy)methyl)-4-ethyl-1H-1,2,4-triazol-5(4H)-one (Example 1, step B, 1.04 g, 4.47 mmol) Cs₂CO₃ (2.02 g, 6.19 mmol), KI (399.35 mg, 2.41 mmol), trans-N¹,N²-dimethylcyclohexane-1,2-diamine (293.31 mg, 2.06 mmol) and CuI (327.26 mg, 1.72 mmol). The mixture was degassed and purged with N₂ and stirred at 100° C. for 16 h.

The mixture was filtered and filtrate was concentered in vacuo. The residue was purified by RP HPLC (Condition A) to give the title compound (1.2 g, 2.22 mmol, 64.64% yield, 81% purity) as yellow oil. MS (ESI): mass calcd. for C₂₄H₂₈FN₅O₂, 437.2; m/z found, 438.2 [M+H]⁺.

Step C. (S)-4-(3-((Benzyloxy)methyl)-4-ethyl-5-oxo-4,5-dihydro-lH-1,2,4-triazol-1-yl)-5-fluoro-2-(pentan-2-ylamino)benzoic acid. To a solution of (S)-4-(3-((benzyloxy)methyl)-4-ethyl-5-oxo-4,5-dihydro-1H-1,2,4-triazol-1-yl)-5-fluoro-2-(pentan-2-ylamino)benzonitrile (1.2 g, 2.22 mmol) in EtOH (10 mL) was added NaOH solution (4.04 g, 22.2 mmol, 22%). The mixture was stirred at 100° C. for 16 h. The mixture was poured into water (100 mL) and added HCl (1 N) to pH=6. The aqueous phase was extracted with ethyl acetate (50 mL×3). The combined organic layers were washed with brine (20 mL×2), dried over Na₂SO₄, filtered and concentrated in vacuo. The residue was purified by column chromatography (SiO₂, petroleum ether/ethyl acetate=10/1 to 2/1) to give the title compound (730 mg, 1.60 mmol, 72% yield) as a yellow oil. MS (ESI): mass calcd. For C₂₄H₂₉FN₄O₄, 456.2; m/z found, 457.2 [M+H]⁺.¹H NMR (400 MHz, CDCl₃) δ=7.70 (d, J=11.6 Hz, 1H), 7.34-7.26 (m, 5H), 6.87 (br d, J=5.8 Hz, 1H), 4.54 (s, 2H), 4.45 (s, 2H), 3.79 (q, J=7.4 Hz, 2H), 3.53-3.44 (m, 1H), 1.60-1.32 (m, 4H), 1.29 (t, J=7.2 Hz, 3H), 1.22-1.14 (m, 3H), 0.87 (t, J=7.2 Hz, 3H).

Step D. (S)-7-(3-((Benzyloxy)methyl)-4-ethyl-5-oxo-4,5-dihydro-1H-1,2,4-triazol-1-yl)-6-fluoro-1-(pentan-2-yl)-1H-benzo[d][1,3]oxazine-2,4-dione. To a solution of (S)-4-(3-((benzyloxy)methyl)-4-ethyl-5-oxo-4,5-dihydro-1H-1,2,4-triazol-1-yl)-5-fluoro-2-(pentan-2-ylamino)benzoic acid (350 mg, 766.69 μmol) in DCM (3 mL) was added triphosgene(455.03 mg, 1.53 mmol) at 0° C. The mixture was warmed to 30° C. and stirred at 30° C. for 18 h. The mixture was poured into water (100 mL) and added HCl (1 N) to pH=6. The mixture was purified quickly by column chromatography (SiO₂, petroleum ether/ethyl acetate=10/1 to 2/1) to give the title compound (170 mg, 281.86 lama 37% yield, 80% purity) as a yellow oil. MS (ESI): mass calcd. For C₂₅H₂₇FN₄O₅, 482.2; m/z found, 483.2 [M+H]⁺. ¹H NMR (400 MHz, CDCl₃) δ=7.99 (d, J=10.0 Hz, 1H), 7.91 (br d, J=4.0 Hz, 1H), 7.44-7.34 (m, 5H), 4.65 (s, 2H), 4.55 (s, 2H), 3.89 (q, J=7.2 Hz, 2H), 2.26-2.15 (m, 1H), 1.94-1.81 (m, 1H), 1.62 (d, J=6.8 Hz, 3H), 1.41-1.28 (m, 6H), 0.96 (t, J=7.4 Hz, 3H).

Step E. (S)-4-(3-((Benzyloxy)methyl)-4-ethyl-5-oxo-4,5-dihydro-1H-1,2,4-triazol-1-yl)-N-(2-chloro-6-fluorophenyl)-5-fluoro-2-(pentan-2-ylamino)benzamide. To a solution of 2-chloro-6-fluoro-aniline (61.54 mg, 422.80 μmol) in toluene (2 mL) was added AlMe₃ (2 M, 281.86 μL) at 15° C. under N₂. The mixture was stirred at 15° C. for 0.5 h. Then (S)-7-(3-((benzyloxy)methyl)-4-ethyl-5-oxo-4,5-dihydro-1H-1,2,4-triazol-1-yl)-6-fluoro-1-(pentan-2-yl)-1H-benzo[d][1,3]oxazine-2,4-dione (170 mg, 281.86 μmol) in toluene (1 mL) was added. The mixture was stirred at 50° C. for 16 h. The mixture was poured into HCl (1 N, 20 mL) and stirred for 3 min. The aqueous phase was extracted with ethyl acetate (30 mL×3). The combined organic layers were washed with brine (10 mL×2), dried over Na₂SO₄, filtered and concentrated in vacuo. The residue was purified by column chromatography (SiO₂, petroleum ether/ethyl acetate=10/1 to 4/1) to give the title compound (127 mg, 217.45 μma 77% yield, 100% purity) as a cyan solid. MS (ESI): mass calcd. For C₃₀H₃₂ClF₂N₅O₃, 583.2; m/z found, 584.2 [M+H]⁺. ¹H NMR (400 MHz, CDCl₃) δ=7.53-7.46 (m, 1H), 7.43-7.33 (m, 7H), 7.30-7.27 (m, 1H), 7.25-7.19 (m, 1H), 7.15-7.10 (m, 1H), 6.95 (d, J=6.4 Hz, 1H), 4.61 (s, 2H), 4.52 (s, 2H), 3.85 (q, J=7.2 Hz, 2H), 3.51 (br s, 1H), 1.59-1.57(m, 2H), 1.49-1.41 (m, 2H), 1.35 (t, J=7.2 Hz, 3H), 1.20 (d, J=6.4 Hz, 3H), 0.90 (t, J=6.8 Hz, 3H).

Step F. (S)-7-(3-((Benzyloxy)methyl)-4-ethyl-5-oxo-4,5-dihydro-1H-1,2,4-triazol-1-yl)-3-(2-chloro-6-fluorophenyl)-6-fluoro-1-(pentan-2-yl)-2,3-dihydroquinazolin-4(1H)-one. To a solution (S)-4-(3-((benzyloxy)methyl)-4-ethyl-5-oxo-4,5-dihydro-1H-1,2,4-triazol-1-yl)-N-(2-chloro-6-fluorophenyl)-5-fluoro-2-(pentan-2-ylamino)benzamide (107 mg, 183.20 μmol) in EtOH (1 mL) was added HCHO (148.67 mg, 1.83 mmol, 136.40 μL, 37% purity). The mixture was stirred at 110° C. for 3 h in microwave tube. The mixture was poured into water (30 mL). The aqueous phase was extracted with ethyl acetate (30 mL×3). The combined organic layers were washed with brine (10 mL×2), dried over Na₂SO₄, filtered and concentrated in vacuo to give the title compound (170 mg, crude) as a yellow oil. MS (ESI): mass calcd. for C₃₁H₃₂ClF₂N₅O₃, 595.2; m/z found, 596.2 [M+H]⁺. ¹H NMR (400 MHz, CDCl₃) δ=7.90 (d, J=10.6 Hz, 1H), 7.44-7.28 (m, 7H), 7.20-7.06 (m, 2H), 4.94 (s, 2H), 4.61 (s, 2H), 4.52 (s, 2H), 3.89-3.82 (m, 2H), 3.71-3.69 (m, 1H), 1.68-1.58 (m, 2H), 1.50-1.40 (m, 2H), 1.38-1.34 (m, 3H), 1.26-1.24 (m, 3H), 0.95-0.88 (m, 3H).

Step G. (S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(pentan-2-yl)-2,3-dihydroquinazolin-4(1H)-one. A mixture of (S)-7-(3-((benzyloxy)methyl)-4-ethyl-5-oxo-4,5-dihydro-1H-1,2,4-triazol-1-yl)-3-(2-chloro-6-fluorophenyl)-6-fluoro-1-(pentan-2-yl)-2,3-dihydroquinazolin-4(1H)-one (170 mg, crude) in TFA (1.5 mL) was stirred at 80° C. for 12 h. The mixture was concentrated and purified by RP HPLC (method A) to give the title compound (20.94 mg, 41.39 μmol, 100% purity) as a white solid. MS (ESI): mass calcd. for C₂₄H₂₆ClF₂N₅O₃, 505.2; m/z found, 506.2 [M+H]⁺. ¹ H NMR (400 MHz, CDCl₃) δ=7.90 (d, J=10.8 Hz, 1H), 7.34-7.28 (m, 2H), 7.19 (dd, J=1.8, 5.8 Hz, 1H), 7.16-7.10 (m, 1H), 4.83-4.70 (m, 2H), 4.67 (br d, J=4.4 Hz, 2H), 3.90 (q, J=7.2 Hz, 2H), 3.86-3.79 (m, 1H), 2.13 (br s, 1H), 1.68-1.59 (m, 1H), 1.50-1.38 (m, 6H), 1.23 (dd, J=6.8, 19.4 Hz, 3H), 0.94-0.88 (m, 3H).

### Example 16

### (S)-1-(sec-Butyl)-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydroquinazolin-4(1H)-one

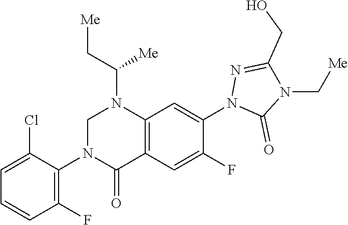 [SMILES: CC[C@H](C)N1CN(c2c(F)cccc2Cl)C(=O)c2cc(F)c(-n3nc(CO)n(CC)c3=O)cc21]

Step A. 2,4,5-Trifluorobenzoyl chloride. To a solution of 2,4,5-trifluorobenzoic acid (4 g, 22.72 mmol) in DCM(35 mL) was added DMF (17 μL) and a solution of (COCl)₂ (4.32 g, 34.07 mmol, 2.98 mL) in DCM (5 mL) at 25° C. under N₂. The mixture was stirred at 25° C. for 1 h. The reaction mixture was concentrated under reduced pressure to the title compound (4.4 g, crude) as yellow solid, which was used directly for the next step.

Step B. N-(2-Chloro-6-fluorophenyl)-2,4,5-trifluorobenzamide. To a mixture of 2-chloro-6-fluoro-aniline (3.86 g, 26.53 mmol) and pyridine (8.74 g, 110.53 mmol) in DCM (45 mL) was added a solution of 2,4,5-trifluorobenzoyl chloride (4.30 g, 22.11 mmol) in DCM (5 mL). The mixture was stirred at 25° C. for 16 h. The reaction mixture was quenched by addition of H₂O (70 mL). The mixture was extracted with DCM (60 mL×2). The combined organic layers were washed with brine (100 mL), dried over Na₂SO₄, filtered and concentrated under reduced pressure. The residue was triturated with petroleum ether/ethyl acetate (10:1,30 mL) to afford the title compound (5.6 g, 18.44 mmol, 83.43% yield) as white solid. MS (ESI): mass calcd. for C₁₃H₆ClF₄NO, 303.0; m/z found, 304.0 [M+H]⁺. ¹H NMR (400 MHz, CDCl₃) δ=8.06 (ddd, J=7.2, 8.9, 10.4 Hz, 2H), 7.33-7.27 (m, 2H), 7.19-7.06 (m, 2H).

Step C. 4-(3-((Benzyloxy)methyl)-4-ethyl-5-oxo-4,5-dihydro-1H-1,2,4-triazol-1-yl)-N-(2-chloro-6-fluorophenyl)-2,5-difluorobenzamide. To a mixture of N-(2-chloro-6-fluoro-phenyl)-2,4,5-trifluoro-benzamide (500 mg, 1.65 mmol) and 3-(benzyloxymethyl)-4-ethyl-1H-1,2,4-triazol-5-one (423.38 mg, 1.82 mmol) in DMSO (10 mL) was added K₂CO₃ (274 mg, 1.98 mmol). The mixture was stirred at 80° C. for 28 hours. The reaction mixture was quenched by addition of 1 N HCl (20 mL) at 0° C., and then extracted with EtOAc (30 mL×2). The combined organic layers was washed with brine (30 mL×2), dried over Na₂SO₄ and concentrated under reduced pressure. The residue was purified by column chromatography (SiO₂, petroleum ether/ethyl acetate=10/13/1) to afford the title compound. MS (ESI): mass calcd. for C₂₅H₂₀ClF₃N₄O₃, 516.1; m/z found, 517.2 [M+H]⁺.

Step D. (S)-4-(3-((Benzyloxy)methyl)-4-ethyl-5-oxo-4,5-dihydro-1H-1,2,4-triazol-1-yl)-2-(sec-butylamino)-N-(2-chloro-6-fluorophenyl)-5-fluorobenzamide. To a mixture of 4-(3-((benzyloxy)methyl)-4-ethyl-5-oxo-4,5-dihydro-1H-1,2,4-triazol-1-yl)-N-(2-chloro-6-fluorophenyl)-2,5-difluorobenzamide (416 mg, 804 μmol) in (25)-butan-2-amine (1 g, 13.67 mmol, 1.37 mL) was heated to 140° C. for 16 h in sealed tube. The reaction mixture was quenched by addition H₂O (20 mL), and then extracted with EtOAc (25 mL×2). The combined organic layer was washed with brine (40 mL), dried over Na₂SO₄, filtered and concentrated under reduced pressure. The residue was purified by column chromatography (SiO₂, petroleum ether/ethyl acetate=10/1 to 3/1) to afford the title compound (272 mg, 343.56 μmol, 42.72% yield, 72% purity) as yellow solid. MS (ESI): mass calcd. for C₂₉H₃₀ClF₂N₅O₃, 569.2; m/z found, 570.2 [M+H]⁺.

Step E. (S)-7-(3-((Benzyloxy)methyl)-4-ethyl-5-oxo-4,5-dihydro-1H-1,2,4-triazol-1-yl)-1-(sec-butyl)-3-(2-chloro-6-fluorophenyl)-6-fluoro-2,3-dihydroquinazolin-4(1H)-one. To a solution of (S)-4-(3-((benzyloxy)methyl)-4-ethyl-5-oxo-4,5-dihydro-1H-1,2,4-triazol-1-yl)-2-(sec-butylamino)-N-(2-chloro-6-fluorophenyl)-5-fluorobenzamide (120 mg, 151 μmol) in EtOH (2 mL) was added formaldehyde (123.02 mg, 1.52 mmol, 112.864, 37% purity). The mixture was stirred at 80° C. for 16 h. The reaction mixture was filtered and concentrated under reduced pressure. The residue was purified by prep-TLC (SiO₂, petroleum ether : ethyl acetate=1:1) to afford the title compound (75 mg, 100.51 μmol, 66.31% yield, 78% purity) as colorless oil. MS (ESI): mass calcd. for C₃₀H₃₀ClF₂N₅O₃, 581.2; m/z found, 582.2 [M+H]⁺. ¹H NMR (400 MHz, CDCl₃) δ=7.92 (d, J=10.8 Hz, 1H), 7.43-7.34 (m, 5H), 7.34-7.28 (m, 2H), 7.21-7.10 (m, 2H), 4.92-4.66 (m, 2H), 4.62 (s, 2H), 4.56-4.49 (m, 2H), 3.87 (q, J=7 .2 Hz, 2H), 3.76-3.62 (m, 1H), 1.75-1.63 (m, 1H), 1.30-1.10 (m, 6H), 1.00 (td, J=7.4, 11.6 Hz, 2H), 0.92 (t, J=7.6 Hz, 2H).

Step F. (S)-1-(sec-Butyl)-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydroquinazolin-4(1H)-one. To a solution of (S)-7-(3-((benzyloxy)methyl)-4-ethyl-5-oxo-4,5-dihydro-1H-1,2,4-triazol-1-yl)-1-(sec-butyl)-3-(2-chloro-6-fluorophenyl)-6-fluoro-2,3-dihydroquinazolin-4(1H)-one (75 mg, 100.51 μmol) in DCM (2 mL) was added BCl₃ (1 M, 301 μL) at −78° C. for 2 h under N₂. Another BCl₃ (1 M, 2014) was added at −78° C. for 0.5 h. The reaction mixture was quenched by addition H₂O (15 mL) at 0° C., and then extracted with DCM (10 mL×2). The combined organic layer was washed with brine (15 mL), dried over Na₂SO₄, filtered and concentrated under reduced pressure. The residue was purified by RP HPLC (Condition A) to afford the title compound (20.3 mg, 41.19 μmol, 41% yield, 100% purity) as white solid. MS (ESI): mass calcd. for C₂₃H₂₄ClF₂N₅O₃, 491.2; m/z found, 492.2 [M+H]⁺. ¹H NMR (400 MHz, CDCl₃) δ=7.91 (d, J=10.7 Hz, 1H), 7.35-7.29 (m, 2H), 7.22-7.10 (m, 2H), 4.84-4.71 (m, 2H), 4.69 (br s, 2H), 3.92 (q, J=7.2 Hz, 2H), 3.74 (td, J=7.0, 14.0 Hz, 1H), 2.09 (br s, 1H), 1.74-1.57 (m, 2H), 1.43 (t, J=7.2 Hz, 3H), 1.24 (dd, J=6.7, 19.6 Hz, 3H), 0.99 (td, J=7.4, 11.6 Hz, 3H).

### Example 17

### (S)-3-(2-Chloro-6-fluorophenyl)-1-(1-cyclohexylethyl)-7-(4-ethyl-3-(hydroxymethyl)-5-oxo-4,5-dihydro-1H-1,2,4-triazol-1-yl)-6-fluoro-2,3-dihydroquinazolin-4(1H)-one

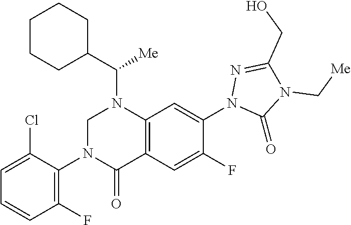 [SMILES: CCn1c(CO)nn(-c2cc3c(cc2F)C(=O)N(c2c(F)cccc2Cl)CN3[C@@H](C)C2CCCCC2)c1=O]

The title compound was prepared in a manner analogous to Example 16, however using (S)-1-cyclohexylethanamine instead of (2S)-butan-2-amine in Step D.

¹H NMR (400 MHz, CDCl₃) δ=7.90 (d, J=10.6 Hz, 1H), 7.35-7.29 (m, 2H), 7.19-7.06 (m, 2H), 4.84-4.73 (m, 2H), 4.69 (br d, J=4.3 Hz, 2H), 3.92 (q, J=7.2 Hz, 2H), 3.58-3.34 (m, 1H), 2.16-1.94 (m, 2H), 1.85-1.62 (m, 4H), 1.43 (t, J=7.2 Hz, 3H), 1.30-1.09 (m, 6H), 1.05-0.88 (m, 2H).

### Example 18

### 3-(2-Chloro-6-fluorophenyl)-7-(4-ethyl-3-(hydroxymethyl)-5-oxo-4,5-dihydro-1H-1,2,4-triazol-1-yl)-6-fluoro-1-isobutyl-2,3-dihydroquinazolin-4(1H)-one

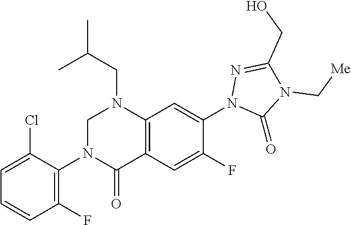 [SMILES: CCn1c(CO)nn(-c2cc3c(cc2F)C(=O)N(c2c(F)cccc2Cl)CN3CC(C)C)c1=O]

The title compound was prepared in a manner analogous to Example 16, however using 2-methylpropan-1-amine instead of (25)-butan-2-amine in Step D. MS (ESI): mass calcd. for C₂₃H₂₄ClF₂N₅O₃, 491.2; m/z found, 492.4 [M+H]⁺. ¹H NMR (400 MHz, CDCl₃) δ=7.89 (d, J=10.8 Hz, 1H), 7.31-7.29 (m, 2H), 7.14-7.10 (m, 2H), 4.85-4.83 (m, 1H), 4.76-4.73 (m, 1H), 4.66 (s, 1H), 3.93-3.88 (m, 2H), 3.18-3.04 (m, 2H), 2.22 (s, 1H), 2.03-1.98 (m, 1H), 1.41 (t, J=7.2 Hz, 1H), 1.01-0.98 (m, 6H).

### Example 19

### 3-(2-Chloro-6-fluorophenyl)-1-cyclobutyl-7-(4-ethyl-3-(hydroxymethyl)-5-oxo-4,5-dihydro-1H-1,2,4-triazol-1-yl)-6-fluoro-2,3-dihydroquinazolin-4(1H)-one

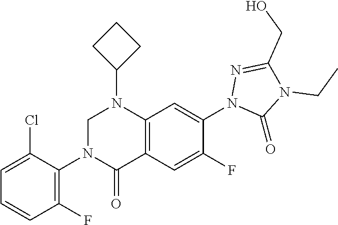 [SMILES: CCn1c(CO)nn(-c2cc3c(cc2F)C(=O)N(c2c(F)cccc2Cl)CN3C2CCC2)c1=O]

The title compound was prepared in a manner analogous to Example 16, however using cyclobutanamine instead of (2S)-butan-2-amine in Step D. MS (ESI): mass calcd. for C₂₃H₂₂ClF₂N₅O₃, 489.1; m/z found, 490.2 [M+H]⁺. ¹H NMR (400 MHz, CDCl₃) δ=7.89 (d, J=10.8 Hz, 1H), 7.33-7.30 (m, 2H), 7.19-7.12 (m, 2H), 4.72-4.66 (m, 2H), 3.98-3.87 (m, 3H), 2.33-2.29 (m, 2H), 2.16-2.11 (m, 2H), 1.79-1.74 (m, 2H), 1.41 (t, J=7.2 Hz, 1H).

### Example 20

### 1-Butyl-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydroquinazolin-4(1H)-one

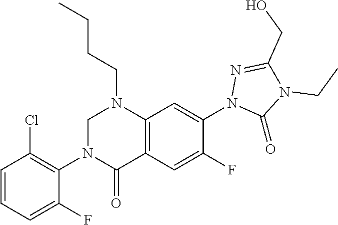 [SMILES: CCCCN1CN(c2c(F)cccc2Cl)C(=O)c2cc(F)c(-n3nc(CO)n(CC)c3=O)cc21]

The title compound was prepared in a manner analogous to Example 16, however using butan-1-amine instead of (25)-butan-2-amine in Step D. MS (ESI): mass calcd. for C₂₃H₂₄ClF₂N₅O₃, 491.2; m/z found, 492.1 [M+H]⁺. ¹H NMR (400 MHz, CDCl₃) δ=7.89 (d, J=10.6 Hz, 1H), 7.33-7.29 (m, 2H), 7.16-7.11 (m, 2H), 4.85-4.73 (m, 2H), 4.67 (d, J=6.0 Hz, 2H), 3.91 (q, J=7.2 Hz, 2H), 3.41-3.28 (m, 2H), 2.12 (t, J=6.4 Hz, 1H), 1.69-1.60 (m, 2H), 1.45-1.38 (m, 5H), 0.95 (t, J=7.2 Hz, 3H).

### Example 21

### 3-(2-Chloro-6-fluorophenyl)-1-(cyclohexylmethyl)-7-(4-ethyl-3-(hydroxymethyl)-5-oxo-4,5-dihydro-1H-1,2,4-triazol-1-yl)-6-fluoro-2,3-dihydroquinazolin-4(1H)-one

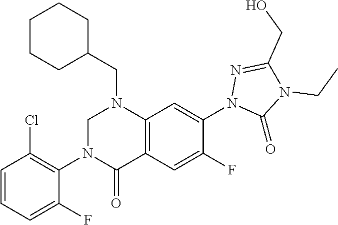 [SMILES: CCn1c(CO)nn(-c2cc3c(cc2F)C(=O)N(c2c(F)cccc2Cl)CN3CC2CCCCC2)c1=O]

The title compound was prepared in a manner analogous to Example 16, however using cyclohexylmethanamine instead of (2S)-butan-2-amine in Step D. MS (ESI): mass calcd. For C₂₆H₂₈ClF₂N₅O₃, 531.2; m/z found, 532.2 [M+H]⁺. ¹H NMR (400 MHz, CDCl₃) δ=7.88 (d, J=10.6 Hz, 1H), 7.33-7.29 (m, 2H), 7.16-7.07 (m, 2H), 4.86-4.72 (m, 2H), 4.67 (br d, J=4.8 Hz, 2H), 3.91 (q, J=7.2 Hz, 2H), 3.23-3.08 (m, 2H), 2.13 (br s, 1H), 1.88-1.80 (m, 2H), 1.76-1.64 (m, 4H), 1.42 (t, J=7.2 Hz, 3H), 1.29-1.13 (m, 3H), 1.02-0.90 (m, 2H).

### Example 22

### 3-(2-Chloro-6-fluorophenyl)-7-(4-ethyl-3-(hydroxymethyl)-5-oxo-4,5-dihydro-1H-1,2,4-triazol-1-yl)-6-fluoro-1-propyl-2,3-dihydroquinazolin-4(1H)-one

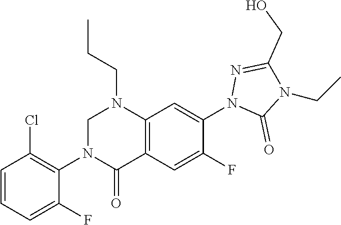 [SMILES: CCCN1CN(c2c(F)cccc2Cl)C(=O)c2cc(F)c(-n3nc(CO)n(CC)c3=O)cc21]

The title compound was prepared in a manner analogous to Example 16, however using propan-1-amine instead of (2S)-butan-2-amine in Step D. MS (ESI): mass calcd. for C₂₂H₂₂ClF₂N₅O₃, 477.1; m/z found, 478.1 [M+H]⁺. ¹H NMR (400 MHz, CDCl₃) δ=7.89 (d, J=10.8 Hz, 1H), 7.33-7.29 (m, 2H), 7.16-7.10 (m, 2H), 4.86-4.74 (m, 2H), 4.67 (d, J=6.2 Hz, 2H), 3.90 (q, J=7.2 Hz, 2H), 3.38-3.25 (m, 2H), 2.16 (t, J=6.4 Hz, 1H), 1.74-1.65 (m, 2H), 1.42 (t, J=7.4 Hz, 3H), 0.99 (t, J=7.4 Hz, 3H).

### Example 23

### (S)-1-(1-(1,3-Dioxan-2-yflethyl)-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one

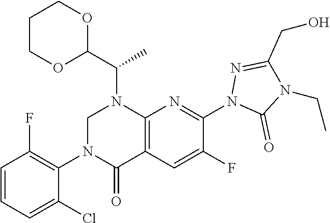 [SMILES: CCn1c(CO)nn(-c2nc3c(cc2F)C(=O)N(c2c(F)cccc2Cl)CN3[C@@H](C)C2OCCCO2)c1=O]

Step A. (S)-Benzyl (1-oxopropan-2-yl)carbamate. To a mixture of (S)-benzyl (1-hydroxypropan-2-yl)carbamate (2 g, 9.56 mmol) in DCM (80 mL) was added DMP (4.46 g, 10.51 mmol, 3.26 mL) at 0° C. Then the mixture was stirred at 0° C. for 2 h. The reaction mixture was quenched with saturated sodium thiosulfate solution (100 mL), and then diluted with saturated sodium bicarbonate solution (100 mL) and extracted with ethyl acetate (100 mL×3). The combined organic layers were washed with brine (50 mL×1), dried over Na₂SO₄, filtered and concentrated under reduced pressure to give the title compound (2.14g, crude) as a white oil.

Step B. (S)-Benzyl (1-(1,3-dioxan-2-yl)ethyl)carbamate. To a solution of (S)-benzyl (1-oxopropan-2-yl)carbamate (2.14 g, 10.33 mmol) and propane-1,3-diol (1.89 g, 24.78 mmol) in DCM (80 mL) was added p-toluenesulfonic acid (tosic acid, PTSA or pTsOH) (711.32 mg, 4.13 mmol). The mixture was stirred at 25° C. for 2 hr. The reaction mixture was quenched with saturated sodium bicarbonate solution (100 mL) and extracted with DCM (50 mL×3). The combined organic layer was washed with brine, dried over anhydrous Na₂SO₄, filtered and concentrated under reduced pressure. The residue was purified by column chromatography (SiO₂, petroleum ether/ethyl acetate=50/1 to 1/1) to give title compound (1.75 g, 6.53 mmol, 63.24% yield, 99% purity) as white solid. MS (ESI): mass calcd. for C₁₄H₁₉NO₄, 265.1; m/z found, 266.3 [M+H]⁺.

Step C. (S)-1-(1,3-Dioxan-2-yl)ethanamine. To a solution of benzyl N-[(1S)-1-(1,3-dioxan-2-yl)ethyl]carbamate (1.7 g, 6.41 mmol) in THF (10 mL) was added Pd/C (170 mg) under N₂ atmosphere. The suspension was degassed and purged with H₂ for three times. The mixture was stirred under H₂ at 25° C. for 12 hr. The reaction mixture was filtered. The filtrate was concentrated under reduced pressure at 25° C. to give 4 mL THF solution, which was used directly in the next step.

Step D. (S)-2-((1-(1,3-Dioxan-2-yflethyl)amino)-6-(3-((benzyloxy)methyl)-4-ethyl-5-oxo-4,5-dihydro-1H-1,2,4-triazol-1-yl)-N-(2-chloro-6-fluorophenyl)-5-fluoronicotinamide. To a mixture of 6-(3-((benzyloxy)methyl)-4-ethyl-5-oxo-4,5-dihydro-1H-1,2,4-triazol-1-yl)-2-chloro-N-(2-chloro-6-fluorophenyl)-5-fluoronicotinamide (200 mg, 374.29 μmol) and (S)-1-(1,3-dioxan-2-ypethanamine (6.40 mmol, 4 mL THF solution) in DMSO (4 mL) was added CsF (227.42 mg, 1.50 mmol), followed by DIPEA (145.12 mg, 1.12 mmol). The mixture was stirred at 100° C. for 12 h. The reaction mixture was diluted with water (20 mL) and extracted with EtOAc (15 mL×3). The combined organic layers were washed with brine (15 mL×5), dried over anhydrous Na₂SO₄, and concentrated under reduced pressure. The residue was purified by column chromatography (SiO₂, petroleum ether/ethyl acetate=100/1 to 10/1) to give the title compound (140 mg, 218.11 μmol, 58.27% yield, 98% purity) as white solid. MS (ESI): mass calcd. for C₃₀H₃₁F₂N₆O₅Cl, 628.2; m/z found,629.3 [M+H]⁺.

Step E. (S)-1-(1-(1,3-Dioxan-2-yl)ethyl)-7-(3-((benzyloxy)methyl)-4-ethyl-5-oxo-4,5-dihydro-1H-1,2,4-triazol-1-yl)-3-(2-chloro-6-fluorophenyl)-6-fluoro-2,3-dihydropyrido[2,3-d]pyrimidin-4(1H)-one. A solution of (S)-2-((1-(1,3-dioxan-2-yl)ethyl)amino)-6-(3-((benzyloxy)methyl)-4-ethyl-5-oxo-4,5-dihydro-1H-1,2,4-triazol-1-yl)-N-(2-chloro-6-fluorophenyl)-5-fluoronicotinamide (120 mg, 190.76 μmol) and HCHO (57.28 mg, 1.91 mmol, 52.554) was added into a microwave tube in EtOH (1.5 mL). The sealed tube was heated at 110° C. for 2 hr. The reaction mixture was concentrated under reduced pressure to remove solvent. The residue was diluted with water (20 mL) and extracted with EtOAc (20 mL×3). The combined organic layers were washed with brine, dried over anhydrous Na₂SO₄, filtered and concentrated under reduced pressure. The residue was purified by column chromatography (SiO₂, petroleum ether/ethyl acetate=50/1 to 1/1) to give the title compound (90 mg, 127.76 μmol, 66.97% yield, 91% purity) as white solid. MS (ESI): mass calcd. for C₃₁H₃₁F₂O₅Cl, 640.2; m/z found, 641.2 [M+H]⁺.

Step F. (S)-1-(1-(1,3-Dioxan-2-yl)ethyl)-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one. To a solution of (S)-1-(1-(1,3-dioxan-2-yl)ethyl)-7-(3-((benzyloxy)methyl)-4-ethyl-5-oxo-4,5-dihydro-1H-1,2,4-triazol-1-yl)-3-(2-chloro-6-fluorophenyl)-6-fluoro-2,3-dihydropyrido[2,3-d]pyrimidin-4(1H)-one (90 mg, 140.39 μmol) in EtOAc (30 mL) was added Pd/C (5 mg) under N₂ atmosphere. Additional Pd/C (10%, 25 mg) was added, the mixture was stirred at 25° C. for 20 hr. The reaction mixture was filtered. The filter cake was washed with methanol (100 mL). Then the combined organic solution was concentrated under reduced pressure. The residue was purified by RP HPLC (Condition D) to give the title compound (18 mg, 32.67 μmol, 23% yield, 100% purity) as white solid. MS (ESI): mass calcd. for C₂₄H₂₅F₂N₆O₅Cl, 550.1; m/z found, 551.3 [M+H]⁺. ¹ H NMR(400 MHz, CDCl₃) δ=8.16 (d, J=9.0 Hz, 1H), 7.35-7.30 (m, 2H), 7.17-7.12 (m, 1H), 5.10-5.04 (m, 1H), 4.96-4.75 (m, 3H), 4.68 (br s, 2H), 4.72-4.65 (m, 1H), 4.71-4.64 (m, 2H), 4.10-4.05 (m, 1H), 4.04-3.97 (m, 1H), 3.93-3.87 (m, 2H), 3.83-3.71 (m, 2H), 2.13 (br s, 1H), 2.05-1.93 (m, 1H), 1.43 (t, J=7.2 Hz, 3H), 1.36-1.28 (m, 4H).

### Example 24

### (S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(pentan-2-yl)-2,3-dihydropyrido[2,3-d]pyrimidin-4(1H)-one

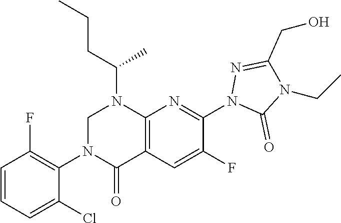 [SMILES: CCC[C@H](C)N1CN(c2c(F)cccc2Cl)C(=O)c2cc(F)c(-n3nc(CO)n(CC)c3=O)nc21]

Step A. (S)-Isopropyl 6-(3-((benzyloxy)methyl)-4-ethyl-5-oxo-4,5-dihydro-1H-1,2,4-triazol-1-yl)-5-fluoro-2-(pentan-2-ylamino)nicotinate. To a solution of isopropyl 6-(3-((benzyloxy)methyl)-4-ethyl-5-oxo-4,5 -dihydro-1H-1,2,4-triazol-1-yl)-2-chloro-5-fluoronicotinate (1 g, 2.23 mmol) in DMSO (12 mL) was added (25)-pentan-2-amine (302.95 mg, 2.45 mmol, HCl salt), DIPEA (863.78 mg, 6.68 mmol) and CsF (1.35 g, 8.91 mmol). The mixture was stirred at 100° C. for 6 hr. The reaction mixture was diluted with H₂O (20 mL) and extracted with EtOAc (20 mL×3). The combined organic layer was washed with brine (40 mL), dried over Na₂SO₄, filtered and concentrated under reduced pressure. The residue was purified by column chromatography (SiO₂, petroleum ether/ethyl acetate=1/0 to 10/1) to give the title compound (800 mg, 1.59 mmol, 71% yield, 99% purity) as a yellow oil. MS (ESI): mass calcd. for C₂₆H₃₄FN₅O₄, 499.3; m/z found, 500.3 [M+H]⁺.

Step B. (S)-6-(3-((Benzyloxy)methyl)-4-ethyl-5-oxo-4,5-dihydro-lH-1,2,4-triazol-1-yl)-N-(2-chloro-6-fluorophenyl)-5-fluoro-2-(pentan-2-ylamino)nicotinamide. To a solution of 2-chloro-6-fluoroaniline (349.65 mg, 2.40 mmol) in DCM (4 mL) was added dropwise AlMe₃ (2 M, 1.60 mL) at 25° C. The mixture was stirred at this temperature for 0.5 hr. Then a solution of (S)-isopropyl 6-(3-((benzyloxy)methyl)-4-ethyl-5-oxo-4,5-dihydro-1H-1,2,4-triazol-1-yl)-5-fluoro-2-(pentan-2-ylamino)nicotinate (400 mg, 800.68 μmol) in DCM (2 mL) was added into the mixture. The resulting mixture was heated to 60° C. for 12 hr. Another Al(CH₃)₃ (2 M, 1.60 mL) was added, and the mixture was stirred at 60° C. for another 24 hr. The reaction mixture was quenched with HCl (2M, 10 mL) at 20° C., and then diluted with H₂O (20 mL). The mixture was extracted with EtOAc 60 mL (20 mL×3). The combined organic layer was washed with brine (5 mL) dried over Na₂SO₄, filtered and concentrated under reduced pressure. The residue was purified by column chromatography (SiO₂, petroleum ether/ethyl acetate=1/0 to 5/1) to afford the title compound (240 mg, 340.49 μmol, 42.52% yield, 83% purity) as a yellow solid. MS (ESI): mass calcd. for C₂₉H₃₁ClF₂N₆O₃, 584.2; m/z found, 585.2 [M+H]⁺.

Step C. (S)-7-(3-((Benzyloxy)methyl)-4-ethyl-5-oxo-4,5-dihydro-1H-1,2,4-triazol-1-yl)-3-(2-chloro-6-fluorophenyl)-6-fluoro-1-(pentan-2-yl)-2,3-dihydropyrido[2,3-d]pyrimidin-4(1H)-one. To a solution of (S)-6-(3-((benzyloxy)methyl)-4-ethyl-5-oxo-4,5-dihydro-1H-1,2,4-triazol-1-yl)-N-(2-chloro-6-fluorophenyl)-5-fluoro-2-(pentan-2-ylamino)nicotinamide (100 mg, 170.93 μmol) in EtOH (8 mL) was added HCHO (138.71 mg, 1.71 mmol, 127 μL, 37% purity). The mixture was stirred at 80° C. for 12 hr. The reaction mixture was concentrated under reduced pressure. The residue was dissolved in HCHO (2.5 mL, 37% purity) and EtOH (0.5 mL). The reaction vessel was sealed and heated in microwave at 110° C. for 3 hr. The reaction mixture was diluted with H₂O (10 mL) and extracted with EtOAc (15 mL×2). The combined organic layer was washed with brine (20 mL), dried over Na₂SO₄, filtered and concentrated under reduced pressure. The residue was purified by prep-TLC (SiO₂, PE:EtOAc=3:2) to afford the title compound (82 mg, 104.38 μmol, 61.07% yield, 76% purity) as a yellow oil. MS (ESI): mass calcd. for C30H₃₁ClF₂N₆O₃, 596.2; m/z found, 597.2 [M+H]⁺. ¹H NMR (400 MHz, CDCl₃) δ=7.42-7.29 (m, 7H), 7.20-7.11 (m, 1H), 4.97-4.81 (m, 1H), 4.62 (s, 2H), 4.54 (s, 2H), 3.92-3.82 (m, 2H), 3.77-3.62 (m, 1H), 1.39-1.34 (m, 4H), 1.29-1.22 (m, 6H), 0.94-0.89 (m, 3H).

Step D. (S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(pentan-2-yl)-2,3-dihydropyrido[2,3-d]pyrimidin-4(1H)-one. To a mixture of (S)-7-(3-((benzyloxy)methyl)-4-ethyl-5-oxo-4,5-dihydro-1H-1,2,4-triazol-1-yl)-3-(2-chloro-6-fluorophenyl)-6-fluoro-1-(pentan-2-yl)-2,3-dihydropyrido[2,3-d]pyrimidin-4(1H)-one (80 mg, 133.99 μmol) and in DCM (3 mL) was added BCl₃ (1 M, 401.97 μL) in one portion at −78° C. under N₂. The mixture was stirred at −78° C. for 2 hr. The reaction mixture was diluted with H₂O (10 mL) and extracted with DCM (10 mL×3). The combined organic layers were washed with brine (20 mL), dried over Na₂SO₄, filtered and concentrated under reduced pressure. The residue was purified by RP HPLC (Condition A) to give the title compound (20 mg, 39.06 ρmol, 29.15% yield, 99% purity) as an off-white solid. MS (ESI): mass calcd. for C₂₃H₂₅ClF₂N₆O₃, 506.2; m/z found, 507.2 [M+H]⁺. ¹H NMR (400 MHz, CDCl₃) δ=8.14 (d, J=9.0 Hz, 1H), 7.36-7.31 (m, 2H), 7.20-7.12 (m, 1H), 4.93-4.83 (m, 2H), 4.81-4.71 (m, 1H), 4.68 (d, J=6.5 Hz, 2H), 3.91 (q, J=7.3 Hz, 2H), 2.11 (t, J=6.5 Hz, 1H), 1.69-1.57 (m, 1H), 1.49-1.36 (m, 6H), 1.23 (dd, J=6.8, 19.8 Hz, 3H), 0.94-0.89 (m, 3H).

### Example 25

### Racemic-2-(4-Chloro-2-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

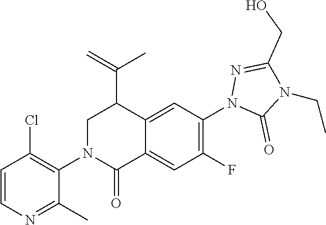 [SMILES: C=C(C)C1CN(c2c(Cl)ccnc2C)C(=O)c2cc(F)c(-n3nc(CO)n(CC)c3=O)cc21]

Step A: 3-Methylbut-2-enyl 4,5-difluoro-2-iodo-benzoate. To a solution of 4,5-difluoro-2-iodo-benzoic acid (11.5 g, 40.49 mmol) and 1-bromo-3-methyl-but-2-ene (5.91 g, 39.68 mmol, 4.58 mL) in MeCN (240 mL) was added K₂CO₃ (11.19 g, 80.99 mmol). The mixture was stirred at 15° C. for 16 hrs. The residue was diluted with water (300 mL) and extracted with EtOAc (200 mL×2). The combined organic layer was washed with brine (300 mL), dried over anhydrous Na₂SO₄, filtered and concentrated under reduced pressure to give the title compound (12 g, 34.08 mmol, 84 16% yield) as a yellow oil. ¹H NMR (400 MHz, CDCl₃) δ=7.80 (dd, J₁=7.6 Hz, J₂=9.6 Hz, 1H), 7.73 (dd, J₁=8.0 Hz, J₂=10.8 Hz, 1H), 5.50-5.45 (m, 1H), 4.83 (d, J=7.2 Hz, 2H), 1.79 (d, J=8.0 Hz, 6H).

Step B: 3-Methylbut-2-enyl 4-[3-(benzyloxymethyl)-4-ethyl-5-oxo-1,2,4-triazol-1-yl]-5-fluoro-2-iodo-benzoate. To a solution of 3-methylbut-2-enyl 4,5-difluoro-2-iodo-benzoate (5 g, 14.20 mmol) and 3-(benzyloxymethyl)-4-ethyl-1H-1,2,4-triazol-5-one (4.97 g, 21.30 mmol) in MeCN (100 mL) and DMF (50 mL) was added K₂CO₃ (3.93 g, 28.40 mmol). The mixture was stirred at 80° C. for 24 hrs. The reaction mixture was diluted with H₂O (200 mL) and extracted with EtOAc (200 mL×2). The combined organic layers were washed with brine (200 mL×4), dried over anhydrous Na₂SO₄, filtered and concentrated under reduced pressure. The residue was purified by column chromatography (1000 mesh silica gel, petroleum ether/ethyl acetate=6/1 to 3/1) to give the title compound (7.3 g, 12.51 mmol, 88.11% yield, 96 9% purity) as white solid. MS (ESI): mass calcd for C₂₄H₂₅FIN₃O₄, 565.1; m/z found, 566.0 [M+H]⁺. ¹H NMR (400 MHz, CDCl₃) δ=8.21 (d, J=7.2 Hz, 1H), 7.73 (dd, J=10.8 Hz, 1H), 7.41-7.23 (m, 5H), 5.51-5.49 (m, 1H), 4.85 (d, J=7.2 Hz, 2H), 4.61 (s, 1H), 4.51 (s, 1H), 3.84 (dd, J₁=11.2 Hz, J₂=14.4 Hz, 2H), 1.80 (d, J=8.0 Hz, 6H), 1.53 (t, J=6.4 Hz, 3H).

Step C: 5-((Benzyloxy)methyl)-4-ethyl-2-(7-fluoro-1-oxo-4-(prop-1-en-2-yl)isochroman-6-yl)-2,4-dihydro-3H-1,2,4-triazol-3-one. A mixture of 3-methylbut-2-en-1-yl4-(3-((benzyloxy)methyl)-4-ethyl-5-oxo-4,5-dihydro-1H-1,2,4-triazol-1-yl)-5-fluoro-2-iodobenzoate (4 g, 6.86 mmol), tBu₃P-Pd-G₂ (351 28 mg, 685 μmol), N-cyclohexyl-N-methyl-cyclohexanamine (1.47 g, 7.54 mmol, 1.60 mL) in toluene (200 mL) was degassed and purged with N₂ for 3 times. The mixture was stirred at 80° C. for 18 hrs under N₂ atmosphere. N-cyclohexyl-N-methyl-cyclohexanamine (669.60 mg, 3.43 mmol) and chloro[(tri-tert-butylphosphine)-2-(2-aMinobiphenyl]palladiuM(II) (tBu₃PPdG₂) (175.64 mg, 342 μmol) was added to the mixture at 15° C. The mixture was degassed and purged with N₂ for 3 times and was stirred at 80° C. for 16 hrs under N₂ atmosphere. The reaction mixture was concentrated under reduced pressure to remove toluene then diluted with H₂O (200 mL) and extracted with EtOAc (150 mL×3) The combined organic layers were washed with brine (100 mL×2), dried over anhydrous Na₂SO₄, filtered and concentrated under reduced pressure. The residue was purified by column chromatography (1000 mesh silica gel, petroleum ether/ethyl acetate=5/1 to 3/1) to give the title compound (1.2 g, 2.68 mmol, 39.09% yield, 97.7% purity) as yellow oil and 3-((benzyloxy)methyl)-4-ethyl-1-(7-fluoro-4-isopropyl-1-oxo-1H-isochromen-6-yl)-1H-1,2,4-triazol-5(4H)-one (1.1 g, 2.40 mmol, 34.95% yield, 95.3% purity) as yellow oil. Title compound: 5-((benzyloxy)methyl)-4-ethyl-2-(7-fluoro-1-oxo-4-(prop-1-en-2-yl)isochroman-6-yl)-2,4-dihydro-3H-1,2,4-triazol-3-one: MS (ESI): mass calcd for C₂₄H₂₄FN₃O₄, 437.2; m/z found, 438.5 [M+H]⁺: MS (ESI): mass calcd for C₂₄H₂₄FN₃O₄, 437.2; m/z found, 438.5 [M+H]⁺. MS (ESI): mass calcd for C₂₄H₂₄FN₃O₄, 437.2; m/z found, 438.5 [M+H]⁺. ¹H NMR (400 MHz, CDCl₃) δ=7.96 (d, J=10.4 Hz, 1H), 7.56 (d, J=6.8 Hz, 1H), 7.41-7.33 (m, 5H), 5.09 (s, 1H), 4.76 (s, 1H), 4.61 (s, 2H), 4.58-4.56 (m, 2H), 4.51 (s, 2H), 3.85 (dd, J₁=7.2 Hz, J₂=14.0 Hz, 2H), 3.70 (t, J=4.8 Hz, 1H), 1.82 (s, 3H), 1.36 (t, J=7.2 Hz, 3H).

Step D. 4-(3-((Benzyloxy)methyl)-4-ethyl-5-oxo-4,5-dihydro-1H-1,2,4-triazol-1-yl)-N-(4-chloro-2-methylpyridin-3-yl)-5-fluoro-2-(1-hydroxy-3-methylbut-3-en-2-yl)benzamide. To a solution of 4-chloro-2-methyl-pyridin-3-amine (191 mg, 1.34 mmol) in DCM (2 mL) was added Al(CH₃)₃ (2 M, 692.33 μL) at 0° C. The mixture was stirred at 0° C. for 15 min, and 15° C. for 15 min, then cooled to 0° C. A solution of 5-((benzyloxy)methyl)-4-ethyl-2-(7-fluoro-1-oxo-4-(prop-1-en-2-yl)isochroman-6-yl)-2,4-dihydro-3H-1,2,4-triazol-3-one (200 mg, 446.67 μmol) in DCM (2 mL) was added. The resulting mixture was stirred at 15° C. for 18.5 hrs. The reaction mixture was added to iced aq. HCl (1 N) slowly, and then extracted with EtOAc (40 mL×3). The combined organic layers were washed with anhydrous brine (20 mL×2), dried over anhydrous Na₂SO₄, filtered and concentrated under reduced pressure. The residue was purified by column chromatography (SiO₂, petroleum ether/ethyl acetate=1/1 to 0/1) to give title compound (250 mg. 99.58% purity) as yellow solid. MS (ESI): mass calcd. for C₃₀H₃₁ClFN₅O₄, 579.2; m/z found, 580.2 [M+H]⁺. ¹H NMR (400 MHz, DMSO-d6) δ=8.38 (d, J=5.2 Hz, 1H), 7.60-7.51 (m, 3H), 7.44-7.38 (m, 4H), 7.37-7.31 (m, 1H), 4.96 (m, 1H), 4.89 (m, 1H), 4.61 (m, 2H), 4.57 (m, 2H), 4.04-4.01 (m, 1H), 3.85-3.81 (m, 1H), 3.80-3.33 (m, 3H), 2.52 (m, 3H), 1.63(m, 3H), 1.24 (t, J=7.2 Hz, 3H).

Step E. 6-(3-((Benzyloxy)methyl)-4-ethyl-5-oxo-4,5-dihydro-1H-1,2,4-triazol-1-yl)-2-(4-chloro-2-methylpyridin-3-yl)-7-fluoro-4-(prop-1-en-2-yl)-3,4-dihydroisoquinolin-1(2H)-one. To a solution of 3-((benzyloxy)methyl)-4-ethyl-1-(7-fluoro-1-oxo-4-(prop-1-en-2-yOisochroman-6-yl)-1H-1,2,4-triazol-5(4H)-one (0.2 g, 343.35 μmol) in DCM (6 mL) was added methanesulfonyl chloride (59.00 mg, 515.03 μmol, 39.86 μL), TEA (104.23 mg, 1.03 mmol, 143.37 μL) and DMAP (4.19 mg, 34.34 μmol) at 0° C. The mixture was warmed to 15° C. and then stirred at 15° C. for 18 hrs. The reaction mixture was poured into H₂O (10 mL), and then extracted with EtOAc (10 mL×3). The combined organic layers were washed with brine (10 mL×2), dried over anhydrous Na₂SO₄, filtered and concentrated under reduced pressure to. The residue was purified by prep-TLC (SiO₂, Petroleum ether/Ethyl acetate=1/2) to give title compound (152 mg, 86.6% purity) as white solid. MS (ESI): mass calcd. for C₃₀H₂₉ClFN₅O₃, 561.2; m/z found, 562.3[M+H]⁺. ¹H NMR (400 MHz, CDCl₃) δ=8.41 (d, J=5.2 Hz, 1H), 8.03 (d, J=10.8 Hz, 1H), 7.52 (d, J=6.4 Hz, 1H), 7.39-7.34 (m, 6H), 5.19 (s, 1H), 4.69 (s, 1H), 4.62 (s, 3H), 4.53 (s, 2H), 4.08-4.05 (m, 1H), 3.89-3.84 (m, 3H), 3.78-3.73 (m, 1H), 2.52 (s, 3H), 1.88 (s, 3H), 1.37 (t, J=7.2 Hz, 3H).

Step F. Racemic 2-(4-Chloro-2-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. To a solution of 6-(3-((benzyloxy)methyl)-4-ethyl-5-oxo-4,5-dihydro-1H-1,2,4-triazol-1-yl)-2-(4-chloro-2-methylpyridin-3-yl)-7-fluoro-4-(prop-1-en-2-yl)-3,4-dihydroisoquinolin-1(2H)-one (90 mg, 138.68 μmol) in DCM (1 mL) was added BCl₃ (1 M, 832.05 μL) at −78° C. The mixture was stirred at −78° C. for 2 hrs. The reaction mixture poured into H₂O (10 mL), and then diluted extracted with DCM (10 mL×6). The combined organic layers were washed with brine (10 mL×2), dried over anhydrous Na₂SO₄, filtered and concentrated under reduced pressure. The residue was purified by RP HPLC (Condition D) to give title compound (4.63 mg) obtained as white solid. MS (ESI): mass calcd. C23H₂₃ClFN₅O₃, 471.2; m/z found, 472.2[M+H]⁺. ¹ H NMR(400 MHz, CDCl₃) δ=8.41 (d, J=5.2 Hz, 1H), 8.02 (d, J=10.8 Hz, 1H,), 7.53 (d, J=6.8 Hz, 1H), 7.32 (d, J=5.2 Hz, 1H), 5.19 (s, 1H), 4.69-4.68 (m, 3H), 4.07-4.03 (m, 1H), 3.94-3.91 (m, 3H), 3.78-3.7 (m, 1H), 2.52 (s, 3H), 1.88 (s, 3H), 1.43 (t, J=7.2 Hz, 3H).

### Example 26

### 7-(4-Ethyl-3-(hydroxymethyl)-5-oxo-4,5-dihydro-1H-1,2,4-triazol-1-yl)-6-fluoro-3-(2-fluoro-5-methylphenyl)-1-isopropyl-2,3-dihydroquinazolin-4(1H)-one

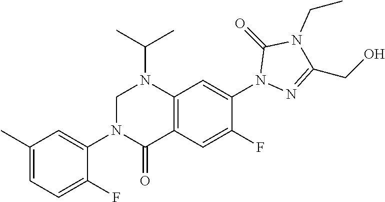 [SMILES: CCn1c(CO)nn(-c2cc3c(cc2F)C(=O)N(c2cc(C)ccc2F)CN3C(C)C)c1=O]

Step A: (S)-Isopropyl 6-(3-((benzyloxy)methyl)-4-ethyl-5-oxo-4,5-dihydro-1H-1,2,4-triazol-1-yl)-5-fluoro-2-((1,1,1-trifluoropropan-2-yfloxy)nicotinate. To a solution of 2-fluoro-5-methylaniline (74 u,L, 1.109 g/mL, 0.66 mmol) in DCM (2 mL) was added a solution of potassium bis(trimethylsilyl)amide in THF (LiHMDS 1 M in THF) (880 μL, 1 M, 0.88 mmol) at −78° C. under N₂ flow. The mixture was stirred at −78° C. for 0.5 h, then a solution of 7-(3-((benzyloxy)methyl)-4-ethyl-5-oxo-4,5-dihydro-1H-1,2,4-triazol-1-yl)-6-fluoro-1-isopropyl-2H-benzo[d][1,3]oxazine-2,4(1H)-dione (Example 1, Step F, 100 mg, 0.22 mmol) in DCM (2 mL) was added to the mixture and the reaction mixture was stirred at −78° C. for 0.5 h, then stirred at rt for 2h00. The mixture was poured into 1N HCl. The aqueous phase was extracted with EtOAc. The combined organic phase was washed with brine, dried with anhydrous MgSO₄, filtered and concentrated in vacuum. The residue was purified by silica gel chromatography (Stationary phase: irregular SiOH 15-40 μm 12g, Mobile phase: gradient from 100% heptane to 20% heptane, 80% EtOAc) to afford the title compound (30 mg, yield 25.455%). MS (ESI): mass calcd. for C₂₉H₃₁F₂N₅O₃, 535.2; m/z found, 536 [M+H]⁺.

Step B: 7-(3-((Benzyloxy)methyl)-4-ethyl-5-oxo-4,5-dihydro-1H-1,2,4-triazol-1-yl)-6-fluoro-3-(2-fluoro-5-methylphenyl)-1-isopropyl-2,3-dihydroquinazolin-4(1H)-one. The title compound was prepared according to the representative procedure Example 1, Step H, except using 7-(3-((benzyloxy)methyl)-4-ethyl-5-oxo-4,5-dihydro-1H-1,2,4-triazol-1-yl)-6-fluoro-3-(2-fluoro-5-methylphenyl)-1-isopropyl-2,3-dihydroquinazolin-4(1H)-one instead of 7-(3-((benzyloxy)methyl)-4-ethyl-5-oxo-4,5-dihydro-1H-1,2,4-triazol-1-yl)-3-(2-chloro-6-fluorophenyl)-6-fluoro-1-isopropyl-2,3-dihydroquinazolin-4(1H)-one. MS (ESI): mass calcd. for C30H₃₁F₂N₅O₃, 547.2; m/z found, 548.3 [M+H]⁺.

Step C: 7-(4-Ethyl-3-(hydroxymethyl)-5-oxo-4,5-dihydro-1H-1,2,4-triazol-1-yl)-6-fluoro-3-(2-fluoro-5-methylphenyl)-1-isopropyl-2,3-dihydroquinazolin-4(1H)-one. To a mixture of 7-(3-((benzyloxy)methyl)-4-ethyl-5-oxo-4,5-dihydro-1H-1,2,4-triazol-1-yl)-6-fluoro-3-(2-fluoro-5-methylphenyl)-1-isopropyl-2,3-dihydroquinazolin-4(1H)-one (31 mg, 0.0566 mmol) in DCM (0.465 mL) was added dropwise boron trichloride (1M in methylene chloride (0.170 mL) at −78° C. under nitrogen. The mixture was stirred at −78° C. for 1 h. Few drops of MeOH were added to the mixture. The mixture was diluted with DCM and water. The aqueous phase was extracted with DCM. The combined organic phase was dried with anhydrous MgSO₄, filtered and concentrated in vacuum. The residue was purified by column chromatography (Normal-phase silica gel chromatography (FCC) was performed on silica gel (SiO₂) using prepacked cartridge), mobile phase Gradient from 100% heptane to 70% heptane, 30% EtOAc) to afford desired product (18 mg, yield 35%). MS (ESI): mass calcd. for C₂₃H₂₅F₂N₇O₃, 457.2; m/z found, 458.3 [M+H]⁺. ¹ H NMR(400 MHz, CDCl₃) δ=7.91 (d, J=10.76 Hz, 1H) 7.24 (s, 2H) 7.14-7.20 (m, 1H) 7.06-7.12 (m, 1H) 4.79 (s, 2H) 4.68 (d, J=6.36 Hz, 2H) 3.75-3.99 (m, 3H) 2.35 (s, 2H) 1.42 (t, J=7 .34 Hz, 3H) 1.18-1.30 (m, 5H).

### Example 27

### 4-(7-(4-Ethyl-3-(hydroxymethyl)-5-oxo-4,5-dihydro-1H-1,2,4-triazol-1-yl)-6-fluoro-1-isopropyl-4-oxo-1,4-dihydroquinazolin-3(2H)-yl)-5-fluoronicotinonitrile

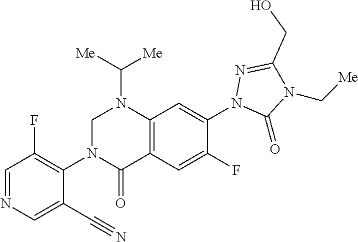 [SMILES: CCn1c(CO)nn(-c2cc3c(cc2F)C(=O)N(c2c(F)cncc2C#N)CN3C(C)C)c1=O]

Step A: 4-(3-((Benzyloxy)methyl)-4-ethyl-5-oxo-4,5-dihydro-1H-1,2,4-triazol-1-yl)-N-(3-cyano-5-fluoropyridin-4-yl)-5-fluoro-2-(isopropylamino)benzamide. To a solution 4-(3-((benzyloxy)methyl)-4-ethyl-5-oxo-4,5-dihydro-1H-1,2,4-triazol-1-yl)-5-fluoro-2-(isopropylamino)benzoic acid (Example 1, Step E, 150 mg, 350 μmol) in THF (0.5 mL) was added TEA (243 μL, 1.75 mmol) and propylphosphonic anhydride (T3P) (50% weight solution in THF) (408 μL, 0.7 mmol) respectively. After 1 min, the activated carboxylic acid derived intermediate was added into the solution of 4-amino-5-fluoronicotinonitrile (72 mg, 525 μmot) in THF (1.2 mL). The reaction mixture was stirred at 90° C. for 18 h, after which time it was concentrated then diluted with EtOAc and water. The mixture was extracted with EtOAc and washed with brine, dried over MgSO₄, filtered and concentrated. The residue was purified by column chromatography (Normal-phase silica gel chromatography (FCC) was performed on silica gel (SiO₂) using prepacked cartridge), Mobile phase: gradient from 100% heptane to 70% heptane, 30% EtOAc) to afford the title compound (43 mg, yield 22.4%). MS (ESI): mass calcd. for C₂₈H₂₇F₂N₇O₃, 547.2; m/z found, 548 [M+H]⁺.

Step B: 4-(7-(3-((Benzyloxy)methyl)-4-ethyl-5-oxo-4,5-dihydro-1H-1,2,4-triazol-1-yl)-6-fluoro-1-isopropyl-4-oxo-1,4-dihydroquinazolin-3(2H)-yl)-5-fluoronicotinonitrile. The title compound was prepared according to the representative procedure Example 1, Step H, except using 4-(7-(3-((benzyloxy)methyl)-4-ethyl-5-oxo-4,5-dihydro-1H-1,2,4-triazol-1-yl)-6-fluoro-1-isopropyl-4-oxo-1,4-dihydroquinazolin-3(2H)-yl)-5-fluoronicotinonitrile instead of 7-(3-((benzyloxy)methyl)-4-ethyl-5-oxo-4,5-dihydro-1H-1,2,4-triazol-1-yl)-3-(2-chloro-6-fluorophenyl)-6-fluoro-1-isopropyl-2,3-dihydroquinazolin-4(1H)-one. MS (ESI): mass calcd. for C₂₉H₂₇F₂N₇O₃, 559.2; m/z found, 560.3 [M+H]⁺.

Step C: 4-(7-(4-Ethyl-3-(hydroxymethyl)-5-oxo-4,5-dihydro-1H-1,2,4-triazol-1-yl)-6-fluoro-1-isopropyl-4-oxo-1,4-dihydroquinazolin-3(2H)-yl)-5-fluoronicotinonitrile. The title compound was prepared according to the representative procedure of Error! Reference source not found.26, Step C, except using 2-amino-3-fluorobenzonitrile instead of 2-fluoro-5-methylaniline (10 mg, yield 27%). MS (ESI): mass calcd. for C₂₂H₂₁F₂N₇O₃, 469.2; m/z found, 470.3 [M+H]⁺. ¹H NMR (400 MHz, CDCl₃) δ=7.93 (d, J=10 .7 6 Hz, 1H) 7.54-7.63 (m, 1H) 7.46 (s, 2H) 4.88 (s, 2H) 4.68 (d, J=6.36 Hz, 2H) 3.99-4.09 (m, 1H) 3.94-4.13 (m, 1H) 3.85-3.95 (m, 1H) 3.91 (d, J=7.34 Hz, 2H) 1.42 (t, J=7.34 Hz, 3H) 1.32 (d, J=6.36 Hz, 3H) 1.26 (d, J=6.85 Hz, 3H).

### Example 28

### 3-(2-Chloro-4-methylpyridin-3-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one

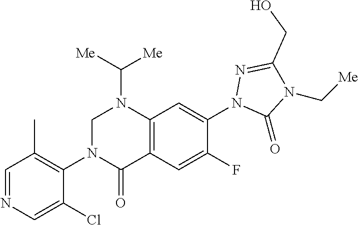 [SMILES: CCn1c(CO)nn(-c2cc3c(cc2F)C(=O)N(c2c(C)cncc2Cl)CN3C(C)C)c1=O]

Step A: 4-(3-((Benzyloxy)methyl)-4-ethyl-5-oxo-4,5-dihydro-1H-1,2,4-triazol-1-yl)-N-(2-chloro-4-methylpyridin-3-yl)-5-fluoro-2-(isopropylamino)benzamide. The title compound was prepared according to the representative procedure Example 127, Step A, except using 3-amino-2-chloro-4-methylpyridine instead of 4-amino-5-fluoronicotinonitrile. MS (ESI): mass calcd. for C₂₈H₃₀ClFN₆O₃, 552.2; m/z found, 553.0 [M+H]⁺.

Step B: 7-(3-((Benzyloxy)methyl)-4-ethyl-5-oxo-4,5-dihydro-1H-1,2,4-triazol-1-yl)-3-(2-chloro-4-methylpyridin-3-yl)-6-fluoro-1-isopropyl-2,3-dihydroquinazolin-4(1H)-one. The title compound was prepared according to the representative procedure Example 1, Step H, except using 7-(3-((benzyloxy)methyl)-4-ethyl-5-oxo-4,5-dihydro-1H-1,2,4-triazol-1-yl)-3-(2-chloro-4-methylpyridin-3-yl)-6-fluoro-1-isopropyl-2,3-dihydroquinazolin-4(1H)-one instead of 7-(3-((benzyloxy)methyl)-4-ethyl-5-oxo-4,5-dihydro-1H-1,2,4-triazol-1-yl)-3 -(2-chloro-6-fluorophenyl)-6-fluoro-1-isopropyl-2,3-dihydroquinazolin-4(1H)-one. MS (ESI): mass calcd. for C₂₉H₃₀ClFN₆O₃, 564.2; m/z found, 565.2 [M+H]⁺.

Step C: 4-(7-(4-Ethyl-3-(hydroxymethyl)-5-oxo-4,5-dihydro-1H-1,2,4-triazol-1-yl)-6-fluoro-1-isopropyl-4-oxo-1,4-dihydroquinazolin-3(2H)-yl)-5-fluoronicotinonitrile. The title compound was prepared according to the representative procedure of Error! Reference source not found.26, Step C, except using 2-amino-3-fluorobenzonitrile instead of 2-fluoro-5-methylaniline (27 mg, yield 23%). MS (ESI): mass calcd. for C₂₂H₂₄ClFN₆O₃, 474.2; m/z found, 475.2 [M+H]⁺. ¹H NMR (400 MHz, CDCl₃) δ=8.27 (d, J=4.89 Hz, 1H) 7.91 (d, J=10.76 Hz, 1H) 7.21 (d, J=5.38 Hz, 1H) 7.17 (d, J=5.87 Hz, 1H) 4.93 (d, J=9.78 Hz, 1H) 4.69 (s, 2H) 4.52 (d, J=10.27 Hz, 1H) 4.06-4.16 (m, 1H) 3.92 (q, J=7.34 Hz, 2H) 2.33 (s, 3H) 1.43 (t, J=7.34 Hz, 3H) 1.28 (d, J=6.36 Hz, 6H).

### Example 29

### 3-(3-Chloro-5-fluoropyridin-4-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one

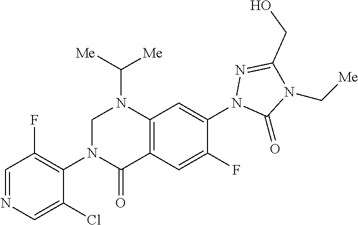 [SMILES: CCn1c(CO)nn(-c2cc3c(cc2F)C(=O)N(c2c(F)cncc2Cl)CN3C(C)C)c1=O]

The title compound was prepared in a manner analogous to Error! Reference source not found.26, Steps A to C, except using 3-chloro-5-fluoropyridin-4-amine instead of 2-fluoro-5-methylaniline in Step A. MS (ESI): mass calcd. for C₂₁H₂₁ClF₂N₆O₃, 478.1; m/z found, 479.1 [M+H]⁺. ¹H NMR (400 MHz, CDCl₃) δ=8.57 (s, 1H) 8.52 (s, 1H) 7.91 (d, J=10.76 Hz, 1H) 7.27 (br s, 1H) 4.83 (s, 2H) 4.69 (d, J=6.36 Hz, 2H) 3.98-4.06 (m, 1H) 3.91 (q, J=7.17 Hz, 2H) 1.43 (t, J=7 .34 Hz, 3H) 1.29 (d, J=6.85 Hz, 3H) 1.24 (d, J=6.85 Hz, 3H).

### Example 30

### 3-(3-Chloro-6-methoxypyridin-2-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one

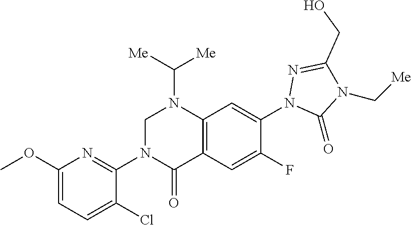 [SMILES: CCn1c(CO)nn(-c2cc3c(cc2F)C(=O)N(c2nc(OC)ccc2Cl)CN3C(C)C)c1=O]

The title compound was prepared in a manner analogous to Error! Reference source not found.26, Steps A to C, except using 2-amino-3-chloro-6-methoxypyridine instead of 2-fluoro-5-methylaniline in Step A. MS (ESI): mass calcd. for C₂₂H₂₄ClFN₆O₄, 490.2; m/z found, 491.3 [M+H]⁺. ¹H NMR (400 MHz, CDCl₃) δ=7.95 (d, J=10.76 Hz, 1H) 7.66 (d, J=8.80 Hz, 1H) 7.28 (s, 1H) 6.69 (d, J=8.31 Hz, 1H) 4.87-5.07 (m, 2H) 4.68 (d, J=6.36 Hz, 2H) 3.94-4.00 (m, 1H) 3.93-3.99 (m, 2H) 3.92 (s, 1H) 3.91 (s, 3H) 3.90 (s, 1H) 3.90-3.91 (m, 1H) 1.42 (t, J=7 .34 Hz, 3H) 1.28 (br d, J=6.36 Hz, 6H).

### Example 31

### (5)-1 -(s ec-Butyl)-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydropyrido[2,3-d] pyrimidin-4(1H)-one

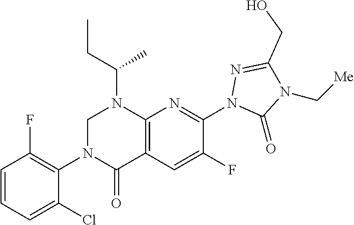 [SMILES: CC[C@H](C)N1CN(c2c(F)cccc2Cl)C(=O)c2cc(F)c(-n3nc(CO)n(CC)c3=O)nc21]

The title compound was prepared in a manner analogous to Error! Reference source not found.5, Steps D to G, except using (S)-butan-2-amine instead of isopropylamine in Step E. MS (ESI): mass calcd. for C₂₁H₂₁ClF₂N₆O₃, 492.2; m/z found, 493.2 [M+H]⁺. ¹H NMR (400 MHz, CDCl₃) δ=8.14 (d, J=9.29 Hz, 1H) 7.30-7.36 (m, 2H) 7.12-7.18 (m, 1H) 4.84-4.94 (m, 1H) 4.70-4.81 (m, 2H) 4.68 (d, J=6.85 Hz, 2H) 3.91 (d, J=7.34 Hz, 2H) 2.13 (s, 1H) 1.58-1.69 (m, 1H) 1.50-1.56 (m, 1H) 1.42 (t, J=7.34 Hz, 3H) 1.22 (dd, J=19 .56, 6.85 Hz, 3H) 0.91-1.02 (m, 3H).

### Example 32

### 3-(3-Chloro-2-methoxy-5-methylpyridin-4-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one

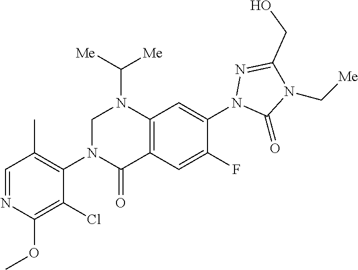 [SMILES: CCn1c(CO)nn(-c2cc3c(cc2F)C(=O)N(c2c(C)cnc(OC)c2Cl)CN3C(C)C)c1=O]

The title compound was prepared in a manner analogous to Error! Reference source not found.27, Steps A to C, except using 3-chloro-2-methoxy-5-methylpyridin-4-amine instead of 4-amino-5-fluoronicotinonitrile in Step A. MS (ESI): mass calcd. for C₂₃H₂₆ClFN₆O₄, 504.2; m/z found, 505.2 [M+H]⁺. ¹H NMR (400 MHz, CDCl₃) δ=8.01 (s, 1H) 7.90 (d, J=10 .7 6 Hz, 1H) 7.16 (d, J=5.87 Hz, 1H) 4.89 (d, J=10.27 Hz, 1H) 4.68 (d, J=6.36 Hz, 2H) 4.52 (d, J=10 .27 Hz, 1H) 4.04-4.11 (m, 1H) 4.03 (s, 3H) 3.91 (d, J=7.34 Hz, 2H) 2.18 (s, 2H) 1.42 (t, J=7.09 Hz, 3H) 1.26 (dd, J=6.60, 1.71 Hz, 6H).

### Example 33

### (S*)-6-[4-Ethyl-3-(hydroxymethyl)-5-oxo-1,2,4-triazol-1-yl]-7-fluoro-4-isopropenyl-2-(o-tolyl)-3,4-dihydroisoquinolin-1-one

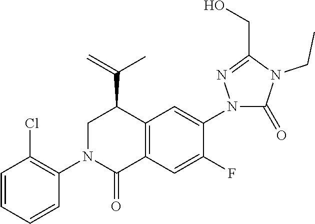 [SMILES: C=C(C)[C@@H]1CN(c2ccccc2Cl)C(=O)c2cc(F)c(-n3nc(CO)n(CC)c3=O)cc21]

Step A: 4-(3-((Benzyloxy)methyl)-4-ethyl-5-oxo-4,5-dihydro-1H-1,2,4-triazol-1-yl)-N-(2-chlorophenyl)-5-fluoro-2-(1-hydroxy-3-methylbut-3-en-2-yl)benzamide. To a solution of 2-chloroaniline (205 mg, 1.61 mmol) in 1.5 mL of DCM at 4° C. was added trimethylaluminum in toluene (2.0 M, 0.78 mL, 1.6 mmol). After stirring for 27 min at 4° C., the cooling bath was removed, and the mixture was stirred at RT for 15 min and re-cooled to 4° C. To the mixture was added a solution of 5-((benzyloxy)methyl)-4-ethyl-2-(7-fluoro-1-oxo-4-(prop-1-en-2-yl)isochroman-6-yl)-2,4-dihydro-3H-1,2,4-triazol-3-one (Example 25, Step C, 234 mg, 0.530 mmol) in DCM (2 mL) and stirred at 4° C. for 50 min. The reaction mixture was slowly poured into a mixture of ice and 1.5 mL of 2N HCl and extracted with DCM. The organic layer was dried over Na₂SO₄, filtered, concentrated, and purified by flash column chromatography (20-70% EtOAc in heptane) and then RF-HPLC (Gemini C18 110A, 5 μM 100×30 mm, 20-100% CH₃CN in water with 10 mM NH₄OH, 60 mL/min) to give the title compound (164 mg, 54%). LCMS (ES-API): mass calcd. for C₃₀H₃₀ClFN₄O₄, 564.19; m/z found, 565.2 [M+H]⁺. ¹ H NMR(400 MHz, CDCl₃) δ 8.66 (s, 1H), 8.36 (d, J=8.3 Hz, 1H), 7.62 (d, J=7.3 Hz, 1H), 7.52-7.30 (m, 7H), 7.14 (dt, J=1.5, 7.6 Hz, 1H), 5.02 (s, 1H), 4.85 (s, 1H), 4.61 (s, 2H), 4.51 (s, 2H), 4.22-4.10 (m, 1H), 4.07-3.96 (m, 1H), 3.96-3.76 (m, 4H), 2.84-2.72 (m, 1H), 2.17 (s, 1H), 1.35 (t, J=7.3 Hz, 3H).

Step B: 6-(3-((Benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. To a mixture of 4-(3-((benzyloxy)methyl)-4-ethyl-5-oxo-4,5-dihydro-1H-1,2,4-triazol-1-yl)-N-(2-chlorophenyl)-5-fluoro-2-(1-hydroxy-3-methylbut-3-en-2-yl)benzamide (128 mg, 0.230 mmol) and DMAP (4.0 mg, 0.033 mmol) in DCM (3.5 mL) at 4° C. was added MsCl (304, 0.39 mmol) and Et3N (0.10 mL, 0.72 mmol), and the mixture was stirred at 4° C. to RT for 15 h. After concentration, the residue was purified by flash column chromatography (20-70% EtOAc in heptane) to give the title compound (110 mg, 89%). LCMS (ES-API): mass calcd. for C₃₀H ₂₈ClFN₄O₃, 546.18; m/z found, 547.2 [M+H]⁺. ¹H NMR (400 MHz, CDCl₃) δ 8.11-7.90 (m, 1H), 7.62-7.44 (m, 2H), 7.44-7.28 (m, 8H), 5.13 (s, 1H), 4.84 (s, 0.3H), 4.72 (s, 0.7H), 4.61 (s, 2H), 4.56-4.45 (m, 2H), 4.18-4.02 (m, 1H), 3.96-3.74 (m, 4H), 1.84 (s, 3H), 1.42-1.30 (m, 3H).

Step C: 2-(2-Chlorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. To a solution of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one (82 mg, 0.15 mmol) in DCM (2.5 mL) at −78° C. was added a solution of 1.0M boron trichloride in DCM (0.50 mL, 0.50 mmol). The mixture was stirred at the same temperature for 1 h, and a few drops of MeOH was added. After warming up to RT, the mixture was partitioned between NH₄Cl aqueous solution and DCM. The organic layer was dried over Na₂SO₄, filtered, concentrated, and purified by RF-HPLC (Gemini C18 110A, 5 uM 100×30 mm, 20-100% CH₃CN in water with 10 mM NH₄OH, 60 mL/min) to give the title compound (63 mg, 92%). LCMS (ES-API): mass calcd. for C₂₃H₂₂ClFN₄O₃, 456.14; m/z found, 457.2 [M+H]⁺. ¹H NMR (400 MHz, CDCl₃) δ 8.09-7.91 (m, 1H), 7.57-7.41 (m, 2H), 7.41-7.26 (m, 3H), 5.13 (s, 1H), 4.82 (s, 0.3H), 4.71 (s, 0.7H), 4.62 (d, J=5.4 Hz, 2H), 4.19-3.97 (m, 1H), 3.97-3.67 (m, 4H), 2.76 (br s, 1H), 1.84 (s, 3H), 1.39 (t, J=7.1 Hz, 3H).

### Example 34

### (S*)-6-[4-Ethyl-3-(hydroxymethyl)-5-oxo-1,2,4-triazol-1-yl]-7-fluoro-4-isopropenyl-2-(o-tolyl)-3,4-dihydroisoquinolin-1-one

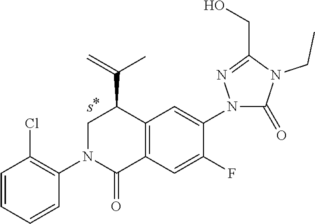 [SMILES: C=C(C)[C@@H]1CN(c2ccccc2Cl)C(=O)c2cc(F)c(-n3nc(CO)n(CC)c3=O)cc21]

A purification of 2-(2-chlorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one (49 mg) was performed via chiral SFC (stationary phase: CHIRACEL AD-H 30×250 mm, mobile phase: 70% CO₂, 30% EtOH) to give the first peak as the title compound (24 mg, 49%). LCMS (ES-API): mass calcd. for C₂₃H₂₂ClFN₄O₃, 456.14; m/z found, 457.2 [M+H]⁺. ¹H NMR (400 MHz, CDCl₃) δ 8.08-7.92 (m, 1H), 7.58-7.42 (m, 2H), 7.42-7.21 (m, 3H), 5.12 (s, 1H), 4.82 (s, 0.3H), 4.70 (s, 0.7H), 4.66-4.50 (m, 2H), 4.18-3.97 (m, 1H), 3.95-3.72 (m, 4H), 3.14-2.86 (m, 1H), 1.84 (s, 3H), 1.38 (t, J=7.1 Hz, 3H).

### Example 35

### (R*)-6-[4-Ethyl-3-(hydroxymethyl)-5-oxo-1,2,4-triazol-1-yl]-7-fluoro-4-isopropyl-2-(o-tolyl)-3,4-dihydroisoquinolin-1-one

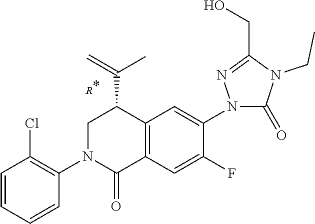 [SMILES: C=C(C)[C@H]1CN(c2ccccc2Cl)C(=O)c2cc(F)c(-n3nc(CO)n(CC)c3=O)cc21]

The title compound (24 mg, 49%) was isolated as the second peak from chiral SFC purification of 2-(2-chlorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one (49 mg, Example 33, Step C). LCMS (ES-API): mass calcd. for C₂₃H₂₂ClFN₄O₃, 456.14; m/z found, 457.2 [M+H]⁺. ¹H NMR (400 MHz, CDCl₃) δ 8.06-7.91 (m, 1H), 7.57-7.42 (m, 2H), 7.40-7.21 (m, 3H), 5.12 (s, 1H), 4.82 (s, 0.3H), 4.70 (s, 0.7H), 4.60 (s, 2H), 4.17-3.99 (m, 1H), 3.97-3.74 (m, 4H), 3.11-2.84 (m, 1H), 1.83 (s, 3H), 1.38 (t, J=7.3 Hz, 3H).

### Example 36

### (S*)-2-(2-Chlorophenyl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one

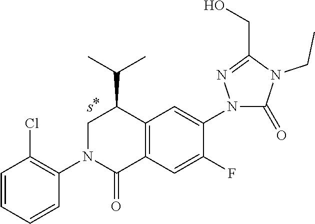 [SMILES: CCn1c(CO)nn(-c2cc3c(cc2F)C(=O)N(c2ccccc2Cl)C[C@H]3C(C)C)c1=O]

A mixture of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one (Example 33, Step B, 249 mg, 0.460 mmol) and 10% Pd/C (48 mg, 0.046 mmol) in EtOAc (50 mL) was hydrogenated under 13 psi H₂ for 6 h. After filtering off the solid through a pad of Celite®, the filtrate was concentrated to give a mixture (202 mg) of 6-(3-((benzyloxy)methyl)-4-ethyl-5-oxo-4,5-dihydro-1H-1,2,4-triazol-1-yl)-2-(2-chlorophenyl)-7-fluoro-4-isopropyl-3,4-dihydroisoquinolin-1(2H)-one and 2-(2-chlorophenyl)-6-(4-ethyl-3 -(hydroxymethyl)-5-oxo-4,5 -dihydro-1H-1,2,4-triazol-1-yl)-7-fluoro-4-isopropyl-3,4-dihydroisoquinolin-1(2H)-one in 3:1 ratio.

This mixture of two compounds (158 mg) in TFA (1 mL) was heated at 75° C. for 10 h and concentrated. The residue was dissolved in 2 mL of THF and treated with 1 mL of 2N K₂CO₃ for 2 h. After removal of solvents, the residue was partitioned between DCM and water. The organic layer was dried over Na₂SO₄, filtered, concentrated, and purified by RF-HPLC (Gemini C18 110A, 5 μM 100×30 mm, 20-100% CH₃CN in water with 10 mM NH₄OH, 60 mL/min) to give racemate 2-(2-chlorophenyl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one as a white foam (124 mg, -90%). A purification of this racemate was performed via chiral SFC (stationary phase: CHIRACEL AD-H 30×250 mm, mobile phase: 70% CO₂, 30% EtOH) to give the first peak as the title compound (52 mg, 39%). LCMS (ES-API): mass calcd. for C₂₃H₂₄ClFN₄O₃, 458.18; m/z found, 459.1 [M+H]⁺. ¹ H NMR(400 MHz, CDCl₃) δ 7.98 (d, J=11.2 Hz, 1H), 7.61-7.48 (m, 2H), 7.44-7.28 (m, 3H), 4.64 (br s, 2H), 4.28 (dd, J=3.9, 13.2 Hz, 0.6H), 4.16 (dd, J=3.9, 12.2 Hz, 0.4H), 3.90 (q, J=7.3 Hz, 2H), 3.85-3.73 (m, 0.4H), 3.70-3.59 (m, 0.6H), 2.77-2.49 (m, 2H), 2.46-2.26 (m, 0.4H), 2.26-2.09 (m, 0.6H), 1.41 (t, J=7.3 Hz, 3H), 1.10-1.01 (m, 3H), 1.01-0.85 (m, 3H).

### Example 37

### (R *)-2-(2-Chlorophenyl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one

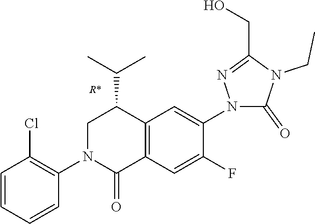 [SMILES: CCn1c(CO)nn(-c2cc3c(cc2F)C(=O)N(c2ccccc2Cl)C[C@@H]3C(C)C)c1=O]

The title compound (52 mg, 39%) was isolated as the second peak from chiral SFC purification of 2-(2-chlorophenyl)-6-(4-ethyl-3-(hydroxymethyl)-5-oxo-4,5-dihydro-1H-1,2,4-triazol-1 -yl)-7-fluoro-4-isopropyl-3,4-dihydroisoquinolin-1(2H)-one. LCMS (ES-API): mass calcd. for C₂₃H₂₄ClFN₄O₃, 458.18; m/z found, 459.1 [M+H]⁺. ¹H NMR (400 MHz, CDCl₃) δ 7.98 (d, J=10.8 Hz, 1H), 7.59-7.45 (m, 2H), 7.45-7.28 (m, 3H), 4.66 (d, J=5.9 Hz, 2H), 4.29 (dd, J=3.9, 12.7 Hz, 0.6H), 4.16 (dd, J=4.2, 12.5 Hz, 0.4H), 3.91 (q, J=7.0 Hz, 2H), 3.80 (dd, J=2.4, 12.7 Hz, 0.4H), 3.66 (dd, J=2.4, 12.7 Hz, 0.6H), 2.60-2.60 (m, 0.6H), 2.60-2.51 (m, 0.4H), 2.48-2.29 (m, 1.4H), 2.26-2.10 (m, 0.6H), 1.41 (t, J=7.1 Hz, 3H), 1.14-1.00 (m, 3H), 1.00-0.83 (m, 3H).

### Example 38

### Racemic-6-(4-ethyl-3-(hydroxymethyl)-5-oxo-4,5-dihydro-1H-1,2,4-triazol-1-yl)-7-fluoro-2-(2-fluoro-5-methylphenyl)-4-(prop-1-en-2-yl)-3,4-dihydroisoquinolin-1(2H)-one

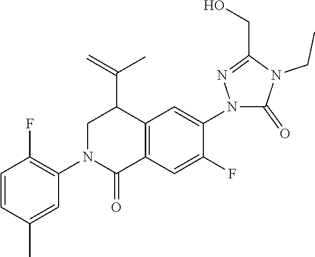 [SMILES: C=C(C)C1CN(c2cc(C)ccc2F)C(=O)c2cc(F)c(-n3nc(CO)n(CC)c3=O)cc21]

Step A: 4-(3-((Benzyloxy)methyl)-4-ethyl-5-oxo-4,5-dihydro-1H-1,2,4-triazol-1-yl)-5-fluoro-N-(2-fluoro-5-methylphenyl)-2-(1-hydroxy-3-methylbut-3-en-2-yl)benzamide. The title compound (340 mg, 85%) was prepared in a manner analogous to Example 33, Step A using 2-fluoro-5-methylaniline instead of 2-chloroaniline. LCMS (ES-API): mass calcd. for C₃₁H₃₂F₂N₄O₄, 562.24; m/z found, 563.3 [M+H]⁺. ¹H NMR (400 MHz, CDCl₃) δ 9.33-9.09 (m, 1H), 8.19 (d, J=7.3 Hz, 1H), 7.54 (d, J=6.8 Hz, 1H), 7.45 (d, J=10.8 Hz, 1H), 7.42-7.29 (m, 5H), 7.06-6.82 (m, 2H), 4.97 (s, 1H), 4.78 (s, 1H), 4.60 (s, 2H), 4.49 (s, 2H), 4.23-4.03 (m, 1H), 4.03-3.73 (m, 4H), 3.46-3.04 (m, 1H), 2.36 (s, 3H), 1.58 (s, 3H), 1.33 (t, J=6.9 Hz, 3H).

Step B: 6-(3-((Benzyloxy)methyl)-4-ethyl-5-oxo-4,5-dihydro-1H-1,2,4-triazol-1-yl)-7-fluoro-2-(2-fluoro-5-methylphenyl)-4-(prop-1-en-2-yl)-3,4-dihydroisoquinolin-1(2H)-one. To a solution of 4-(3-((benzyloxy)methyl)-4-ethyl-5-oxo-4,5-dihydro-1H-1,2,4-triazol-1-yl)-5-fluoro-N-(2-fluoro-5-methylphenyl)-2-(1-hydroxy-3-methylbut-3-en-2-yl)benzamide (280 mg, 0.500 mmol) in THF (50 mL) under Argon at 4° C. was added Ph₃P (166 mg, 0.630 mmol) followed by diisopropyl azodicarboxylate (0.12 mL, 0.61 mmol). The mixture was stirred at 4° C. for 2 h and concentrated. The residue was purified by flash column chromatography (0-60% EtOAc in heptane) to afford the title compound as a clear oil (81 mg, 30%). LCMS (ES-API): mass calcd. for C₃₁H₃₀F₂N₄O₃, 544.23; m/z found, 545.3 [M+H]⁺. ¹H NMR (400 MHz, CDCl₃) δ 8.02 (d, J=10.8 Hz, 1H), 7.49 (d, J=6.8 Hz, 1H), 7.44-7.29 (m, 5H), 7.18-6.98 (m, 3H), 5.09 (s, 1H), 4.76 (s, 1H), 4.61 (s, 2H), 4.52 (s, 2H), 4.06 (dd, J=4.9, 12.2 Hz, 1H), 3.97-3.75 (m, 4H), 2.34 (s, 3H), 1.83 (s, 3H), 1.36 (t, J=7.3 Hz, 3H).

Step C: Racemic-6-(4-Ethyl-3-(hydroxymethyl)-5-oxo-4,5-dihydro-1H-1,2,4-triazol-1-yl)-7-fluoro-2-(2-fluoro-5-methylphenyl)-4-(prop-1-en-2-yl)-3,4-dihydroisoquinolin-1(2H)-one. The title compound (98 mg, 98%) was prepared in a manner analogous to Example 33, Step C using 6-(3-((benzyloxy)methyl)-4-ethyl-5-oxo-4,5-dihydro-1H-1,2,4-triazol-1-yl)-7-fluoro-2-(2-fluoro-5-methylphenyl)-4-(prop-1-en-2-yl)-3,4-dihydroisoquinolin-1(2H)-one instead of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. LCMS (ES-API): mass calcd. for C₂₄H₂₄F₂N₄O₃, 454.18; m/z found, 455.2 [M+H]⁺. ¹ H NMR(400 MHz, CDCl₃) δ 7.99 (d, J=10.8 Hz, 1H), 7.48 (d, J=6.8 Hz, 1H), 7.17-6.99 (m, 3H), 5.08 (s, 1H), 4.75 (s, 1H), 4.62 (s, 2H), 4.06 (dd, J=4.9, 12.7 Hz, 1H), 3.96-3.85 (m, 3H), 3.85-3.73 (m, 1H), 2.76 (br s, 1H), 2.34 (s, 3H), 1.82 (s, 3H), 1.39 (t, J=7.3 Hz, 3H).

### Example 39

### (S *)-6-(4-Ethyl-3-(hydroxymethyl)-5-oxo-4,5-dihydro-1H-1,2,4-triazol-1-yl)-7-fluoro-2-(2-fluoro-5 -methylphenyl)-4-(prop-1-en-2-yl)-3,4-dihydroisoquinolin-1(2H)-one

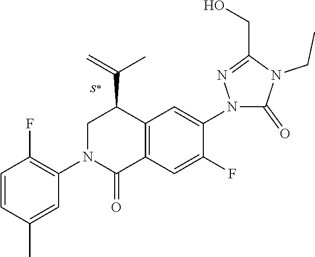 [SMILES: C=C(C)[C@@H]1CN(c2cc(C)ccc2F)C(=O)c2cc(F)c(-n3nc(CO)n(CC)c3=O)cc21]

A purification of 6-(4-ethyl-3-(hydroxymethyl)-5-oxo-4,5-dihydro-1H-1,2,4-triazol-1-yl)-7-fluoro-2-(2-fluoro-5-methylphenyl)-4-(prop-1-en-2-yl)-3,4-dihydroisoquinolin-1(2H)-one (87 mg) was performed via chiral SFC (stationary phase: CHIRACEL AD-H 30×250 mm, mobile phase: 60% CO₂, 40% EtOH) to give the first peak as the title compound (34 mg, 39%). LCMS (ES-API): mass calcd. for C₂₄H₂₄F₂N₄O₃, 454.18; m/z found, 455.3 [M+H]⁺. ¹H NMR (400 MHz, CDCl₃) δ 8.00 (d, J=11.2 Hz, 1H), 7.48 (d, J=6.8 Hz, 1H), 7.16-6.97 (m, 3H), 5.08 (s, 1H), 4.75 (s, 1H), 4.69-4.57 (m, 2H), 4.06 (dd, J=4.9, 12.7 Hz, 1H), 3.96-3.85 (m, 3H), 3.85-3.74 (m, 1H), 2.72-2.51 (m, 1H), 2.34 (s, 3H), 1.82 (s, 3H), 1.40 (t, J=7.1 Hz, 3H).

### Example 40

### (R *)-6-(4-Ethyl-3-(hydroxymethyl)-5-oxo-4,5-dihydro-1H-1,2,4-triazol-1-yl)-7-fluoro-2-(2-fluoro-5 -methylphenyl)-4-(prop-1-en-2-yl)-3,4-dihydroisoquinolin-1(2H)-one

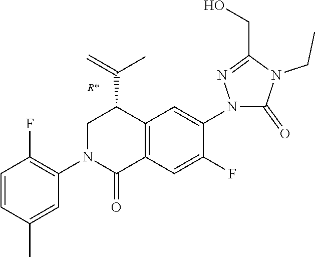 [SMILES: C=C(C)[C@H]1CN(c2cc(C)ccc2F)C(=O)c2cc(F)c(-n3nc(CO)n(CC)c3=O)cc21]

The title compound (37 mg, 43%) was isolated as the second peak from chiral SFC purification of 6-(4-ethyl-3-(hydroxymethyl)-5-oxo-4,5-dihydro-1H-1,2,4-triazol-1-yl)-7-fluoro-2-(2-fluoro-5-methylphenyl)-4-(prop-1-en-2-yl)-3,4-dihydroisoquinolin-1(2H)-one (87 mg, Example 38, Step C). LCMS (ES-API): mass calcd. for C₂₄H₂₄F₂N₄O₃, 454.18; m/z found, 455.1 [M+H]⁺. ¹H NMR (400 MHz, CDCl₃) δ 7.99 (d, J=11.2 Hz, 1H), 7.48 (d, J=6.8 Hz, 1H), 7.18-6.98 (m, 3H), 5.08 (s, 1H), 4.75 (s, 1H), 4.62 (br s, 2H), 4.05 (dd, J=4.9, 12.2 Hz, 1H), 3.97-3.83 (m, 3H), 3.83-3.74 (m, 1H), 2.83 (br s, 1H), 2.34 (s, 3H), 1.82 (s, 3H), 1.39 (t, J=7.1 Hz, 3H).

### Example 41

### (S*)-6-(4-Ethyl-3-(hydroxymethyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one

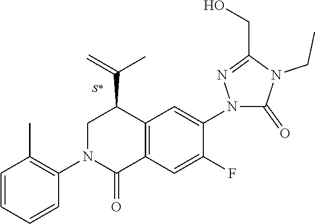 [SMILES: C=C(C)[C@@H]1CN(c2ccccc2C)C(=O)c2cc(F)c(-n3nc(CO)n(CC)c3=O)cc21]

Step A: 4-(3-((Benzyloxy)methyl)-4-ethyl-5-oxo-4,5-dihydro-1H-1,2,4-triazol-1-yl)-5-fluoro-2-(1-hydroxy-3-methylbut-3-en-2-yl)-N-(o-tolyl)benzamide. The title compound (433 mg, 87%) was prepared in a manner analogous to Example 33, Step A using o-toluidine instead of 2-chloroaniline. LCMS (ES-API): mass calcd. for C₃₁H₃₃FN₄O₄, 544.25; m/z found, 545.3 [M+H]⁺. ¹H NMR (400 MHz, CDCl₃) δ 8.59-8.40 (m, 1H), 7.87 (d, J=8.3 Hz, 1H), 7.57 (d, J=7.3 Hz, 1H), 7.48 (d, J=10.3 Hz, 1H), 7.45-7.33 (m, 4H), 7.33-7.20 (m, 3H), 7.20-7.09 (m, 1H), 4.99 (s, 1H), 4.82 (s, 1H), 4.61 (s, 2H), 4.50 (s, 2H), 4.22-4.10 (m, 1H), 4.07-3.99 (m, 1H), 3.96-3.86 (m, 1H), 3.83 (q, J=7.3 Hz, 2H), 3.10-2.89 (m, 1H), 2.29 (s, 3H), 1.76-1.65 (m, 2H), 1.34 (t, J=7.3 Hz, 3H).

Step B: 6-(3-((Benzyloxy)methyl)-4-ethyl-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one. The title compound (179 mg, 58%) was prepared in a manner analogous to Example 33, Step B using 4-(3-((benzyloxy)methyl)-4-ethyl-5-oxo-4,5-dihydro-1H-1,2,4-triazol-1-yl)-5-fluoro-2-(1-hydroxy-3-methylbut-3-en-2-yl)-N-(o-tolyl)benzamide instead of 4-(3-((benzyloxy)methyl)-4-ethyl-5-oxo-4,5-dihydro-1H-1,2,4-triazol-1-yl)-N-(2-chlorophenyl)-5-fluoro-2-(1-hydroxy-3-methylbut-3-en-2-yl)benzamide. LCMS (ES-API): mass calcd. for C₃₁H₃₁FN₄O₃, 526.24; m/z found, 527.1 [M+H]⁺. ¹H NMR (400 MHz, CDCl₃) δ 8.02 (dd, J=2.2, 11.0 Hz, 1H), 7.48 (dd, J=2.4, 6.8 Hz, 1H), 7.42-7.21 (m, 8H), 7.21-7.11 (m, 1H), 5.15 (s, 0.6H), 5.11 (s, 0.4H), 4.75 (s, 0.6H), 4.61 (s, 2H), 4.57 (s, 0.4H), 4.52 (s, 2H), 4.22-4.05 (m, 1H), 3.97-3.68 (m, 4H), 2.27 (s, 1.7H), 2.22 (s, 1.3H), 1.85 (d, J=7.8 Hz, 3H), 1.36 (t, J=7.3 Hz, 3H).

Step C: (S*)-6-(4-Ethyl-3-(hydroxymethyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one. Racemate 6-(4-ethyl-3-(hydroxymethyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one (139 mg, 94%) was prepared in a manner analogous to Example 33, Step C using 6-(3-((benzyloxy)methyl)-4-ethyl-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one instead of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. A purification of this racemate was performed via chiral SFC (stationary phase: CHIRACEL AD-H 20×250 mm, mobile phase: 70% CO₂, 30% EtOH) to give the first peak as the title compound (56 mg, 40%). LCMS (ES-API): mass calcd. for C₂₄H₂₅FN₄O_{3B}, 436.19; m/z found, 437.3 [M+H]⁺. ¹H NMR (400 MHz, CDCl₃) δ 8.02 (d, J=10.1 Hz, 1H), 7.49 (dd, J=3.4, 6.8 Hz, 1H), 7.34-7.21 (m, 3H), 7.22-7.09 (m, 1H), 5.14 (s, 0.6H), 5.11 (s, 0.4H), 4.74 (s, 0.6H), 4.66 (d, J=6.4 Hz, 2H), 4.57 (s, 0.4H), 4.14 (dd, J=4.6, 12.5 Hz, 0.4H), 3.97-3.77 (m, 4H), 3.76-3.68 (m, 0.6H), 2.32-2.25 (m, 1H), 2.27 (s, 2H), 2.22 (s, 1H), 1.84 (d, J=7.8 Hz, 3H), 1.41 (t, J=7.1 Hz, 3H).

### Example 42

### (R *)-6-(4-Ethyl-3-(hydroxymethyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one

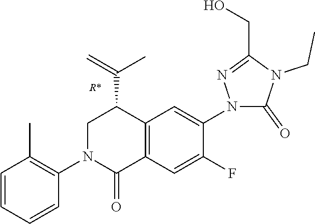 [SMILES: C=C(C)[C@H]1CN(c2ccccc2C)C(=O)c2cc(F)c(-n3nc(CO)n(CC)c3=O)cc21]

The title compound (62 mg, 45%) was isolated as the second peak from chiral SFC purification of 6-(4-ethyl-3-(hydroxymethyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one (139 mg). LCMS (ES-API): mass calcd. for C₂₄H₂₅FN₄O₃, 436.19; m/z found, 437.3 [M+H]⁺. ¹H NMR (400 MHz, CDCl₃) δ 8.02 (d, J=10.3 Hz, 1H), 7.49 (dd, J=3.9, 6.8 Hz, 1H), 7.37-7.22 (m, 3H), 7.21-7.09 (m, 1H), 5.15 (s, 0.5H), 5.11 (s, 0.5H), 4.75 (s, 0.5H), 4.67 (d, J=6.4 Hz, 2H), 4.57 (s, 0.5H), 4.14 (dd, J=5.1, 12.5 Hz, 0.5H), 3.99-3.77 (m, 4H), 3.72 (t, J=4.4 Hz, 0.5H), 2.27 (s, 1.5H), 2.22 (s, 1.5H), 2.21-2.14 (m, 1H), 1.84 (d, J=7.8 Hz, 3H), 1.42 (t, J=7.3 Hz, 3H).

### Example 43

### (S*)-6-(4-Ethyl-3-(hydroxymethyl)-5-oxo-4,5-dihydro-1H-1,2,4-triazol-1-yl)-7-fluoro-4-isopropyl-2-(o-tolyl)-3,4-dihydroisoquinolin-1(2H)-one

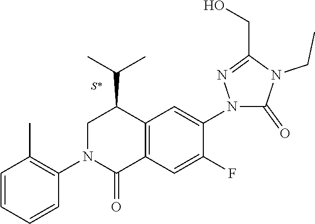 [SMILES: CCn1c(CO)nn(-c2cc3c(cc2F)C(=O)N(c2ccccc2C)C[C@H]3C(C)C)c1=O]

A mixture of (S*)-6-(4-ethyl-3-(hydroxymethyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one (Example 41, Step C, 34 mg, 0.078 mmol) and 10% Pd/C (9.0 mg, 0.0085 mmol) in EtOAc (20 mL) was hydrogenated under 1 atm H₂ balloon for 16 h. After filtering off the solid through a syringe filter, the filtrate was concentrated and purified by RF-HPLC (Gemini C18 110A, 5 μM 100×30 mm, 20-100% CH₃CN in water with 10 mM NH₄OH, 60 mL/min) to give the title compound as a white foam (29 mg, 85%). LCMS (ES-API): mass calcd. for C₂₄H₂₇FN₄O₃, 438.21; m/z found, 439.2 [M+H]⁺. ¹H NMR (400 MHz, CDCl₃) δ 7.98 (dd, J=2.4, 11.2 Hz, 1H), 7.51 (d, J=7.3 Hz, 1H), 7.36-7.19 (m, 3H), 7.19-7.09 (m, 1H), 4.66 (d, J=6.4 Hz, 2H), 4.24 (dd, J=3.7, 12.5 Hz, 0.5H), 4.07 (dd, J=4.2, 13.0 Hz, 0.5H), 3.91 (q, J=7.2 Hz, 2H), 3.75 (dt, J=2.9, 12.5 Hz, 1H), 2.67-2.57 (m, 0.5H), 2.57-2.47 (m, 0.5H), 2.40-2.29 (m, 2H), 2.22 (s, 3H), 1.42 (t, J=7.1 Hz, 3H), 1.09 (d, J=6.8 Hz, 2H), 1.02 (d, J=6.4 Hz, 1H), 0.97 (d, J=6.4 Hz, 3H).

### Example 44

### (R *)-6-(4-Ethyl-3-(hydroxymethyl)-5-oxo-4,5-dihydro-1H-1,2,4-triazol-1-yl)-7-fluoro-4-isopropyl-2-(o-tolyl)-3,4-dihydroisoquinolin-1(2H)-one

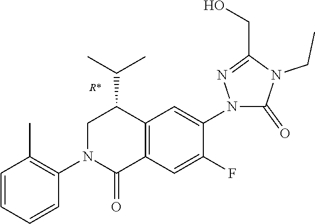 [SMILES: CCn1c(CO)nn(-c2cc3c(cc2F)C(=O)N(c2ccccc2C)C[C@@H]3C(C)C)c1=O]

A mixture of (R *)-6-(4-ethyl-3-(hydroxymethyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one (Example 42, 36 mg, 0.082 mmol) and 10% Pd/C (10 mg, 0.0094 mmol) in EtOAc (20 mL) was hydrogenated under 1 atm H₂ balloon for 16 h. After filtering off the solid through a syringe filter, the filtrate was concentrated and purified by RF-HPLC (Gemini C18 110A, 5 μM 100×30 mm, 20-100% CH₃CN in water with 10 mM NH₄OH, 60 mL/min) to give the title compound as a white foam (25 mg, 69%). LCMS (ES-API): mass calcd. for C₂₄H₂₇FN₄O₃, 438.21; m/z found, 439.2 [M+H]⁺. ¹ H NMR(400 MHz, CDCl₃) δ 7.96 (d, J=10.8 Hz, 1H), 7.51 (d, J=6.8 Hz, 1H), 7.35-7.09 (m, 4H), 4.59 (br s, 2H), 4.23 (dd, J=3.9, 12.7 Hz, 0.4H), 4.07 (dd, J=3.9, 12.7 Hz, 0.6H), 3.89 (q, J=7.3 Hz, 2H), 3.82-3.65 (m, 1H), 3.19-2.97 (m, 1H), 2.67-2.58 (m, 0.6H), 2.58-2.47 (m, 0.4H), 2.31 (s, 1H), 2.21 (s, 3H), 1.39 (t, J=7.1 Hz, 3H), 1.09 (d, J=6.8 Hz, 2H), 1.02 (d, J=6.8 Hz, 1H), 0.96 (d, J=6.8 Hz, 3H).

### Example 45

### Racemic-2-(2-chloro-4-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

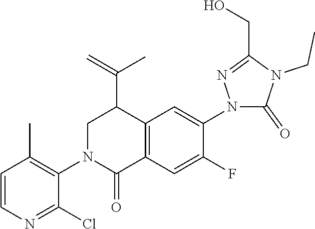 [SMILES: C=C(C)C1CN(c2c(C)ccnc2Cl)C(=O)c2cc(F)c(-n3nc(CO)n(CC)c3=O)cc21]

Step A: 4-(3-((Benzyloxy)methyl)-4-ethyl-5-oxo-4,5-dihydro-1H-1,2,4-triazol-1-yl)-N-(2-chloro-4-methylpyridin-3-yl)-5-fluoro-2-(1-hydroxy-3-methylbut-3-en-2-yl)benzamide. The title compound (253 mg, 62%) was prepared in a manner analogous to Example 33, Step A using 3-amino-2-chloro-4-methylpyridine instead of 2-chloroaniline. LCMS (ES-API): mass calcd. for C₃₀H₃₁ClFN₅O₄, 579.20; m/z found, 580.2 [M+H]⁺. ¹1-1 NMR (400 MHz, CDCl₃) δ 9.54-9.37 (m, 1H), 8.18 (d, J=4.9 Hz, 1H), 7.59 (d, J=6.8 Hz, 1H), 7.53 (d, J=10.3 Hz, 1H), 7.44-7.29 (m, 5H), 7.20 (d, J=4.9 Hz, 1H), 5.01 (s, 1H), 4.83 (s, 1H), 4.60 (s, 2H), 4.50 (s, 2H), 4.29-4.15 (m, 2H), 4.01-3.88 (m, 1H), 3.81 (q, J=7.3 Hz, 2H), 3.40 (br s, 1H), 2.40 (s, 3H), 2.17 (s, 1H), 2.01 (s, 2H), 1.32 (t, J=7.1 Hz, 3H).

Step B: 6-(3-((Benzyloxy)methyl)-4-ethyl-5-oxo-4,5-dihydro-1H-1,2,4-triazol-1-yl)-2-(2-chloro-4-methylpyridin-3-yl)-7-fluoro-4-(prop-1-en-2-yl)-3,4-dihydroisoquinolin-1(2H)-one. The title compound (54 mg, 59%) was prepared in a manner analogous to Example 33, Step B using 4-(3-((benzyloxy)methyl)-4-ethyl-5-oxo-4,5-dihydro-1H-1,2,4-triazol-1-yl)-N-(2-chloro-4-methylpyridin-3-yl)-5-fluoro-2-(1-hydroxy-3-methylbut-3-en-2-yl)benzamide instead of 4-(3-((benzyloxy)methyl)-4-ethyl-5-oxo-4,5-dihydro-1H-1,2,4-triazol-1-yl)-N-(2-chlorophenyl)-5-fluoro-2-(1-hydroxy-3-methylbut-3-en-2-yl)benzamide. LCMS (ES-API): mass calcd. for C₃₀H₂₉ClFN₅O₃, 561.19; m/z found, 562.3 [M+H]⁺. ¹H NMR (400 MHz, CDCl₃) δ 8.25 (d, J=4.9 Hz, 1H), 8.02 (d, J=10.8 Hz, 1H), 7.52 (d, J=6.8 Hz, 1H), 7.43-7.30 (m, 5H), 7.19 (d, J=4.9 Hz, 1H), 5.17 (s, 1H), 4.68 (s, 1H), 4.62 (s, 2H), 4.52 (s, 2H), 4.21-4.07 (m, 1H), 3.93 (t, J=5.6 Hz, 1H), 3.86 (q, J=7.0 Hz, 2H), 3.71 (dd, J=6.4, 12.7 Hz, 1H), 2.30 (s, 3H), 1.86 (s, 3H), 1.36 (t, J=7.3 Hz, 3H).

Step C: 2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. The title compound (108 mg, 92%) was prepared in a manner analogous to Example 33, Step C using 6-(3-((benzyloxy)methyl)-4-ethyl-5-oxo-4,5-dihydro-1H-1,2,4-triazol-1-yl)-2-(2-chloro-4-methylpyridin-3-yl)-7-fluoro-4-(prop-1-en-2-yl)-3,4-dihydroisoquinolin-1(2H)-one instead of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. LCMS (ES-API): mass calcd. for C₂₃H₂₃ClFN₅O₃, 471.15; m/z found, 472.2 [M+H]⁺. ¹H NMR (400 MHz, CDCl₃) δ 8.25 (d, J=4.9 Hz, 1H), 8.01 (d, J=10.8 Hz, 1H), 7.52 (d,J=6.8 Hz, 1H), 7.19 (d,J=4.9 Hz, 1H), 5.16 (s, 1H), 4.68 (s, 2H), 4.66 (s, 1H), 4.15 (dd, J=5.1, 12.5 Hz, 1H), 3.98-3.84 (m, 3H), 3.71 (dd, J=6.6, 12.5 Hz, 1H), 2.45-2.32 (m, 1H), 2.29 (s, 3H), 1.86 (s, 3H), 1.41 (t, J=7.3 Hz, 3H).

### Example 46

### (S *)-2-(2-Chloro-4-methy 1py ri din-3 -yl)-6-(4-ethyl-3 -(hydroxymethyl)-5-oxo-4,5 -dihydro-1H-1,2,4-triazol-1 -yl)-7-fluoro-4-(prop-1 -en-2-yl)-3,4-dihydroisoquinolin-1 (2H)-one

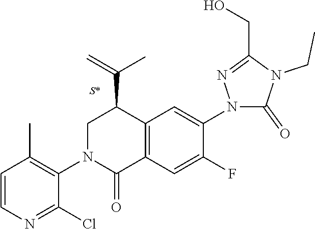 [SMILES: C=C(C)[C@@H]1CN(c2c(C)ccnc2Cl)C(=O)c2cc(F)c(-n3nc(CO)n(CC)c3=O)cc21]

A purification of 2-(2-chloro-4-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one (128 mg) was performed via chiral SFC (stationary phase: CHIRACEL AD-H 20×250 mm, mobile phase: 70% CO₂, 30% EtOH) to give the first peak as the title compound (59 mg, 46%). LCMS (ES-API): mass calcd. for C₂₃H₂₃ClFN₅O₃, 471.15; m/z found, 472.2 [M+H]⁺. ¹H NMR (400 MHz, CDCl₃) δ 8.34-8.18 (m, 1H), 8.06-7.96 (m, 1H), 7.58-7.44 (m, 1H), 7.25-7.14 (m, 1H), 5.18 (d, 1H), 4.74-4.60 (m, 3H), 4.27-4.09 (m, 1H), 4.06-3.83 (m, 3H), 3.78-3.64 (m, 0.5H), 3.51 (dd, J=5.4, 12.2 Hz, 0.5H), 2.56 (br s, 1H), 2.35 (s, 1H), 2.29 (s, 2H), 1.85 (s, 3H), 1.41 (t, J=7.3 Hz, 3H).

### Example 47

### (R *)-2-(2-Chloro-4-methylpyridin-3-yl)-6-(4-ethyl-3-(hydroxymethyl)-5-oxo-4,5 -dihydro-1H-1,2,4-triazol-1-yl)-7-fluoro-4-(prop-1-en-2-yl)-3,4-dihydroisoquinolin-1 (2H)-one

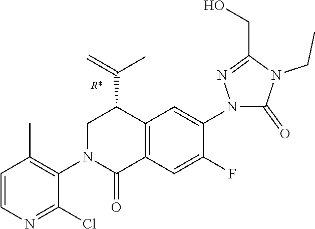 [SMILES: C=C(C)[C@H]1CN(c2c(C)ccnc2Cl)C(=O)c2cc(F)c(-n3nc(CO)n(CC)c3=O)cc21]

The title compound (56 mg, 44%) was isolated as the second peak from chiral SFC purification of 2-(2-chloro-4-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one (128 mg, Example 45, Step C). LCMS (ES-API): mass calcd. for C₂₃H₂₃ClFN₅O₃, 471.15; m/z found, 472.2 [M+H]⁺. ¹H NMR (400 MHz, CDCl₃) δ 8.31-8.19 (m, 1H), 8.07-7.95 (m, 1H), 7.57-7.47 (m, 1H), 7.25-7.15 (m, 1H), 5.23-5.12 (m, 1H), 4.71-4.59 (m, 3H), 4.25-4.08 (m, 1H), 4.05-3.81 (m, 3H), 3.78-3.63 (m, 1H), 3.12 (br s, 1H), 2.35 (s, 1H), 2.29 (s, 2H), 1.91-1.70 (m, 3H), 1.40 (t, J=7.3 Hz, 3H).

### Example 48

### (S*)-2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

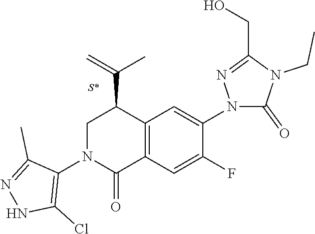 [SMILES: C=C(C)[C@@H]1CN(c2c(C)n[nH]c2Cl)C(=O)c2cc(F)c(-n3nc(CO)n(CC)c3=O)cc21]

Step A: 4-(3-((Benzyloxy)methyl)-4-ethyl-5-oxo-4,5-dihydro-1H-1,2,4-triazol-1-yl)-N-(5-chloro-3-methyl-1H-pyrazol-4-yl)-5-fluoro-2-(1-hydroxy-3-methylbut-3-en-2-yl)benzamide. To a suspension of 5-chloro-3-methyl-4-amino-1H-pyrazole (186 mg, 1.41 mmol) in 2.5 mL of DCM at 4° C. was added trimethylaluminum in toluene (2.0 M, 0.76 mL, 1.5 mmol), and the suspension became a clear solution. After stirring for 1.5 h at 4° C., to the mixture was added a solution of 5-((benzyloxy)methyl)-4-ethyl-2-(7-fluoro-1-oxo-4-(prop-1-en-2-yl)isochroman-6-yl)-2,4-dihydro-3H-1,2,4-triazol-3-one (250 mg, 0.570 mmol) in DCM (2.5 mL). The mixture was stirred at RT for 2 h and then heated at 60° C. for 12 h. Ice was added to the reaction mixture followed by 1.8 mL of 2 M HCl slowly. The mixture was extracted with DCM, and the organic layer was dried over Na₂SO₄, filtered, concentrated, and purified by flash column chromatography (30-100% EtOAc in heptane) to give the title compound as a clear oil. LCMS (ES-API): mass calcd. for C₂₈H₃₀ClFN₆O_{4, 568.20}; m/z found, 569.3 [M+H]⁺. ¹H NMR (400 MHz, CDCl₃) δ 9.11-8.93 (m, 1H), 7.54 (dd, J=2.0, 7.3 Hz, 1H), 7.45 (dd, J=3.4, 10.3 Hz, 1H), 7.42-7.30 (m, 4H), 4.99 (s, 1H), 4.83 (s, 1H), 4.61 (s, 2H), 4.51 (s, 2H), 4.26-4.17 (m, 1H), 4.17-4.08 (m, 1H), 4.12 (q, J=7.0 Hz, 2H), 3.96-3.76 (m, 3H), 2.32-2.16 (m, 3H), 2.05 (s, 3H), 1.26 (t, J=7.1 Hz, 3H).

Step B: 6-(3-((Benzyloxy)methyl)-4-ethyl-5-oxo-4,5-dihydro-1H-1,2,4-triazol-1-yl)-2-(5-chloro-3-methyl-1H-pyrazol-4-yl)-7-fluoro-4-(prop-1-en-2-yl)-3,4-dihydroisoquinolin-1(2H)-one. The title compound was prepared in a manner analogous to Example 38, Step B using 4-(3-((benzyloxy)methyl)-4-ethyl-5-oxo-4,5-dihydro-1H-1,2,4-triazol-1-yl)-N-(5-chloro-3-methyl-1H-pyrazol-4-yl)-5-fluoro-2-(1-hydroxy-3-methylbut-3-en-2-yl)benzamide instead of 4-(3-((benzyloxy)methyl)-4-ethyl-5-oxo-4,5-dihydro-1H-1,2,4-triazol-1-yl)-5-fluoro-N-(2-fluoro-5-methylphenyl)-2-(1-hydroxy-3-methylbut-3-en-2-yl)benzamide. LCMS (ES-API): mass calcd. for C₂₈H₂₈ClFN₆O₃, 550.19; m/z found, 551.3 [M+H]⁺. ¹H NMR (400 MHz, CDCl₃) δ 8.00 (d, J=10.3 Hz, 1H), 7.53-7.44 (m, 1H), 7.44-7.28 (m, 5H), 5.10 (s, 1H), 4.67-4.54 (m, 1H), 4.61 (s, 2H), 4.52 (s, 2H), 3.92-3.79 (m, 3H), 3.79-3.61 (m, 2H), 2.25-2.11 (m, 4H), 1.84 (s, 3H), 1.36 (t, J=7.1 Hz, 3H).

Step C: (S*)-2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. Racemate 2-(5-chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one was prepared in a manner analogous to Example 33, Step C using 6-(3-((benzyloxy)methyl)-4-ethyl-5-oxo-4,5-dihydro-1H-1,2,4-triazol-1-yl)-2-(5-chloro-3-methyl-1H-pyrazol-4-yl)-7-fluoro-4-(prop-1-en-2-yl)-3,4-dihydroisoquinolin-1(2H)-one instead of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. A purification of this racemate was performed via chiral SFC (stationary phase: CHIRACEL AD-H 30×250 mm, mobile phase: 80% CO₂, 20% MeOH) to give the first peak as the title compound. LCMS (ES-API): mass calcd. for C₂₁H₂₂ClFN₆O₃, 460.14; m/z found, 461.1 [M+H]⁺. ¹H NMR (400 MHz, CDCl₃) δ 8.00 (d, J=10.3 Hz, 1H), 7.51 (d, J=6.8 Hz, 2H), 5.10 (s, 1H), 4.68 (d, J=6.4 Hz, 2H), 3.91 (q, J=6.8 Hz, 2H), 3.86-3.63 (m, 3H), 2.21 (s, 3H), 2.13-2.02 (m, 1H), 1.84 (s, 3H), 1.42 (t, J=7.1 Hz, 3H).

### Example 49

### (R *)-2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

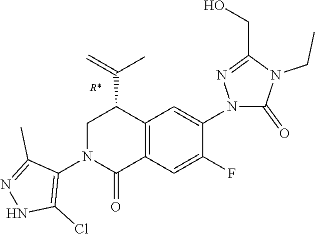 [SMILES: C=C(C)[C@H]1CN(c2c(C)n[nH]c2Cl)C(=O)c2cc(F)c(-n3nc(CO)n(CC)c3=O)cc21]

The title compound was isolated as the second peak from chiral SFC purification of 2-(5-chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1 -en-2-yl)-3,4-dihydroisoquinolin-1(2H)-one. LCMS (ES-API): mass calcd. for C₂₁H₂₂ClFN₆O₃, 460.14; m/z found, 461.0 [M+H]⁺. ¹ H NMR(400 MHz, CDCl₃) δ 10.01-9.58 (m, 1H), 8.00 (d, J=11.2 Hz, 1H), 7.51 (d, J=6.8 Hz, 1H), 5.10 (s, 1H), 4.68 (d, J=5.9 Hz, 2H), 3.91 (q, J=6.8 Hz, 2H), 3.84-3.66 (m, 3H), 2.20 (s, 3H), 2.19-2.10 (m, 1H), 1.84 (s, 3H), 1.42 (t, J=7.4 Hz, 3H).

### Example 50

### Atropisomer 1, (S*)-6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one

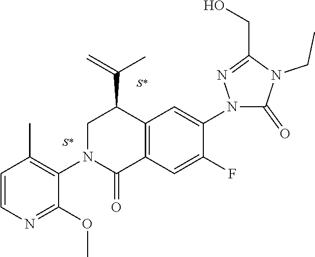 [SMILES: C=C(C)[C@@H]1CN(c2c(C)ccnc2OC)C(=O)c2cc(F)c(-n3nc(CO)n(CC)c3=O)cc21]

Step A: 4-(3-((Benzyloxy)methyl)-4-ethyl-5-oxo-4,5-dihydro-1H-1,2,4-triazol-1-yl)-5-fluoro-2-(1-hydroxy-3-methylbut-3-en-2-yl)-N-(2-methoxy-4-methylpyridin-3-yl)benzamide. The title compound was prepared in a manner analogous to Example 33, Step A using 2-methoxy-4-methylpyridin-3-amine instead of 2-chloroaniline. LCMS (ES-API): mass calcd. for C₃₁H₃₄FN₅O₅, 575.25; m/z found, 576.2 [M+H]⁺. ¹H NMR (400 MHz, CDCl₃) δ 8.39 (s, 1H), 7.98 (d, J=4.9 Hz, 1H), 7.59 (d, J=7.3 Hz, 1H), 7.51 (d, J=10.3 Hz, 1H), 7.44-7.29 (m, 5H), 6.84 (d, J=5.4 Hz, 1H), 5.01 (s, 1H), 4.85 (s, 1H), 4.61 (s, 2H), 4.51 (s, 2H), 4.26-4.16 (m, 2H), 3.98-3.88 (m, 5H), 3.84 (q, J=6.9 Hz, 2H), 3.01 (br s, 1H), 2.34 (s, 3H), 1.69 (s, 2H), 1.35 (t, J=7.1 Hz, 3H).

Step B: 6-(3-((Benzyloxy)methyl)-4-ethyl-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one. The title compound was prepared in a manner analogous to Example 33, Step B using 4-(3-((benzyloxy)methyl)-4-ethyl-5-oxo-4,5-dihydro-1H-1,2,4-triazol-1-yl)-5-fluoro-2-(1-hydroxy-3-methylbut-3-en-2-yl)-N-(2-methoxy-4-methylpyridin-3-yl)benzamide instead of 4-(3-((benzyloxy)methyl)-4-ethyl-5-oxo-4,5-dihydro-1H-1,2,4-triazol-1-yl)-N-(2-chlorophenyl)-5-fluoro-2-(1-hydroxy-3-methylbut-3-en-2-yl)benzamide. LCMS (ES-API): mass calcd. for C₃₁H₃₂FN₅O₄, 557.24; m/z found, 558.3 [M+F]⁺. ¹H NMR (400 MHz, CDCl₃) δ 8.07-7.93 (m, 2H), 7.54-7.43 (m, 1H), 7.43-7.30 (m, 5H), 6.89-6.74 (m, 1H), 5.15 (s, 0.2H), 5.11 (s, 0.8H), 4.94 (s, 0.2H), 4.61 (s, 2H), 4.57-4.42 (m, 2.8H), 4.21 (dd, J=4.6, 12.5 Hz, 0.8H), 4.16-4.04 (m, 0.2H), 3.99-3.90 (m, 3H), 3.86 (q, J=7.3 Hz, 2H), 3.73 (br t, J=4.6 Hz, 1H), 3.59 (dd, J=4.9, 12.7 Hz, 0.8H), 3.48 (dd, J=5.4, 0.2 Hz), 2.26 (s, 0.7H), 2.21 (s, 2.3H), 1.84 (s, 3H), 1.36 (t, J=7.3 Hz, 3H).

Step C: (S*)-6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one. Racemate 6-(4-ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one was prepared in a manner analogous to Example 33, Step C using 6-(3-((benzyloxy)methyl)-4-ethyl-5-oxo-4,5-dihydro-1H-1,2,4-triazol-1-yl)-7-fluoro-2-(2-methoxy -4-methylpy ridin-3 -yl)-4-(prop-1-en-2-yl)-3,4-dihydroisoquinolin-1(2H)-one instead of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. A purification of this racemate was performed via chiral SFC (stationary phase: CHIRACEL AD-H 30×250 mm, mobile phase: 75% CO₂, 25% iPrOH) to give the first peak as the title compound. LCMS (ES-API): mass calcd. for C₂₄H₂₆FN₅O₄, 467.20; m/z found, 468.3 [M+H]⁺. ¹H NMR (400 MHz, CDCl₃) δ 8.10-7.94 (m, 2H), 7.47 (d, J=6.8 Hz, 1H), 6.84 (d, J=4.9 Hz, 1H), 5.15 (s, 1H), 4.93 (s, 1H), 4.65 (s, 2H), 4.16-4.02 (m, 1H), 4.01-3.82 (m, 6H), 3.48 (dd, J=5.4, 12.2 Hz, 1H), 2.78-2.51 (m, 1H), 2.26 (s, 3H), 1.83 (s, 3H), 1.41 (t, J=7.1 Hz, 3H).

### Example 51

### Atropisomer 2, (S *)-6-(4-Ethyl-3 -(hydroxymethyl)-5-oxo-4,5-dihydro-1H-1,2,4-triazol-1 -yl)-7-fluoro-2-(2-methoxy -4-methy 1py ri din-3 -yl)-4-(prop-1-en-2-yl)-3,4-dihydroisoquinolin-1 (2H)-one

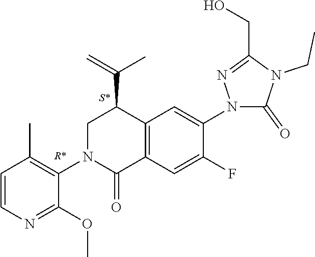 [SMILES: C=C(C)[C@@H]1CN(c2c(C)ccnc2OC)C(=O)c2cc(F)c(-n3nc(CO)n(CC)c3=O)cc21]

The title compound was isolated as the second peak from chiral SFC purification of 6-(4-ethyl-3 -(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one. LCMS (ES-API): mass calcd. for C₂₄H₂₆FN₅O₄, 467.20; m/z found, 468.3 [M+H]⁺. ¹H NMR (400 MHz, CDCl₃) δ 8.05-7.92 (m, 2H), 7.49 (d, J=6.8 Hz, 1H), 6.82 (d, J=5.4 Hz, 1H), 5.10 (s, 1H), 4.67-4.57 (m, 2H), 4.53 (s, 1H), 4.20 (dd, J=4.4, 12.7 Hz, 1H), 4.00-3.81 (m, 5H), 3.73 (br t, J=4.4 Hz, 1H), 3.60 (dd, J=4.4, 12.7 Hz, 1H), 3.17-3.01 (m, 1H), 2.20 (s, 3H), 1.84 (s, 3H), 1.39 (t, J=7.3 Hz, 3H).

### Example 52

### Atropisomer 1, (R *)-6-(4-Ethyl-3 -(hydroxymethyl)-5-oxo-4,5 -dihydro-1H-1,2,4-triazol-1-yl)-7-fluoro-2-(2-methoxy-4-methylpyridin-3-yl)-4-(prop-1-en-2-yl)-3,4-dihydroisoquinol in-1 (2H)-one

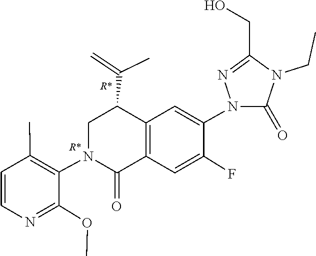 [SMILES: C=C(C)[C@H]1CN(c2c(C)ccnc2OC)C(=O)c2cc(F)c(-n3nc(CO)n(CC)c3=O)cc21]

The title compound was isolated as the third peak from chiral SFC purification of 6-(4-ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one. LCMS (ES-API): mass calcd. for C₂₄H₂₆FN₅O₄, 467.20; m/z found, 468.3 [M+H]⁺. ¹H NMR (400 MHz, CDCl₃) δ 8.02 (s, 1H), 8.00 (d, J=5.9 Hz, 1H), 7.47 (d, J=6.8 Hz, 1H), 6.84 (d, J=5.4 Hz, 1H), 5.15 (s, 1H), 4.93 (s, 1H), 4.64 (s, 2H), 4.15-4.03 (m, 1H), 4.00-3.80 (m, 6H), 3.48 (dd, J=4.9, 12.2 Hz, 1H), 2.84-2.57 (m, 1H), 2.26 (s, 3H), 1.83 (s, 3H), 1.40 (t, J=7.1 Hz, 3H).

### Example 53

### Atropisomer 2, (R*)-6-(4-Ethyl-3-(hydroxymethyl)-5-oxo-4,5-dihydro-azol-1 -yl)-7-fluoro-2-(2-methoxy-4-methylpyridin-3 -yl)-4-(prop-1-en-2-yl)-3,4-dihydroisoquinolin-1 (2H)-one

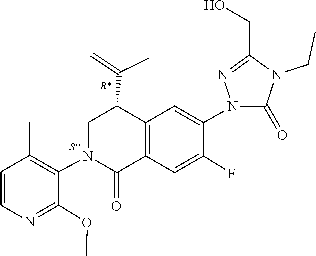 [SMILES: C=C(C)[C@H]1CN(c2c(C)ccnc2OC)C(=O)c2cc(F)c(-n3nc(CO)n(CC)c3=O)cc21]

The title compound was isolated as the fourth peak from chiral SFC purification of 6-(4-ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one. LCMS (ES-API): mass calcd. for C₂₄H₂₆FN₅O₄, 467.20; m/z found, 468.1 [M+H]⁺. ¹H NMR (400 MHz, CDCl₃) δ 8.06-7.92 (m, 2H), 7.49 (d, J=6.8 Hz, 1H), 6.82 (d, J=5.4 Hz, 1H), 5.10 (s, 1H), 4.61 (br s, 2H), 4.52 (s, 1H), 4.20 (dd, J=4.4, 12.7 Hz, 1H), 4.00-3.79 (m, 5H), 3.78-3.68 (m, 1H), 3.60 (dd, J=4.4, 12.7 Hz, 1H), 3.16 (br s, 1H), 2.20 (s, 3H), 1.84 (s, 3H), 1.39 (t, J=7.3 Hz, 3H).

### Example 54

### Atropisomer 1, (S*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1 (2H)-one

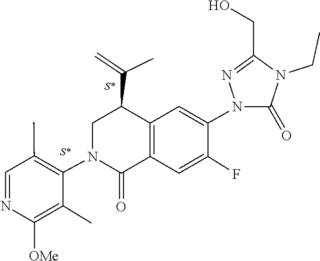 [SMILES: C=C(C)[C@@H]1CN(c2c(C)cnc(OC)c2C)C(=O)c2cc(F)c(-n3nc(CO)n(CC)c3=O)cc21]

Step A: 4-(3-((Benzyloxy)methyl)-4-ethyl-5-oxo-4,5-dihydro-1H-1,2,4-triazol-1-yl)-5-fluoro-2-(1-hydroxy-3-methylbut-3-en-2-yl)-N-(2-methoxy-3,5-dimethylpyridin-4-yl)benzamide. The title compound was prepared in a manner analogous to Example 33, Step A using 2-methoxy-3,5-dimethylpyridin-4-amine instead of 2-chloroaniline. LCMS (ES-API): mass calcd. for C₃₂H₃₆FN₅O₅, 589.27; m/z found, 590.2 [M+H]⁺. ¹H NMR (400 MHz, CDCl₃) δ 9.30-9.16 (m, 1H), 7.86 (s, 1H), 7.61-7.45 (m, 2H), 7.45-7.29 (m, 5H), 5.00 (s, 1H), 4.81 (s, 1H), 4.60 (s, 2H), 4.49 (s, 2H), 4.22-4.07 (m, 2H), 4.00-3.85 (m, 4H), 3.79 (q, J=7.0 Hz, 2H), 3.70-3.52 (m, 1H), 2.18 (s, 3H), 2.13 (s, 3H), 1.96-1.83 (m, 2H), 1.31 (t, J=6.9 Hz, 3H).

Step B: 6-(3-((Benzyloxy)methyl)-4-ethyl-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one, Atropisomers 1 and 2. The title compound was prepared in a manner analogous to Example 33, Step B using 4-(3-((benzyloxy)methyl)-4-ethyl-5-oxo-4,5-dihydro-1H-1,2,4-triazol-1-yl)-5-fluoro-2-(1-hydroxy-3-methylbut-3-en-2-yl)-N-(2-methoxy-3,5-dimethylpyridin-4-yl)benzamide instead of 4-(3-((benzyloxy)methyl)-4-ethyl-5-oxo-4,5-dihydro-1H-1,2,4-triazol-1-yl)-N-(2-chlorophenyl)-5-fluoro-2-(1-hydroxy-3-methylbut-3-en-2-yl)benzamide. LCMS (ES-API): mass calcd. for C₃₂H₃₄FN₅O₄, 571.26; m/z found, 572.3 [M+H]⁺. ¹ H NMR(400 MHz, CDCl₃) δ 8.03 (d, J=10.8 Hz, 1H), 7.93 (s, 1H), 7.50 (d, J=6.8 Hz, 1H), 7.44-7.29 (m, 5H), 5.17 (s, 1H), 4.75 (s, 1H), 4.62 (s, 2H), 4.52 (s, 2H), 3.95 (s, 3H), 3.91-3.81 (m, 3H), 3.77 (d, J=6.4 Hz, 2H), 2.11 (s, 6H), 1.84 (s, 3H), 1.36 (t, J=7.3 Hz, 3H).

Step C: Atropisomer 1, (S*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one. Racemate 6-(4-ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one was prepared in a manner analogous to Example 33, Step C using 6-(3-((benzyloxy)methyl)-4-ethyl-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one instead of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. A purification of this racemate was performed via chiral SFC (stationary phase:

CHIRACEL AD-H 30×250 mm, mobile phase: 60% CO₂, 40% MeOH) to give the first peak as the title compound. LCMS (ES-API): mass calcd. for C₂₅H₂₈FN₅O₄, 481.21; m/z found, 482.3 [M+H]⁺. ¹H NMR (400 MHz, CDCl₃) δ 8.02 (d, J=10.8 Hz, 1H), 7.92 (s, 1H), 7.50 (d, J=6.4 Hz, 1H), 5.17 (s, 1H), 4.74 (s, 1H), 4.65 (d, J=5.4 Hz, 2H), 4.01-3.83 (m, 6H), 3.77 (d, J=6.4 Hz, 2H), 2.66 (br t, J=6.1 Hz, 1H), 2.10 (s, 6H), 1.84 (s, 3H), 1.41 (t, J=7.3 Hz, 3H).

### Example 55

### Atropisomer 1, (R *)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one

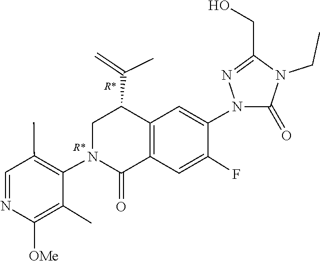 [SMILES: C=C(C)[C@H]1CN(c2c(C)cnc(OC)c2C)C(=O)c2cc(F)c(-n3nc(CO)n(CC)c3=O)cc21]

The title compound was isolated as the second peak from chiral SFC purification of racemate 6-(4-ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one. LCMS (ES-API): mass calcd. for C₂₅H₂₈FN₅O₄, 481.21; m/z found, 482.3 [M+H]⁺. ¹H NMR (400 MHz, CDCl₃) δ 8.03 (d, J=10.8 Hz, 1H), 7.93 (s, 1H), 7.50 (d, J=6.8 Hz, 1H), 5.17 (s, 1H), 4.75 (s, 1H), 4.66 (d, J=5.4 Hz, 2H), 4.01-3.82 (m, 6H), 3.77 (d, J=6.4 Hz, 2H), 2.38 (br t, J=5.9 Hz, 1H), 2.11 (s, 6H), 1.84 (s, 3H), 1.41 (t, J=7.1 Hz, 3H).

### Example 56

### Atropisomer 2, (S *)-6-(4-Ethyl-3 -(hydroxymethyl)-5-oxo-4,5-dihydro-1H-1,2,4-triazol-1 -yl)-7-fluoro-2-(2-methoxy -3,5 -dimethy 1py ri din-4-yl)-4-(prop-1-en-2-yl)-3,4-dihydroisoquinolin-1 (2H)-one

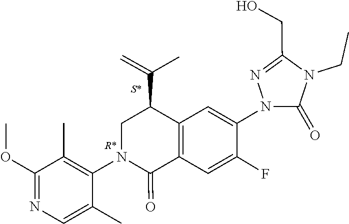 [SMILES: C=C(C)[C@@H]1CN(c2c(C)cnc(OC)c2C)C(=O)c2cc(F)c(-n3nc(CO)n(CC)c3=O)cc21]

Step A: 6-(3-((Benzyloxy)methyl)-4-ethyl-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one. The title compound was isolated as the second fraction from the same reaction in Example 54, Step B. LCMS (ES-API): mass calcd. for C₃₂H₃₄FN₅O₄, 571.26; m/z found, 572.3 [M+H]⁺. ¹H NMR (400 MHz, CDCl₃) δ 8.03 (d, J=10.8 Hz, 1H), 7.94 (s, 1H), 7.50 (d, J=6.8 Hz, 1H), 7.43-7.30 (m, 5H), 5.18 (s, 1H), 4.73 (s, 1H), 4.62 (s, 2H), 4.52 (s, 2H), 3.95 (s, 3H), 3.91-3.81 (m, 3H), 3.81-3.70 (m, 2H), 2.14 (s, 3H), 2.07 (s, 3H), 1.85 (s, 3H), 1.36 (t, J=7.1 Hz, 3H).

Step B: Atropisomer 2, (S*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one. Racemate 6-(4-ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one was prepared in a manner analogous to Example 33, Step C using 6-(3-((benzyloxy)methyl)-4-ethyl-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one instead of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. A purification of this racemate was performed via chiral SFC (stationary phase: CHIRACEL AD-H 30×250 mm, mobile phase: 70% CO₂, 30% EtOH) to give the first peak as the title compound. LCMS (ES-API): mass calcd. for C₂₅H₂₈FN₅O₄, 481.21; m/z found, 482.1 [M+H]⁺. ¹H NMR (400 MHz, CDCl₃) δ 8.02 (d, J=10.8 Hz, 1H), 7.94 (s, 1H), 7.50 (d, J=6.8 Hz, 1H), 5.17 (s, 1H), 4.73 (s, 1H), 4.64 (d, J=4.9 Hz, 2H), 4.01-3.82 (m, 6H), 3.82-3.68 (m, 2H), 2.87-2.73 (m, 1H), 2.14 (s, 3H), 2.07 (s, 3H), 1.84 (s, 3H), 1.40 (t, J=7.1 Hz, 3H).

### Example 57

### Atropisomer 2, (R*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one

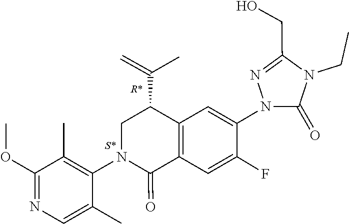 [SMILES: C=C(C)[C@H]1CN(c2c(C)cnc(OC)c2C)C(=O)c2cc(F)c(-n3nc(CO)n(CC)c3=O)cc21]

The title compound was isolated as the second peak from chiral SFC purification of racemate 6-(4-ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one. LCMS (ES-API): mass calcd. for C₂₅H₂₈FN₅O₄, 481.21; m/z found, 482.1 [M+H]⁺. ¹H NMR (400 MHz, CDCl₃) δ 8.02 (d, J=10.8 Hz, 1H), 7.94 (s, 1H), 7.50 (d, J=6.8 Hz, 1H), 5.17 (s, 1H), 4.73 (s, 1H), 4.65 (br s, 2H), 4.03-3.83 (m, 6H), 3.83-3.68 (m, 2H), 2.59 (br s, 1H), 2.14 (s, 3H), 2.07 (s, 3H), 1.84 (s, 3H), 1.41 (t, J=7.1 Hz, 3H).

### Example 58

### Atropisomer 1, (S*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

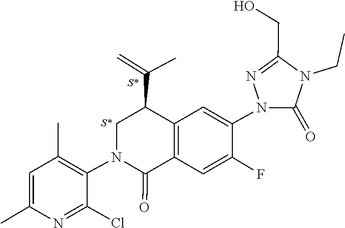 [SMILES: C=C(C)[C@@H]1CN(c2c(C)cc(C)nc2Cl)C(=O)c2cc(F)c(-n3nc(CO)n(CC)c3=O)cc21]

Step A: 4-(3-((Benzyloxy)methyl)-4-ethyl-5-oxo-4,5-dihydro-1H-1,2,4-triazol-1-yl)-N-(2-chloro-4,6-dimethylpyridin-3-yl)-5-fluoro-2-(1-hydroxy-3-methylbut-3-en-2-yl)benzamide. The title compound was prepared in a manner analogous to Example 33, Step A using 2-chloro-4,6-dimethylpyridin-3-amine instead of 2-chloroaniline. LCMS (ES-API): mass calcd. for C₃₁H₃₃ClFN₅O₄, 593.22; m/z found, 594.2 [M+H]⁺. ¹H NMR (400 MHz, CDCl₃) δ 9.49-9.31 (m, 1H), 7.57 (d, J=6.9 Hz, 1H), 7.51 (d, J=10.3 Hz, 1H), 7.46-7.29 (m, 5H), 7.10-6.97 (m, 1H), 5.00 (s, 1H), 4.82 (s, 1H), 4.60 (s, 2H), 4.49 (s, 2H), 4.28-4.14 (m, 2H), 3.98-3.84 (m, 1H), 3.80 (q, J=7.3 Hz, 2H), 3.73-3.53 (m, 1H), 2.48 (s, 3H), 2.34 (s, 3H), 1.95-1.78 (m, 1H), 1.55-1.51 (m, 1H), 1.32 (t, J=7.3 Hz, 3H).

Step B: 6-(3-((Benzyloxy)methyl)-4-ethyl-5-oxo-4,5-dihydro-1H-1,2,4-triazol-1-yl)-2-(2-chloro-4,6-dimethylpyridin-3-yl)-7-fluoro-4-(prop-1-en-2-yl)-3,4-dihydroisoquinolin-1(2H)-one. The title compound was prepared in a manner analogous to Example 33, Step B using 4-(3-((benzyloxy)methyl)-4-ethyl-5-oxo-4,5-dihydro-1H-1,2,4-triazol-1-yl)-N-(2-chloro-4,6-dimethylpy ridin-3-yl)-5-fluoro-2-(1-hydroxy-3-methylbut-3-en-2-yl)benzamide instead of 4-(3-((benzyloxy)methyl)-4-ethyl-5-oxo-4,5-dihydro-1H-1,2,4-triazol-1-yl)-N-(2-chlorophenyl)-5-fluoro-2-(1-hydroxy-3-methylbut-3-en-2-yl)benzamide. LCMS (ES-API): mass calcd. for C₃₁H₃₁ClFN₅O₃, 575.21; m/z found, 576.3 [M+H]⁺. ¹ H NMR(400 MHz, CDCl₃) δ 8.02 (d, J=10.8 Hz, 1H), 7.51 (d, J=6.8 Hz, 1H), 7.44-7.30 (m, 5H), 7.03 (s, 1H), 5.16 (s, 1H), 4.68 (s, 1H), 4.61 (s, 2H), 4.52 (s, 2H), 4.21-4.05 (m, 1H), 3.97-3.78 (m, 3H), 3.69 (dd, J=6.1, 12.5 Hz, 1H), 2.52 (s, 3H), 2.24 (s, 3H), 1.85 (s, 3H), 1.36 (t, J=7.1 Hz, 3H).

Step C: Atropisomer 1, (S*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. Racemate 2-(2-chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one was prepared in a manner analogous to Example 33, Step C using 6-(3-((benzyloxy)methyl)-4-ethyl-5-oxo-4,5-dihydro-1H-1,2,4-triazol-1-yl)-2-(2-chloro-4,6-dimethylpyridin-3-yl)-7-fluoro-4-(prop-1-en-2-yl)-3,4-dihydroisoquinolin-1(2H)-one instead of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. A purification of this racemate was performed via chiral SFC (stationary phase: CHIRACEL AD-H 30×250 mm, mobile phase: 75% CO₂, 25% EtOH) to give the first peak as the title compound. LCMS (ES-API): mass calcd. for C₂₄H₂₅ClFN₅O₃, 485.16; m/z found, 486.1 [M+H]⁺. ¹ H NMR(400 MHz, CDCl₃) δ 8.00 (d, J=10.8 Hz, 1H), 7.51 (d, J=6.8 Hz, 1H), 7.04 (s, 1H), 5.15 (s, 1H), 4.74-4.57 (m, 3H), 4.12 (dd, J=5.1, 12.5 Hz, 1H), 3.90 (q, J=7.2 Hz, 3H), 3.69 (dd, J=6.4, 12.7 Hz, 1H), 2.78-2.62 (m, 1H), 2.52 (s, 3H), 2.24 (s, 3H), 1.85 (s, 3H), 1.41 (t, J=7.1 Hz, 3H).

### Example 59

### Atropisomer 1, (R *)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

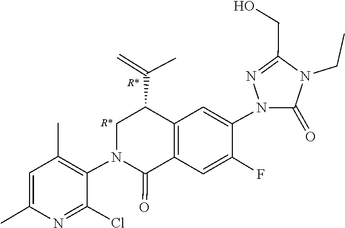 [SMILES: C=C(C)[C@H]1CN(c2c(C)cc(C)nc2Cl)C(=O)c2cc(F)c(-n3nc(CO)n(CC)c3=O)cc21]

The title compound was isolated as the second peak from chiral SFC purification of racemate 2-(2-chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. LCMS (ES-API): mass calcd. for C₂₄H₂₅ClFN₅O₃, 485.16; m/z found, 486.1 [M+H]⁺. ¹ H NMR(400 MHz, CDCl₃) δ 8.01 (d, J=10.8 Hz, 1H), 7.51 (d, J=6.8 Hz, 1H), 7.04 (s, 1H), 5.15 (s, 1H), 4.66 (br d, J=7.3 Hz, 3H), 4.12 (dd, J=4.9, 12.2 Hz, 1H), 3.90 (q, J=7.2 Hz, 3H), 3.69 (dd, J=6.6, 12.5 Hz, 1H), 2.71-2.48 (m, 1H), 2.52 (s, 3H), 2.24 (s, 3H), 1.85 (s, 3H), 1.41 (t, J=7.3 Hz, 3H).

### Example 60

### Atropisomer 1, (S*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

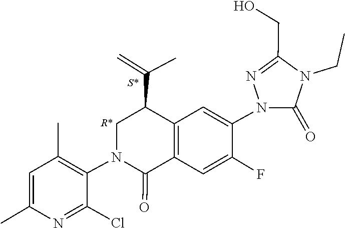 [SMILES: C=C(C)[C@@H]1CN(c2c(C)cc(C)nc2Cl)C(=O)c2cc(F)c(-n3nc(CO)n(CC)c3=O)cc21]

Step A: 6-(3-((Benzyloxy)methyl)-4-ethyl-5-oxo-4,5-dihydro-1H-1,2,4-triazol-1-yl)-2-(2-chloro-4,6-dimethylpyridin-3-yl)-7-fluoro-4-(prop-1-en-2-yl)-3,4-dihydroisoquinolin-1(2H)-one. The title compound was isolated as the second fraction from the same reaction in Example 58, Step B. LCMS (ES-API): mass calcd. for C₃₁H₃₁ClFN₅O₃, 575.21; m/z found, 576.3 [M+H]⁺. ¹ H NMR(400 MHz, CDCl₃) δ 8.07-8.01 (m, 1H), 7.49 (d, J=6.8 Hz, 1H), 7.42-7.31 (m, 5H), 7.06 (s, 1H), 5.19 (s, 1H), 4.94 (s, 1H), 4.61 (s, 2H), 4.52 (s, 2H), 4.23-4.12 (m, 1H), 3.98 (dd, J=5.4, 10.3 Hz, 1H), 3.86 (q, J=6.8 Hz, 2H), 3.50 (dd, J=5.4, 12.2 Hz, 1H), 2.52 (s, 3H), 2.29 (s, 3H), 1.85 (s, 3H), 1.36 (t, J=7.1 Hz, 3H).

Step B: Atropisomer 1, (S*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. Racemate 2-(2-chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one was prepared in a manner analogous to Example 33, Step C using 6-(3-((benzyloxy)methyl)-4-ethyl-5-oxo-4,5-dihydro-1H-1,2,4-triazol-1-yl)-2-(2-chloro-4,6-dimethylpyridin-3-yl)-7-fluoro-4-(prop-1-en-2-yl)-3,4-dihydroisoquinolin-1(2H)-one instead of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. A purification of this racemate was performed via chiral SFC (stationary phase: CHIRACEL AD-H 30×250 mm, mobile phase: 75% CO₂, 25% EtOH) to give the first peak as the title compound (8 mg, 23%). LCMS (ES-API): mass calcd. for C₂₄H₂₅ClFN₅O₃, 485.16; m/z found, 486.1 [M+H]⁺. ¹ H NMR(400 MHz, CDCl₃) δ 8.03 (d, J=10.3 Hz, 1H), 7.49 (d, J=6.8 Hz, 1H), 7.06 (s, 1H), 5.19 (s, 1H), 4.93 (s, 1H), 4.66 (s, 2H), 4.24-4.09 (m, 1H), 3.98 (dd, J=5.4, 9.8 Hz, 1H), 3.91 (q, J=7.3 Hz, 2H), 3.50 (dd, J=5.4, 12.2 Hz, 1H), 2.70-2.43 (m, 1H), 2.52 (s, 3H), 2.29 (s, 3H), 1.84 (s, 3H), 1.41 (t, J=7.1 Hz, 3H).

### Example 61

### Atropisomer 2, (R*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

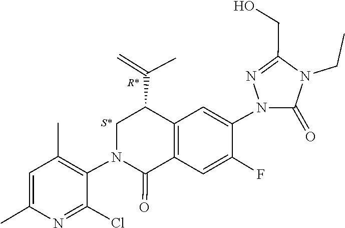 [SMILES: C=C(C)[C@H]1CN(c2c(C)cc(C)nc2Cl)C(=O)c2cc(F)c(-n3nc(CO)n(CC)c3=O)cc21]

The title compound was isolated as the second peak from chiral SFC purification of racemate 2-(2-chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. LCMS (ES-API): mass calcd. for LCMS (ES-API): mass calcd. for C₂₄H₂₅ClFN₅O₃, 485.16; m/z found, 486.1 [M+H]⁺. ¹H NMR (400 MHz, CDCl₃) δ 8.02 (d, J=10.8 Hz, 1H), 7.49 (d, J=6.8 Hz, 1H), 7.06 (s, 1H), 5.19 (s, 1H), 4.93 (s, 1H), 4.65 (s, 2H), 4.23-4.09 (m, 1H), 3.98 (dd, J=5.4, 10.3 Hz, 1H), 3.90 (q, J=7.2 Hz, 2H), 3.50 (dd, J=5.4, 12.2 Hz, 1H), 2.88-2.61 (m, 1H), 2.52 (s, 3H), 2.29 (s, 3H), 1.84 (s, 3H), 1.41 (t, J=7.1 Hz, 3H).

### Example 62

### Atropisomer 1, (S*)-2-(3-Chloro-2-methoxy-5-methylpyridin-4-yl)-6-(4-ethyl-3 -(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

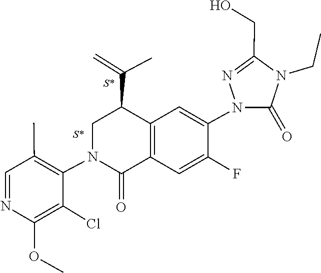 [SMILES: C=C(C)[C@@H]1CN(c2c(C)cnc(OC)c2Cl)C(=O)c2cc(F)c(-n3nc(CO)n(CC)c3=O)cc21]

Step A: 4-(3-((Benzyloxy)methyl)-4-ethyl-5-oxo-4,5-dihydro-1H-1,2,4-triazol-1-yl)-N-(3-chloro-2-methoxy-5-methylpyridin-4-yl)-5-fluoro-2-(1-hydroxy-3-methylbut-3-en-2-yl)benzamide. The title compound was prepared in a manner analogous to Example 33, Step A using 3-chloro-2-methoxy-5-methylpyridin-4-amine instead of 2-chloroaniline. LCMS (ES-API): mass calcd. for C₃₁H₃₃ClFN₅O₅, 609.22; m/z found, 610.2 [M+H]⁺. ¹ H NMR(400 MHz, CDCl₃) δ 9.60-9.50 (m, 1H), 7.94 (s, 1H), 7.60-7.51 (m, 2H), 7.41-7.30 (m, 5H), 4.98 (s, 1H), 4.78 (s, 1H), 4.59 (s, 2H), 4.48 (s, 2H), 4.26-4.05 (m, 2H), 3.99 (s, 3H), 3.92-3.73 (m, 3H), 3.58 (br s, 1H), 2.24 (s, 3H), 1.58 (s, 3H), 1.30 (t, J=6.9 Hz, 3H).

Step B: 6-(3-((Benzyloxy)methyl)-4-ethyl-5-oxo-4,5-dihydro-1H-1,2,4-triazol-1-yl)-2-(3-chloro-2-methoxy-5-methylpyridin-4-yl)-7-fluoro-4-(prop-1-en-2-yl)-3,4-dihydroisoquinolin-1(2H)-one. The title compound was prepared in a manner analogous to Example 33, Step B using 4-(3-((benzyloxy)methyl)-4-ethyl-5-oxo-4,5-dihydro-1H-1,2,4-triazol-1-yl)-N-(3-chloro-2-methoxy-5-methylpyridin-4-yl)-5-fluoro-2-(1-hydroxy-3-methylbut-3-en-2-yl)benzamide instead of 4-(3-((benzyloxy)methyl)-4-ethyl-5-oxo-4,5-dihydro-1H-1,2,4-triazol-1-yl)-N-(2-chlorophenyl)-5-fluoro-2-(1-hydroxy-3-methylbut-3-en-2-yl)benzamide. LCMS (ES-API): mass calcd. for C₃₁H₃₁ClFN₅O₄, 591.20; m/z found, 592.2 [M+H]⁺. ¹H NMR (400 MHz, CDCl₃) δ 8.07-7.94 (m, 2H), 7.51 (d, J=6.8 Hz, 1H), 7.44-7.30 (m, 5H), 5.16 (s, 1H), 4.69 (s, 1H), 4.61 (s, 2H), 4.52 (s, 2H), 4.08 (dd, J=4.9, 12.2 Hz, 1H), 4.02 (s, 3H), 3.96-3.80 (m, 3H), 3.70 (dd, J=6.6, 12.5 Hz, 1H), 2.15 (s, 3H), 1.85 (s, 3H), 1.36 (t, J=7.1 Hz, 3H).

Step C: Atropisomer 1, (S*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. Racemate 2-(3-chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one was prepared in a manner analogous to Example 33, Step C using 6-(3-((benzyloxy)methyl)-4-ethyl-5-oxo-4,5-dihydro-1H-1,2,4-triazol-1-yl)-2-(3-chloro-2-methoxy-5-methylpyridin-4-yl)-7-fluoro-4-(prop-1-en-2-yl)-3,4-dihydroisoquinolin-1(2H)-one instead of 6-(3-((benzyloxy)methyl)-4-ethyl-5-oxo-4,5-dihydro-1H-1,2,4-triazol-1-yl)-2-(2-chlorophenyl)-7-fluoro-4-(prop-1-en-2-yl)-3,4-dihydroisoquinolin-1(2H)-one. A purification of this racemate was performed via chiral SFC (stationary phase: CHIRACEL AD-H 30×250 mm, mobile phase: 60% CO₂, 40% EtOH) to give the first peak as the title compound. LCMS (ES-API): mass calcd. for C₂₄H₂₅ClFN₅O₄, 501.16; m/z found, 502.0 [M+H]⁺. ¹ H NMR(400 MHz, CDCl₃) δ 8.01 (d, J=10.8 Hz, 1H), 7.98 (s, 1H), 7.51 (d, J=6.8 Hz, 1H), 5.16 (s, 1H), 4.77-4.59 (m, 3H), 4.07 (dd, J=5.4, 12.2 Hz, 1H), 4.02 (s, 3H), 3.90 (q, J=7.3 Hz, 3H), 3.70 (dd, J=6.8, 12.2 Hz, 1H), 2.48 (t, J=6.1 Hz, 1H), 2.15 (s, 3H), 1.84 (s, 3H), 1.41 (t, J=7.3 Hz, 3H).

### Example 63

### Atropisomer 1, (R *)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

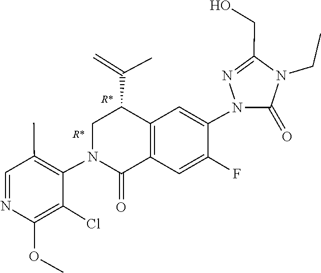 [SMILES: C=C(C)[C@H]1CN(c2c(C)cnc(OC)c2Cl)C(=O)c2cc(F)c(-n3nc(CO)n(CC)c3=O)cc21]

The title compound was isolated as the second peak from chiral SFC purification of racemate 2-(3-chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. LCMS (ES-API): mass calcd. for C₂₄H₂₅ClFN₅O₄, 501.16; m/z found, 502.0 [M+H]⁺. ¹H NMR (400 MHz, CDCl₃) δ 8.01 (d, J=10.8 Hz, 1H), 7.98 (s, 1H), 7.51 (d, J=6.8 Hz, 1H), 5.16 (s, 1H), 4.74-4.60 (m, 3H), 4.14-3.97 (m, 4H), 3.90 (q, J=7.0 Hz, 3H), 3.70 (dd, J=6.6, 12.5 Hz, 1H), 2.54 (br t, J=5.4 Hz, 1H), 2.15 (s, 3H), 1.84 (s, 3H), 1.41 (t, J=7.1 Hz, 3H).

### Example 64

### Atropisomer 2, (S*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

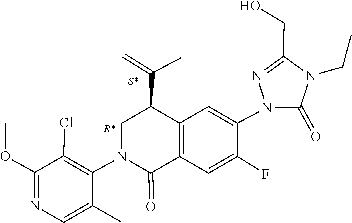 [SMILES: C=C(C)[C@@H]1CN(c2c(C)cnc(OC)c2Cl)C(=O)c2cc(F)c(-n3nc(CO)n(CC)c3=O)cc21]

Step A: 6-(3-((Benzyloxy)methyl)-4-ethyl-5-oxo-4,5-dihydro-1H-1,2,4-triazol-1-yl)-2-(3-chloro-2-methoxy-5-methylpyridin-4-yl)-7-fluoro-4-(prop-1-en-2-yl)-3,4-dihydroisoquinolin-1(2H)-one. The title compound was isolated as the second fraction from the reaction in Example 62, Step B. LCMS (ES-API): mass calcd. for C₃₁H₃₁ClFN₅O4, 591.20; m/z found, 592.0 [M+H]⁺. ¹H NMR (400 MHz, CDCl₃) δ 8.04 (d, J=10.8 Hz, 1H), 8.01 (s, 1H), 7.50 (d, J=6.4 Hz, 1H), 7.44-7.29 (m, 5H), 5.20 (d, J=1.5 Hz, 1H), 4.94 (s, 1H), 4.61 (s, 2H), 4.52 (s, 2H), 4.13 (dd, J=10.3, 12.2 Hz, 1H), 4.02 (s, 3H), 4.01-3.94 (m, 1H), 3.86 (q, J=7.2 Hz, 2H), 3.51 (dd, J=5.4, 12.2 Hz, 1H), 2.21 (s, 3H), 1.84 (s, 3H), 1.43-1.31 (m, J=7.1 Hz, 3H).

Step B: Atropisomer 2, (S*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. Racemate 2-(3-chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one was prepared in a manner analogous to Example 33, Step C using 6-(3-((benzyloxy)methyl)-4-ethyl-5-oxo-4,5-dihydro-1H-1,2,4-triazol-1-yl)-2-(3-chloro-2-methoxy-5-methylpyridin-4-yl)-7-fluoro-4-(prop-1-en-2-yl)-3,4-dihydroisoquinolin-1(2H)-one instead of 6-(3-((benzyloxy)methyl)-4-ethyl-5-oxo-4,5 -dihydro-1H-1,2,4-triazol-1-yl)-2-(2-chlorophenyl)-7-fluoro-4-(prop-1-en-2-yl)-3,4-dihydroisoquinolin-1(2H)-one. A purification of this racemate was performed via chiral SFC (stationary phase: Lux Cellulose-4 20×250 mm, mobile phase: 70% CO₂, 30% MeOH) to give the first peak as the title compound. LCMS (ES-API): mass calcd. for C₂₄H₂₅ClFN₅O₄, 501.16; m/z found, 502.0 [M+H]⁺. ¹H NMR (400 MHz, CDCl₃) δ 8.09-7.93 (m, 2H), 7.50 (d, J=6.8 Hz, 1H), 5.19 (s, 1H), 4.94 (s, 1H), 4.66 (s, 2H), 4.21-4.05 (m, 1H), 4.05-3.95 (m, 4H), 3.90 (q, J=7.0 Hz, 2H), 3.51 (dd, J=5.1, 12.0 Hz, 1H), 2.40 (br s, 1H), 2.20 (s, 3H), 1.83 (s, 3H), 1.41 (t, J=7.1 Hz, 3H).

### Example 65

### Atropisomer 2, (R*)-2-(3-Chloro-2-methoxy-5 -methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

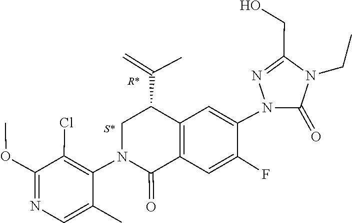 [SMILES: C=C(C)[C@H]1CN(c2c(C)cnc(OC)c2Cl)C(=O)c2cc(F)c(-n3nc(CO)n(CC)c3=O)cc21]

The title compound was isolated as the second peak from chiral SFC purification of racemate 2-(3-chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. LCMS (ES-API): mass calcd. for C₂₄H₂₅ClFN₅O₄, 501.16; m/z found, 502.0 [M+H]⁺. ¹H NMR (400 MHz, CDCl₃) δ 8.08-7.94 (m, 2H), 7.50 (d, J=6.8 Hz, 1H), 5.19 (s, 1H), 4.93 (s, 1H), 4.65 (s, 2H), 4.20-4.06 (m, 1H), 4.06-3.94 (m, 4H), 3.90 (q, J=7.2 Hz, 2H), 3.52 (dd, J=5.1, 12.0 Hz, 1H), 2.64 (br s, 1H), 2.20 (s, 3H), 1.83 (s, 3H), 1.40 (t, J=7.1 Hz, 3H).

### Example 66

### (S*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one

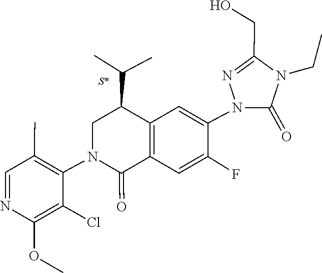 [SMILES: CCn1c(CO)nn(-c2cc3c(cc2F)C(=O)N(c2c(C)cnc(OC)c2Cl)C[C@H]3C(C)C)c1=O]

A mixture 2-(3-chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one (racemate, Example 62, Step C, 170 mg, 0.340 mmol) and 10% Pd/C (38 mg, 0.036 mmol) in EtOAc (80 mL) was hydrogenated under 1 atm H₂ balloon for 3 h. After filtering off the solid through a pad of Celite®, the filtrate was concentrated and purified by flash column chromatography (30-70% EtOAc in heptane) and chiral SFC (stationary phase: CHIRACEL AD-H 20×250 mm, mobile phase: 88% CO₂, 12% MeOH) to give the first peak as the title compound (51 mg, 30%). LCMS (ES-API): mass calcd. for C₂₄H₂₇ClFN₅O₄, 503.17; m/z found, 504.3 [M+H]⁺. ¹H NMR (400 MHz, CDCl₃) δ 8.11-7.91 (m, 2H), 7.56 (d, J=6.8 Hz, 1H), 4.65 (s, 2H), 4.25 (dd, J=3.9, 12.7 Hz, 1H), 4.02 (s, 3H), 3.91 (q, J=6.8 Hz, 2H), 3.56 (dd, J=3.9, 12.7 Hz, 1H), 2.83-2.70 (m, 1H), 2.33-2.17 (m, 1H), 2.24 (s, 3H), 1.41 (t, J=6.8 Hz, 3H), 1.04 (d, J=6.9 Hz, 3H), 0.99 (d, J=6.9 Hz, 3H).

### Example 67

### (R *)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one

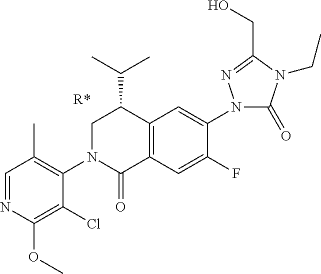 [SMILES: CCn1c(CO)nn(-c2cc3c(cc2F)C(=O)N(c2c(C)cnc(OC)c2Cl)C[C@@H]3C(C)C)c1=O]

The title compound (67 mg, 39%) was isolated as the second peak from chiral SFC purification of 2-(3-chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one (Example 66). LCMS (ES-API): mass calcd. for C₂₄H₂₇ClFN₅O₄, 503.17; m/z found, 504.3 [M+H]+¹H NMR (400 MHz, CDCl₃) δ 8.01 (s, 1H), 7.98 (d, J=10.8 Hz, 1H), 7.56 (d, J=6.8 Hz, 1H), 4.65 (s, 2H), 4.25 (dd, J=4.2, 12.5 Hz, 1H), 4.02 (s, 3H), 3.91 (q, J=7.2 Hz, 2H), 3.56 (dd, J=4.4, 12.7 Hz, 1H), 2.82-2.66 (m, 2H), 2.33-2.17 (m, 1H), 2.24 (s, 3H), 1.41 (t, J=7.3 Hz, 3H), 1.04 (d, J=6.8 Hz, 3H), 0.98 (d, J=6.8 Hz, 3H).

### Example 68

### (R*)-2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one

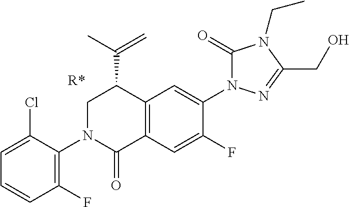 [SMILES: C=C(C)[C@H]1CN(c2c(F)cccc2Cl)C(=O)c2cc(F)c(-n3nc(CO)n(CC)c3=O)cc21]

Step A. tert-Butyl 4,5-difluoro-2-iodobenzoate. 4,5-Difluoro-2-iodobenzoic acid (3 g, 10.56 mmol) was dissolved in THF (30 mL), then Boc₂O (4.6 g, 21.13 mmol) was added followed by DMAP (645 mg, 5.28 mmol). The reaction mixture was stirred at 50° C. overnight under N₂, then cooled down to room temperature. The solvent was removed under vacuum. The residue was diluted with 100 mL ethyl acetate, then washed with brine (20 mL×2). The organic layer was dried with Na₂SO₄, filtered and concentrated under vacuum. The crude product was purified by column chromatography (SiO₂, gradient elution: 0-5% ethyl acetate in petroleum ether) to give the title compound (2.85 g, 8.38 mmol, 79.33% yield) as yellow oil. ¹ H NMR(400 MHz, CDCl₃) δ=7.77 (dd, J=7.6, 9.5 Hz, 1H), 7.63 (dd, J=8.1, 10.8 Hz, 1H), 1.62 (s, 9H); ¹⁹F NMR (376 MHz, CDCl₃) δ=−131.13-−131.55 (m, 1F), −136.65-−136.97 (m, 1F).

Step B. tert-Butyl 4-(3-((benzyloxy)methyl)-4-ethyl-5-oxo-4,5-dihydro-1H-1,2,4-triazol-1-yl)-5-fluoro-2-iodobenzoate. A mixture of tert-butyl 4,5-difluoro-2-iodobenzoate (3.18 g, 9.35 mmol), 3-((benzyloxy)methyl)-4-ethyl-1H-1,2,4-triazol-5(4H)-one (2.62 g, 11.22 mmol) and Cs₂CO₃ (6.09 g, 18.70 mmol) in DMF (30 mL) was stirred at 75° C. for 1 hour then cooled down to room temperature. The reaction mixture was filtered through a pad of Celite® and the solid was rinsed with 200 mL ethyl acetate. The filtrate was washed with brine (50 mL×3). The organic layer was dried with Na₂SO₄, filtered and concentrated under vacuum. The crude product was purified by column chromatography (SiO₂, gradient elution: 0-40% ethyl acetate in petroleum ether) to give the title compound (4.98 g, 9.00 mmol, 96.25% yield) as colorless sticky. MS (ESI): mass calcd. for C₂₃H₂₅FIN₃O₄, 553.1; m/z found, 554.1 [M+H]⁺. ¹H NMR (400 MHz, CDCl₃) δ=8.16 (d, J=7.1 Hz, 1H), 7.62 (d, J=10.8 Hz, 1H), 7.45-7.29 (m, 5H), 4.61 (s, 2H), 4.50 (s, 2H), 3.84 (q, J=7 .1 Hz, 2H), 1.63 (s, 9H), 1.35 (t, J=7.2 Hz, 3H); ¹⁹F NMR (376 MHz, CDCl₃) δ=−119.09 (dd, J=7.0, 10.6 Hz, 1F).

Step C. 4-(3-((Benzyloxy)methyl)-4-ethyl-5-oxo-4,5-dihydro-1H-1,2,4-triazol-1-yl)-5-fluoro-2-iodobenzoic acid. TFA (10 mL) was slowly added to a solution of tent-butyl 4-(3-((benzyloxy) methyl)-4-ethyl-5-oxo-4,5-dihydro-1H-1,2,4-triazol-1-yl)-5-fluoro-2-iodobenzoate (4.98 g, 9.00 mmol) in DCM (50 mL), and the reaction mixture was stirred at room temperature overnight. The reaction mixture was concentrated under vacuum. The obtained residue was triturated with 60 mL petroleum ether at room temperature for 30 minutes. The mixture was filtered and the solid was rinsed with 20 mL petroleum ether. The solid was collected and dried under vacuum to give the title compound (4.05 g, 8.15 mmol, 90.50% yield) as white solid. MS (ESI): mass calcd. for C₁₉H₁₇FIN₃O₄, 497.0; m/z found, 498.0 [M+H]⁺. ¹H NMR (400 MHz, DMSO-d₆) δ=8.16 (d, J=7.3 Hz, 1H), 7.78 (d, J=11.0 Hz, 1H), 7.43-7.28 (m, 5H), 4.60 (s, 2H), 4.57 (s, 2H), 3.74 (q, J=7 .2 Hz, 2H), 1.23 (t, J=7.2 Hz, 3H); ¹⁹F NMR (376 MHz, DMSO-d₆) δ=−119.91 (s, 1F).

Step D. 4-(3-((Benzyloxy)methyl)-4-ethyl-5-oxo-4,5-dihydro-1H-1,2,4-triazol-1-yl)-5-fluoro-2-iodobenzoyl chloride. A solution of 4-(3-((benzyloxy)methyl)-4-ethyl-5-oxo-4,5-dihydro-1H-1,2,4-triazol-1-yl)-5-fluoro-2-iodobenzoic acid (3.5 g, 7.04 mmol) in SOCl₂ (14 mL) was stirred at reflux for 15 minutes. The reaction mixture was concentrated under vacuum. The residue was co-evaporated with anhydrous toluene (25 mL×2) to give the title compound (3.63 g crude, 7.04 mmol) as yellow sticky, which was directly used for the next step.

Step E. 4-(3-((Benzyloxy)methyl)-4-ethyl-5-oxo-4,5-dihydro-1H-1,2,4-triazol-1-yl)-N-(2-chloro-6-fluorophenyl)-5-fluoro-2-iodo-N-(3-methylbut-2-en-1-yl)benzamide. 4-(3-((Benzyloxy)methyl)-4-ethyl-5-oxo-4,5-dihydro-1H-1,2,4-triazol-1-yl)-5-fluoro-2-iodobenzoyl chloride (3.63 g crude, 7.04 mmol) was dissolved into DCM (7 mL), and added slowly drop wise to a precooled solution of 2-chloro-6-fluoro-N-(3-methylbut-2-en-1-yl)aniline (1 g, 4.68 mmol) and Et₃N (1.42 g, 14.04 mmol) in DCM (14 mL) at 0° C. with ice bath, followed by the addition of DMAP (57 mg, 466.57 μmol). The reaction mixture was warmed to room temperature and stirred at room temperature for 3 hours. The reaction mixture was slowly added to 100 mL sat. aq. NaHCO₃. The organic layer was separated, and the aqueous layer was extracted with DCM (100 mL×3). The combined organic extract was washed with brine, dried over Na₂SO₄, filtered and concentrated under vacuum. The crude product was purified by column chromatography (SiO₂, gradient elution: 0-70% ethyl acetate in petroleum ether) to give the title compound (3.14 g, 4.53 mmol, 96.83% yield, 100% purity) as yellow sticky. MS (ESI): mass calcd. for C₃₀H₂₈ClF₂IN₄O₃, 692.1; m/z found, 693.1 [M+H]⁺. ¹ H NMR(400 MHz, CDCl₃) δ=8.13-7.97 (m, 1H), 7.42-7.28 (m, 6H), 7.21-6.90 (m, 3H), 5.36-5.18 (m, 1H), 4.81 (dd, J=6 .8, 14.3 Hz, 1H), 4.65-4.55 (m, 2H), 4.54-4.44 (m, 2H), 4.32-4.20 (m, 1H), 3.92-3.75 (m, 2H), 1.64 (d, J=16.3 Hz, 3H), 1.49 (s, 3H), 1.39-1.30 (m, 3H); ¹⁹F NMR (376 MHz, CDCl₃) δ=−110.71 (br s, 1F), −118.52-−119.88 (m, 1F).

Step F. 6-(3-((Benzyloxy)methyl)-4-ethyl-5-oxo-4,5-dihydro-1H-1,2,4-triazol-1-yl)-2-(2-chloro-6-fluorophenyl)-7-fluoro-4-(prop-1-en-2-yl)-3,4-dihydroisoquinolin-1(2H)-one. To a solution of 4-(3-((benzyloxy)methyl)-4-ethyl-5-oxo-4,5-dihydro-1H-1,2,4-triazol-1-yl)-N-(2-chloro-6-fluorophenyl)-5-fluoro-2-iodo-N-(3-methylbut-2-en-1-yl)benzamide (3.34 g, 4.82 mmol) in DMF (35 mL) was added tetrabutylammonium bromide (4.66 g, 14.46 mmol), potassium acetate (946 mg, 9.64 mmol) and Pd(OAc)₂ (1.08 g, 4.82 mmol) respectively, and the reaction mixture was heated at 80° C. overnight under N₂. The reaction mixture was cooled down to room temperature, then filtered through a pad of Celite®. The solid was rinsed with 200 mL ethyl acetate. The filtrate was washed with brine (50 mL×3), dried with Na₂SO₄, filtered and concentrated under vacuum. The crude product was purified by column chromatography (SiO₂, gradient elution: 0-70% ethyl acetate in petroleum ether). It was further purified by preparative reversed phase HPLC (Stationary phase: YMC-Triart Prep C18, 7 μm, 150×40 mm; Mobile phase: H₂O (0.05% NH₃H₂O) (A)—MeCN (B), isocratic elution: 73% B in A over 11 min, flow rate: 25 mL/min) to give the title compound (1.2 g, 2.12 mmol, 44% yield, 100% purity) as white solid. MS (ESI): mass calcd. for C₃₀H₂₇ClF₂N₄O₃, 564.2; m/z found, 565.2 [M+H]⁺. ¹ H NMR(400 MHz, CDCl₃) δ=8.03 (d, J=10 .8 Hz, 1H), 7.50 (d, J=6 .8 Hz, 1H), 7.42-7.34 (m, 5H), 7.33-7.28 (m, 2H), 7.16-7.09 (m, 1H), 5.14 (d, J=1.3 Hz, 1H), 4.90-4.78 (m, 1H), 4.61 (s, 2H), 4.52 (s, 2H), 4.04-3.96 (m, 1H), 3.95-3.82 (m, 4H), 1.84 (d, J=11.8 Hz, 3H), 1.36 (t, J=7 .3 Hz, 3H); ¹⁹F NMR (376 MHz, CDCl₃) δ=−115.83 (s, 1F), −116.65 (s, 1F), −120.75 (s, 1F), −120.82 (s, 1F).

Step G. (S*)-2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2M-one and (R)-2-(2-chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one. A solution of 6-(3-((benzyloxy)methyl)-4-ethyl-5-oxo-4,5-dihydro-1H-1,2,4-triazol-1-yl)-2-(2-chloro-6-fluorophenyl)-7-fluoro-4-(prop-1-en-2-yl)-3,4-dihydroisoquinolin-1(2H)-one (1.2 g, 2.12 mmol) in TFA (12 mL) was stirred at 70° C. overnight under N₂. The mixture was concentrated and dissolved in MeOH (10 mL), then K₂CO₃ (800 mg) was added and the mixture was stirred at room temperature for 1 hour. The mixture was filtered through a pad of Celite® and filter cake was washed with 30 mL MeOH. The filtrate was concentrated. The crude product was purified by column chromatography (SiO₂, elution: 0-100% ethyl acetate in petroleum ether). It was purified by preparative reversed phase HPLC (Stationary phase: Welch diol, 5 μm, 150×25 mm; Mobile phase: Hexane (A) EtOH (0.5% NH₃H₂O) (B), gradient elution: 5-95% B in A over 13 min, flow rate: 30 mL/min) to give a mixture of racemic title compound and an isomeric side product, 2-(2-chloro-6-fluorophenyl)-6-(4-ethyl-3-(hydroxymethyl)-5-oxo-4,5-dihydro-1H-1,2,4-triazol-1-yl)-7-fluoro-4-(propan-2-ylidene)-3,4-dihydroisoquinolin-1(2H)-one (500 mg, 1.05 mmol, 49% yield) as white solid and carried on without further purification.

The above mixture (400 mg) was separated by SFC (Stationary phase: DAICEL CHIRALPAK IG, 10 μm, 250×50 mm; Mobile phase: Supercritical CO₂ (A)—EtOH (0.1% NH₃H₂O) (B), isocratic elution: 35% B in A, flow rate: 80 mL/min) to give: The first eluting fraction: impure mixture of (S)-2-(2-chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one (238 mg); The second eluting fraction: title, compound, (R)-2-(2-chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one (150 mg, 315.86 μmol, 37.50% yield, 100% purity) as white powder. MS (ESI): mass calcd. for C₂₃H₂₁ClF₂N₄O₃, 474.1; m/z found, 475.2 [M+H]⁺. ¹H NMR (400 MHz, CDCl₃) δ=8.04 (d, J=10 .8 Hz, 1H), 7.51 (d, J=6 .8 Hz, 1H), 7.33-7.29 (m, 2H), 7.16-7.10 (m, 1H), 5.15 (s, 1H), 4.90-4.79 (m, 1H), 4.68 (d, J=6 .4 Hz, 2H), 4.05-3.96 (m, 1H), 3.95-3.87 (m, 4H), 2.07 (t, J=6.2 Hz, 1H), 1.84 (d, J=11.7 Hz, 3H), 1.42 (t, J=7 .2 Hz, 3H); ¹⁹F NMR (376 MHz, CDCl₃) δ=−115.84-−115.90 (m, 1F), −116.69 (dd, J=11 .0, 2.9 Hz, 1F), −120.85-−120.98 (m, 1F).

### Example 69

### (S*)-2-(2-Chloro-6-fluorophenyl)-6-(4-ethyl-3-(hydroxymethyl)-5-oxo-4,5 -dihydro-1H-1,2,4-triazol-1-yl)-7-fluoro-4-(prop-1-en-2-yl)-3,4-dihydroisoquinolin-1(2H)-one

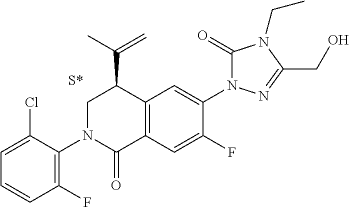 [SMILES: C=C(C)[C@@H]1CN(c2c(F)cccc2Cl)C(=O)c2cc(F)c(-n3nc(CO)n(CC)c3=O)cc21]

The first eluting fraction from Example 68, Step G (238 mg) was separated by SFC (Stationary phase: Phenomenex-Cellulose-2, 5 nm, 250×30 mm; Mobile phase: Supercritical CO₂ (A)—MeOH (0.1% NH₃H₂O) (B), isocratic elution: 35% B in A, flow rate: 60 mL/min) to give:

The first eluting fraction: (S*)-2-(2-chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one (140 mg, 294.81 nmol, 35.00% yield, 100% purity) as white powder. MS (ESI): mass calcd. for C₂₃H₂₁ClF₂N₄O₃, 474.1; m/z found, 475.2 [M+H]⁺. ¹ H NMR(400 MHz, CDCl₃) δ=8.04 (d, J=10.8 Hz, 1H), 7.51 (d, J=6.8 Hz, 1H), 7.31 (dd, J=6.4, 3.2 Hz, 2H), 7.17-7.10 (m, 1H), 5.15 (s, 1H), 4.90-4.79 (m, 1H), 4.68 (d, J=6 .4 Hz, 2H), 4.05-3.97 (m, 1H), 3.95-3.87 (m, 4H), 2.08 (t, J=6.6 Hz, 1H), 1.84 (d, J=11.7 Hz, 3H), 1.42 (t, J=7.2 Hz, 3H); ¹⁹F NMR (376 MHz, CDCl₃) δ=−115.84-−115.90 (m, 1F), −116.69 (dd, J=11 .0, 2.9 Hz, 1F), −120.85-−120.99 (m, 1F).

### Example 70

### (S*)-4-((RS)-sec-Butyl)-2-(2-chloro-6-fluorophenyl)-6-(4-ethyl-3-(hydroxymethyl)-5-oxo-4,5 -dihydro-1H-1,2,4-triazol-1-yl)-7-fluoro-3,4-dihydroisoquinolin-1(21-1)-one

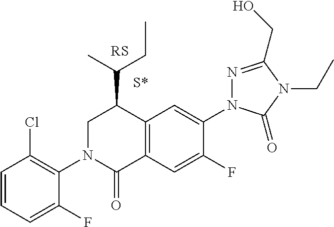 [SMILES: CCC(C)[C@@H]1CN(c2c(F)cccc2Cl)C(=O)c2cc(F)c(-n3nc(CO)n(CC)c3=O)cc21]

Step A. Ethyl 3-methylpent-2-enoate.To a solution of 2-butanone (51.74 g, 717.62 mmol) and (carbethoxymethylene) triphenylphosphorane (50 g, 143.52 mmol) in toluene (65 mL) was added benzoic acid (3.5 g, 28.71 mmol). The reaction mixture was heated at reflux for 16 hours. The mixture was diluted with hexane (100 mL) and filtered. The filter cake was washed with hexane (50 mL). The filtrate was concentrated under vacuum at 0-2° C. The crude was purified by column chromatography (SiO₂, elution: 0-10% ethyl acetate in petroleum ether) to give the title compound (25 g crude, 60% purity) as colorless liquid. ¹H NMR (400 MHz,CDCl₃) δ=5.70-5.57 (m, 1H), 4.18-4.08 (m, 2H), 2.62 (q, J=7 .6 Hz, 1H), 2.35 (s, 3H), 2.19-2.11 (m, 3H), 1.87 (d, J=1.2 Hz, 1H), 1.30-1.24 (m, 3H), 1.09-1.03 (m, 3H).Step B. 3-methylpent-2-en-1-ol.

Step B: 3-Methylpent-2-en-1-ol. Diisobutylaluminum hydride (1 M solution in toluene, 118 mL, 118 mmol) was cold to −78° C. Ethyl 3-methylpent-2-enoate (20 g crude, 70% purity) in toluene (40 mL) was added drop wise under N₂. The reaction mixture was stirred at −78° C. for 2 hours. The mixture was warmed to room temperature and slowly poured into 500 mL sat. aq. potassium sodium tartrate at 0° C. The mixture was stirred for 2 hours and filtered over Celite®. The solid was rinsed with DCM/ethyl acetate (v/v, 3/1, 300 mL), and the filtrate was extracted with DCM (200 mL×3). The organic extracts were dried over Na₂SO₄, filtered and concentrated. The crude was purified by column chromatography (SiO₂, elution: 0-100% DCM in petroleum ether, then 0-30% ethyl acetate in DCM) to give the title compound (7 g, 69.89 mmol, 56.81% yield of two steps) as colorless liquid. ¹H NMR (400 MHz, CDCl₃) δ=5.46-5.35 (m, 1H), 4.21-4.11 (m, 2H), 2.13-2.02 (m, 2H), 1.76-1.67 (m, 3H), 1.06-0.98 (m, 3H).

Step C. 3-Methylpent-2-enal. Dess-martin periodinane (10.2 g, 23.96 mmol) was added to a solution of 3-methylpent-2-en-1-ol (2 g, 19.97 mmol) in DCM (20 mL). The reaction mixture was stirred at room temperature for 1 hour. The mixture was filtered over Celite®, and the solid was rinsed with 50 mL DCM. The filtrate was washed with sat. aq. NaHCO₃ (50 mL×2). The organic layer was dried with Na₂SO₄, filtered and concentrated under vacuum at 0-2° C. The crude was purified by column chromatography (SiO₂, isocratic elution: DCM) to give the title compound (1.5 g, 15.28 mmol, 76.54% yield) as colorless liquid. ¹ H NMR(400 MHz, CDCl₃) δ=10.04-9.91 (m, 1H), 5.90-5.78 (m, 1H), 2.58 (q, J=7.6 Hz, 1H), 2.23 (d, J=7.3 Hz, 1H), 2.16 (s, 2H), 1.96 (d, J=1.1 Hz, 1H), 1.16 (t, J=7.6 Hz, 1H), 1.09 (t, J=7.4 Hz, 2H). Step D. 2-Chloro-6-fluoro-N-(3-methylpent-2-en-1-ylidene)aniline. Et₃N (4.6 mL, 32.98 mmol) was added to a mixture of 2-chloro-6-fluoroaniline (1.2 g, 8.24 mmol) and 3-methylpent-2-enal (971 mg, 9.89 mmol) in DCM (18 mL) under N₂ at 0° C. TiCl₄ (1 M solution in DCM, 5 mL, 5 mmol) was then added drop wise and the resulting mixture was stirred at 0° C. for 1 hour before warming to room temperature and stirring for further 4 hours. The mixture was poured into 100 mL sat. aq. NH₄Cl. The mixture became cloudy and filtered through a pad of Celite®. The solid was rinsed with DCM (100 mL). The filtrate was separated, and aqueous layer was extracted with DCM (50 mL×3). The organic layers were washed with brine (100 mL), dried with Na₂SO₄, filtered and concentrated. The crude product was purified by column chromatography (SiO₂, gradient elution: 0-5% DCM in petroleum ether) to give the title compound (1.3 g, 5.76 mmol, 69.87% yield) as pale yellow oil.

Step E. 2-Chloro-6-fluoro-N-(3-methylpent-2-en-1-yl)aniline. To a solution of 2-chloro-6-fluoro-N-(3-methylpent-2-en-1-ylidene)aniline (1.3 g, 5.76 mmol) in MeOH (20 mL) at room temperature was added NaBH₄ (218 mg, 5.76 mmol), and after 1 hour interval, another batch of NaBH₄ (218 mg, 5.76 mmol) was added. A total of NaBH₄ (1.09 g, 28.80 mmol) was added. The reaction mixture was stirred at room temperature overnight. To the mixture was added NaBH₄ (218 mg, 5.76 mmol), and after 1 h interval, another batch of NaBH₄ (218 mg, 5.76 mmol) was added. A total of NaBH₄ (1.09 g, 28.80 mmol) was added. The reaction mixture was stirred at room temperature for 2 hours. The mixture was concentrated to dryness. The mixture was diluted with water (30 mL) and extracted with ethyl acetate (50 mL×3). The organic layer was washed with 50 mL brine, dried with Na₂SO₄, filtered and concentrated. The crude product was purified by column chromatography (SiO₂, eluent: 0-5% DCM in petroleum ether) to give the title compound (430 mg, 1.89 mmol, 32.78% yield) as yellow oil. ¹H NMR (400 MHz, CDCl₃) δ=7.02-6.95 (m, 1H), 6.84 (ddd, J=1.3, 8.3, 12.2 Hz, 1H), 6.63-6.52 (m, 1H), 5.28-5.17 (m, 1H), 3.85 (d, J=5.6 Hz, 2H), 3.73 (s, 1H), 2.07-1.91 (m, 2H), 1.68-1.58 (m, 3H), 0.96-0.89 (m, 3H).

Step F. 4-(3-((Benzyloxy)methyl)-4-ethyl-5-oxo-4,5-dihydro-1H-1,2,4-triazol-1-yl)-N-(2-chloro-6-fluorophenyl)-5-fluoro-2-iodo-N-(3-methylpent-2-en-1-yl)benzamide. 4-(3-((benzyloxy)methyl)-4-ethyl-5-oxo-4,5-dihydro-1H-1,2,4-triazol-1-yl)-5-fluoro-2-iodobenzoyl chloride (917 mg crude, 1.78 mmol) was dissolved into DCM (4 mL), and added slowly drop wise to a precooled solution of 2-chloro-6-fluoro-N-(3-methylpent-2-en-1-yl)aniline (270 mg, 1.19 mmol) and Et₃N (360 mg, 3.56 mmol) in DCM (5 mL) at 0° C. with ice bath, followed by the addition of DMAP (14.5 mg, 118.69 umol) under N₂. The reaction mixture was warmed to room temperature and stirred at room temperature for 3 hours. The reaction was quenched by the addition of sat. aq. NaHCO₃ (20 mL). The organic layer was separated, and the aqueous layer was extracted with DCM (20 mL×3). The combined organic extract was washed with brine (10 mL), dried over Na₂SO₄, filtered and concentrated under vacuum. The crude product was purified by column chromatography (SiO₂, eluent: 0-40% ethyl acetate in petroleum ether) to give the title compound (730 mg, 947.35 umol, 79.90% yield, 92% purity) as yellow sticky. MS (ESI): mass calcd. for C₃₁H₃₀ClF₂IN₄O₃, 706.1; m/z found, 707.1 [M+H]⁺.

Step G. 6-(3-((Benzyloxy)methyl)-4-ethyl-5-oxo-4,5-dihydro-1H-1,2,4-triazol-1-yl)-4-(but-1-en-2-yl)-2-(2-chloro-6-fluorophenyl)-7-fluoro-3,4-dihydroisoquinolin-1(2H)-one. To a solution of 4-(3-((benzyloxy)methyl)-4-ethyl-5-oxo-4,5-dihydro-1H-1,2,4-triazol-1-yl)-N-(2-chloro-6-fluorophenyl)-5-fluoro-2-iodo-N-(3-methylpent-2-en-1-yl)benzamide (730 mg, 947.35 umol, 92% purity) in DMF (7.3 mL) was added TBAB (916 mg, 2.84 mmol), AcOK (186 mg, 1.90 mmol), Pd(OAc)₂ (212.7 mg, 947.40 umol) respectively, and the reaction mixture was heated at 80° C. overnight under N₂. The reaction mixture was cooled down to room temperature, then filtered through a pad of Celite®. The solid was rinsed with 200 mL ethyl acetate. The filtrate was washed with brine (50 mL×3), dried with Na₂SO₄, filtered and concentrated under vacuum. The crude product was purified by column chromatography (SiO₂, eluent: 0-70% ethyl acetate in petroleum ether). It was further purified by preparative reversed phase HPLC (Stationary phase: Boston Uni C18, 5 μm, 150×40 mm; Mobile phase: water (0.04% NH₃H₂O+10 mM NH₄HCO₃) (A)—MeCN (B), gradient elution: 70-100% B in A over 8 min, flow rate: 25 mL/min) to give the title compound that was contaminated with additional isomers (290 mg, 500.83 μmol, 52.87% yield) as pale yellow solid. MS (ESI): mass calcd. for C₃₁H₂₉ClF₂N₄O₃, 578.2; m/z found, 579.1 [M+H]⁺.

Step H. 4-(sec-Butyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one and 4-(sec-butyl)-2-(2-chloro-6-fluorophenyl)-6-(4-ethyl-3-(hydroxymethyl)-5-oxo-4,5-dihydro-1H-1,2,4-triazol-1-yl)-7-fluoroisoquinolin-1(2H)-one. To a solution of the mixture of 6-(3-((benzyloxy)methyl)-4-ethyl-5-oxo-4,5-dihydro-1H-1,2,4-triazol-1-yl)-4-(but-1-en-2-yl)-2-(2-chloro-6-fluorophenyl)-7-fluoro-3,4-dihydroisoquinolin-1(2H)-one and isomers (290 mg, 500.83 μmol) from the previous step, in ethyl acetate (18 mL) was added Pd/C (10%, 133 mg, 124.98 μmop. The suspension was degassed under vacuum and purged with H₂ several times. The mixture was stirred under H₂ (15 psi) at room temperature for 4 hours. The mixture was filtered and the filtrate was concentrated under vacuum. The residue was purified by preparative reversed phase HPLC (Stationary phase: Boston Uni C18, 5 μm, 150×40 mm; Mobile phase: water (0.04% NH₃H₂O+10 mM NH₄HCO₃) (A)—MeCN (B), gradient elution: 45-75% B in A over 8 min, flow rate: 25 mL/min) to give the mixture of 4-(sec-butyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one and 4-(sec-butyl)-2-(2-chloro-6-fluorophenyl)-6-(4-ethyl-3-(hydroxymethyl)-5-oxo-4,5-dihydro-1H-1,2,4-triazol-1-yl)-7-fluoroisoquinolin-1(2H)-one (, 165 mg, 336.10 μmol, 67% yield) as white solid. The mixture154 mg, 313.69 μmol) was separated by SFC (Stationary phase: DAICEL CHIRALPK AY-H, 5 μm, 250×30 mm; Mobile phase: Supercritical CO₂ (A)—EtOH (0.1% NH₃.H₂O) (B), isocratic elution: 30% B in A at 70 mL/min) give the first eluting fraction (fraction 1, 80 mg) and the second eluting mixture (fraction 2, 60 mg) both as white solid.

Fraction 1 (80 mg) was separated by SFC (Stationary phase: Phenomenex-Cellulose-2, 10 μm, 250×30 mm; Mobile phase: Supercritical CO₂ (A)—MeOH (0.1% NH₃.H₂O) (B), isocratic elution: 30% B in A at 60 mL/min) to give: (S*)-4-((RS)-sec-butyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one (EXAMPLE 70, 55 mg, 112.03 μma 35.71% yield, 100% purity) as white powder. MS (ESI): mass calcd. for C₂₄H₂₅ClF₂N₄O₃, 490.2; m/z found, 491.2 [M+H]⁺. ¹H NMR (400 MHz, CDCl₃) δ=8.02-7.95 (m, 1H), 7.56-7.50 (m, 1H), 7.35-7.27 (m, 2H), 7.18-7.08 (m, 1H), 4.66 (d, J=5 .5 Hz, 2H), 4.28-4.04 (m, 1H), 3.91 (q, J=7.1 Hz, 2H), 3.79-3.63 (m, 1H), 2.97-2.65 (m, 1H), 2.48 (br t, J=5.3 Hz, 1H), 2.26-2.02 (m, 1H), 1.42 (t, J=7 .3 Hz, 3H), 1.26-1.13 (m, 1H), 1.00 (dd, J=6.7 , 12.7 Hz, 2H), 0.95-0.86 (m, 4H); ¹⁹F NMR (376 MHz, CDCl₃) δ=−115.36-−117.45 (m, 1F), −121.48-−121.56 (m, 1F).

### Example 71

### (R*)-4-((S *)-sec-B utyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one

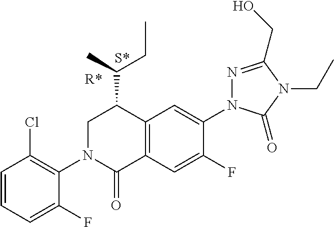 [SMILES: CC[C@H](C)[C@H]1CN(c2c(F)cccc2Cl)C(=O)c2cc(F)c(-n3nc(CO)n(CC)c3=O)cc21]

Fraction 2 (Example 70, Step H, 60 mg) was separated by SFC (Stationary phase: DAICEL CHIRALPAK AY-H, 5 μm, 250×30 mm; Mobile phase: Supercritical CO₂ (A)—EtOH (0.1% NH_{3.}H₂O) (B), isocratic elution: 30% B in A at 70 mL/min) and the first eluting compound was isolated and yielded the title compound, (32 mg, 64.92 μmol, 20.69% yield, 99.59% purity) as white powder. MS (ESI): mass calcd. for C₂₄H₂₅ClF₂N₄O_{3B}, 490.2; m/z found, 491.2 [M+H]+.¹ H NMR(400 MHz, CDCl₃) δ=7.99 (dd, J=6.1, 10.9 Hz, 1H), 7.53 (dd, J=6.9, 9.9 Hz, 1H), 7.33-7.28 (m, 2H), 7.18-7.11 (m, 1H), 4.68 (d, J=6.0 Hz, 2H), 4.29-4.07 (m, 1H), 3.92 (q, J=7.3 Hz, 2H), 3.73-3.63 (m, 1H), 2.97-2.73 (m, 1H), 2.24 (t, J=6.3 Hz, 1H), 2.09 (s, 1H), 1.43 (t, J=7.2 Hz, 3H), 1.24-1.16 (m, 1H), 0.99 (d, J=6.8 Hz, 2H), 0.94-0.87 (m, 4H); ¹⁹F NMR (376 MHz, CDCl₃) δ=−115.43-−116.54 (m, 1F), −121.15-−121.93 (m, 1F).

### Example 72

### (R *)-4-(R *)-sec-Butyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one

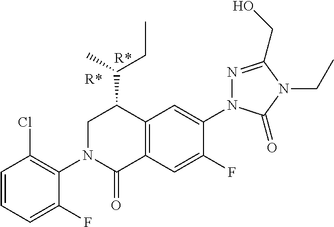 [SMILES: CC[C@@H](C)[C@H]1CN(c2c(F)cccc2Cl)C(=O)c2cc(F)c(-n3nc(CO)n(CC)c3=O)cc21]

The title compound was prepared as described in Example 71, expect the second eluting compound was isolated and yielded the title compound. MS (ESI): mass calcd. for C₂₄H₂₅ClF₂N₄O₃, 490.2; m/z found, 491.2 [M+H]⁺. ¹H NMR (400 MHz, CDCl₃) δ=7.99 (dd, J=3 .8, 11.0 Hz, 1H), 7.56-7.51 (m, 1H), 7.35-7.28 (m, 2H), 7.12 (dt, J=1.9, 8.5 Hz, 1H), 4.68 (d, J=3 .8 Hz, 2H), 4.22-4.05 (m, 1H), 3.92 (q, J=7 .3 Hz, 2H), 3.79-3.68 (m, 1H), 2.86-2.66 (m, 1H), 2.29 (s, 1H), 2.25-2.13 (m, 1H), 1.43 (t, J=7.2 Hz, 3H), 1.24-1.11 (m, 1H), 1.02 (d, J=6.5 Hz, 2H), 0.94-0.87 (m, 4H); ¹⁹F NMR (376 MHz, CDCl₃) δ=−116.57 (br d, J=37.2 Hz, 1F), −121.48-−121.60 (m, 1F).

### Example 73

### Racemic 2-(2-Chloro-6-fluorophenyl)-6-(4-ethyl-3-(hydroxymethyl)-5-oxo-4,5 -dihydro-1H-1,2,4-triazol-1-yl)-4-(3,3,3-trifluoroprop-1-en-2-yl)-3,4-dihydroisoquinolin-1(2H)-one

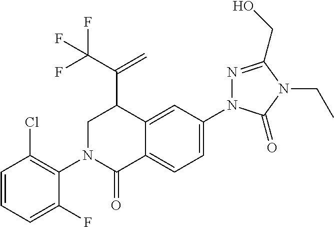 [SMILES: C=C(C1CN(c2c(F)cccc2Cl)C(=O)c2ccc(-n3nc(CO)n(CC)c3=O)cc21)C(F)(F)F]

Step A. 4,4,4-Trifluoro-3-methylbut-2-enal. To a solution of ethyl 3-(trifluoromethyl)crotonate (4.9 g, 26.9 mmol) in anhydrous ethyl ether (96 mL) at −78° C. was added a hexane solution (1 M) of diisobutylaluminium hydride (32.3 mL, 32.3 mmol) dropwise. The reaction mixture was stirred at −78° C. for 0.5 h, and then quenched with aqueous saturated NH₄Cl solution (50 mL). The organic layer was separated, and the aqueous layer was extracted with ethyl ether (50 mL). The combined organic extract was washed with water, brine, dried over MgSO₄ and concentrated to give the crude desired product as a colorless oil (yield, 2.05 g, 55%), which was used crude in the next step without further purification. ¹H NMR(400 MHz, CDCl₃) δ 10.11 (d, J=6.61 Hz, 1H), 6.41 (br d, J=6.61 Hz, 1H), 2.29 (s, 3H) ppm.

Step B. N-(2-Chloro-6-fluorophenyl)-4,4,4-trifluoro-3-methylbut-2-en-1-imine. To a solution of 2-chloro-6-fluoroaniline (1.8 g, 12.4 mmol) and 4,4,4-trifluoro-3-methylbut-2-enal (2.05 g, 14.8 mmol) in anhydrous dichloromethane (30 mL) at 0° C. was added triethylamine (5.2 mL, 37 mmol), followed by the slow addition of TiCl₄ (1.9 g, 9.9 mmol) dropwise. The reaction mixture was stirred at 0° C. for 1 h, then warmed to 25° C. and stirred for 4 h. The mixture was filtered through a short pad of Celite®, and the filtrate was partitioned between dichloromethane and water. The organic layer was separated, and the aqueous layer was extracted with dichloromethane. The combined organic extract was dried over MgSO₄ and concentrated. The residue was purified by ISCO chromatography (20-40% EtOAc in heptane) to afford the desired product as a yellow oil (yield, 1.8 g, 55%). ¹H NMR (400 MHz, CDCl₃) δ 8.46 (dd, J=2.45, 9.29 Hz, 1H), 7.20-7.28 (m, 1H), 7.01-7.10 (m, 1H), 6.85-6.97 (m, 1H), 6.61 (dt, J=5.62, 8.19 Hz, 1H), 2.14 (d, J=0.98 Hz, 3H) ppm.

Step C. 2-Chloro-6-fluoro-N-(4,4,4-trifluoro-3-methylbut-2-en-1-yl)aniline. To a solution of N-(2-chloro-6-fluorophenyl)-4,4,4-trifluoro-3-methylbut-2-en-1-imine (1.8 g, 6.8 mmol) in methanol (10 mL) was added NaBH₄ (0.26 g, 6.8 mmol) at 25° C., and after 1 h interval, another batch of NaBH₄ (0.26 g, 6.8 mmol) was added. A total of three equivalents of NaBH₄ (0.77 g, 20 mmol) was added. The reaction mixture was stirred at 25° C. for 5 h. The mixture was concentrated to dryness. The mixture was partitioned between ethyl acetate and water. The organic layer was separated, and the aqueous layer was extracted with ethyl acetate. The combined organic extract was dried over MgSO₄ and concentrated. The residue was purified by chromatography (5-25% EtOAc in heptane) to give the desired product as a light-yellow oil (yield, 1.4 g, 77%). LCMS (ES-API): mass calcd. for C₁₁H₁₀ClF₄N, 267.0; m/z found, 268.0 [M+H]⁺. ¹H NMR (400 MHz, CDCl₃) δ 7.00-7.13 (m, 1H), 6.86-6.98 (m, 1H), 6.66-6.78 (m, 1H), 6.52-6.69 (m, 1H), 6.17 (m, 1H), 4.01-4.07 (m, 2H), 1.82 (s, 3H) ppm.

Step D. 4-Bromo-N-(2-chloro-6-fluorophenyl)-2-iodo-N-(4,4,4-trifluoro-3-methylbut-2-en-1-yl)benzamide. To a flask charged with 4-bromo-2-iodobenzoic acid (2.6 g, 7.8 mmol) was added SOCl₂ (7 mL). The mixture was heated at 80° C. for 15 min, then cooled to 25° C. and concentrated to afford the crude 4-bromo-2-iodobenzoyl chloride as an off white solid. The crude 4-bromo-2-iodobenzoyl chloride was dissolved into dichloromethane (10 mL), and then added slowly dropwise to a pre-cooled dichloromethane solution (20 mL) of 2-chloro-6-fluoro-N-(4,4,4-trifluoro-3-methylbut-2-en-1-yl)aniline (1.4 g, 5.2 mmol) and triethylamine (2.2 mmol, 16 mmol) at 0° C., followed by the addition of a catalytic amount of 4-dimethylaminopyridine (DMAP) (6 mg, 0.05 mmol). The reaction mixture was warmed and stirred at 25° C. for 3 h. The reaction was quenched by the addition of aqueous saturated NaHCO₃ solution. The organic layer was separated, and the aqueous layer was extracted with ethyl acetate. The combined organic extract was washed with water, brine, dried over MgSO₄, and concentrated. The residue was purified by chromatography (10-30% EtOAc in heptane) to to afford the desired product as a brown oil (2.7 g, 90%). LCMS (ES-API): mass calcd. for C₁₈H₁₂BrClF₄INO, 574.9; m/z found, 576.0 [M+H]⁺. ¹ H NMR(400 MHz, CDCl₃) δ 7.90-8.02 (m, 1H), 7.45-7.56 (m, 1H), 7.29-7.39 (m, 1H), 7.12-7.23 (m, 2H), 6.88-7.04 (m, 1H), 6.18-6.33 (m, 1H), 4.90 (br dd, J=6.36, 15.16 Hz, 1H), 4.28 (br dd, J=7.34, 15.16 Hz, 1H), 1.71 (s, 3H) ppm.

Step E: Racemic-6-Bromo-2-(2-chloro-6-fluorophenyl)-4-(3,3,3-trifluoroprop-1-en-2-yl)-3,4-dihydroisoquinolin-1(2H)-one. To a mixture of 4-bromo-N-(2-chloro-6-fluorophenyl)-2-iodo-N-(4,4,4-trifluoro-3-methylbut-2-en-1-yl)benzamide (2.7 g, 4.7 mmol), tetrabutylammonium bromide (4.5 g, 14 mmol) and potassium acetate (0.92 g, 9.4 mmol) in anhydrous N,N-dimethylformamide (40 mL) was added palladium(II) acetate (1.0 g, 4.7 mmol) at 25° C. The reaction mixture was heated and stirred under nitrogen at 85° C. for 2 h, then cooled to 25° C. and water (100 mL) was added. The mixture was extracted with ethyl ether (100 mL) and ethyl acetate (100 mL). The combined organic extract was washed with brine, dried with anhydrous Na₂SO₄, and concentrated. The residue was purified by Chromatography (25-30% EtOAc in heptane) to afford the desired product as an amorphous (750 mg, 36%). LCMS (ES-API): mass calcd. for C₁₈H₁₁BrClF₄NO, 447.0; m/z found, 448.0 [M+H]⁺. ¹ H NMR(400 MHz, CDCl₃) δ 8.10 (dd, J=5.87, 8.31 Hz, 1H), 7.62 (dd, J=1.96, 8.31 Hz, 1H), 7.39 (t, J=2.45 Hz, 1H), 7.27-7.35 (m, 2H), 7.06-7.16 (m, 1H), 6.09 (d, J=0.98 Hz, 1H), 5.21-5.31 (m, 1H), 4.27 (dd, J=4.40, 12.72 Hz, 1H), 4.07-4.16 (m, 1H), 3.70-3.87 (m, 1H) ppm.

Step F: Racemic-6-(3-(((tert-Butyldiphenylsilyl)oxy)methyl)-4-ethyl-5-oxo-4,5-dihydro-1H-1,2,4-triazol-1-yl)-2-(2-chloro-6-fluorophenyl)-4-(3,3,3-trifluoroprop-1-en-2-yl)-3,4-dihydroisoquinolin-1(2H)-one. To a mixture of racemic-6-bromo-2-(2-chloro-6-fluorophenyl)-4-(3,3,3-trifluoroprop-1-en-2-yl)-3,4-dihydroisoquinolin-1(2H)-one (750 mg, 1.62 mmol) and K3PO4 (1065 mg, 5.0 mmol) in anhydrous 1,4-dioxane (8 mL) was added 5-(((tert-butyldiphenylsily0oxy)methyl)-4-ethyl-2,4-dihydro-3H-1,2,4-triazol-3-one (1275 mg, 3.3 mmol), CuI (318 mg, 1.67 mmol), and trans-N,N′-dimethylcyclohexane-1,2-diamine (238 mg, 1.67 mmol), respectively. The reaction mixture was slowly heated and stirred under nitrogen at 95° C. for 3 h, then cooled to 25° C. and quenched with addition of water (40 mL). The mixture was extracted with ethyl acetate (100 mL×2). The combined organic extract was washed with brine, dried with anhydrous Na₂SO₄, and concentrated. The residue was purified by chromatography (0-40% EtOAc in heptane) to afford the desired product as a white foam (720 mg, 57%). LCMS (ES-API): mass calcd. for C₃₉H₃₇ClF₄N₄O₃Si, 748.2; m/z found, 749.3 [M+H]⁺. ¹ H NMR(400 MHz, CDCl₃) δ 8.20-8.30 (m, 1H), 8.02-8.08 (m, 1H), 7.87-7.95 (m, 1H), 7.63-7.73 (m, 4H), 7.36-7.51 (m, 6H), 7.26-7.31 (m, 2H), 7.03-7.17 (m, 1H), 6.05 (s, 1H), 5.20-5.25 (m, 1H), 4.65 (s, 2H), 4.35 (br dd, J=4.65, 12.96 Hz, 1H), 4.06-4.16 (m, 1H), 3.90 (q, J=7 .01 Hz, 2H), 3.74 (br dd, J=4.40, 12.72 Hz, 1H), 1.33-1.42 (m, 3H), 1.09 (s, 9H) ppm.

Step G: Racemic-2-(2-chloro-6-fluorophenyl)-6-(4-ethyl-3-(hydroxymethyl)-5-oxo-4,5-dihydro-1H-1,2,4-triazol-1-yl)-4-(3,3,3-trifluoroprop-1-en-2-yl)-3,4-dihydroisoquinolin-1(2H)-one. To a solution of rac-6-(3-(((tert-Butyldiphenylsilyl)oxy)methyl)-4-ethyl-5 -oxo-4,5 -dihydro-1H-1,2,4-triazol-1-yl)-2-(2-chloro-6-fluorophenyl)-4-(3,3,3-trifluoroprop-1-en-2-yl)-3,4-dihydroisoquinolin-1(2H)-one (680 mg, 0.9 mmol) in tetrahydrofuran (THF) (20 mL) was added a tetrahydrofuran (THF) solution (1 M) of tetrabutylammonium fluoride (0.9 mL, 0.9 mmol). The reaction mixture was stirred at 25° C. for 0.5 h. The mixture was diluted with H₂O and extracted with ethyl acetate. The combined organic extract washed with brine, dried over Na₂SO₄, and concentrated. The residue was purified by chromatography (50-100% EtOAc in heptane), then further purified by prep-HPLC (C18 column, 20-80% gradient MeCN in water) to afford the title compound as a white solid (440 mg, yield 94%). LCMS (ES-API): mass calcd. for C₂₃H₁₉ClF₄N₄O₃, 510.1; m/z found, 511.2[M+H]⁺. ¹ H NMR(400 MHz, CDCl₃) δ 8.16-8.24 (m, 1H), 8.01-8.11 (m, 1H), 7.90 (dd, J=2.20, 5.62 Hz, 1H), 7.29 (quin, J=3.18 Hz, 2H), 7.06-7.16 (m, 1H), 6.04 (s, 1H), 5.21 (d, J=4.40 Hz, 1H), 4.63 (d, J=6.36 Hz, 2H), 4.23-4.39 (m, 1H), 4.09 (td, J=4.28, 12.96 Hz, 1H), 3.89 (q, J=7.34 Hz, 2H), 3.67-3.82 (m, 1H), 2.85 (td, J=6.42, 10.15 Hz, 1H), 1.40 (t, J=7.34 Hz, 3H) ppm.

### Example 74

### 6-(4-Ethyl-3-(hydroxymethyl)-5-oxo-4,5-dihydro-1H-1,2,4-triazol-1-yl)-2-(2-fluorophenyl)-4-(1,1,1-trifluoropropan-2-yl)-3,4-dihydroisoquinolin-1(2H)-one

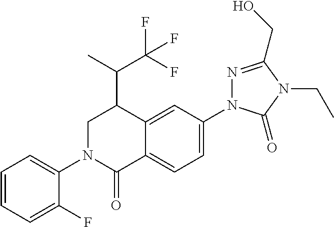 [SMILES: CCn1c(CO)nn(-c2ccc3c(c2)C(C(C)C(F)(F)F)CN(c2ccccc2F)C3=O)c1=O]

To a solution of 2-(2-chloro-6-fluorophenyl)-6-(4-ethyl-3-(hydroxymethyl)-5-oxo-4,5-dihydro-1H-1,2,4-triazol-1-yl)-4-(3,3,3-trifluoroprop-1-en-2-yl)-3,4-dihydroisoquinolin-1(2H)-one (Example 73, 90 mg, 0.18 mmol) in methanol (10 mL) was added Pd/C (10%, 37 mg). The mixture was degassed and purged with hydrogen gas for 3 times. The reaction mixture was then stirred under hydrogen atmosphere (15 psi) at 25° C. for 15 h. The mixture was filtered through a short pad of Celite®. The filtrate was concentrated. The residue was purified by prep-HPLC (C18 column, 20-80% gradient MeCN in water) to afford the title compound as a white solid (25 mg, yield 30%). LCMS (ES-API): mass calcd. for C₂₃H₂₂F₄N₄O₃, 478.2; m/z found, 479.3 [M+H]⁺. ¹H NMR (400 MHz, CDCl₃) δ 8.25 (d, J=8.31 Hz, 1H), 8.01-8.11 (m, 2H), 7.29-7.40 (m, 2H), 7.16-7.25 (m, 2H), 4.69 (s, 2H), 4.27 (br dd, J=4.65, 13.45 Hz, 1H), 3.81-3.97 (m, 3H), 3.52 (br d, J=3.91 Hz, 1H), 2.74-2.92 (m, 2H), 1.41 (t, J=7.34 Hz, 3H), 1.24 (d, J=7.34 Hz, 3H) ppm.

### Example 75

### 2-(2-Chloro-6-fluorophenyl)-6-(4-ethyl-3 -(hydroxymethyl)-5-oxo-4,5 -dihydro-1H-1,2,4-triazol-1-yl)-4-(1,1,1-trifluoropropan-2-yl)-3,4-dihydroisoquinolin-1(2H)-one

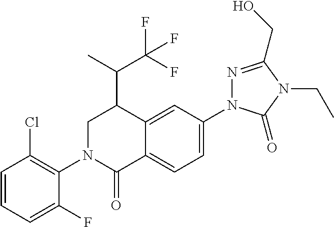 [SMILES: CCn1c(CO)nn(-c2ccc3c(c2)C(C(C)C(F)(F)F)CN(c2c(F)cccc2Cl)C3=O)c1=O]

In the preparation of (4-Ethyl-3-(hydroxymethyl)-5-oxo-4,5-dihydro-1H-1,2,4-triazol-1-yl)-2-(2-fluorophenyl)-4-(1,1,1-trifluoropropan-2-yl)-3,4-dihydroisoquinolin-1(2H)-one (Example 74), the title compound was obtained as the second product: a white solid (24 mg, yield 27%). LCMS (ES-API): mass calcd. for C23H₂₁ClF₄N₄O₃, 512.1; m/z found, 513.2 [M+H]⁺. ¹ H NMR(400 MHz, CDCl₃) 8 8.20-8.28 (m, 1H), 8.09 (s, 1H), 8.00-8.06 (m, 1H), 7.28-7.37 (m, 2H), 7.10-7.19 (m, 1H), 4.70 (s, 2H), 4.06-4.21 (m, 2H), 3.91 (q, J=7.17 Hz, 2H), 3.77-3.84 (m, 1H), 3.48 (br dd, J=5.14, 11.00 Hz, 1H), 3.03 (br s, 1H), 1.42 (t, J=7.09 Hz, 3H), 1.28 (d, J=7.09 Hz, 3H) ppm.

Biological Data

It was discovered that compounds of the present invention demonstrate DHODH inhibitory activity. DHODH inhibitory activities of the compounds of Examples 1-75 were assessed using the following assays. The half maximal inhibitory concentration values (IC₅₀) are summarized in Table 3.

Biological Assays

In vitro Assay: DHODH enzymatic assay

To detect DHODH enzyme activities, dichloroindophenol (DCIP) is added as the final electron acceptor in the assay. DCIP can accept electrons from the reduced coenzyme Q generated in the assay, or from dihydroorotate (DHO) via FMN by binding presumably to the ubiquinone pocket. DCIP solutions are blue, with an intense absorbance around 600 nm, but becomes colorless upon reduction (J. Biol. Chem. (1986) 261, 11386). The assay buffer contained 50 nM HEPES, pH 7.5, 150 mM NaCl, 0.5 mM EDTA, and 0.1% Triton X-100 in MilliQ water. Substrate consisting of 20 mM DHO, 5 mM CoQ₆, and 1 mM DCIP in assay buffer, initiates the reaction. The assay is run in end-point mode by quenching the reaction with the potent DHODH inhibitor brequinar. Absorbance measurements were obtained using the BMG Phera Star plate-reading spectrophotometer. Purified human DHODH was purchased from Proteros (cat. No. PR-0044). Chemicals were purchased from Sigma-Aldrich, Teknova, and Avanti Polar Lipids. Liquid handling was performed using Labcyte Echo and Formulatrix Tempest.

In Vitro Assay: MOLM-13 Cellular Assay

MOLM-13 cells were obtained from DSMZ and were maintained in RPMI 1640+Glutamax+25 mM HEPES (Invitrogen, catalog number 72400) supplemented with 10% heat inactivated fetal bovine serum (FBS; Invitrogen, catalog number 16140). The day prior to assay set-up, cells were pelleted, resuspended in fresh media, counted, and cells were plated at 0.4×10⁶ cell/mL in a T150 flask. On the day of the assay, cells were pelleted, resuspend in fresh media, counted and seeded at 5,000 cells/well in white opaque 96-well tissue culture treated microplates (Perkin Elmer, catalog number 6005680). Cells were exposed to different concentrations of test compounds at 37° C., 5% CO₂ for 72 hours immediately after seeding. Cell viability was acquired on a Perkin Elmer Envision 2104 multilabel reader using the CellTiter-Glo assay (Promega) according to the manufacturer's instructions.

TABLE 3

| Example | | MOLM-13 | hDHODH |
|---|---|---|---|
| # | Compound Name | IC₅₀ (nM) | IC₅₀ (nM) |
| 1 | 3-(2-Chloro-6-fluorophenyl)-7-(4-ethyl-3- | 0.85 | 1.5 |
| | (hydroxymethyl)-5-oxo-4,5-dihydro-1H- | | |
| | 1,2,4-triazol-1-yl)-6-fluoro-1-isopropyl-2,3- | | |
| | dihydroquinazolin-4(1H)-one; | | |
| 2 | 3-(2-Chloro-6-fluorophenyl)-7-(4-ethyl-3- | 8.4 | 35 |
| | (hydroxymethyl)-5-oxo-4,5-dihydro-1H- | | |
| | 1,2,4-triazol-1-yl)-6-fluoro-1-isopropyl-2- | | |
| | methyl-2,3-dihydroquinazolin-4(1H)-one; | | |
| 3 | 3-(2-Chloro-6-fluorophenyl)-7-(4-ethyl-3- | 440 | 1040 |
| | (hydroxymethyl)-5-oxo-4,5-dihydro-1H- | | |
| | 1,2,4-triazol-1-yl)-6-fluoro-1-isopropyl-2,2- | | |
| | dimethyl-2,3-dihydroquinazolin-4(1H)-one; | | |
| 4 | 3-(2-Chloro-6-fluorophenyl)-7-(4-ethyl-3- | 4.4 | 110 |
| | (hydroxymethyl)-5-oxo-4,5-dihydro-1H- | | |
| | 1,2,4-triazol-1-yl)-6-fluoro-1- | | |
| | isopropylquinazoline-2,4(1H,3H)-dione. | | |
| 5 | 3-(2-Chloro-6-fluorophenyl)-7-(4-ethyl-3- | 1.2 | 1.4 |
| | (hydroxymethyl)-5-oxo-4,5-dihydro-1H- | | |
| | 1,2,4-triazol-1-yl)-6-fluoro-1-isopropyl-2,3- | | |
| | dihydropyrido[2,3-d]pyrimidin-4(1H)-one; | | |
| 6 | 3-(2-Chloro-6-fluorophenyl)-7-(4-ethyl-3- | 50 | 57 |
| | (hydroxymethyl)-5-oxo-4,5-dihydro-1H- | | |
| | 1,2,4-triazol-1-yl)-6-fluoro-1-(tetrahydro- | | |
| | 2H-pyran-4-yl)-2,3-dihydropyrido[2,3-d] | | |
| | pyrimidin-4(1H)-one; | | |
| 7 | 3-(2-chloro-6-fluorophenyl)-1-cyclobutyl- | 1.7 | 2.6 |
| | 7-(4-ethyl-3-(hydroxymethyl)-5-oxo-4,5- | | |
| | dihydro-1H-1,2,4-triazol-1-yl)-6-fluoro-2,3- | | |
| | dihydropyrido[2,3-d]pyrimidin-4(1H)-one; | | |
| 8 | (S)-3-(2-Chloro-6-fluorophenyl)-7-(4-ethyl- | 1.7 | <0.5 |
| | 3-(hydroxymethyl)-5-oxo-4,5-dihydro-1H- | | |
| | 1,2,4-triazol-1-yl)-6-fluoro-1-(1,1,1- | | |
| | trifluoropropan-2-yl)-2,3-dihydropyrido | | |
| | [2,3-d]pyrimidin-4(1H)-one; | | |
| 9 | 3-(2-Chloro-6-fluorophenyl)-1-cyclopropyl- | 1.7 | 11 |
| | 7-(4-ethyl-3-(hydroxymethyl)-5-oxo-4,5- | | |
| | dihydro-1H-1,2,4-triazol-1-yl)-6-fluoro-2,3- | | |
| | dihydropyrido[2,3-d]pyrimidin-4(1H)-one; | | |
| 10 | Racemic-6-(4-Ethyl-3-(hydroxymethyl)-5- | >3.0 | 190 |
| | oxo-4,5-dihydro-1H-1,2,4-triazol-1-yl)-2- | | |
| | (3-fluorophenyl)-4-isopropyl-3,4- | | |
| | dihydroisoquinolin-1(2H)-one; | | |
| 11 | Racemic-2-(2-Chloro-6-fluorophenyl)-6- | 1 | 1.7 |
| | (4-ethyl-3-(hydroxymethyl)-5-oxo-4,5- | | |
| | dihydro-1H-1,2,4-triazol-1-yl)-4-(prop-1- | | |
| | en-2-yl)-3,4-dihydroisoquinolin-1(2H)-one; | | |
| 12 | Racemic-6-(4-Ethyl-3-(hydroxymethyl)-5- | 58 | 140 |
| | oxo-4,5-dihydro-1H-1,2,4-triazol-1-yl)-2- | | |
| | (2-fluorophenyl)-4-isopropyl-3,4- | | |
| | dihydroisoquinolin-1(2H)-one; | | |
| 13 | Racemic-2-(2-Chloro-6-fluorophenyl)-6- | 7.2 | 20 |
| | (4-ethyl-3-(hydroxymethyl)-5-oxo-4,5- | | |
| | dihydro-1H-1,2,4-triazol-1-yl)-4-isopropyl- | | |
| | 3,4-dihydroisoquinolin-1(2H)-one; | | |
| 14 | Racemic-6-(4-Ethyl-3-(hydroxymethyl)-5- | 758 | 1030 |
| | oxo-4,5-dihydro-1H-1,2,4-triazol-1-yl)-2- | | |
| | (3-fluorophenyl)-4-phenyl-3,4- | | |
| | dihydroisoquinolin-1(2H)-one; | | |
| 15 | (S)-3-(2-Chloro-6-fluorophenyl)-7-(4-ethyl- | <1.69 | 0.3 |
| | 3-(hydroxymethyl)-5-oxo-4,5-dihydro-1H- | | |
| | 1,2,4-triazol-1-yl)-6-fluoro-1-(pentan-2-yl)- | | |
| | 2,3-dihydroquinazolin-4(1H)-one; | | |
| 16 | (S)-1-(sec-Butyl)-3-(2-chloro-6- | <1.69 | 0.3 |
| | fluorophenyl)-7-(4-ethyl-3- | | |
| | (hydroxymethyl)-5-oxo-4,5-dihydro- | | |
| | 1H-1,2,4-triazol-1-yl)-6-fluoro-2,3- | | |
| | dihydroquinazolin-4(1H)-one; | | |
| 17 | (S)-3-(2-Chloro-6-fluorophenyl)-1- | <1.69 | 0.1 |
| | (1-cyclohexylethyl)-7-(4-ethyl-3- | | |
| | (hydroxymethyl)-5-oxo-4,5-dihydro- | | |
| | 1,2,4-triazol-1-yl)-6-fluoro-2,3- | | |
| | 1H-dihydroquinazolin-4(1H)-one; | | |
| 18 | 3-(2-Chloro-6-fluorophenyl)-7-(4-ethyl-3- | 3.99 | 2 |
| | (hydroxymethyl)-5-oxo-4,5-dihydro-1H- | | |
| | 1,2,4-triazol-1-yl)-6-fluoro-1-isobutyl- | | |
| | 2,3-dihydroquinazolin-4(1H)-one; | | |
| 19 | 3-(2-Chloro-6-fluorophenyl)-1-cyclobutyl- | 2.57 | 3 |
| | 7-(4-ethyl-3-(hydroxymethyl)-5-oxo-4,5- | | |
| | dihydro-1H-1,2,4-triazol-1-yl)-6-fluoro- | | |
| | 2,3-dihyroquinazolin-4(1H)-one; | | |
| 20 | 1-Butyl-3-(2-chloro-6-fluorophenyl)-7- | 10.2 | 5 |
| | (4-ethyl-3-(hydroxymethyl)-5-oxo-4,5- | | |
| | dihydro-1H-1,2,4-triazol-1-yl)-6-fluoro- | | |
| | 2,3-dihydroquinazolin-4(1H)-one; | | |
| 21 | 3-(2-Chloro-6-fluorophenyl)-1- | 22.9 | 6 |
| | (cyclohexylmethyl)-7-(4-ethyl-3- | | |
| | (hydroxymethyl)-5-oxo-4,5-dihydro- | | |
| | 1H-1,2,4-triazol-1-yl)-6-fluoro-2,3- | | |
| | dihydroquinazolin-4(1H)-one; | | |
| 22 | 3-(2-Chloro-6-fluorophenyl)-7-(4-ethyl-3- | 5.49 | 4 |
| | (hydroxymethyl)-5-oxo-4,5-dihydro-1H- | | |
| | 1,2,4-triazol-1-yl)-6-fluoro-1-propyl-2,3- | | |
| | dihydroquinazolin-4(1H)-one; | | |
| 23 | (S)-1-(1-(1,3-Dioxan-2-yl)ethyl)-3-(2- | 6.64 | 3 |
| | chloro-6-fluorophenyl)-7-(4-ethyl-3- | | |
| | (hydroxymethyl)-5-oxo-4,5-dihydro- | | |
| | 1H-1,2,4-triazol-1-yl)-6-fluoro-2,3- | | |
| | dihydropyrido[2,3-d] pyrimidin-4(1H)-one; | | |
| 24 | (S)-3-(2-Chloro-6-fluorophenyl)-7-(4-ethyl- | <1.69 | 0.4 |
| | 3-(hydroxymethyl)-5-oxo-4,5-dihydro-1H- | | |
| | 1,2,4-triazol-1-yl)-6-fluoro-1-(pentan-2-yl)- | | |
| | 2,3-dihydropyrido[2,3-d]pyrimidin-4(1H)-one; | | |
| 25 | 2-(4-Chloro-2-methylpyridin-3-yl)-6-(4- | 62.5 | >100 |
| | ethyl-3-(hydroxymethyl)-5-oxo-4,5-dihydro- | | |
| | 1H-1,2,4-triazol-1-yl)-7-fluoro-4-(prop-1- | | |
| | en-2-yl)-3,4-dihydroisoquinolin-1(2H)-one; | | |
| 26 | 7-(4-Ethyl-3-(hydroxymethyl)-5-oxo-4,5- | 27.5 | 20 |
| | dihydro-1H-1,2,4-triazol-1-yl)-6-fluoro-3- | | |
| | (2-fluoro-5-methylphenyl)-1-isopropyl-2,3- | | |
| | dihydroquinazolin-4(1H)-one; | | |
| 27 | 4-(7-(4-Ethyl-3-(hydroxymethyl)-5-oxo-4,5- | 48.1 | 30 |
| | dihydro-1H-1,2,4-triazol-1-yl)-6-fluoro-1- | | |
| | isopropyl-4-oxo-1,4-dihydroquinazolin- | | |
| | 3(2H)-yl)-5-fluoronicotinonitrile; | | |
| 28 | 3-(2-Chloro-4-methylpyridin-3-yl)-7-(4- | 36.1 | 20 |
| | ethyl-3-(hydroxymethyl)-5-oxo-4,5- | | |
| | dihydro-1H-1,2,4-triazol-1-yl)-6-fluoro-1- | | |
| | isopropyl-2,3-dihydroquinazolin-4(1H)-one; | | |
| 29 | 3-(3-Chloro-5-fluoropyridin-4-yl)-7-(4- | 27.3 | 20 |
| | ethyl-3-(hydroxymethyl)-5-oxo-4,5- | | |
| | dihydro-1H-1,2,4-triazol-1-yl)-6-fluoro-1- | | |
| | isopropyl-2,3-dihydroquinazolin-4(1H)-one; | | |
| 30 | 3-(3-Chloro-6-methoxypyridin-2-yl)-7-(4- | 3.39 | 3 |
| | ethyl-3-(hydroxymethyl)-5-oxo-4,5-dihydro- | | |
| | 1H-1,2,4-triazol-1-yl)-6-fluoro-1- | | |
| | isopropyl-2,3-dihydroquinazolin-4(1H)-one; | | |
| 31 | (S)-1-(sec-Butyl)-3-(2-chloro-6-fluorophenyl)- | 0.962 | 0.7 |
| | 7-(4-ethyl-3-(hydroxymethyl)-5-oxo-4,5- | | |
| | dihydro-1H-1,2,4-triazol-1-yl)-6-fluoro-2,3- | | |
| | dihydropyrido[2,3-d]pyrimidin-4(1H)-one; | | |
| 32 | 3-(3-Chloro-2-methoxy-5-methylpyridin-4- | 5.96 | 9 |
| | yl)-7-(4-ethyl-3-(hydroxymethyl)-5-oxo- | | |
| | 4,5-dihydro-1H-1,2,4-triazol-1-yl)-6-fluoro- | | |
| | 1-isopropyl-2,3-dihydroquinazolin-4(1H)-one; | | |
| 33 | (S*)-6-[4-Ethyl-3-(hydroxymethyl)-5-oxo- | 1 | 1 |
| | 1,2,4-triazol-1-yl]-7-fluoro-4-isopropenyl- | | |
| | 2-(o-tolyl)-3,4-dihydroisoquinolin-1-one; | | |
| 34 | (S*)-6-[4-Ethyl-3-(hydroxymethyl)-5-oxo- | 1.06 | 0.9 |
| | 1,2,4-triazol-1-yl]-7-fluoro-4-isopropenyl- | | |
| | 2-(o-tolyl)-3,4-dihydroisoquinolin-1-one; | | |
| 35 | (R*)-6-[4-Ethyl-3-(hydroxymethyl)-5-oxo- | 7.59 | 4 |
| | 1,2,4-triazol-1-yl]-7-fluoro-4-isopropyl-2- | | |
| | (o-tolyl)-3,4-dihydroisoquinolin-1-one; | | |
| 36 | (S*)-2-(2-Chlorophenyl)-6-(4-ethyl-3- | 31.6 | 30 |
| | (hydroxymethyl)-5-oxo-4,5-dihydro-1H- | | |
| | 1,2,4-triazol-1-yl)-7-fluoro-4-isopropyl- | | |
| | 3,4-dihydroisoquinolin-1(2H)-one; | | |
| 37 | (R*)-2-(2-Chlorophenyl)-6-(4-ethyl-3- | 15.5 | 20 |
| | (hydroxymethyl)-5-oxo-4,5-dihydro-1H- | | |
| | 1,2,4-triazol-1-yl)-7-fluoro-4-isopropyl-3,4- | | |
| | dihydroisoquinolin-1(2H)-one; | | |
| 38 | Racemic-6-(4-Ethyl-3-(hydroxymethyl)-5- | 36.3 | 30 |
| | oxo-4,5-dihydro-1H-1,2,4-triazol-1-yl)-7- | | |
| | fluoro-2-(2-fluoro-5-methylphenyl)-4- | | |
| | (prop-1-en-2-yl)-3,4-dihydroisoquinolin- | | |
| | 1(2H)-one; | | |
| 39 | (S*)-6-(4-Ethyl-3-(hydroxymethyl)-5-oxo- | 14.1 | 20 |
| | 4,5-dihydro-1H-1,2,4-triazol-1-yl)-7-fluoro- | | |
| | 2-(2-fluoro-5-methylphenyl)-4-(prop-1-en- | | |
| | 2-yl)-3,4-dihydroisoquinolin-1(2H)-one; | | |
| 40 | (R*)-6-(4-Ethyl-3-(hydroxymethyl)-5-oxo- | 52.9 | 40 |
| | 4,5-dihydro-1H-1,2,4-triazol-1-yl)-7-fluoro- | | |
| | 2-(2-fluoro-5-methylphenyl)-4-(prop-1-en- | | |
| | 2-yl)-3,4-dihydroisoquinolin-1(2H)-one; | | |
| 41 | (S*)-6-(4-Ethyl-3-(hydroxymethyl)-5-oxo- | 1.99 | 1 |
| | 4,5-dihydro-1H-1,2,4-triazol-1-yl)-7-fluoro- | | |
| | 4-(prop-1-en-2-yl)-2-(o-tolyl)-3,4- | | |
| | dihydroisoquinolin-1(2H)-one; | | |
| 42 | (R*)-6-(4-Ethyl-3-(hydroxymethyl)-5-oxo- | 6.79 | 4 |
| | 4,5-dihydro-1H-1,2,4-triazol-1-yl)-7-fluoro- | | |
| | 4-(prop-1-en-2-yl)-2-(o-tolyl)-3,4- | | |
| | dihydroisoquinolin-1(2H)-one; | | |
| 43 | (S*)-6-(4-Ethyl-3-(hydroxymethyl)-5-oxo- | 15 | 20 |
| | 4,5-dihydro-1H-1,2,4-triazol-1-yl)-7-fluoro- | | |
| | 4-isopropyl-2-(o-tolyl)-3,4- | | |
| | dihydroisoquinolin-1(2H)-one; | | |
| 44 | (R*)-6-(4-Ethyl-3-(hydroxymethyl)-5-oxo- | 6.85 | 9 |
| | 4,5-dihydro-1H-1,2,4-triazol-1-yl)-7-fluoro- | | |
| | 4-isopropyl-2-(o-tolyl)-3,4- | | |
| | dihydroisoquinolin-1(2H)-one; | | |
| 45 | Racemic-2-(2-Chloro-4-methylpyridin-3- | 6.98 | 7 |
| | yl)-6-(4-ethyl-3-(hydroxymethyl)-5-oxo- | | |
| | 4,5-dihydro-1H-1,2,4-triazol-1-yl)-7-fluoro- | | |
| | 4-(prop-1-en-2-yl)-3,4-dihydroisoquinolin- | | |
| | 1(2H)-one; | | |
| 46 | (S*)-2-(2-Chloro-4-methylpyridin-3-yl)-6- | 5.46 | NT |
| | (4-ethyl-3-(hydroxymethyl)-5-oxo-4,5- | | |
| | dihydro-1H-1,2,4-triazol-1-yl)-7-fluoro-4- | | |
| | (prop-1-en-2-yl)-3,4-dihydroisoquinolin- | | |
| | 1(2H)-one; | | |
| 47 | (R*)-2-(2-Chloro-4-methylpy ridin-3-yl)-6- | 298 | NT |
| | (4-ethyl-3-(hydroxymethyl)-5-oxo-4,5- | | |
| | dihydro-1H-1,2,4-triazol-1-yl)-7-fluoro-4- | | |
| | (prop-1-en-2-yl)-3,4-dihydroisoquinolin- | | |
| | 1(2H)-one; | | |
| 48 | (S*)-2-(5-Chloro-3-methyl-1H-pyrazol-4- | 0.834 | 20 |
| | yl)-6-(4-ethyl-3-(hydroxymethyl)-5-oxo- | | |
| | 4,5-dihydro-1H-1,2,4-triazol-1-yl)-7-fluoro- | | |
| | 4-(prop-1-en-2-yl)-3,4-dihydroisoquinolin- | | |
| | 1(2H)-one; | | |
| 49 | (R*)-2-(5-Chloro-3-methyl-1H-pyrazol-4- | 2.28 | 10 |
| | yl)-6-(4-ethyl-3-(hydroxymethyl)-5-oxo-4,5- | | |
| | dihydro-1H-1,2,4-triazol-1-yl)-7-fluoro-4- | | |
| | (prop-1-en-2-yl)-3,4-dihydroisoquinolin- | | |
| | 1(2H)-one; | | |
| 50 | Atropisomer 1, (S*)-6-(4-Ethyl-3- | 8.72 | NT |
| | (hydroxymethyl)-5-oxo-4,5-dihydro-1H- | | |
| | 1,2,4-triazol-1-yl)-7-fluoro-2-(2-methoxy- | | |
| | 4-methylpyridin-3-yl)-4-(prop-1-en-2-yl)- | | |
| | 3,4-dihydroisoquinolin-1(2H)-one; | | |
| 50 | Atropisomer 2, (S*)-6-(4-Ethyl-3- | 629 | NT |
| | (hydroxymethyl)-5-oxo-4,5-dihydro-1H- | | |
| | 1,2,4-triazol-1-yl)-7-fluoro-2-(2-methoxy- | | |
| | 4-methylpyridin-3-yl)-4-(prop-1-en-2-yl)- | | |
| | 3,4-dihydroisoquinolin-1(2H)-one; | | |
| 52 | Atropisomer 1, (R*)-6-(4-Ethyl-3- | 105 | NT |
| | (hydroxymethyl)-5-oxo-4,5-dihydro-1H- | | |
| | 1,2,4-triazol-1-yl)-7-fluoro-2-(2-methoxy- | | |
| | 4-methylpyridin-3-yl)-4-(prop-1-en-2-yl)- | | |
| | 3,4-dihydroisoquinolin-1(2H)-one; | | |
| 53 | Atropisomer 2, (R*)-6-(4-Ethyl-3- | 3.36 | NT |
| | (hydroxymethyl)-5-oxo-4,5-dihydro-1H- | | |
| | 1,2,4-triazol-1-yl)-7-fluoro-2-(2-methoxy- | | |
| | 4-methylpyridin-3-yl)-4-(prop-1-en-2-yl)- | | |
| | 3,4-dihydroisoquinolin-1(2H)-one; | | |
| 54 | Atropisomer 1, (S*)-6-(4-Ethyl-3- | 2.2 | 20 |
| | (hydroxymethyl)-5-oxo-4,5-dihydro-1H- | | |
| | 1,2,4-triazol-1-yl)-7-fluoro-2-(2-methoxy- | | |
| | 3,5-dimethylpyridin-4-yl)-4-(prop-1-en-2- | | |
| | yl)-3,4-dihydroisoquinolin-1(2H)-one; | | |
| 55 | Atropisomer 1, (R*)-6-(4-Ethyl-3- | 1164 | >100 |
| | (hydroxymethyl)-5-oxo-4,5-dihydro-1H- | | |
| | 1,2,4-triazol-1-yl)-7-fluoro-2-(2-methoxy- | | |
| | 3,5-dimethylpyridin-4-yl)-4-(prop-1-en-2- | | |
| | yl)-3,4-dihydroisoquinolin-1(2H)-one; | | |
| 56 | Atropisomer 2, (S*)-6-(4-Ethyl-3- | 101 | >100 |
| | (hydroxymethyl)-5-oxo-4,5-dihydro-1H- | | |
| | 1,2,4-triazol-1-yl)-7-fluoro-2-(2-methoxy- | | |
| | 3,5-dimethylpyridin-4-yl)-4-(prop-1-en-2- | | |
| | yl)-3,4-dihydroisoquinolin-1(2H)-one; | | |
| 57 | Atropisomer 2, (R*)-6-(4-Ethyl-3- | 29.7 | >100 |
| | (hydroxymethyl)-5-oxo-4,5-dihydro-1H- | | |
| | 1,2,4-triazol-1-yl)-7-fluoro-2-(2-methoxy- | | |
| | 3,5-dimethylpyridin-4-yl)-4-(prop-1-en-2- | | |
| | yl)-3,4-dihydroisoquinolin-1(2H)-one; | | |
| 58 | Atropisomer 1, (S*)-2-(2-Chloro-4,6- | 2.93 | 4 |
| | dimethylpyridin-3-yl)-6-(4-ethyl-3- | | |
| | (hydroxymethyl)-5-oxo-4,5-dihydro-1H- | | |
| | 1,2,4-triazol-1-yl)-7-fluoro-4-(prop-1-en-2- | | |
| | yl)-3,4-dihydroisoquinolin-1(2H)-one; | | |
| 59 | Atropisomer 1, (R*)-2-(2-Chloro-4,6- | 1859 | >100 |
| | dimethylpyridin-3-yl)-6-(4-ethyl-3- | | |
| | (hydroxymethyl)-5-oxo-4,5-dihydro-1H- | | |
| | 1,2,4-triazol-1-yl)-7-fluoro-4-(prop-1-en-2- | | |
| | yl)-3,4-dihydroisoquinolin-1(2H)-one; | | |
| 60 | Atropisomer 1, (S*)-2-(2-Chloro-4,6- | 26.8 | >100 |
| | dimethylpyridin-3-yl)-6-(4-ethyl-3- | | |
| | (hydroxymethyl)-5-oxo-4,5-dihydro-1H- | | |
| | 1,2,4-triazol-1-yl)-7-fluoro-4-(prop-1-en-2- | | |
| | yl)-3,4-dihydroisoquinolin-1(2H)-one; | | |
| 61 | Atropisomer 2(R*)-2-(2-Chloro-4,6- | 114 | 90 |
| | dimethylpyridin-3-yl)-6-(4-ethyl-3- | | |
| | (hydroxymethyl)-5-oxo-4,5-dihydro-1H- | | |
| | 1,2,4-triazol-1-yl)-7-fluoro-4-(prop-1-en-2- | | |
| | yl)-3,4-dihydroisoquinolin-1(2H)-one; | | |
| 62 | Atropisomer 1, (S*)-2-(3-Chloro-2- | 0.793 | 2 |
| | methoxy-5-methylpyridin-4-yl)-6-(4-ethyl- | | |
| | 3-(hydroxymethyl)-5-oxo-4,5-dihydro-1H- | | |
| | 1,2,4-triazol-1-yl)-7-fluoro-4-(prop-1-en-2- | | |
| | yl)-3,4-dihydroisoquinolin-1(2H)-one; | | |
| 63 | Atropisomer 1, (R*)-2-(3-Chloro-2- | 359 | >100 |
| | methoxy-5-methylpyridin-4-yl)-6-(4-ethyl- | | |
| | 3-(hydroxymethyl)-5-oxo-4,5-dihydro-1H- | | |
| | 1,2,4-triazol-1-yl)-7-fluoro-4-(prop-1-en-2- | | |
| | yl)-3,4-dihydroisoquinolin-1(2H)-one; | | |
| 64 | Atropisomer 2, (S*)-2-(3-Chloro-2- | 15.3 | 60 |
| | methoxy-5-methylpyridin-4-yl)-6-(4-ethyl- | | |
| | 3-(hydroxymethyl)-5-oxo-4,5-dihydro-1H- | | |
| | 1,2,4-triazol-1-yl)-7-fluoro-4-(prop-1-en-2- | | |
| | yl)-3,4-dihydroisoquinolin-1(2H)-one; | | |
| 65 | Atropisomer 2, (R*)-2-(3-Chloro-2- | 20.7 | 100 |
| | methoxy-5-methylpyridin-4-yl)-6-(4-ethyl- | | |
| | 3-(hydroxymethyl)-5-oxo-4,5-dihydro-1H- | | |
| | 1,2,4-triazol-1-yl)-7-fluoro-4-(prop-1-en-2- | | |
| | yl)-3,4-dihydroisoquinolin-1(2H)-one; | | |
| 66 | (S*)-2-(3-Chloro-2-methoxy-5- | 2.96 | 2 |
| | methylpyridin-4-yl)-6-(4-ethyl-3- | | |
| | (hydroxymethyl)-5-oxo-4,5-dihydro-1H- | | |
| | 1,2,4-triazol-1-yl)-7-fluoro-4-isopropyl- | | |
| | 3,4-dihydroisoquinolin-1(2H)-one; | | |
| 67 | (R*)-2-(3-Chloro-2-methoxy-5- | 351 | <100 |
| | methylpyridin-4-yl)-6-(4-ethyl-3- | | |
| | (hydroxymethyl)-5-oxo-4,5-dihydro-1H- | | |
| | 1,2,4-triazol-1-yl)-7-fluoro-4-isopropyl- | | |
| | 3,4-dihydroisoquinolin-1(2H)-one; | | |
| 68 | (R*)-2-(2-Chloro-6-fluorophenyl)-6-(4- | 4.66 | 2 |
| | ethyl-3-(hydroxymethyl)-5-oxo-4,5- | | |
| | dihydro-1H-1,2,4-triazol-1-yl)-7-fluoro-4- | | |
| | (prop-1-en-2-yl)-3,4-dihydroisoquinolin- | | |
| | 1(2H)-one; | | |
| 69 | (S*)-2-(2-Chloro-6-fluorophenyl)-6-(4- | <1.69 | 0.3 |
| | ethyl-3-(hydroxymethyl)-5-oxo-4,5- | | |
| | dihydro-1H-1,2,4-triazol-1-yl)-7-fluoro-4- | | |
| | (prop-1-en-2-yl)-3,4-dihydroisoquinolin- | | |
| | 1(2H)-one; | | |
| 70 | (S*)-4-((RS)-sec-Butyl)-2-(2-chloro-6- | 14.5 | 6 |
| | fluorophenyl)-6-(4-ethyl-3- | | |
| | (hydroxymethyl)-5-oxo-4,5-dihydro- | | |
| | 1H-1,2,4-triazol-1-yl)-7-fluoro-3,4- | | |
| | dihydroisoquinolin-1(2H)-one; | | |
| 71 | (R*)-4-((S*)-sec-Butyl)-2-(2-chloro-6- | 4.3 | 4 |
| | fluorophenyl)-6-(4-ethyl-3- | | |
| | (hydroxymethyl)-5-oxo-4,5-dihydro- | | |
| | 1H-1,2,4-triazol-1-yl)-7-fluoro-3,4- | | |
| | dihydroisoquinolin-1(2H)-one; | | |
| 72 | (R*)-4-((R*)-sec-Butyl)-2-(2-chloro-6- | 6.57 | 5 |
| | fluorophenyl)-6- (4-ethyl-3- | | |
| | (hydroxymethyl)-5-oxo-4,5-dihydro- | | |
| | 1H-1,2,4-triazol-1-yl)-7-fluoro-3,4- | | |
| | dihydroisoquinolin-1(2H)-one; | | |
| 73 | Racemic-2-(2-Chloro-6-fluorophenyl)-6- | 12.2 | 8 |
| | (4-ethyl-3-(hydroxymethyl)-5-oxo-4,5- | | |
| | dihydro-1H-1,2,4-triazol-1-yl)-4-(3,3,3- | | |
| | trifluoroprop-1-en-2-yl)-3,4- | | |
| | dihydroisoquinolin-1(2H)-one; | | |
| 74 | 6-(4-Ethyl-3-(hydroxymethyl)-5-oxo-4,5- | 102 | NT |
| | dihydro-1H-1,2,4-triazol-1-yl)-2-(2- | | |
| | fluorophenyl)-4-(1,1,1-trifluoropropan-2- | | |
| | yl)-3,4-dihydroisoquinolin-1(2H)-one; and | | |
| 75 | 2-(2-Chloro-6-fluorophenyl)-6-(4-ethyl-3- | 16.1 | NT |
| | (hydroxymethyl)-5-oxo-4,5-dihydro-1H- | | |
| | 1,2,4-triazol-1-yl)-4-(1,1,1-trifluoropropan- | | |
| | 2-yl)-3,4-dihydroisoquinolin-1(2H)-one. | | |

NT means Not Tested

What is claimed is:

1. A compound having the structure of Formula (I): 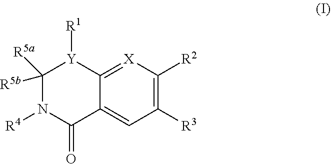
wherein
X is CH or N;
Y is CH or N;
R¹ is selected from the group consisting of: C₁₋₆alkyl; C₁₋₆alkyl substituted with OH, OCH₃, C₃₋₆cycloalkyl or C₃₋₆heterocycloalkyl; C₂₋₆alkenyl; C₂₋₆alkenyl substituted with one, two or three halo members; C₁₋₆haloalkyl; C₁₋₆haloalkyl substituted with OH, or OCH₃; C₃₋₆cycloalkyl; C₃₋₆heterocycloalkyl; and phenyl;
R² is 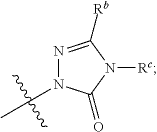
wherein
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, and C₃₋₆cycloalkyl;
R³ is H or F;
R⁴ is selected from the group consisting of: 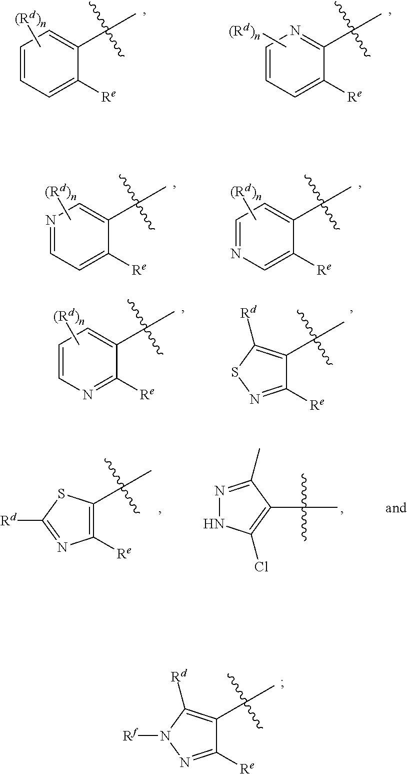
wherein
each R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
R^{e} is selected from the group consisting of: H; halo; CN; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃;
R^{5a} and R^{5b} are each independently H or CH₃; or R^{5a} and R^{5b} come together to form ═O; and
n is 1 or 2;
or a pharmaceutically acceptable salt, solvate, stereoisomer, isotopic variant, or N-oxide thereof.

2. The compound according to claim 1, wherein X is CH; or a pharmaceutically acceptable salt, solvate, stereoisomer, isotopic variant, or N-oxide thereof.

3. The compound according to claim 1, wherein X is N; or a pharmaceutically acceptable salt, solvate, stereoisomer, isotopic variant, or N-oxide thereof.

4. The compound according to claim 1, wherein Y is N; or a pharmaceutically acceptable salt, solvate, stereoisomer, isotopic variant, or N-oxide thereof.

5. The compound according to claim 1, wherein Y is CH; or a pharmaceutically acceptable salt, solvate, stereoisomer, isotopic variant, or N-oxide thereof.

6. The compound according to claim 1, wherein R¹ is selected from the group consisting of: C₁₋₆alkyl; C₁₋₆alkyl substituted with OH, OCH₃, C₃₋₆cycloalkyl or C₃₋₆heterocycloalkyl; C₂₋₆alkenyl; C₂₋₆alkenyl substituted with one, two or three F members; C₁₋₆haloalkyl; C₁₋₆haloalkyl substituted with OH, or OCH₃; C₃₋₆cycloalkyl; tetrahydropyran-4-yl; and phenyl; or a pharmaceutically acceptable salt, solvate, stereoisomer, isotopic variant, or N-oxide thereof.

7. The compound according to claim 1, wherein R¹ is C₁₋₄alkyl; C₃₋₆cycloalkyl or C₃₋₆heterocycloalkyl; 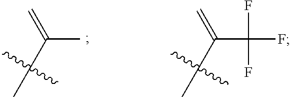
C₁₋₄haloalkyl; C₁₋₄haloalkyl substituted with OH, or OCH₃; tetrahydropyran-4-yl; or C₃₋₆cycloalkyl; or a pharmaceutically acceptable salt, solvate, stereoisomer, isotopic variant, or N-oxide thereof.

8. The compound according to claim 1, wherein Y is N and R¹ is CH(CH₃)₂, CH₂CH(CH₃)₂, CH₂CH₂CH₃, CH₂CH₂CH₂CH₃, CH(CH₃)CH₂CH₃, CH(CH₃)CH₂CH₂CH₃, CH(CH₃)(CF₃), CH(CH₃)CH₂OCH₃, 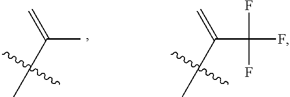
cyclopropyl, cyclobutyl, 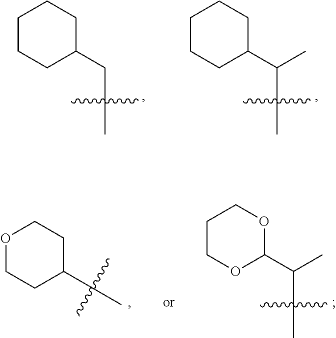
or a pharmaceutically acceptable salt, solvate, stereoisomer, isotopic variant, or N-oxide thereof

9. The compound according to claim 1, wherein Y is CH and R¹ is 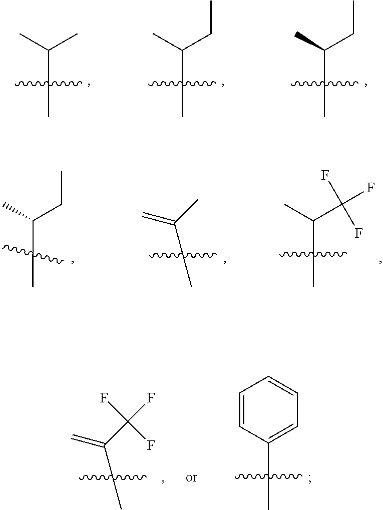
or a pharmaceutically acceptable salt, solvate, stereoisomer, isotopic variant, or N-oxide thereof.

10. The compound according to claim 1, wherein
R² is 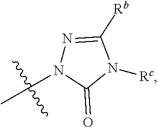
wherein
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, isotopic variant, or N-oxide thereof.

11. The compound according to claim 1, wherein
R² is 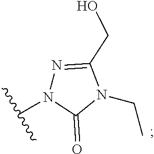 [SMILES: *n1nc(CO)n(CC)c1=O]
or a pharmaceutically acceptable salt, solvate, stereoisomer, isotopic variant, or N-oxide thereof.

12. The compound according to claim 1, wherein R³ is H; or a pharmaceutically acceptable salt, solvate, stereoisomer, isotopic variant, or N-oxide thereof.

13. The compound according to claim 1, wherein R³ is F; or a pharmaceutically acceptable salt, solvate, stereoisomer, isotopic variant, or N-oxide thereof.

14. The compound according to claim 1, wherein
R⁴ is 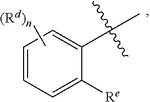
wherein
each R^{d} is independently selected from the group consisting of: H; halo; C₁₋₄alkyl; C₁₋₄alkyl substituted with OH, OCH₃, SCH₃, or OCF₃; C₁₋₄haloalkyl; C₁₋₄haloalkyl substituted with OH, OCH₃; and OC₁₋₄alkyl;
R^{e} is H; halo; CN; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃; and
n is 1 or 2;
or a pharmaceutically acceptable salt, solvate, stereoisomer, isotopic variant, or N-oxide thereof.

15. The compound according to claim 1, wherein
R⁴ is 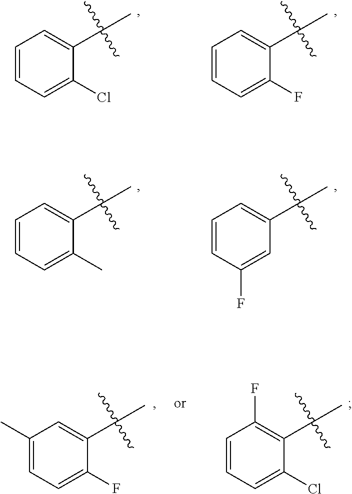
or a pharmaceutically acceptable salt, solvate, stereoisomer, isotopic variant, or N-oxide thereof.

16. The compound according to claim 1, wherein
R⁴ is 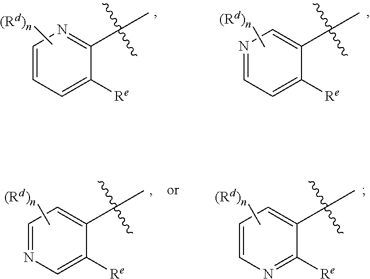
wherein
each R^{d} is independently selected from the group consisting of: H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; and OC₁₋₄alkyl;
R^{e} is H; halo; CN; C₁₋₄alkyl; C₁₋₄alkyl substituted with OH, OCH₃, SCH₃, or OCF₃;
C₁₋₄haloalkyl; or C₁₋₄haloalkyl substituted with OH, or OCH₃; and
n is 1 or 2;
or a pharmaceutically acceptable salt, solvate, stereoisomer, isotopic variant, or N-oxide thereof.

17. The compound according to claim 1, wherein
R⁴ is 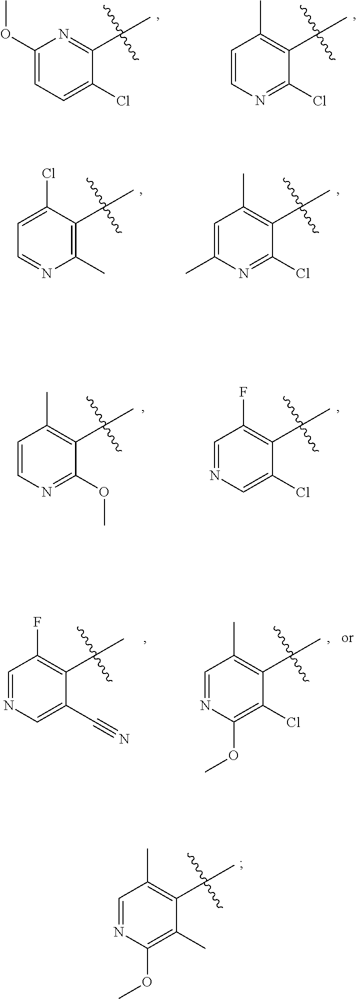
or a pharmaceutically acceptable salt, solvate, stereoisomer, isotopic variant, or N-oxide thereof.

18. The compound according to claim 1, wherein
R⁴ is 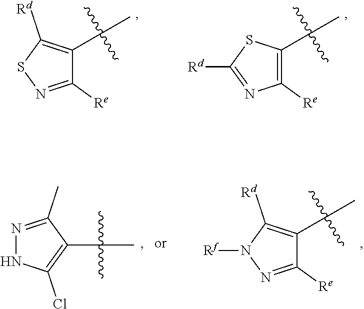
wherein
R^{d} is H; halo; C₁₋₄alkyl; C₁₋₄alkyl substituted with OH, OCH₃, SCH₃, or OCF₃;
C₁₋₄haloalkyl; C₁₋₄haloalkyl substituted with OH, or OCH₃; or OC₁₋₄alkyl;
R^{e} is H; halo; CN; C₁₋₄alkyl; C₁₋₄alkyl substituted with OH, OCH₃, SCH₃, or OCF₃;
C₁₋₄haloalkyl; or C₁₋₄haloalkyl substituted with OH, or OCH₃; and
R^{f} is H; C₁₋₄alkyl; C₁₋₄alkyl substituted with OH, OCH₃, SCH₃, or OCF₃; C₁₋₄haloalkyl;
or C₁₋₄haloalkyl substituted with OH, or OCH₃; or a pharmaceutically acceptable salt, solvate, stereoisomer, isotopic variant, or N-oxide thereof.

19. The compound according to claim 1, wherein
R⁴ is 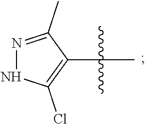 [SMILES: *c1c(C)n[nH]c1Cl]
or a pharmaceutically acceptable salt, solvate, stereoisomer, isotopic variant, or N-oxide thereof.

20. The compound according to claim 1, wherein
R^{5a} and R^{5b} are each H; or a pharmaceutically acceptable salt, solvate, stereoisomer, isotopic variant, or N-oxide thereof.

21. The compound according to claim 1, wherein R^{5a} and R^{5b} are each CH₃; or a pharmaceutically acceptable salt, solvate, stereoisomer, isotopic variant, or N-oxide thereof.

22. The compound according to claim 1, wherein R^{5a} is H, and R^{5b} is CH₃; or a pharmaceutically acceptable salt, solvate, stereoisomer, isotopic variant, or N-oxide thereof.

23. The compound according to claim 1, wherein R^{5a} and R^{5b} come together to form ═O; or a pharmaceutically acceptable salt, solvate, stereoisomer, isotopic variant, or N-oxide thereof.

24. The compound according to claim 1, having the structure of Formula (IA): 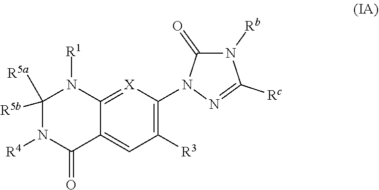
wherein
X is CH or N;
R¹ is selected from the group consisting of: C₁₋₆alkyl; C₁₋₆alkyl substituted with OH, OCH₃, C₃₋₆cycloalkyl or C₃₋₆heterocycloalkyl; C₂₋₆alkenyl; C₂₋₆alkenyl substituted with one, two or three F members; C₁₋₆haloalkyl; C₁₋₆haloalkyl substituted with OH, or OCH₃; C₃₋₆cycloalkyl; C₃₋₆heterocycloalkyl; and phenyl;
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, and C₃₋₆cycloalkyl;
R³ is H or F;
R⁴ is selected from the group consisting of: 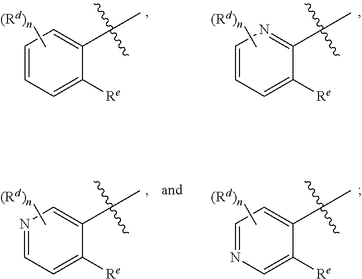
wherein
each R^{d} is independently a member selected from the group consisting of: H; halo; C₁₋₄alkyl; C₁₋₄alkyl substituted with a member selected from the group consisting of: OH, OCH₃, SCH₃, and OCF₃; C₁₋₄haloalkyl; C₁₋₄haloalkyl substituted with OH, or OCH₃; and OC₁₋₄alkyl;
R^{e} is a member selected from the group consisting of: H; halo; CN; C₁₋₄alkyl;
C₁₋₄alkyl substituted with OH, OCH₃, SCH₃, and OCF₃; C₁₋₄haloalkyl; and
C₁₋₄haloalkyl substituted with OH, or OCH₃;
n is 1 or 2; and
R^{5a} and R^{5b} are each independently H or CH₃; or R^{5a} and R^{5b} come together to form ═O;
or a pharmaceutically acceptable salt, solvate, stereoisomer, isotopic variant, or N-oxide thereof.

25. The compound according to claim 24, wherein
R¹ is 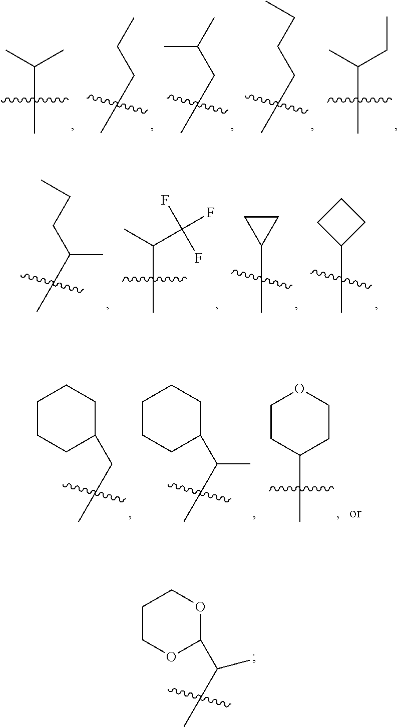
R^{b} is CH₂CH₃;
R^{c} is CH₂OH;
X is CH or N;
R³ is F;
R⁴ is selected from the group consisting of: 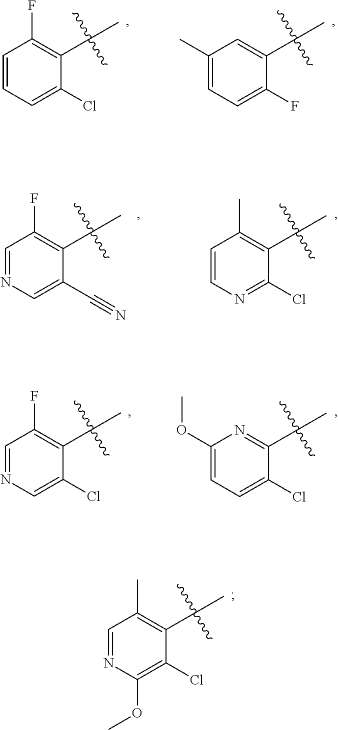
and
R^{5a} and R^{5b} are each independently H or CH₃; or R^{5a} and R^{5b} come together to form ═O; or a pharmaceutically acceptable salt, solvate, stereoisomer, isotopic variant, or N-oxide thereof.

26. The compound according to claim 1, having the structure of Formula (IB): 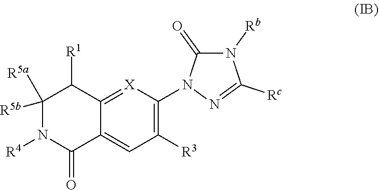
wherein
X is CH or N;
R¹ is selected from the group consisting of: C₁₋₄alkyl; C₁₋₄alkyl substituted with OH, OCH₃, C₃₋₆cycloalkyl or C₃₋₆heterocycloalkyl; C₂₋₆alkenyl; C₂₋₆alkenyl substituted with one, two or three F members; C₁₋₄haloalkyl; C₁₋₄haloalkyl substituted with OH, or OCH₃; C₃₋₆cycloalkyl; C₃₋₆heterocycloalkyl; and phenyl;
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, and C₃₋₆cycloalkyl;
R³ is H or F;
R⁴ is selected from the group consisting of: 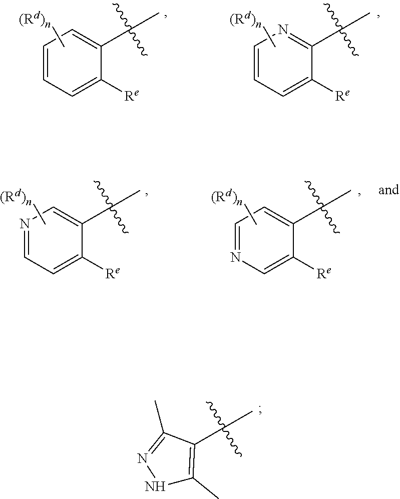
wherein
each R^{d} is independently selected from the group consisting of: H; halo; C₁₋₄alkyl; C₁₋₄alkyl substituted with a member selected from the group consisting of: OH, OCH₃, SCH₃, and OCF₃; C₁₋₄haloalkyl; C₁₋₄haloalkyl substituted with OH, or OCH₃; and OC₁₋₄alkyl; and
R^{e} is selected from the group consisting of: H; halo; CN; C₁₋₄alkyl; C₁₋₄alkyl substituted with OH, OCH₃, SCH₃, and OCF₃; C₁₋₄haloalkyl; and C₁₋₄haloalkyl substituted with OH, or OCH₃;
n is 1 or 2; and
R^{5a} and R^{5b} are each independently H or CH₃; or R^{5a} and R^{5b} come together to form ═O;
or a pharmaceutically acceptable salt, solvate, stereoisomer, isotopic variant, or N-oxide thereof.

27. The compound according to claim 26, wherein
R¹ is 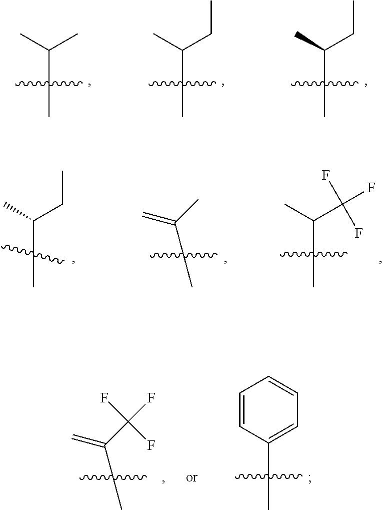
R^{b} is CH₂CH₃;
R^{c} is CH₂OH;
X is CH;
R³ is H or F;
R⁴ is selected from the group consisting of: 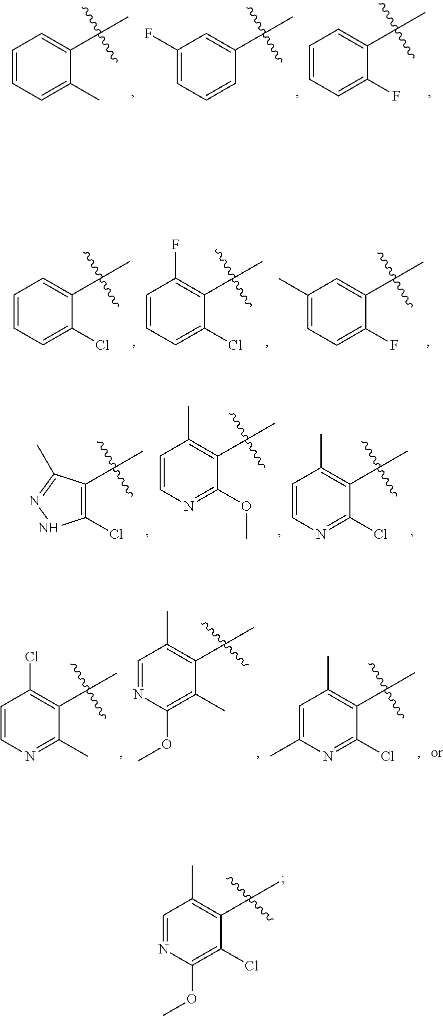
and
R^{5a} and R^{5b} are H;
or a pharmaceutically acceptable salt, solvate, stereoisomer, isotopic variant, or N-oxide thereof.

28. The compound according to claim 24, wherein X is N.

29. The compound according to claim 24, wherein X is CH.

30. The compound according to claim 26, wherein X is N.

31. The compound according to claim 26, wherein X is CH.

32. A compound selected from the group consisting of:
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2-methyl-2,3-dihydroquinazolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,2-dimethyl-2,3-dihydroquinazolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropylquinazoline-2,4(1H,3H)-dione.
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,3-dihydropyrido[2,3-d]pyrimidin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-(tetrahydro-2H-pyran-4-yl)-2,3-dihydropyrido[2,3-d]pyrimidin-4(1H)-one;
3-(2-chloro-6-fluorophenyl)-1-cyclobutyl-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one;
(S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(1,1,1-trifluoropropan-2-yl)-2,3-dihydropyrido[2,3-d]pyrimidin-4(1H)-one;
3 -(2-Chloro-6-fluorophenyl)-1-cyclopropyl-7-(4-ethyl-3-(hydroxymethyl)-5-oxo-4,5-dihydro-1H-1,2,4-triazol-1-yl)-6-fluoro-2,3-dihydropyri do [2,3-d] pyrimidin-4(1H)-one;
Racemic-6-(4-Ethyl-3-(hydroxymethyl)-5-oxo-4,5-dihydro-1H-1,2,4-triazol-1-yl)-2-(3-fluorophenyl)-4-isopropyl-3,4-dihydroisoquinolin-1(2H)-one;
Racemic-2-(2-Chloro-6-fluorophenyl)-6-(4-ethyl-3-(hydroxymethyl)-5-oxo-4,5-dihydro-1H-1,2,4-triazol-1-yl)-4-(prop-1-en-2-yl)-3,4-dihydroisoquinolin-1(2H)-one;
Racemic-6-(4-Ethyl-3-(hydroxymethyl)-5-oxo-4,5-dihydro-1H-1,2,4-triazol-1-yl)-2-(2-fluorophenyl)-4-isopropyl-3,4-dihydroisoquinolin-1(2H)-one;
Racemic-2-(2-Chloro-6-fluorophenyl)-6-(4-ethyl-3-(hydroxymethyl)-5-oxo-4,5-dihydro-1H-1,2,4-triazol-1-yl)-4-isopropyl-3,4-dihydroisoquinolin-1(2H)-one;
Racemic-6-(4-Ethyl-3-(hydroxymethyl)-5-oxo-4,5-dihydro-1H-1,2,4-triazol-1-yl)-2-(3-fluorophenyl)-4-phenyl-3,4-dihydroisoquinolin-1(2H)-one;
(S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(p entan-2-yl)-2,3-dihydro quinazolin-4(1H)-one;
(S)-1-(sec-Butyl)-3-(2-chloro-6-fluorophenyl)-7-(4-ethyl-3-(hydroxymethyl)-5-oxo04,5-dihydro-1H-1,2,4-triazol-1-yl)-6-fluoro-2,3-dihydro quinazolin-4(1H)-one;
(S)-3-(2-Chloro-6-fluorophenyl)-1-(1-cyclohexylethyl)-7-(4-ethyl-3-(hydroxymethyl)-5-oxo-4,5-dihydro-1H-1,2,4-triazol-1-yl)-6-fluoro-2,3-dihydroquinazolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isobutyl-2,3-dihydroquinazolin-4(1H)-one;
3-(2-Chloro-6-fluorophenyl)-1-cyclobutyl-7-(4-ethyl-3-(hydroxymethyl)-5-oxo-4,5-dihydro-1H-1,2,4-triazol-1-yl)-6-fluoro-2,3-dihydro quinazolin-4(1H)-one;
1-Butyl-3-(2-chloro-6-fluoropheny 0-7-(4-ethyl-3-(hydroxymethyl)-5-oxo-4,5-dihydro-1H-1,2,4-triazol-1-y 0-6-fluoro-2,3-dihydroquinazolin-4(1H)-one;
3-(2-Chloro-6-fluorophenyl)-1-(cyclohexy lmethyl)-7-(4-ethyl-3-(hydroxymethyl)-5-oxo-4,5-dihydro-1H-1,2,4-triazol-1-yl)-6-fluoro-2,3-dihydroquinazolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-propyl-2,3-dihydroquinazolin-4(1H)-one;
(S)-1-(1-(1,3-Dioxan-2-yl)ethyl)-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one;
(S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(pentan-2-yl)-2,3-dihydropyrido[2,3-d]pyrimidin-4(1H)-one;
2-(4-Chloro-2-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
7-(4-Ethyl-3-(hydroxymethyl)-5-oxo-4,5-dihydro-1H-1,2,4-triazol-1-yl)-6-fluoro-3-(2-fluoro-5-methylphenyl)-1-isopropyl-2,3-dihydroquinazolin-4(1H)-one;
4-(7-(4-Ethyl-3-(hydroxymethyl)-5-oxo-4,5-dihydro-1H-1,2,4-triazol-1-yl)-6-fluoro-1-isopropyl-4-oxo-1,4-dihydroquinazolin-3(2H)-yl)-5-fluoronicotinonitrile;
3-(2-Chloro-4-methylpyridin-3-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one;
3-(3-Chloro-5-fluoropyridin-4-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one;
3-(3-Chloro-6-methoxypyridin-2-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one;
(S)-1-(sec-Butyl)-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one;
3-(3-Chloro-2-methoxy-5-methylpyridin-4-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one;
(S*)-6-[4-Ethyl-3-(hydroxymethyl)-5-oxo-1,2,4-triazol-1-yl]-7-fluoro-4-isopropenyl-2-(o-tolyl)-3,4-dihydroisoquinolin-1-one;
(S*)-6-[4-Ethyl-3-(hydroxymethyl)-5-oxo-1,2,4-triazol-1-yl]-7-fluoro-4-isopropenyl-2-(o-tolyl)-3,4-dihydroisoquinolin-1-one;
(R*)-6-[4-Ethyl-3-(hydroxymethyl)-5-oxo-1,2,4-triazol-1-yl]-7-fluoro-4-isopropyl-2-(o-tolyl)-3,4-dihydroisoquinolin-1-one;
(S*)-2-(2-Chlorophenyl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one;
(R*)-2-(2-Chlorophenyl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one;
Racemic 6-(4-Ethyl-3-(hydroxymethyl)-5-oxo-4,5-dihydro-1H-1,2,4-triazol-1-yl)-7-fluoro-2-(2-fluoro-5-methylphenyl)-4-(prop-1-en-2-yl)-3,4-dihydroisoquinolin-1(2H)-one;
(S*)-6-(4-Ethyl-3-(hydroxymethyl)-5-oxo-4,5-dihydro-1H-1,2,4-triazol-1-yl)-7-fluoro-2-(2-fluoro-5-methy 1phenyl)-4-(prop-1-en-2-yl)-3,4-dihydroisoquinolin-1(2H)-one;
(R*)-6-(4-Ethyl-3-(hydroxymethyl)-5-oxo-4,5-dihydro-1H-1,2,4-triazol-1-yl)-7-fluoro-2-(2-fluoro-5-methy 1phenyl)-4-(prop-1-en-2-yl)-3,4-dihydroisoquinolin-1(2H)-one;
(S*)-6-(4-Ethyl-3-(hydroxymethyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one;
(R*)-6-(4-Ethyl-3-(hydroxy methyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one;
(S*)-6-(4-Ethyl-3-(hydroxymethyl)-5-oxo-4,5-dihydro-1H-1,2,4-triazol-1-yl)-7-fluoro-4-isopropyl-2-(o-tolyl)-3,4-dihydroisoquinolin-1(2H)-one;
(R*)-6-(4-Ethyl-3-(hydroxymethyl)-5-oxo-4,5-dihydro-1H-1,2,4-triazol-1-yl)-7-fluoro-4-isopropyl-2-(o-tolyl)-3,4-dihydroisoquinolin-1(2H)-one;
Racemic 2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(S*)-2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(R*)-2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(S*)-2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(R*)-2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 1, (S*)-6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one;
Atropisomer 2, (S*)-6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one;
Atropisomer 1, (R*)-6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one;
Atropisomer 2, (R*)-6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one;
Atropisomer 1, (S*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one;
Atropisomer 1, (R*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one;
Atropisomer 2, (S*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one;
Atropisomer 2, (R*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one;
Atropisomer 1, (S*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 1, (R*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 1, (S*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 2, (R*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 1, (S*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 1, (R*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 2, (S*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 2, (R*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(S*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one;
(R*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one;
(R*)-2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(S*)-2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(S*)-4-((RS)-sec-Butyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one;
(R*)-4-((S*)-sec-Butyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one;
(R*)-4-((R*)-sec-Butyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one;
Racemic 2-(2-Chloro-6-fluorophenyl)-6-(4-ethyl-3-(hydroxymethyl)-5-oxo-4,5-dihydro-1H-1,2,4-triazol-1-yl)-4-(3,3,3-trifluoroprop-1-en-2-yl)-3,4-dihydroisoquinolin-1(2H)-one;
6-(4-Ethyl-3-(hydroxymethyl)-5-oxo-4,5-dihydro-1H-1,2,4-triazol-1-yl)-2-(2-fluorophenyl)-4-(1,1,1-trifluoropropan-2-yl)-3,4-dihydroisoquinolin-1(2H)-one; and
2-(2-Chloro-6-fluorophenyl)-6-(4-ethyl-3-(hydroxymethyl)-5-oxo-4,5-dihydro-1H-1,2,4-triazol-1-yl)-4-(1,1,1-trifluoroprop an-2-y 0-3,4-dihydroisoquinolin-1(2H)-one;
and pharmaceutically acceptable salts, solvates, stereoisomers, isotopic variants, and N-oxides thereof.

33. A pharmaceutical composition comprising: (A) an effective amount of a compound according to claim 1, or a pharmaceutically acceptable salt, solvate, stereoisomer, isotopic variant, or N-oxide thereof; and (B) at least one pharmaceutically acceptable excipient.

34. A pharmaceutical composition comprising an effective amount of a compound of claim 32; and at least one pharmaceutically acceptable excipient.

35. A method of treating a subject suffering from or diagnosed with a disease, disorder, or medical condition comprising inhibiting or altering dihydroorotate oxygenase enzyme activity in the subject by administering to the subject an effective amount of at least one compound according to claim 1.

36. The method according to claim 35, wherein the disorder, disease or medical condition is selected from the group consisting of: inflammatory disorders and autoimmune disorders.

37. The method according to claim 35, wherein the disorder, disease or medical condition is cancer.

38. The method according to claim 35, wherein the disorder, disease or medical condition is selected from the group consisting of: lymphomas, leukemias, carcinomas, and sarcomas.

39. The method according to claim 35, wherein the disorder, disease or medical condition is selected from the group consisting of: acute lymphoblastic leukemia, acute myeloid leukemia, (acute) T-cell leukemia, acute lymphoblastic leukemia, acute lymphocytic leukemia, acute monocytic leukemia, acute promyelocytic leukemia, bisphenotypic B myelomonocytic leukemia, chronic lymphocytic leukemia, chronic myelogenous leukemia, chronic myeloid leukemia, chronic myelomonocytic leukemia, large granular lymphocytic leukemia, plasma cell leukemia, and also myelodysplastic syndrome, which can develop into an acute myeloid leukemia.

40. The method according to claim 35, wherein the disorder, disease or medical condition is acute myeloid leukemia.

41. The method according to claim 35, wherein the at least one compound is selected from the group consisting of:
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2-methyl-2,3-dihydroquinazolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,2-dimethyl-2,3-dihydroquinazolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropylquinazoline-2,4(1H,3H)-dione. 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropyl-2,3-dihydropyrido[2,3-d]pyrimidin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-(tetrahydro-2H-pyran-4-yl)-2,3-dihydropyrido[2,3-d]pyrimidin-4(1H)-one;
3-(2-chloro-6-fluorophenyl)-1-cyclobutyl-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one;
(S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(1,1,1-trifluoropropan-2-yl)-2,3-dihydropyrido[2,3-d]pyrimidin-4(1H)-one;
3-(2-Chloro-6-fluorophenyl)-1-cyclopropyl-7-(4-ethyl-3-(hydroxymethyl)-5-oxo-4,5-dihydro-1H-1,2,4-triazol-1-y 0-6-fluoro-2,3-dihydropyri do [2,3-d] pyrimidin-4(1H)-one;
Racemic-6-(4-Ethyl-3-(hydroxymethyl)-5-oxo-4,5-dihydro-1H-1,2,4-triazol-1-yl)-2-(3-fluorophenyl)-4-isopropyl-3,4-dihydroisoquinolin-1(2H)-one;
Racemic-2-(2-Chloro-6-fluorophenyl)-6-(4-ethyl-3-(hydroxymethyl)-5-oxo-4,5-dihydro-1H-1,2,4-triazol-1-yl)-4-(prop-1-en-2-yl)-3,4-dihydroisoquinolin-1(2H)-one;
Racemic-6-(4-Ethyl-3-(hydroxymethyl)-5-oxo-4,5-dihydro-1H-1,2,4-triazol-1-yl)-2-(2-fluorophenyl)-4-isopropyl-3,4-dihydroisoquinolin-1(2H)-one;
Racemic-2-(2-Chloro-6-fluorophenyl)-6-(4-ethyl-3-(hydroxymethyl)-5-oxo-4,5-dihydro-1H-1,2,4-triazol-1-yl)-4-isopropyl-3,4-dihydroisoquinolin-1(2H)-one;
Racemic-6-(4-Ethyl-3-(hydroxymethyl)-5-oxo-4,5-dihydro-1H-1,2,4-triazol-1-yl)-2-(3-fluorophenyl)-4-phenyl-3,4-dihydroisoquinolin-1(2H)-one;
(S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(p entan-2-yl)-2,3-dihydro quinazolin-4(1H)-one;
(S)-1-(s ec-Butyl)-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydro quinazolin-4(1H)-one;
(S)-3-(2-Chloro-6-fluorophenyl)-1-(1-cyclohexylethyl)-7-(4-ethyl-3-(hydroxymethyl)-5-oxo-4,5-dihydro-1H-1,2,4-triazol-1-yl)-6-fluoro-2,3-dihydroquinazolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isobutyl-2,3-dihydroquinazolin-4(1H)-one;
3-(2-Chloro-6-fluorophenyl)-1-cyclobutyl-7-(4-ethyl-3-(hydroxymethyl)-5-oxo-4,5-dihydro-1H-1,2,4-triazol-1-yl)-6-fluoro-2,3-dihydro quinazolin-4(1H)-one;
1-Butyl-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydroquinazolin-4(1H)-one;
3-(2-Chloro-6-fluorophenyl)-1-(cyclohexy lmethyl)-7-(4-ethyl-3-(hydroxymethyl)-5-oxo-4,5-dihydro-1H-1,2,4-triazol-1-yl)-6-fluoro-2,3-dihydroquinazolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-propyl-2,3-dihydroquinazolin-4(1H)-one;
(S)-1-(1-(1,3-Dioxan-2-yl)ethyl)-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one;
(S)-3-(2-Chloro-6-fluorophenyl)-7-(4-ethyl-3-(hydroxymethyl)-5-oxo-4,5-dihydro-1H-1,2,4-triazol-1-yl)-6-fluoro-1-(pentan-2-yl)-2,3-dihydropyrido[2,3-d]pyrimidin-4(1H)-one;
2-(4-Chloro-2-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
7-(4-Ethyl-3-(hydroxymethyl)-5-oxo-4,5-dihydro-1H-1,2,4-triazol-1-yl)-6-fluoro-3-(2-fluoro-5-methylphenyl)-1-isopropyl-2,3-dihydroquinazolin-4(1H)-one;
4-(7-(4-Ethyl-3-(hydroxymethyl)-5-oxo-4,5-dihydro-1H-1,2,4-triazol-1-yl)-6-fluoro-1-isopropyl-4-oxo-1,4-dihydroquinazolin-3(2H)-yl)-5-fluoronicotinonitrile;
3-(2-Chloro-4-methylpyridin-3-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one;
3-(3-Chloro-5-fluoropyridin-4-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one;
3-(3-Chloro-6-methoxypyridin-2-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one;
(S)-1-(sec-Butyl)-3-(2-chloro-6-fluorophenyl)-7-(4-ethyl-3-(hydroxymethyl)-5-oxo-4,5-dihydro-1H-1,2,4-triazol-1-yl)-6-fluoro-2,3-dihydropyrido[2,3-d]pyrimidin-4(1H)-one;
3-(3-Chloro-2-methoxy-5-methylpyridin-4-yl)-7-(4-ethyl-3-(hydroxymethyl)-5-oxo-4,5-dihydro-1H-1,2,4-triazol-1-yl)-6-fluoro-1-isopropyl-2,3-dihydroquinazolin-4(1H)-one;
(S*)-6-[4-Ethyl-3-(hydroxymethyl)-5-oxo-1,2,4-triazol-1-yl]-7-fluoro-4-isopropenyl-2-(o-tolyl)-3,4-dihydroisoquinolin-1-one;
(S*)-6-[4-Ethyl-3-(hydroxymethyl)-5-oxo-1,2,4-triazol-1-yl]-7-fluoro-4-isopropenyl-2-(o-tolyl)-3,4-dihydroisoquinolin-1-one;
(R*)-6-[4-Ethyl-3-(hydroxymethyl)-5-oxo-1,2,4-triazol-1-yl]-7-fluoro-4-isopropyl-2-(o-tolyl)-3,4-dihydroisoquinol in-1-one;
(S*)-2-(2-Chlorophenyl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one;
(R*)-2-(2-Chlorophenyl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one;
Racemic 6-(4-Ethyl-3-(hydroxymethyl)-5-oxo-4,5-dihydro-1H-1,2,4-triazol-1-yl)-7-fluoro-2-(2-fluoro-5-methy 1pheny 0-4-(prop-1-en-2-yl)-3,4-dihydroisoquinolin-1(2H)-one;
(S*)-6-(4-Ethyl-3-(hydroxymethyl)-5-oxo-4,5-dihydro-1H-1,2,4-triazol-1-yl)-7-fluoro-2-(2-fluoro-5-methy 1phenyl)-4-(prop-1-en-2-yl)-3,4-dihydroisoquinolin-1(2H)-one;
(R*)-6-(4-Ethyl-3-(hydroxymethyl)-5-oxo-4,5-dihydro-1H-1,2,4-triazol-1-yl)-7-fluoro-2-(2-fluoro-5-methy 1phenyl)-4-(prop-1-en-2-yl)-3,4-dihydroisoquinolin-1(2H)-one;
(S*)-6-(4-Ethyl-3-(hydroxymethyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one;
(R*)-6-(4-Ethyl-3-(hydroxymethyl)-5-oxo-4,5-dihydro-1H-1,2,4-triazol-1-yl)-7-fluoro-4-(prop-1-en-2-yl)-2-(o-tolyl)-3,4-dihydroisoquinolin-1(2H)-one;
(S*)-6-(4-Ethyl-3-(hydroxymethyl)-5-oxo-4,5-dihydro-1H-1,2,4-triazol-1-yl)-7-fluoro-4-isopropyl-2-(o-tolyl)-3,4-dihydroi s o quinolin-1(2H)-one;
(R*)-6-(4-Ethyl-3-(hydroxymethyl)-5-oxo-4,5-dihydro-1H-1,2,4-triazol-1-yl)-7-fluoro-4-isopropyl-2-(o-tolyl)-3,4-dihydroisoquinolin-1(2H)-one;
Racemic 2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(S*)-2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(R*)-2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(S*)-2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(R*)-2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 1, (S*)-6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one;
Atropisomer 2, (S*)-6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one;
Atropisomer 1, (R*)-6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one;
Atropisomer 2, (R*)-6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)-3,4-dihydroisoquinolin-1(2H)-one;
Atropisomer 1, (S*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one;
Atropisomer 1, (R*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one;
Atropisomer 2, (S*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one;
Atropisomer 2, (R*)-6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)-3,4-dihydroisoquinolin-1(2H)-one;
Atropisomer 1, (S*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 1, (R*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 1, (S*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 2, (R*)-2-(2-Chloro-4,6-dimethylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 1, (S*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 1, (R*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 2, (S*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
Atropisomer 2, (R*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(S*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one;
(R*)-2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-isopropyl-3,4-dihydroisoquinolin-1(2H)-one;
(R*)-2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(S*)-2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)-3,4-dihydroisoquinolin-1(2H)-one;
(S*)-4-((RS)-sec-Butyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one;
(R*)-4-((S*)-sec-Butyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one;
(R*)-4-((R*)-sec-Butyl)-2-(2-chloro-6-fluorophenyl)-6-(4-ethyl-3-(hydroxymethyl)-5-oxo-4,5-dihydro-1H-1,2,4-triazol-1-yl)-7-fluoro-3,4-dihydroisoquinolin-1(2H)-one;
Racemic 2-(2-Chloro-6-fluorophenyl)-6-(4-ethyl-3-(hydroxymethyl)-5-oxo-4,5-dihydro-1H-1,2,4-triazol-1-yl)-4-(3,3,3-trifluoroprop-1-en-2-yl)-3,4-dihydroisoquinolin-1(2H)-one;
6-(4-Ethyl-3-(hydroxymethyl)-5-oxo-4,5-dihydro-1H-1,2,4-triazol-1-yl)-2-(2-fluorophenyl)-4-(1,1,1-trifluoropropan-2-yl)-3,4-dihydroisoquinolin-1(2H)-one; and
2-(2-Chloro-6-fluorophenyl)-6-(4-ethyl-3-(hydroxymethyl)-5-oxo-4,5-dihydro-1H-1,2,4-triazol-1-yl)-4-(1,1,1-trifluoropropan-2-yl)-3,4-dihydroisoquinolin-1(2H)-one;
and pharmaceutically acceptable salts, solvates, stereoisomers, isotopic variants, and N-oxides thereof.
